(19) United States
(12) Patent Application Publication
(10) Pub. No.: US 2026/0234145 A1
(43) Pub. Date: Aug. 13, 2026

(54) COMPOUNDS AS PRMT5 DEGRADERS AND USES THEREOF

(71) Applicant: Oncopia Therapeutics, Inc. d/b/a/ SK Life Science Labs, King of Prussia, PA (US)

(72) Inventors: Guozhang XU, King of Prussia, PA (US); Qiaolin DENG, King of Prussia, PA (US); Xuqing ZHANG, King of Prussia, PA (US)

(21) Appl. No.: 19/537,015

(22) Filed: Feb. 11, 2026

Related U.S. Application Data
(60) Provisional application No. 63/757,030, filed on Feb. 11, 2025.

(51) Int. Cl. C07D 471/04

(52) U.S. Cl. CPC C07D 471/04

(57) ABSTRACT

Described herein are compounds of Formula I and their pharmaceutically acceptable salts, solvates, and stereoisomers, as well as their uses (e.g., as PRMT5 degraders).

### RELATED APPLICATION

This application claims the benefit of and priority to U.S. Provisional Application No. 63/757,030, filed Feb. 11, 2025, the contents of which are incorporated herein by reference in their entireties.

### BACKGROUND

As the predominant member of type II protein arginine methyltransferases (PRMTs), protein arginine methyltransferase 5 (PRMT5) catalyzes monomethylation and symmetric dimethylation of arginine residues of its histone substrates, including H3R2, H3R8, and H4R3, and its nonhistone substrates, including p53, EGFR, N-MYC, SmD3, and RNA polymerase II, when interacting with its binding partner MEP50. Through methylation of these substrates, PRMT5 regulates multiple biological processes, such as chromatin remodeling, gene expression, mRNA splicing, DNA replication and repair, and cell cycle regulation.

The aberrant expression of PRMT5 has been associated with infectious disease, heart disease, and cancers, including breast cancer, lung cancer, and hepatocellular cancer (HCC). Genetic knockdown of PRMT5 was effective to suppress tumor growth in vitro and in vivo. For example, PRMT5 knockdown decreased the proliferation, invasion, and migration of HCC cell lines. The knockdown of PRMT5 in AR-positive LNCaP prostate cancer cells completely suppressed tumor growth in xenograft mouse models. In addition, the knockdown of PRMT5 drastically prolonged the survival in a murine model of BCR-ABL-driven chronic myelogenous leukemia and of MYC-driven B cell lymphoma. Moreover, the PRMT5 knockdown effectively suppressed the growth of patient-derived xenograft glioblastoma (GBM) tumors in vivo. Collectively, these findings suggest that PRMT5 down-regulation may provide a potential therapeutic strategy for treating cancer.

Recently, proteolysis targeting chimera (PROTAC) has emerged as a powerful technology to reduce the protein levels of the target of interest by hijacking the cellular ubiquitin-proteasome system. PROTACs can phenocopy the effects of genetic knockdown or knockout as they not only inhibit the target proteins as traditional inhibitors do but also more importantly eliminate the other functions (e.g., scaffolding function) of the protein targets. Another potential advantage of PROTACs over occupancy-driven small-molecule inhibitors is that PROTACs may potentially overcome the resistance to small-molecule inhibitor treatments.

### SUMMARY

In some aspects, the present disclosure provides compounds of Formula I

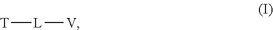

or a pharmaceutically acceptable salt, solvate, or stereoisomer thereof, wherein:
T is of Formula I-1

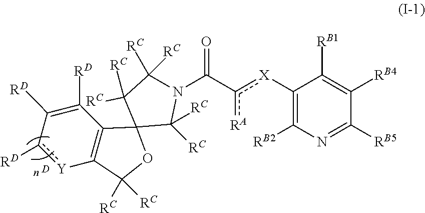

wherein:
each [symbol] is independently a single bond or a double bond, as valency permits;
X is N(R^{X1}), and R^{A} is oxo; or
X is C(R^{X2}), and R^{A} and R^{B2}, together with the intervening atoms to which they are attached, form C₆ aryl or 6-membered heteroaryl, wherein the C₆ aryl or 6-membered heteroaryl is optionally substituted with one or more halogen, —CN, —NO₂, —OH, or —NH₂;
R^{X1} is hydrogen or C₁₋₆ alkyl, wherein the C₁₋₆ alkyl is optionally substituted with one or more halogen, —CN, —NO₂, —OH, or —NH₂;
R^{X2} is hydrogen, halogen, —CN, —NO₂, —OH, —NH₂, or C₁₋₆ alkyl, wherein the C₁₋₆ alkyl is optionally substituted with one or more halogen, —CN, —NO₂, —OH, or —NH₂;
R^{B1}, R^{B2}, R^{B4}, and R^{B5} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, or —NH₂; or
R^{B1} and R^{B4}, together with the intervening atoms to which they are attached, form 3- to 8-membered heterocyclyl or 5- to 6-membered heteroaryl, wherein the 3- to 8-membered heterocyclyl or 5- to 6-membered heteroaryl is optionally substituted with one or more halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl;
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
n^{D} is 0 or 1;
Y is O, S, or C(R^{D}), as valency permits; and
each R^{D} is independently

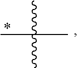

hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂, wherein * denotes attachment to L, and one R^{D} is

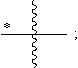

L is of Formula I-2

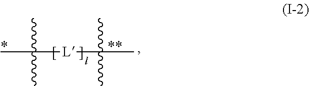

wherein:
* denotes attachment to T, and ** denotes attachment to V;
each L′ is independently —C(═O)—, —C(═O)N(R^{L′})—, —N(R^{L′})C(═O)—, —C(═O)O—, —OC(═O)—, —N(R^{L′})—, —O—, —O—(C₁₋₆ alkylene)-, —(C₁₋₆ alkylene)-O—, —S—, —S(═O)₂—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene, wherein the —O—(C₁₋₆ alkylene)-, —(C₁₋₆ alkylene)-O—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene is optionally substituted with one or more halogen, —CN, —NO₂, —OH, or —NH₂;
each occurrence of R^{L′} is independently hydrogen, C₁₋₆ alkyl, C₂₋₆ alkenyl, C₂₋₆ alkynyl, C₃₋₈ carbocyclyl, 3- to 8-membered heterocyclyl, —S(═O)₂R^{a}, —S(═O)₂OR^{a}, —S(═O)₂N(R^{a})₂, —C(═O)R^{a}, —C(═O)OR^{a}, or —C(═O)N(R^{a})₂, wherein the C₁₋₆ alkyl, C₂₋₆ alkenyl, C₂₋₆ alkynyl, C₃₋₈ carbocyclyl, or 3- to 8-membered heterocyclyl is optionally substituted with one or more halogen, —CN, —NO₂, —OH, or —NH₂; and
each R^{a} independently is hydrogen, C₁₋₆ alkyl, C₂₋₆ alkenyl, or C₂₋₆ alkynyl, wherein the C₁₋₆ alkyl, C₂₋₆ alkenyl, or C₂₋₆ alkynyl is optionally substituted with one or more halogen, —CN, —NO₂, —OH, or —NH₂;
1 is an integer selected from 0 to 10;

V is of Formula 1-3

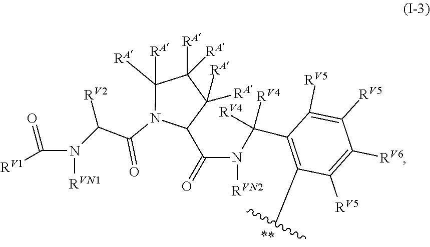

wherein:
** denotes attachment to L;
R^{V1} is hydrogen, C₁₋₆ alkyl, C₃₋₆ carbocyclyl, or 3- to 6-membered heterocyclyl, wherein the C₁₋₆ alkyl, C₃₋₆ carbocyclyl, or 3- to 6-membered heterocyclyl is optionally substituted with one or more halogen, —CN, —NO₂, —OH, or —NH₂;
R^{VN1} is hydrogen or C₁₋₆ alkyl, wherein the C₁₋₆ alkyl is optionally substituted with one or more halogen, —CN, —NO₂, —OH, or —NH₂;
R^{V2} is hydrogen or C₁₋₆ alkyl, wherein the C₁₋₆ alkyl is optionally substituted with one or more halogen, —CN, —NO₂, —OH, or —NH₂;
each R^{A′} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
R^{VN2} is hydrogen or C₁₋₆ alkyl, wherein the C₁₋₆ alkyl is optionally substituted with one or more halogen, —CN, —NO₂, —OH, or —NH₂;
each R^{V4} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
each R^{V5} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂; and
R^{V6} is C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, —NH₂, C₁₋₆ alkyl, C₂₋₆ alkenyl, or C₂₋₆ alkynyl.

In some aspects, the present disclosure provides conjugates comprising a compound disclosed herein, wherein the compound is attached to a conjugate partner.

In some aspects, the present disclosure provides pharmaceutical compositions comprising a compound disclosed herein, and one or more pharmaceutically acceptable excipient.

In some aspects, the present disclosure provides methods of degrading a PRMT5 protein in a subject, comprising administering to the subject a compound disclosed herein.

In some aspects, the present disclosure provides uses of a compound disclosed herein in the manufacture of a medicament for degrading a PRMT5 protein in a subject.

In some aspects, the present disclosure provides compounds disclosed herein for use in degrading a PRMT5 protein in a subject.

In some aspects, the present disclosure provides methods of reducing the amount of a PRMT5 protein in a subject (e.g., in a biological sample (e.g., a cell or a tissue) obtained from the subject), comprising administering to the subject a compound disclosed herein.

In some aspects, the present disclosure provides uses of a compound disclosed herein in the manufacture of a medicament for reducing the amount of a PRMT5 protein in a subject (e.g., in a biological sample (e.g., a cell or a tissue) obtained from the subject).

In some aspects, the present disclosure provides compounds disclosed herein for use in reducing the amount of a PRMT5 protein in a subject (e.g., in a biological sample (e.g., a cell or a tissue) obtained from the subject).

In some aspects, the present disclosure provides methods of treating or preventing a disease or disorder in a subject in need thereof, comprising administering to the subject a compound disclosed herein (e.g., in a therapeutically effective amount).

In some aspects, the present disclosure provides methods of treating a disease or disorder in a subject in need thereof, comprising administering to the subject a compound disclosed herein (e.g., in a therapeutically effective amount).

In some aspects, the present disclosure provides uses of a compound disclosed herein in the manufacture of a medicament for treating or preventing a disease or disorder in a subject in need thereof.

In some aspects, the present disclosure provides uses of a compound disclosed herein in the manufacture of a medicament for treating a disease or disorder in a subject in need thereof.

In some aspects, the present disclosure provides compounds disclosed herein for use in treating or preventing a disease or disorder in a subject in need thereof.

In some aspects, the present disclosure provides compounds disclosed herein for use in treating a disease or disorder in a subject in need thereof.

### DETAILED DESCRIPTION

The present disclosure relates to compounds and compositions that may be useful as PRMT5 protein degraders. The present disclosure also relates to methods of degrading a PRMT5 protein comprising contacting the PRMT5 protein with a compound or composition disclosed herein. The present disclosure also relates to methods of treating or preventing a PRMT5 protein-mediated disease or condition in a subject in need thereof by administering (e.g., in a therapeutically effective amount) a compound or composition disclosed herein.

### Compounds of the Application

In some aspects, the present disclosure provides compounds of Formula I

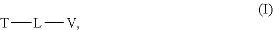

and pharmaceutically acceptable salts, solvates, and stereoisomers thereof, wherein:
T is of Formula I-1

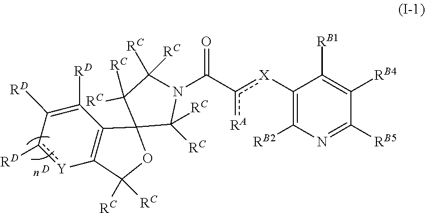

wherein:
each [symbol] is independently a single bond or a double bond, as valency permits;
X is N(R^{X1}), and R^{A} is oxo; or
X is C(R^{X2}), and R^{A} and R^{B2}, together with the intervening atoms to which they are attached, form C₆ aryl or 6-membered heteroaryl, wherein the C₆ aryl or 6-membered heteroaryl is optionally substituted with one or more halogen, —CN, —NO₂, —OH, or —NH₂;
R^{X1} is hydrogen or C₁₋₆ alkyl, wherein the C₁₋₆ alkyl is optionally substituted with one or more halogen, —CN, —NO₂, —OH, or —NH₂;
R^{X2} is hydrogen, halogen, —CN, —NO₂, —OH, —NH₂, or C₁₋₆ alkyl, wherein the C₁₋₆ alkyl is optionally substituted with one or more halogen, —CN, —NO₂, —OH, or —NH₂;
R^{B1}, R^{B2}, R^{B4}, and R^{B5} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, or —NH₂; or
R^{B1} and R^{B4}, together with the intervening atoms to which they are attached, form 3- to 8-membered heterocyclyl or 5- to 6-membered heteroaryl, wherein the 3- to 8-membered heterocyclyl or 5- to 6-membered heteroaryl is optionally substituted with one or more halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl;
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
n^{D} is 0 or 1;
Y is O, S, or C(R^{D}), as valency permits; and
each R^{D} is independently

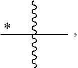

hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂, wherein * denotes attachment to L, and one R^{D} is

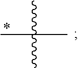

L is of Formula I-2

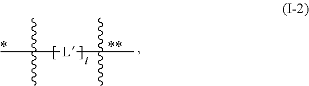

wherein:
* denotes attachment to T, and ** denotes attachment to V;
each L′ is independently —C(═O)—, —C(═O)N(R^{L′})—, —N(R^{L′})C(═O)—, —C(═O)O—, —OC(═O)—, —N(R^{L′})—, —O—, —O—(C₁₋₆ alkylene)-, —(C₁₋₆ alkylene)-O—, —S—, —S(═O)₂—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene, wherein the —O—(C₁₋₆ alkylene)-, —(C₁₋₆ alkylene)-O)—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene is optionally substituted with one or more halogen, —CN, —NO₂, —OH, or —NH₂;
each occurrence of R^{L′} is independently hydrogen, C₁₋₆ alkyl, C₂₋₆ alkenyl, C₂₋₆ alkynyl, C₃₋₈ carbocyclyl, 3- to 8-membered heterocyclyl, —S(═O)₂R^{a}, —S(═O)₂OR^{a}, —S(═O)₂N(R^{a})₂, —C(═O)R^{a}, —C(═O)OR^{a}, or —C(═O)N(R^{a})₂, wherein the C₁₋₆ alkyl, C₂₋₆ alkenyl, C₂₋₆ alkynyl, C₃₋₈ carbocyclyl, or 3- to 8-membered heterocyclyl is optionally substituted with one or more halogen, —CN, —NO₂, —OH, or —NH₂; and
each R^{a} independently is hydrogen, C₁₋₆ alkyl, C₂₋₆ alkenyl, or C₂₋₆ alkynyl, wherein the C₁₋₆ alkyl, C₂₋₆ alkenyl, or C₂₋₆ alkynyl is optionally substituted with one or more halogen, —CN, —NO₂, —OH, or —NH₂;
1 is an integer selected from 0 to 10;
V is of Formula 1-3

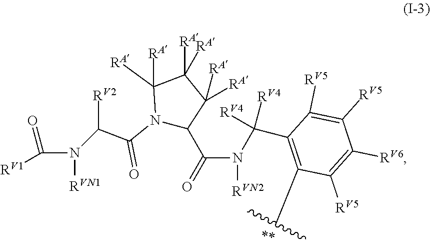

wherein:
** denotes attachment to L;
R^{V1} is hydrogen, C₁₋₆ alkyl, C₃₋₆ carbocyclyl, or 3- to 6-membered heterocyclyl, wherein the C₁₋₆ alkyl, C₃₋₆ carbocyclyl, or 3- to 6-membered heterocyclyl is optionally substituted with one or more halogen, —CN, —NO₂, —OH, or —NH₂;
R^{VN1} is hydrogen or C₁₋₆ alkyl, wherein the C₁₋₆ alkyl is optionally substituted with one or more halogen, —CN, —NO₂, —OH, or —NH₂;
R^{V2} is hydrogen or C₁₋₆ alkyl, wherein the C₁₋₆ alkyl is optionally substituted with one or more halogen, —CN, —NO₂, —OH, or —NH₂;
each R^{A′} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
R^{VN2} is hydrogen or C₁₋₆ alkyl, wherein the C₁₋₆ alkyl is optionally substituted with one or more halogen, —CN, —NO₂, —OH, or —NH₂;
each R^{V4} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
each R^{V5} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂; and
R^{V6} is C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, —NH₂, C₁₋₆ alkyl, C₂₋₆ alkenyl, or C₂₋₆ alkynyl.

In some embodiments, the compound is of Formula (I-a)

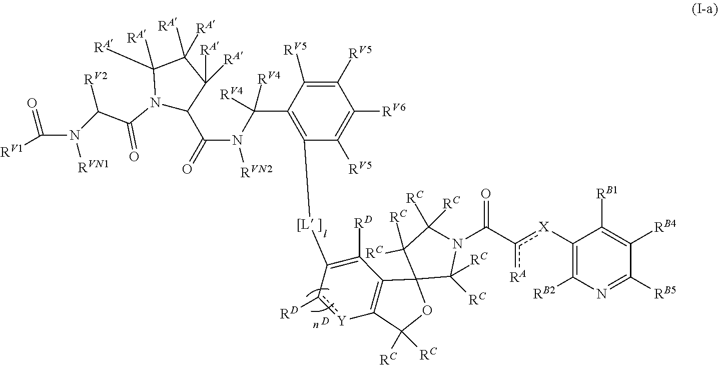

or a pharmaceutically acceptable salt, solvate, or stereoisomer thereof.

In some embodiments, the compound is of Formula (I-b)

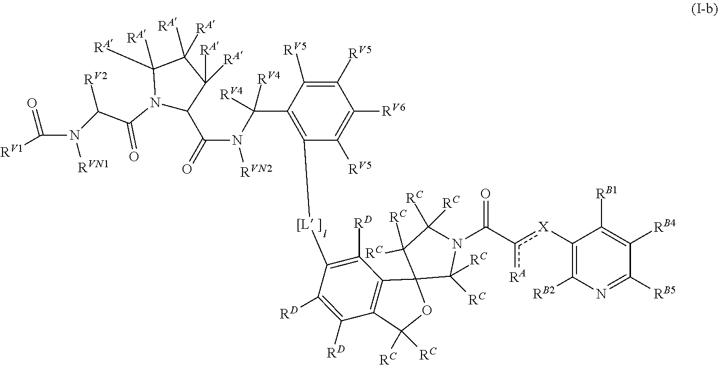

or a pharmaceutically acceptable salt, solvate, or stereoisomer thereof.

In some embodiments, the compound is of Formula (I-b-i), (I-b-ii), or (I-b-iii)

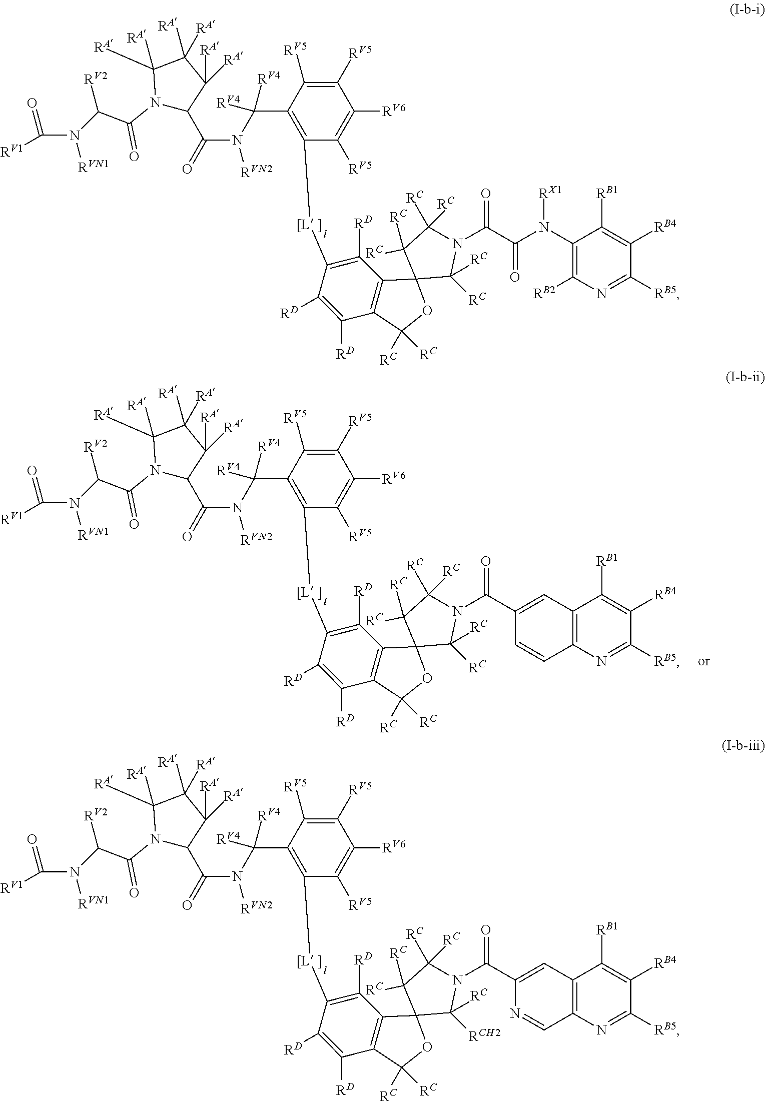

or a pharmaceutically acceptable salt, solvate, or stereoisomer thereof.

In some embodiments, the compound is of Formula (I-c)

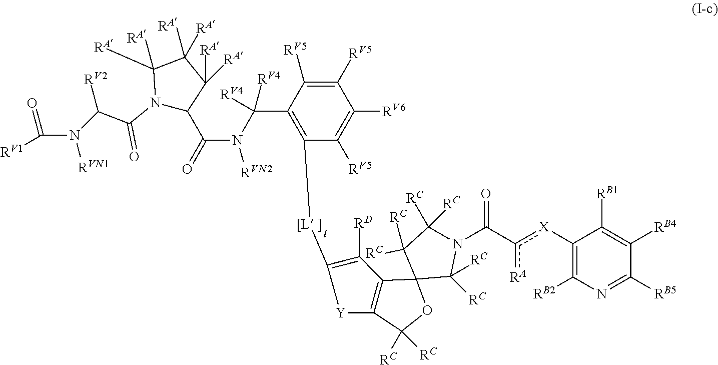

or a pharmaceutically acceptable salt, solvate, or stereoisomer thereof.

In some embodiments, the compound is of Formula (I-c-i), (I-c-ii), or (I-c-iii)

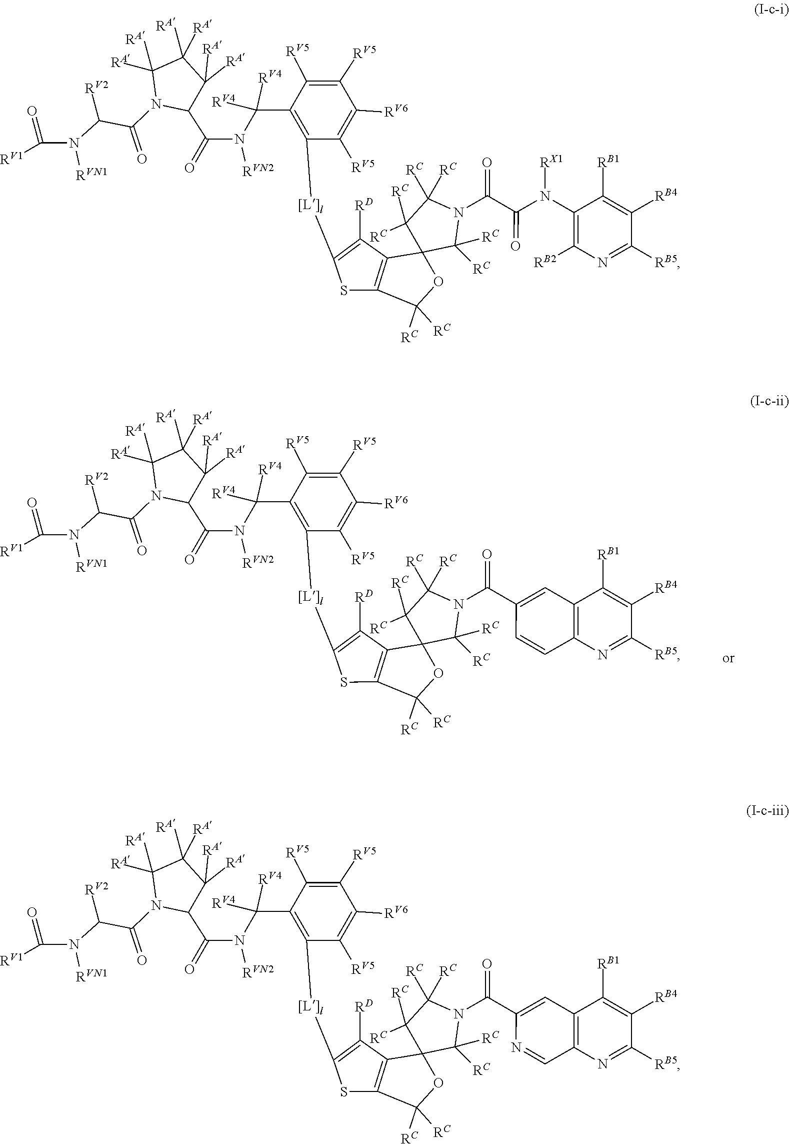

or a pharmaceutically acceptable salt, solvate, or stereoisomer thereof.

### Variable T

In some embodiments, at least one [symbol] is a single bond, as valency permits.

In some embodiments, at least one [symbol] is a double bond, as valency permits.

In some embodiments, each [symbol] is a single bond, as valency permits.

In some embodiments, each [symbol] is a double bond, as valency permits.

In some embodiments, X is N(R^{X1}), and R^{A} is oxo.

In some embodiments, X is NH, and R^{A} is oxo.

In some embodiments, X is C(R^{X2}), and R^{A} and R^{B2}, together with the intervening atoms to which they are attached, form C₆ aryl optionally substituted with one or more halogen (e.g., F, Cl, Br, or I), —CN, —NO₂, —OH, or —NH₂.

In some embodiments, X is CH, and R^{A} and R^{B2}, together with the intervening atoms to which they are attached, form Ce aryl.

In some embodiments, X is C(R^{X2}), and R^{A} and R^{B2}, together with the intervening atoms to which they are attached, form 6-membered heteroaryl (e.g., heteroaryl comprising one 6-membered ring and 1-3 heteroatoms selected from N, O, and S) optionally substituted with one or more halogen (e.g., F, Cl, Br, or I), —CN, —NO₂, —OH, or —NH₂.

In some embodiments, X is C(R^{X2}), and R^{A} and R^{B2}, together with the intervening atoms to which they are attached, form pyridinyl.

In some embodiments, X is CH, and R^{A} and R^{B2}, together with the intervening atoms to which they are attached, form pyridinyl.

In some embodiments, R^{X1} is hydrogen.

In some embodiments, R^{X1} is C₁₋₆ alkyl (e.g., methyl (C₁), ethyl (C₂), n-propyl (C₃), i-propyl (C₃), n-butyl (C₄), i-butyl (C₄), s-butyl (C₄), f-butyl (C₄), pentyl (C₅), or hexyl (C₆)) optionally substituted with one or more halogen (e.g., F, Cl, Br, or I), —CN, —NO₂, —OH, or —NH₂.

In some embodiments, R^{X2} is hydrogen.

In some embodiments, R^{X2} is halogen (e.g., F, Cl, Br, or I).

In some embodiments, R^{X2} is —CN.

In some embodiments, R^{X2} is —NO₂.

In some embodiments, R^{X2} is —OH.

In some embodiments, R^{X2} is —NH₂.

In some embodiments, R^{X2} is C₁₋₆ alkyl (e.g., methyl (C₁), ethyl (C₂), n-propyl (C₃), i-propyl (C₃), n-butyl (C₄), i-butyl (C₄), s-butyl (C₄), 1-butyl (C₄), pentyl (C₅), or hexyl (C₆)), wherein the C₁₋₆ alkyl is optionally substituted with one or more halogen (e.g., F, Cl, Br, or I), —CN, —NO₂, —OH, or —NH₂.

In some embodiments, R^{B1} is hydrogen.

In some embodiments, R^{B1} is halogen (e.g., F, Cl, Br, or I).

In some embodiments, R^{B1} is —CN.

In some embodiments, R^{B1} is —NO₂.

In some embodiments, R^{B1} is —OH.

In some embodiments, R^{B1} is —NH₂.

In some embodiments, R^{B1} is C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., F, Cl, Br, or I), —CN, —NO₂, —OH, or —NH₂.

In some embodiments, R^{B1} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)) optionally substituted with one or more halogen (e.g., F, Cl, Br, or I), —CN, —NO₂, —OH, or —NH₂.

In some embodiments, R^{B1} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino) optionally substituted with one or more halogen (e.g., F, Cl, Br, or I), —CN, —NO₂, —OH, or —NH₂.

In some embodiments, R^{B1} is C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)) optionally substituted with one or more halogen (e.g., F, Cl, Br, or I), —CN, —NO₂, —OH, or —NH₂.

In some embodiments, R^{B1} is C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)) optionally substituted with one or more halogen (e.g., F, Cl, Br, or I), —CN, —NO₂, —OH, or —NH₂.

In some embodiments, R^{B1} is C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₂), cycloheptenyl (C₇), cycloheptadienyl (C₂), cycloheptatrienyl (C₂), cyclooctyl (Ca), cyclooctenyl (Ca), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)) optionally substituted with one or more halogen (e.g., F, Cl, Br, or I), —CN, —NO₂, —OH, or —NH₂.

In some embodiments, R^{B1} is 3- to 12-membered heterocyclyl (e.g., heterocyclyl comprising one or two 3- to 8-membered rings and 1-5 heteroatoms selected from N, O, and S) optionally substituted with one or more halogen (e.g., F, Cl, Br, or I), —CN, —NO₂, —OH, or —NH₂.

In some embodiments, R^{B1} is C₆₋₁₀ aryl (e.g., phenyl or naphthyl) optionally substituted with one or more halogen (e.g., F, Cl, Br, or I), —CN, —NO₂, —OH, or —NH₂.

In some embodiments, R^{B1} is 5- to 10-membered heteroaryl (e.g., heteroaryl comprising one or two 5- to 6-membered rings and 1-5 heteroatoms selected from N, O, and S) optionally substituted with one or more halogen (e.g., F, Cl, Br, or I), —CN, —NO₂, —OH, or —NH₂.

In some embodiments, R^{B2} is hydrogen.

In some embodiments, R^{B2} is halogen (e.g., F, Cl, Br, or I).

In some embodiments, R^{B2} is —CN.

In some embodiments, R^{B2} is —NO₂.

In some embodiments, R^{B2} is —OH.

In some embodiments, R^{B2} is —NH₂.

In some embodiments, R^{B2} is C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., F, Cl, Br, or I), —CN, —NO₂, —OH, or —NH₂.

In some embodiments, R^{B2} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)) optionally substituted with one or more halogen (e.g., F, Cl, Br, or I), —CN, —NO₂, —OH, or —NH₂.

In some embodiments, R^{B2} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-t-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, f-butylhexylamino, or pentylhexylamino) optionally substituted with one or more halogen (e.g., F, Cl, Br, or I), —CN, —NO₂, —OH, or —NH₂.

In some embodiments, R^{B2} is C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)) optionally substituted with one or more halogen (e.g., F, Cl, Br, or I), —CN, —NO₂, —OH, or —NH₂.

In some embodiments, R^{B2} is C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)) optionally substituted with one or more halogen (e.g., F, Cl, Br, or I), —CN, —NO₂, —OH, or —NH₂.

In some embodiments, R^{B2} is C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)) optionally substituted with one or more halogen (e.g., F, Cl, Br, or I), —CN, —NO₂, —OH, or —NH₂.

In some embodiments, R^{B2} is 3- to 12-membered heterocyclyl (e.g., heterocyclyl comprising one or two 3- to 8-membered rings and 1-5 heteroatoms selected from N, O, and S) optionally substituted with one or more halogen (e.g., F, Cl, Br, or I), —CN, —NO₂, —OH, or —NH₂.

In some embodiments, R^{B2} is C₆₋₁₀ aryl (e.g., phenyl or naphthyl) optionally substituted with one or more halogen (e.g., F, Cl, Br, or I), —CN, —NO₂, —OH, or —NH₂.

In some embodiments, R^{B2} is 5- to 10-membered heteroaryl (e.g., heteroaryl comprising one or two 5- to 6-membered rings and 1-5 heteroatoms selected from N, O, and S) optionally substituted with one or more halogen (e.g., F, Cl, Br, or I), —CN, —NO₂, —OH, or —NH₂.

In some embodiments, R^{B4} is hydrogen.

In some embodiments, R^{B4} is halogen (e.g., F, Cl, Br, or I).

In some embodiments, R^{B4} is —CN.

In some embodiments, R^{B4} is —NO₂.

In some embodiments, R^{B4} is —OH.

In some embodiments, R^{B4} is —NH₂.

In some embodiments, R^{B4} is C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., F, Cl, Br, or I), —CN, —NO₂, —OH, or —NH₂.

In some embodiments, R^{B4} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)) optionally substituted with one or more halogen (e.g., F, Cl, Br, or I), —CN, —NO₂, —OH, or —NH₂.

In some embodiments, R^{B4} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, butylhexylamino, s-butylhexylamino, t-butylhexylamino, or pentylhexylamino) optionally substituted with one or more halogen (e.g., F, Cl, Br, or I), —CN, —NO₂, —OH, or —NH₂.

In some embodiments, R^{B4} is C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)) optionally substituted with one or more halogen (e.g., F, Cl, Br, or I), —CN, —NO₂, —OH, or —NH₂.

In some embodiments, R^{B4} is C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)) optionally substituted with one or more halogen (e.g., F, Cl, Br, or I), —CN, —NO₂, —OH, or —NH₂.

In some embodiments, R^{B4} is C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)) optionally substituted with one or more halogen (e.g., F, Cl, Br, or I), —CN, —NO₂, —OH, or —NH₂.

In some embodiments, R^{B4} is 3- to 12-membered heterocyclyl (e.g., heterocyclyl comprising one or two 3- to 8-membered rings and 1-5 heteroatoms selected from N, O, and S) optionally substituted with one or more halogen (e.g., F, Cl, Br, or I), —CN, —NO₂, —OH, or —NH₂.

In some embodiments, R^{B4} is C₆₋₁₀ aryl (e.g., phenyl or naphthyl) optionally substituted with one or more halogen (e.g., F, Cl, Br, or I), —CN, —NO₂, —OH, or —NH₂.

In some embodiments, R^{B4} is 5- to 10-membered heteroaryl (e.g., heteroaryl comprising one or two 5- to 6-membered rings and 1-5 heteroatoms selected from N, O, and S) optionally substituted with one or more halogen (e.g., F, Cl, Br, or I), —CN, —NO₂, —OH, or —NH₂.

In some embodiments, R^{B5} is hydrogen.

In some embodiments, R^{B5} is halogen (e.g., F, Cl, Br, or I).

In some embodiments, R^{B5} is —CN.

In some embodiments, R^{B5} is —NO₂.

In some embodiments, R^{B5} is —OH.

In some embodiments, R^{B5} is —NH₂.

In some embodiments, R^{B5} is C₁₋₆ alkyl (e.g., methyl (C₁), ethyl (C₂), n-propyl (C₃), i-propyl (C₃), n-butyl (C₄), i-butyl (C₄), s-butyl (C₄), 1-butyl (CA), pentyl (C₅), or hexyl (C₆)) optionally substituted with one or more halogen (e.g., F, Cl, Br, or I), —CN, —NO₂, —OH, or —NH₂.

In some embodiments, R^{B5} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)) optionally substituted with one or more halogen (e.g., F, Cl, Br, or I), —CN, —NO₂, —OH, or —NH₂.

In some embodiments, R^{B5} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, f-butylhexylamino, or pentylhexylamino) optionally substituted with one or more halogen (e.g., F, Cl, Br, or I), —CN, —NO₂, —OH, or —NH₂.

In some embodiments, R^{B5} is C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)) optionally substituted with one or more halogen (e.g., F, Cl, Br, or I), —CN, —NO₂, —OH, or —NH₂.

In some embodiments, R^{B5} is C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)) optionally substituted with one or more halogen (e.g., F, Cl, Br, or I), —CN, —NO₂, —OH, or —NH₂.

In some embodiments, R^{B5} is C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)) optionally substituted with one or more halogen (e.g., F, Cl, Br, or I), —CN, —NO₂, —OH, or —NH₂.

In some embodiments, R^{B5} is 3- to 12-membered heterocyclyl (e.g., heterocyclyl comprising one or two 3- to 8-membered rings and 1-5 heteroatoms selected from N, O, and S) optionally substituted with one or more halogen (e.g., F, Cl, Br, or I), —CN, —NO₂, —OH, or —NH₂.

In some embodiments, R^{B5} is C₆₋₁₀ aryl (e.g., phenyl or naphthyl) optionally substituted with one or more halogen (e.g., F, Cl, Br, or I), —CN, —NO₂, —OH, or —NH₂.

In some embodiments, R^{B5} is 5- to 10-membered heteroaryl (e.g., heteroaryl comprising one or two 5- to 6-membered rings and 1-5 heteroatoms selected from N, O, and S) optionally substituted with one or more halogen (e.g., F, Cl, Br, or I), —CN, —NO₂, —OH, or —NH₂.

In some embodiments, R^{B1} and R^{B4}, together with the intervening atoms to which they are attached, form 3- to 8-membered heterocyclyl (e.g., heterocyclyl comprising one or two 3- to 8-membered rings and 1-5 heteroatoms selected from N, O, and S) optionally substituted with one or more halogen (e.g., F, Cl, Br, or I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), or C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)).

In some embodiments, R^{B1} and R^{B4}, together with the intervening atoms to which they are attached, form 5- to 6-membered heteroaryl (e.g., heterocyclyl comprising one or two 3- to 8-membered rings and 1-5 heteroatoms selected from N, O, and S) optionally substituted with one or more halogen (e.g., F, Cl, Br, or I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), or C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)).

In some embodiments, R^{B1} and R^{B4}, together with the intervening atoms to which they are attached, form dihydrofuran or pyrazole optionally substituted with one or more C₁₋₆ alkyl.

In some embodiments, at least one R^{C} is hydrogen.

In some embodiments, each R^{C} is hydrogen.

In some embodiments, at least one R^{C} is halogen (e.g., F, Cl, Br, or I).

In some embodiments, at least one R^{C} is —CN.

In some embodiments, at least one R^{C} is —NO₂.

In some embodiments, at least one R^{C} is —OH.

In some embodiments, at least one R^{C} is —NH₂.

In some embodiments, at least one R^{C} is C₁₋₆ alkyl (e.g., methyl (C₁), ethyl (C₂), n-propyl (C₃), i-propyl (C₃), n-butyl (CA), i-butyl (C₄), s-butyl (C₄), t-butyl (C₄), pentyl (C₅), or hexyl (C₆)) optionally substituted with one or more halogen (e.g., F, Cl, Br, or I), —CN, —NO₂, —OH, or —NH₂.

In some embodiments, at least one R^{C} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (CA), pentoxy (C₅), or hexoxy (C₆)) optionally substituted with one or more halogen (e.g., F, Cl, Br, or I), —CN, —NO₂, —OH, or —NH₂.

In some embodiments, at least one R^{C} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, butylpentylamino, i-butylpentylamino, s-butylpentylamino, t-butylpentylamino, n-butylhexylamino, i-butylhexylamino, s-butylhexylamino, t-butylhexylamino, or pentylhexylamino) optionally substituted with one or more halogen (e.g., F, Cl, Br, or I), —CN, —NO₂, —OH, or —NH₂.

In some embodiments, at least one R^{C} is C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)) optionally substituted with one or more halogen (e.g., F, Cl, Br, or I), —CN, —NO₂, —OH, or —NH₂.

In some embodiments, at least one R^{C} is C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)) optionally substituted with one or more halogen (e.g., F, Cl, Br, or I), —CN, —NO₂, —OH, or —NH₂.

In some embodiments, n^{D} is 0.

In some embodiments, n^{D} is 1.

In some embodiments, Y is O.

In some embodiments, Y is S.

In some embodiments, Y is C(R^{D}).

In some embodiments, Y is CH.

In some embodiments, n^{D} is 0, and Y is O or S.

In some embodiments, n^{D} is 0, and Y is S.

In some embodiments, n^{D} is 1, and Y is C(R^{D}).

In some embodiments, n^{D} is 1, and Y is CH.

In some embodiments, one R^{D} is

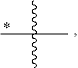

wherein * denotes attachment to L.

In some embodiments, at least one R^{D} is hydrogen.

In some embodiments, at least two R^{D} are hydrogen.

In some embodiments, three R^{D} are hydrogen.

In some embodiments, one R^{D} is

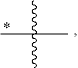

and each of the remaining R^{D} is independently hydrogen.

In some embodiments, at least one R^{D} is halogen (e.g., F, Cl, Br, or I).

In some embodiments, at least one R^{D} is —CN.

In some embodiments, at least one R^{D} is —NO₂.

In some embodiments, at least one R^{D} is —OH.

In some embodiments, at least one R^{D} is —NH₂.

In some embodiments, at least one R^{D} is C₁₋₆ alkyl (e.g., methyl (C₁), ethyl (C₂), n-propyl (C₃), i-propyl (C₃), n-butyl (C₄), i-butyl (C₄), s-butyl (CA), t-butyl (C₄), pentyl (C₅), or hexyl (C₆)) optionally substituted with one or more halogen (e.g., F, Cl, Br, or I), —CN, —NO₂, —OH, or —NH₂.

In some embodiments, at least one R^{D} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), 1-butoxy (C₄), pentoxy (C₅), or hexoxy (C₆)) optionally substituted with one or more halogen (e.g., F, Cl, Br, or I), —CN, —NO₂, —OH, or —NH₂.

In some embodiments, at least one R^{D} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino) optionally substituted with one or more halogen (e.g., F, Cl, Br, or I), —CN, —NO₂, —OH, or —NH₂.

In some embodiments, at least one R^{D} is C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)) optionally substituted with one or more halogen (e.g., F, Cl, Br, or I), —CN, —NO₂, —OH, or —NH₂.

In some embodiments, at least one R^{D} is C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)) optionally substituted with one or more halogen (e.g., F, Cl, Br, or I), —CN, —NO₂, —OH, or —NH₂.

In some embodiments, T is of Formula I-1-a

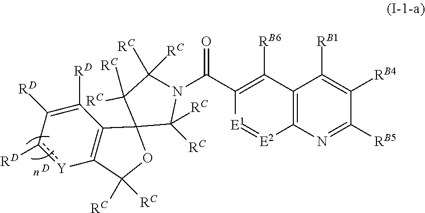

wherein:
E¹ is N or CR^{B7};
E² is N or CR^{B8}, and
R^{B6}, R^{B7}, and R^{B8} are independently hydrogen, halogen, —CN, —NO₂, —OH, or —NH₂.

In some embodiments, E¹ is N.

In some embodiments, E¹ is CR^{B7}.

In some embodiments, E² is N.

In some embodiments, E² is CR^{B8}.

In some embodiments, E¹ is N, and E² is CR^{B8}.

In some embodiments, E¹ is CR^{B7}, and E² is N.

In some embodiments, R^{B6} is hydrogen.

In some embodiments, R^{B6} is halogen (e.g., F, Cl, Br, or I).

In some embodiments, R^{B6} is —CN.

In some embodiments, R^{B6} is —NO₂.

In some embodiments, R^{B6} is —OH.

In some embodiments, R^{B6} is —NH₂.

In some embodiments, R^{B7} is hydrogen.

In some embodiments, R^{B7} is halogen (e.g., F, Cl, Br, or I).

In some embodiments, R^{B7} is —CN.

In some embodiments, R^{B7} is —NO₂.

In some embodiments, R^{B7} is —OH.

In some embodiments, R^{B7} is —NH₂.

In some embodiments, R^{B8} is hydrogen.

In some embodiments, R^{B8} is halogen (e.g., F, Cl, Br, or I).

In some embodiments, R^{B8} is —CN.

In some embodiments, R^{B8} is —NO₂.

In some embodiments, R^{B8} is —OH.

In some embodiments, R^{B8} is —NH₂.

In some embodiments, T is of Formula I-1-b

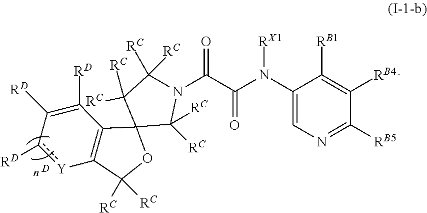

In some embodiments, T is of Formula I-1-c

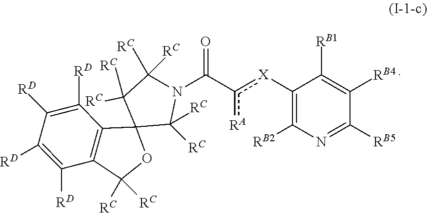

In some embodiments, T is of Formula I-1-d

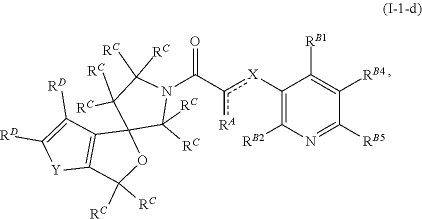

wherein Y is O or S.

### Variable L

In some embodiments, at least one L′ is —C(═O)—.

In some embodiments, at least one L′ is —C(═O)N(R^{L′})—.

In some embodiments, at least one L′ is —N(R^{L′})C(═O)—.

In some embodiments, at least one L′ is —C(═O)O—.

In some embodiments, at least one L′ is —OC(═O)—.

In some embodiments, at least one L′ is —N(R^{L′})—.

In some embodiments, at least one L′ is —O—.

In some embodiments, at least one L′ is —O—(C₁₋₆ alkylene)- (e.g., —O-methylene-(C₁), —O-ethylene-(C₂), —O-n-propylene-(C₃), —O-i-propylene-(C₃), —O-n-butylene-(C₄), —O-i-butylene-(C₄), —O-s-butylene-(C₄), —O-t-butylene-(C₄), —O-pentylene-(C₅), or —O-hexylene-(C₆)) optionally substituted with one or more halogen (e.g., F, Cl, Br, or I), —CN, —NO₂, —OH, or —NH₂.

In some embodiments, at least one L′ is —(C₁₋₆ alkylene)-O— (e.g., -methylene-O—(C₁), -ethylene-O—(C₂), -n-propylene-O—(C₃), -i-propylene-O—(C₃), -n-butylene-O—(C₄), -i-butylene-O—(C₄), -s-butylene-O—(C₄), -t-butylene-O—(C₄), -pentylene-O—(C₅), or -hexylene-O—(C₆)) optionally substituted with one or more halogen (e.g., F, Cl, Br, or I), —CN, —NO₂, —OH, or —NH₂.

In some embodiments, at least one L′ is —S—.

In some embodiments, at least one L′ is —S(═O)₂—.

In some embodiments, at least one L′ is C₁₋₆ alkylene (e.g., methylene (C₁), ethylene (C₂), n-propylene (C₃), i-propylene (C₃), n-butylene (C₄), i-butylene (C₄), s-butylene (C₄), t-butylene (C₄), pentylene (C₅), or hexylene (C₆)) optionally substituted with one or more halogen (e.g., F, Cl, Br, or I), —CN, —NO₂, —OH, or —NH₂.

In some embodiments, at least one L′ is C₁₋₆ heteroalkylene optionally substituted with one or more halogen (e.g., F, Cl, Br, or I), —CN, —NO₂, —OH, or —NH₂.

In some embodiments, at least one L′ is C₂₋₆ alkenylene (e.g., ethenylene (C₂), 1-propenylene (C₃), 2-propenylene (C₃), 1-butenylene (C₄), 2-butenylene (C₄), butadienylene (C₄), pentenylene (C₅), pentadienylene (C₅), or hexenylene (C₆)) optionally substituted with one or more halogen (e.g., F, Cl, Br, or I), —CN, —NO₂, —OH, or —NH₂.

In some embodiments, at least one L′ is C₂₋₆ alkynylene (e.g., ethynylene (C₂), 1-propynylene (C₃), 2-propynylene (C₃), 1-butynylene (C₄), 2-butynylene (C₄), pentynylene (C₅), or hexynylene (C₆)) optionally substituted with one or more halogen (e.g., F, Cl, Br, or I), —CN, —NO₂, —OH, or —NH₂.

In some embodiments, at least one L′ is C₃₋₁₂ carbocyclylene (e.g., cyclopropylene (C₃), cyclopropenylene (C₃), cyclobutylene (C₄), cyclobutenylene (C₄), cyclopentylene (C₅), cyclopentenylene (C₅), cyclohexylene (C₆), cyclohexenylene (C₆), cyclohexadienylene (C₆), cycloheptylene (C₇), cycloheptenylene (C₇), cycloheptadienylene (C₇), cycloheptatrienylene (C₇), cyclooctylene (C₈), cyclooctenylene (C₈), bicyclo[2.2.1]heptanylene (C₇), bicyclo[2.2.2]octanylene (C₈), cyclononylene (C₉), cyclononenylene (C₉), cyclodecylene (C₁₀), cyclodecenylene (C₁₀), octahydro-1H-indenylene (C₉), decahydronaphthalenylene (C₁₀), or spiro[4.5]decanylene (C₁₀)) optionally substituted with one or more halogen (e.g., F, Cl, Br, or I), —CN, —NO₂, —OH, or —NH₂.

In some embodiments, at least one L′ is 3- to 12-membered heterocyclylene (e.g., heterocyclylene comprising one or two 3- to 8-membered rings and 1-5 heteroatoms selected from N, O, and S) optionally substituted with one or more halogen (e.g., F, Cl, Br, or I), —CN, —NO₂, —OH, or —NH₂.

In some embodiments, at least one L′ is C₆₋₁₀ arylene (e.g., phenylene or naphthylene) optionally substituted with one or more halogen (e.g., F, Cl, Br, or I), —CN, —NO₂, —OH, or —NH₂.

In some embodiments, at least one L′ is 5- to 10-membered heteroarylene (e.g., heteroarylene comprising one or two 5- to 6-membered rings and 1-5 heteroatoms selected from N, O, and S) optionally substituted with one or more halogen (e.g., F, Cl, Br, or I), —CN, —NO₂, —OH, or —NH₂.

In some embodiments, at least one R^{L′} is hydrogen.

In some embodiments, each R^{L′} is hydrogen.

In some embodiments, at least one R^{L′} is C₁₋₆ alkyl (e.g., methyl (C₁), ethyl (C₂), n-propyl (C₃), i-propyl (C₃), n-butyl (C₄), i-butyl (C₄), s-butyl (C₄), 1-butyl (C₄), pentyl (C₅), or hexyl (C₆)) optionally substituted with one or more halogen (e.g., F, Cl, Br, or I), —CN, —NO₂, —OH, or —NH₂.

In some embodiments, at least one R^{L′} is C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)) optionally substituted with one or more halogen (e.g., F, Cl, Br, or I), —CN, —NO₂, —OH, or —NH₂.

In some embodiments, at least one R^{L′} is C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)) optionally substituted with one or more halogen (e.g., F, Cl, Br, or I), —CN, —NO₂, —OH, or —NH₂.

In some embodiments, at least one R^{L′} is C₃₋₈ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)) optionally substituted with one or more halogen (e.g., F, Cl, Br, or I), —CN, —NO₂, —OH, or —NH₂.

In some embodiments, at least one R^{L′} is 3- to 8-membered heterocyclyl (e.g., heterocyclyl comprising one or two 3- to 8-membered rings and 1-5 heteroatoms selected from N, O, and S) optionally substituted with one or more halogen (e.g., F, Cl, Br, or I), —CN, —NO₂, —OH, or —NH₂.

In some embodiments, at least one R^{L′} is —S(═O)₂R^{a} optionally substituted with one or more halogen (e.g., F, Cl, Br, or I), —CN, —NO₂, —OH, or —NH₂.

In some embodiments, at least one R^{L′} is —S(═O)₂OR^{a} optionally substituted with one or more halogen (e.g., F, Cl, Br, or I), —CN, —NO₂, —OH, or —NH₂.

In some embodiments, at least one R^{L′} is —S(═O)₂N(R^{a})₂ optionally substituted with one or more halogen (e.g., F, Cl, Br, or I), —CN, —NO₂, —OH, or —NH₂.

In some embodiments, at least one RE is —C(═O)R^{a} optionally substituted with one or more halogen (e.g., F, Cl, Br, or I), —CN, —NO₂, —OH, or —NH₂.

In some embodiments, at least one R^{L′} is —C(═O)OR^{a} optionally substituted with one or more halogen (e.g., F, Cl, Br, or I), —CN, —NO₂, —OH, or —NH₂.

In some embodiments, at least one R^{L′} is —C(═O)N(R^{a})₂ optionally substituted with one or more halogen (e.g., F, Cl, Br, or I), —CN, —NO₂, —OH, or —NH₂.

In some embodiments, at least one R^{a} is hydrogen.

In some embodiments, each R^{a} is hydrogen.

In some embodiments, at least one R^{a} is C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., F, Cl, Br, or I), —CN, —NO₂, —OH, or —NH₂.

In some embodiments, at least one R^{a} is C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)) optionally substituted with one or more halogen (e.g., F, Cl, Br, or I), —CN, —NO₂, —OH, or —NH₂.

In some embodiments, at least one R^{a} is C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)) optionally substituted with one or more halogen (e.g., F, Cl, Br, or I), —CN, —NO₂, —OH, or —NH₂.

In some embodiments, 1 is an integer selected from 1 to 10, an integer selected from 2 to 10, an integer selected from 3 to 10, an integer selected from 4 to 10, an integer selected from 5 to 10, an integer selected from 6 to 10, an integer selected from 7 to 10, or an integer selected from 8 to 10.

In some embodiments, l is an integer selected from 1 to 9, an integer selected from 1 to 8, an integer selected from 1 to 7, an integer selected from 1 to 6, an integer selected from 1 to 5, an integer selected from 1 to 4, or an integer selected from 1 to 3.

In some embodiments, l is an integer selected from 2 to 9, an integer selected from 3 to 8, or an integer selected from 4 to 7.

In some embodiments, l is 0. In some embodiments, l is 1. In some embodiments, l is 2. In some embodiments, l is 3. In some embodiments, l is 4. In some embodiments, l is 5. In some embodiments, l is 6. In some embodiments, l is 7. In some embodiments, l is 8. In some embodiments, l is 9. In some embodiments, l is 10.

In some embodiments, each L′ is independently selected from —C(═O)—, —C(═O)N(R^{L′})—, —N(R^{L′})C(═O)—, —N(R^{L′})—, —O—, —O—(C₁₋₆ alkylene)-, —(C₁₋₆ alkylene)-O—, C₁₋₆ alkylene, C₃₋₁₂ carbocyclylene, and 5- to 10-membered heteroarylene.

In some embodiments, L is **-[L′]ₗ₋₁—N(R^{L′})C(═O)—* or *-(pyridinylene)-[L′]ₗ₋₁-**.

In some embodiments, L is **—[O—(C₁₋₆ alkylene)]₁₋₆-N(R^{L′})C(═O)—*, **—O—[(C₁₋₆ alkylene)]₁₋₃—N(R^{L′})C(═O)—*, *-(5- to 6-membered heteroarylene)-C(═O)NH—[(C₁₋₆ alkylene)-O]₁₋₆—**, *-(5- to 6-membered heteroarylene)-C(═O)N(R^{L′})—(C₃₋₁₂ carbocyclylene)-O—[(C₁₋₆ alkylene)-O]₁₋₃—**, or *-(5- to 6-membered heteroarylene)-C(═O)N(R^{L′})—[(C₁₋₆ alkylene)-O]₁₋₃—**.

In some embodiments, L is **—[O—(C₁₋₆ alkylene)]₁₋₆-N(H)C(═O)—*, **—O—[(C₁₋₆-(alkylene)]₁₋₃—N(H)C(═O)—*, *-(pyridinylene)-C(═O)NH—[(C₁₋₆ alkylene)-O]₁₋₆—**, pyridinylene)-C(═O)NH—(C₃₋₁₂ carbocyclylene)-O—[(C₁₋₆ alkylene)-O]₁₋₃—**, or *-(pyridinylene)-C(═O)NH—[(C₁₋₆ alkylene)-O]₁₋₃—**.

### Variable V

In some embodiments, R^{V1} is hydrogen.

In some embodiments, R^{V1} is C₁₋₆ alkyl (e.g., methyl (C₁), ethyl (C₂), n-propyl (C₃), i-propyl (C₃), n-butyl (Ca), i-butyl (C₄), s-butyl (C₄), t-butyl (C₄), pentyl (C₅), or hexyl (C₆)) optionally substituted with one or more halogen (e.g., F, Cl, Br, or I), —CN, —NO₂, —OH, or —NH₂.

In some embodiments, R^{V1} is C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₂), cycloheptenyl (C₇), cycloheptadienyl (C₇), cycloheptatrienyl (C₂), cyclooctyl (C₅), cyclooctenyl (C₅), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)) optionally substituted with one or more halogen (e.g., F, Cl, Br, or I), —CN, —NO₂, —OH, or —NH₂.

In some embodiments, R^{V1} is C₃₋₆ carbocyclyl optionally substituted with one or more halogen (e.g., F, Cl, Br, or I).

In some embodiments, R^{V1} is C₃₋₆ carbocyclyl substituted with one F.

In some embodiments, R^{V1} is 3- to 6-membered heterocyclyl (e.g., heterocyclyl comprising one 3- to 6-membered ring and 1-3 heteroatoms selected from N, O, and S) optionally substituted with one or more halogen (e.g., F, Cl, Br, or I), —CN, —NO₂, —OH, or —NH₂.

In some embodiments, R^{VN1} is hydrogen.

In some embodiments, R^{VN1} is C₁₋₆ alkyl, wherein the C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) is optionally substituted with one or more halogen (e.g., F, Cl, Br, or I), —CN, —NO₂, —OH, or —NH₂.

In some embodiments, R^{V2} is hydrogen.

In some embodiments, R^{V2} is C₁₋₆ alkyl, wherein the C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) is optionally substituted with one or more halogen (e.g., F, Cl, Br, or I), —CN, —NO₂, —OH, or —NH₂.

In some embodiments, at least one R^{A′} is hydrogen.

In some embodiments, each R^{A′} is hydrogen.

In some embodiments, at least one R^{A′} is halogen (e.g., F, Cl, Br, or I).

In some embodiments, at least one R^{A′} is —CN.

In some embodiments, at least one R^{A′} is —NO₂.

In some embodiments, at least one R^{A′} is —OH.

In some embodiments, one R^{A′} is —OH, and each of the remaining R^{A′} is independently hydrogen.

In some embodiments, at least one R^{A′} is —NH₂.

In some embodiments, at least one R^{A′} is C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., F, Cl, Br, or I), —CN, —NO₂, —OH, or —NH₂.

In some embodiments, at least one R^{A′} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), 1-butoxy (C₄), pentoxy (C₅), or hexoxy (C₆)) optionally substituted with one or more halogen (e.g., F, Cl, Br, or I), —CN, —NO₂, —OH, or —NH₂.

In some embodiments, at least one R^{A′} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino) optionally substituted with one or more halogen (e.g., F, Cl, Br, or I), —CN, —NO₂, —OH, or —NH₂.

In some embodiments, at least one R^{A′} is C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)) optionally substituted with one or more halogen (e.g., F, Cl, Br, or I), —CN, —NO₂, —OH, or —NH₂.

In some embodiments, at least one R^{A′} is C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)) optionally substituted with one or more halogen (e.g., F, Cl, Br, or I), —CN, —NO₂, —OH, or —NH₂. In some embodiments, R^{VN2} is hydrogen.

In some embodiments, R^{VN2} is C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., F, Cl, Br, or I), —CN, —NO₂, —OH, or —NH₂.

In some embodiments, at least one R^{V4} is hydrogen.

In some embodiments, each R^{V4} is hydrogen.

In some embodiments, at least one R^{V4} is halogen (e.g., F, Cl, Br, or I).

In some embodiments, at least one R^{V4} is —CN.

In some embodiments, at least one R^{V4} is —NO₂.

In some embodiments, at least one R^{V4} is —OH.

In some embodiments, at least one R^{V4} is —NH₂.

In some embodiments, at least one R^{V4} is C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., F, Cl, Br, or I), —CN, —NO₂, —OH, or —NH₂. In some embodiments, at least one R^{V4} is C₁₋₆ alkoxy (e.g., methoxy (C₁), ethoxy (C₂), propoxy (C₃), i-propoxy (C₃), n-butoxy (C₄), i-butoxy (Ca), s-butoxy (C₄), t-butoxy (C₄), pentoxy (C₅), or hexoxy (C₆)) optionally substituted with one or more halogen (e.g., F, Cl, Br, or I), —CN, —NO₂, —OH, or —NH₂.

In some embodiments, at least one R^{V4} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino) optionally substituted with one or more halogen (e.g., F, Cl, Br, or I), —CN, —NO₂, —OH, or —NH₂.

In some embodiments, at least one R^{V4} is C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)) optionally substituted with one or more halogen (e.g., F, Cl, Br, or I), —CN, —NO₂, —OH, or —NH₂.

In some embodiments, at least one R^{V4} is C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)) optionally substituted with one or more halogen (e.g., F, Cl, Br, or I), —CN, —NO₂, —OH, or —NH₂.

In some embodiments, at least one R^{V5} is hydrogen.

In some embodiments, each R^{V5} is hydrogen.

In some embodiments, at least one R^{V5} is halogen (e.g., F, Cl, Br, or I).

In some embodiments, at least one R^{V5} is —CN.

In some embodiments, at least one R^{V5} is —NO₂.

In some embodiments, at least one R^{V5} is —OH.

In some embodiments, at least one R^{V5} is —NH₂.

In some embodiments, at least one R^{V5} is C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., F, Cl, Br, or I), —CN, —NO₂, —OH, or —NH₂.

In some embodiments, at least one R^{V5} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), 1-butoxy (C₄), pentoxy (C₅), or hexoxy (C₆)) optionally substituted with one or more halogen (e.g., F, Cl, Br, or I), —CN, —NO₂, —OH, or —NH₂.

In some embodiments, at least one R^{V5} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino) optionally substituted with one or more halogen (e.g., F, Cl, Br, or I), —CN, —NO₂, —OH, or —NH₂.

In some embodiments, at least one R^{V5} is C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)) optionally substituted with one or more halogen (e.g., F, Cl, Br, or I), —CN, —NO₂, —OH, or —NH₂.

In some embodiments, at least one R^{V5} is C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)) optionally substituted with one or more halogen (e.g., F, Cl, Br, or I), —CN, —NO₂, —OH, or —NH₂.

In some embodiments, each R^{a} is independently C₁₋₆ alkyl (e.g., methyl (C₁), ethyl (C₂), n-propyl (C₃), i-propyl (C₃), n-butyl (C₄), i-butyl (C₄), s-butyl (C₄), f-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- to 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In some embodiments, R^{V6} is C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more halogen (e.g., F, Cl, Br, or I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), or C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)).

In some embodiments, R^{V6} is C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)) optionally substituted with one or more halogen (e.g., F, Cl, Br, or I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), S-butyl (C₄), t-butyl (C₄), pentyl (C₅), or hexyl (C₆), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), or C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)).

In some embodiments, R^{V6} is C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)) optionally substituted with one or more halogen (e.g., F, Cl, Br, or I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), or C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)).

In some embodiments, R^{V6} is C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)) optionally substituted with one or more halogen (e.g., F, Cl, Br, or I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), or C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)).

In some embodiments, R^{V6} is 3- to 12-membered heterocyclyl (e.g., heterocyclyl comprising one or two 3- to 8-membered rings and 1-5 heteroatoms selected from N, O, and S) optionally substituted with one or more halogen (e.g., F, Cl, Br, or I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), 1-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), or C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)).

In some embodiments, R^{V6} is C₆₋₁₀ aryl (e.g., phenyl or naphthyl) optionally substituted with one or more halogen (e.g., F, Cl, Br, or I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), 1-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), or C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)).

In some embodiments, R^{V6} is 5- to 10-membered heteroaryl (e.g., heteroaryl comprising one or two 5- to 6-membered rings and 1-5 heteroatoms selected from N, O, and S) optionally substituted with one or more halogen (e.g., F, Cl, Br, or I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), or C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)).

In some embodiments, R^{V6} is C₂₋₆ alkynyl or 5- to 10-membered heteroaryl (e.g., heteroaryl comprising one or two 5- to 6-membered rings and 1-5 heteroatoms selected from N, O, and S) optionally substituted with one or more C₁₋₆ alkyl e.g., methyl (C₁), ethyl (C₂), n-propyl (C₃), i-propyl (C₃), n-butyl (C₄), i-butyl (C₄), s-butyl (C₄), 1-butyl (C₄), pentyl (C₅), or hexyl (C₆)).

In some embodiments, V is of Formula I-3-a

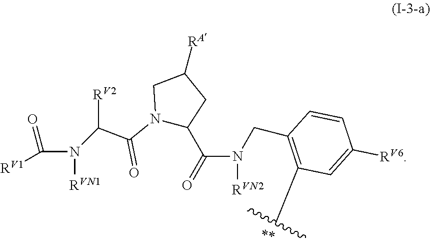

In some embodiments, V is of Formula I-3-a-i

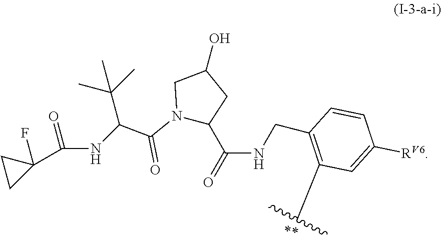

In some embodiments, V is

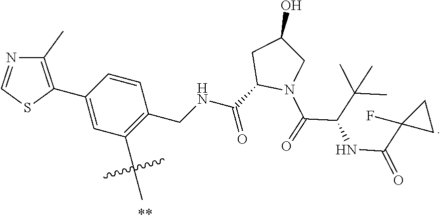 [SMILES: *c1cc(-c2scnc2C)ccc1CNC(=O)[C@@H]1C[C@@H](O)CN1C(=O)[C@@H](NC(=O)C1(F)CC1)C(C)(C)C]

Embodiments of the variables in any of the Formulae described herein, e.g., Formulae I-1, I-2, and I-3, as applicable, are described below. Any of the variables can be any moiety as described in the embodiments below. In addition, the combination of any moieties described for any of the variables, as applicable, with any moieties described for any of the remaining variables, are also contemplated.

Without wishing to be limited by this statement, while various options for variables are described herein, it is understood that the present disclosure intends to encompass operable embodiments having combinations of the options. The disclosure may be interpreted as excluding the non-operable embodiments caused by certain combinations of the options.

When a range of values is listed, each discrete value and sub-range within the range are also contemplated. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

In some embodiments, the compound is selected from the compounds in Table 1.

In some embodiments, the compound is selected from the compounds in Table 1, or a pharmaceutically acceptable salt thereof.

In some embodiments, the compound is selected from the compounds in Table 1, or a pharmaceutically acceptable salt, solvate, or stereoisomer thereof.

TABLE 1

| Example | |
|---|---|
| # | Structure |
| B1 | 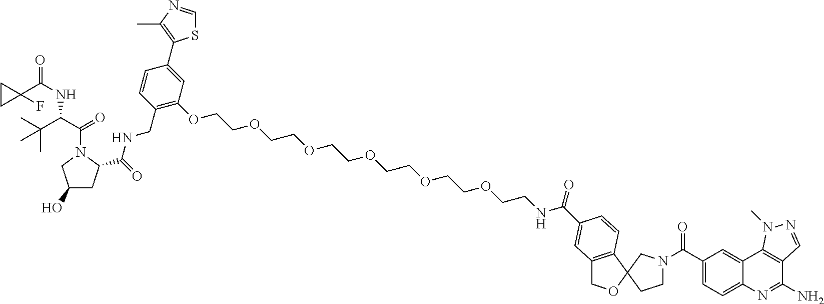 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCOCCNC(=O)c2ccc3c(c2)COC32CCN(C(=O)c3ccc4nc(N)c5cnn(C)c5c4c3)C2)c1] |
| B2 | 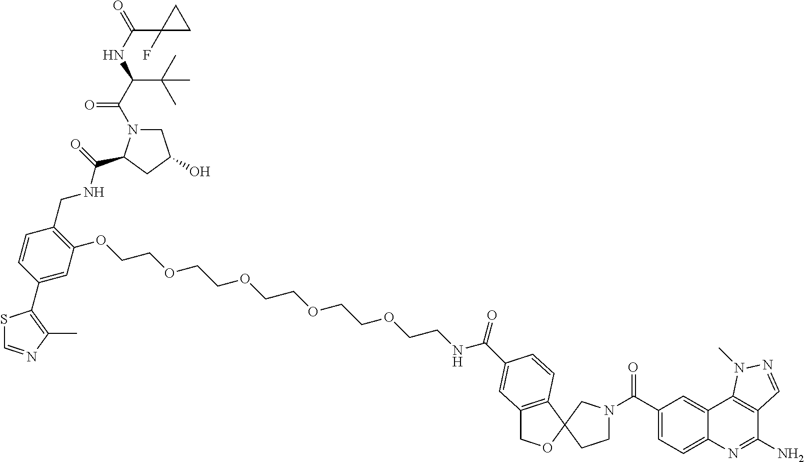 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCNC(=O)c2ccc3c(c2)COC32CCN(C(=O)c3ccc4nc(N)c5cnn(C)c5c4c3)C2)c1] |
| B3 | 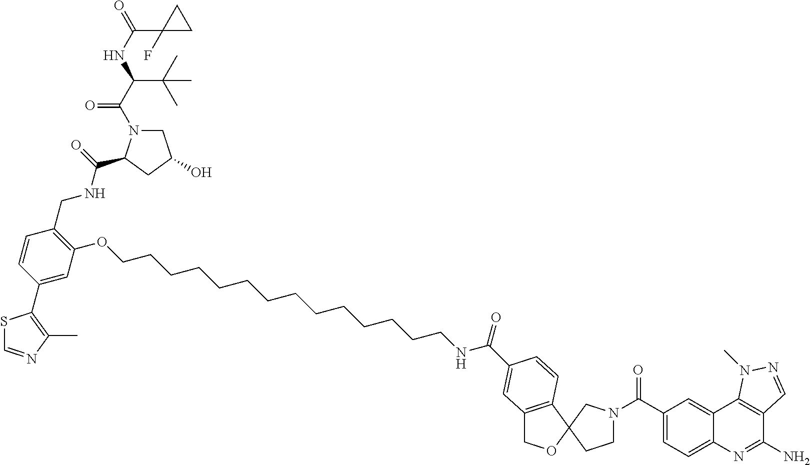 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCCCCNC(=O)c2ccc3c(c2)COC32CCN(C(=O)c3ccc4nc(N)c5cnn(C)c5c4c3)C2)c1] |
| B4 | 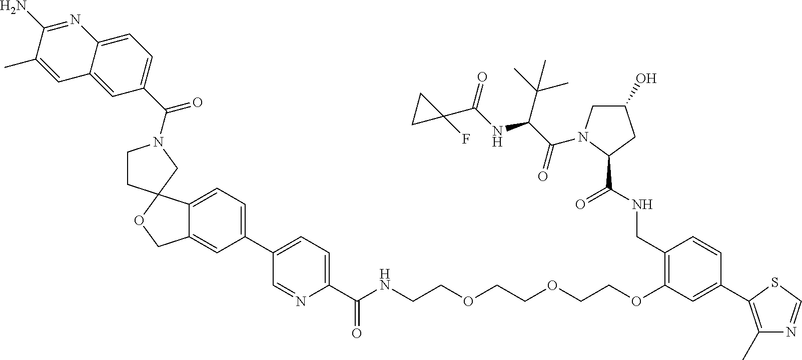 [SMILES: Cc1cc2cc(C(=O)N3CCC4(C3)OCc3cc(-c5ccc(C(=O)NCCOCCOCCOc6cc(-c7scnc7C)ccc6CNC(=O)[C@@H]6C[C@@H](O)CN6C(=O)[C@@H](NC(=O)C6(F)CC6)C(C)(C)C)nc5)ccc34)ccc2nc1N] |
| B5 | 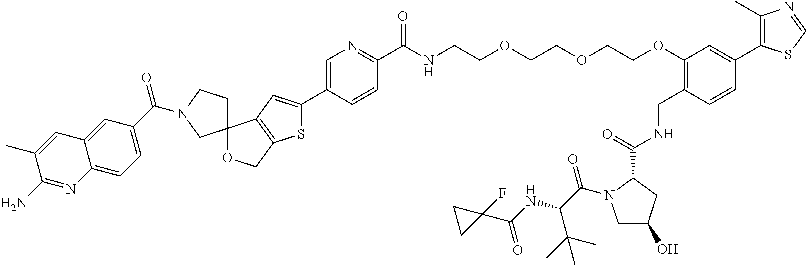 [SMILES: Cc1cc2cc(C(=O)N3CCC4(C3)OCc3sc(-c5ccc(C(=O)NCCOCCOCCOc6cc(-c7scnc7C)ccc6CNC(=O)[C@@H]6C[C@@H](O)CN6C(=O)[C@@H](NC(=O)C6(F)CC6)C(C)(C)C)nc5)cc34)ccc2nc1N] |
| B6 | 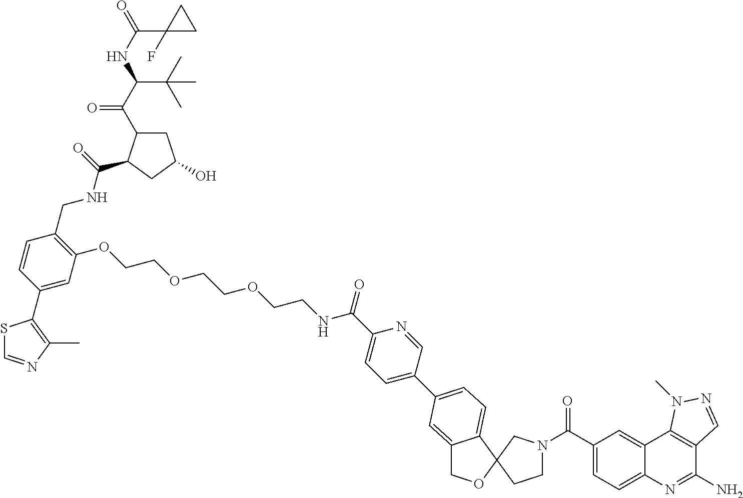 [SMILES: Cc1ncsc1-c1ccc(CNC(=O)[C@@H]2C[C@@H](O)CC2C(=O)[C@@H](NC(=O)C2(F)CC2)C(C)(C)C)c(OCCOCCOCCNC(=O)c2ccc(-c3ccc4c(c3)COC43CCN(C(=O)c4ccc5nc(N)c6cnn(C)c6c5c4)C3)cn2)c1] |
| B7 | 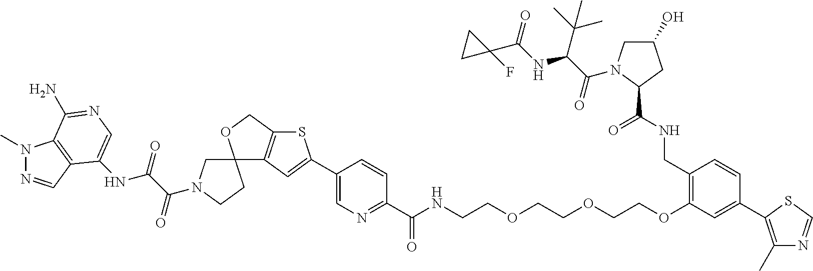 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3cc4c(s3)COC43CCN(C(=O)C(=O)Nc4cnc(N)c5c4cnn5C)C3)cn2)c1] |
| B8 | 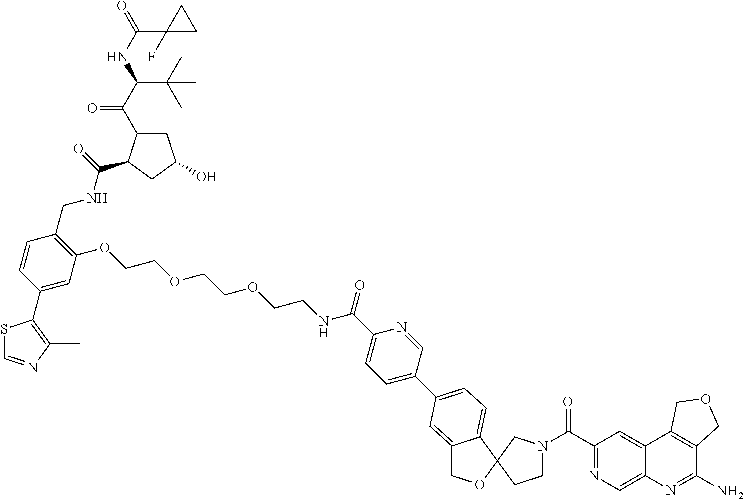 [SMILES: Cc1ncsc1-c1ccc(CNC(=O)[C@@H]2C[C@@H](O)CC2C(=O)[C@@H](NC(=O)C2(F)CC2)C(C)(C)C)c(OCCOCCOCCNC(=O)c2ccc(-c3ccc4c(c3)COC43CCN(C(=O)c4cc5c6c(c(N)nc5cn4)COC6)C3)cn2)c1] |
| B9 | 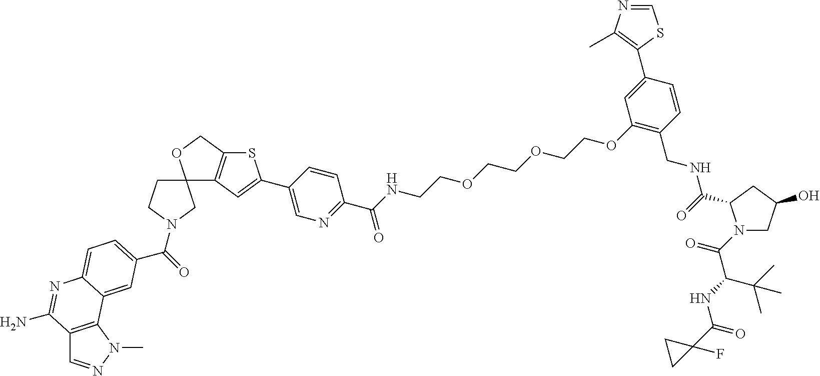 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3cc4c(s3)COC43CCN(C(=O)c4ccc5nc(N)c6cnn(C)c6c5c4)C3)cn2)c1] |
| B10 | 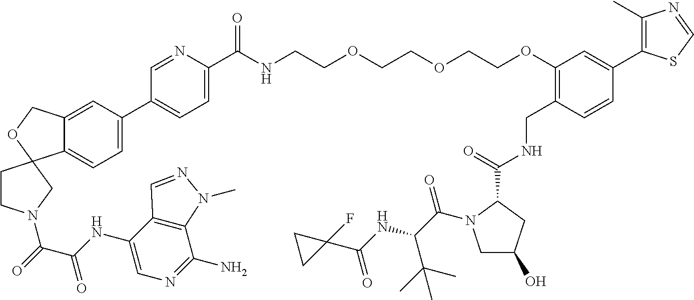 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)COC43CCN(C(=O)C(=O)Nc4cnc(N)c5c4cnn5C)C3)cn2)c1] |
| B11 | 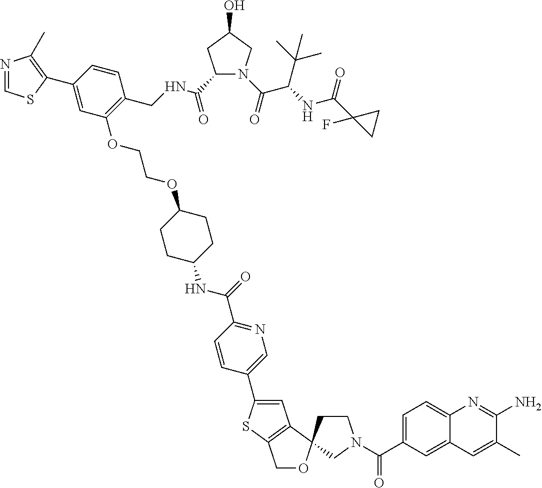 [SMILES: Cc1cc2cc(C(=O)N3CC[C@@]4(C3)OCc3sc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)cc34)ccc2nc1N] |
| B12 | 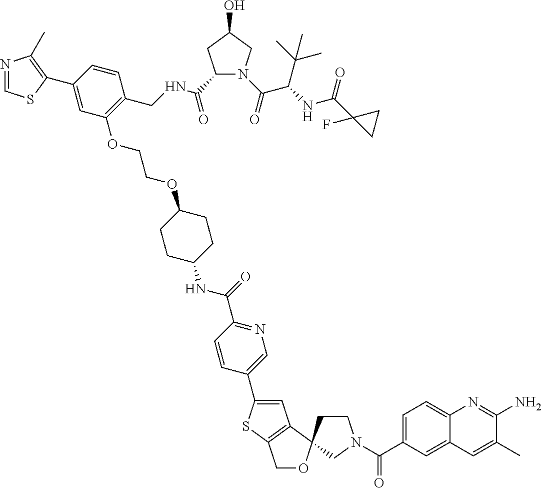 [SMILES: Cc1cc2cc(C(=O)N3CC[C@@]4(C3)OCc3sc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)cc34)ccc2nc1N] |
| B13 | 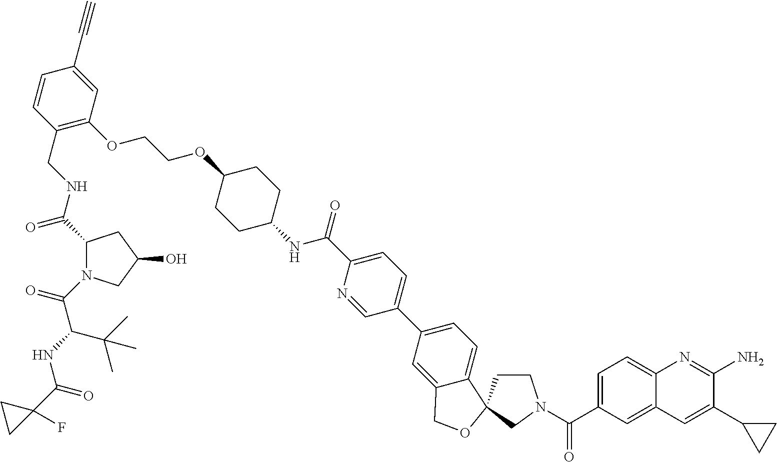 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)CO[C@@]54CCN(C(=O)c5ccc6nc(N)c(C7CC7)cc6c5)C4)cn3)CC2)c1] |
| B14 | 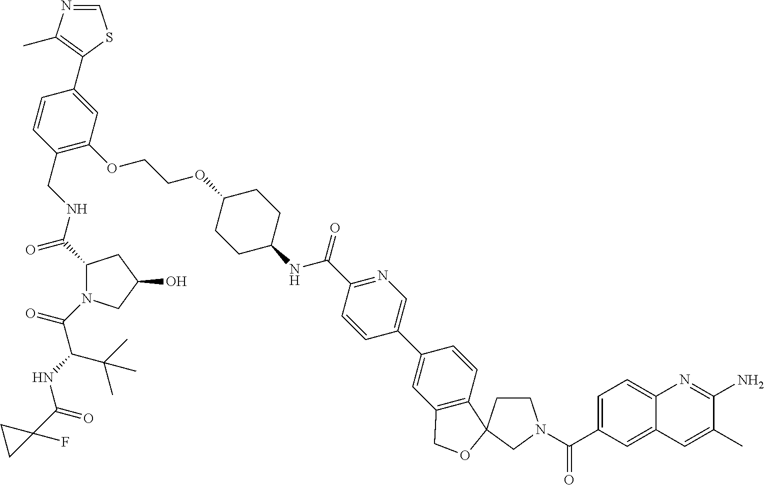 [SMILES: Cc1cc2cc(C(=O)N3CCC4(C3)OCc3cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc34)ccc2nc1N] |
| B15 | 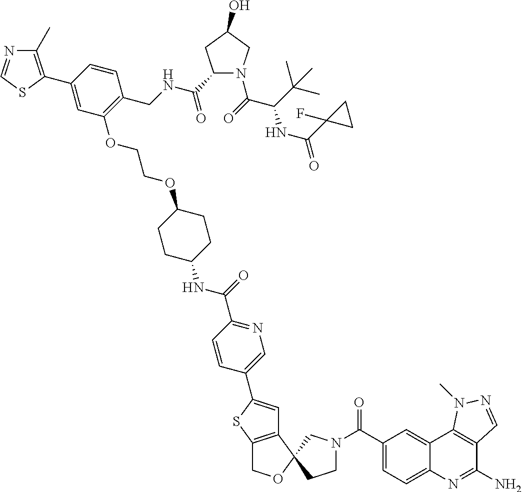 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4cc5c(s4)CO[C@]54CCN(C(=O)c5ccc6nc(N)c7cnn(C)c7c6c5)C4)cn3)CC2)c1] |
| B16 | 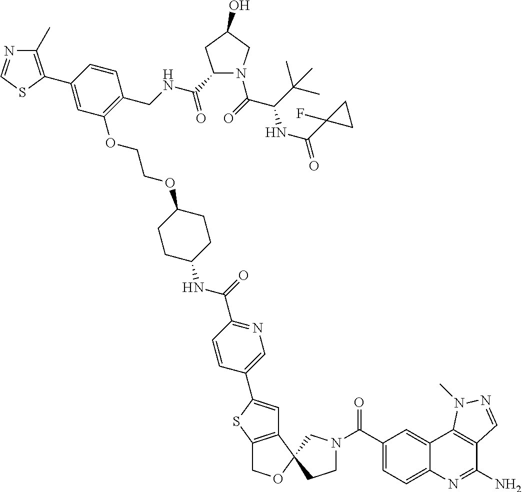 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4cc5c(s4)CO[C@]54CCN(C(=O)c5ccc6nc(N)c7cnn(C)c7c6c5)C4)cn3)CC2)c1] |
| B17 | 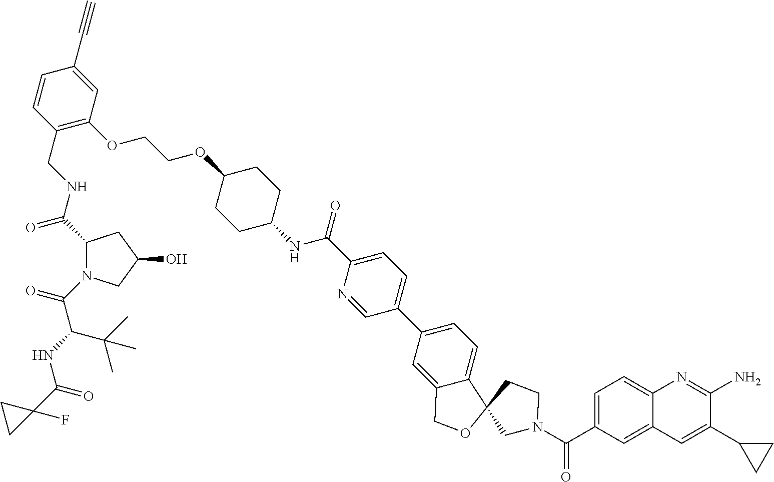 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)CO[C@]54CCN(C(=O)c5ccc6nc(N)c(C7CC7)cc6c5)C4)cn3)CC2)c1] |
| B18 | 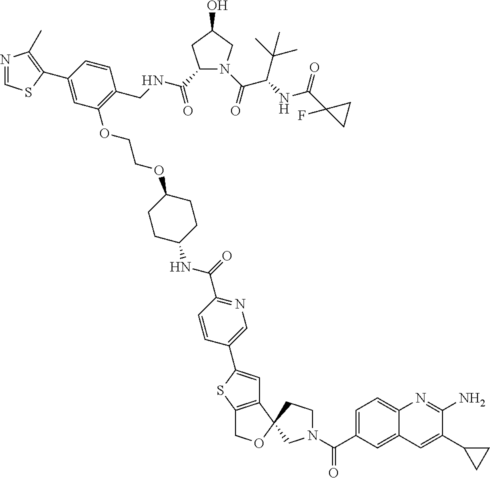 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4cc5c(s4)CO[C@]54CCN(C(=O)c5ccc6nc(N)c(C7CC7)cc6c5)C4)cn3)CC2)c1] |
| B19 | 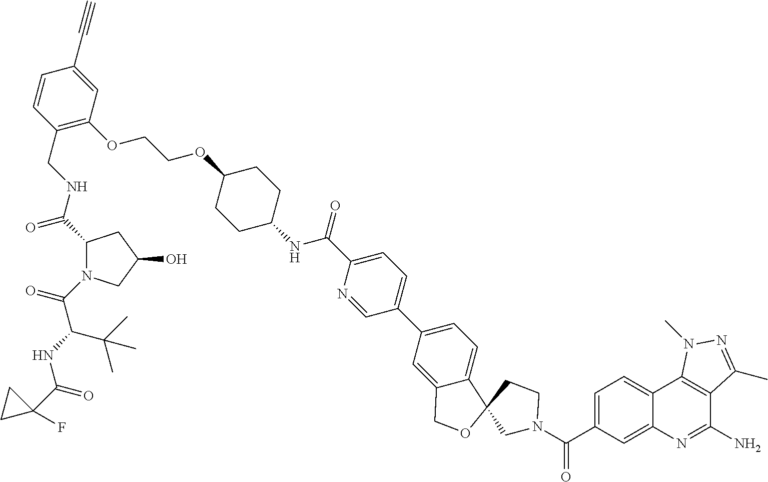 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)CO[C@]54CCN(C(=O)c5ccc6c(c5)nc(N)c5c(C)nn(C)c56)C4)cn3)CC2)c1] |
| B20 | 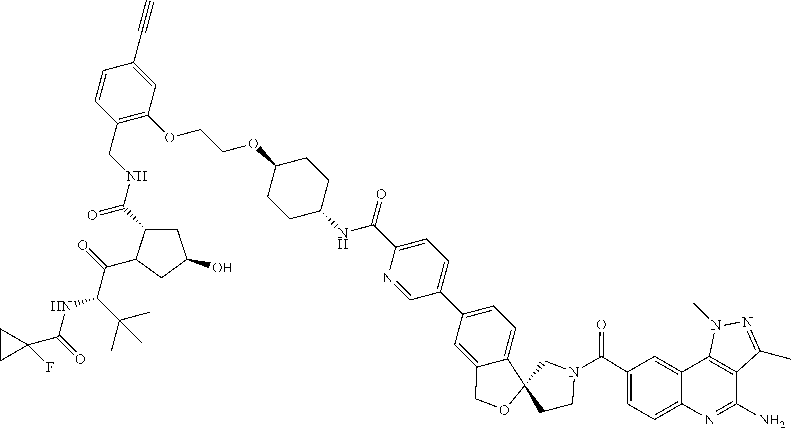 [SMILES: C#Cc1ccc(CNC(=O)[C@@H]2C[C@@H](O)CC2C(=O)[C@@H](NC(=O)C2(F)CC2)C(C)(C)C)c(OCCO[C@H]2CC[C@H](NC(=O)c3ccc(-c4ccc5c(c4)CO[C@]54CCN(C(=O)c5ccc6nc(N)c7c(C)nn(C)c7c6c5)C4)cn3)CC2)c1] |
| B21 | 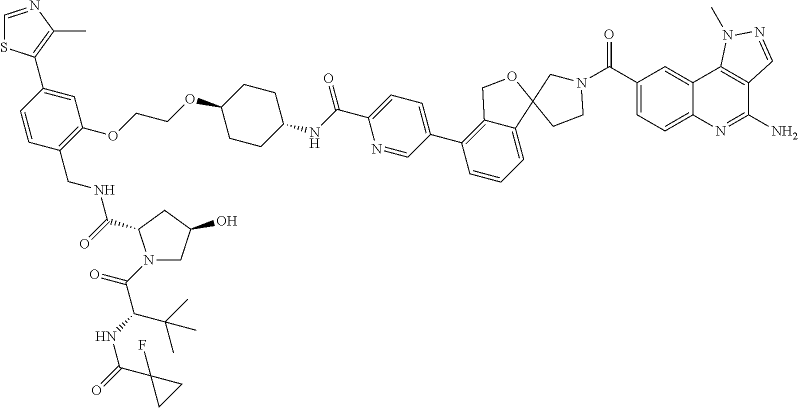 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4cccc5c4COC54CCN(C(=O)c5ccc6nc(N)c7cnn(C)c7c6c5)C4)cn3)CC2)c1] |
| B22 | 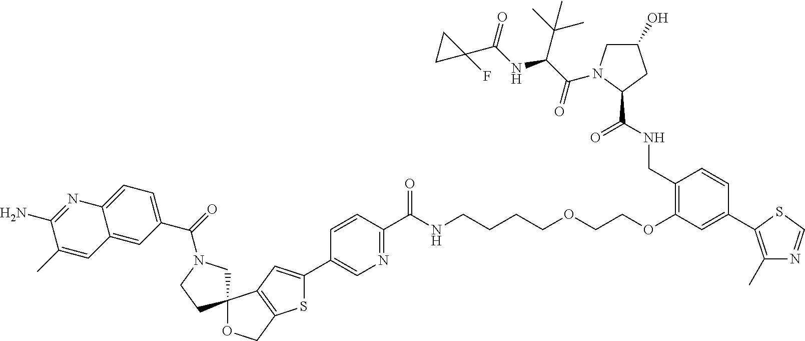 [SMILES: Cc1cc2cc(C(=O)N3CC[C@]4(C3)OCc3sc(-c5ccc(C(=O)NCCCCOCCOc6cc(-c7scnc7C)ccc6CNC(=O)[C@@H]6C[C@@H](O)CN6C(=O)[C@@H](NC(=O)C6(F)CC6)C(C)(C)C)nc5)cc34)ccc2nc1N] |
| B23 | 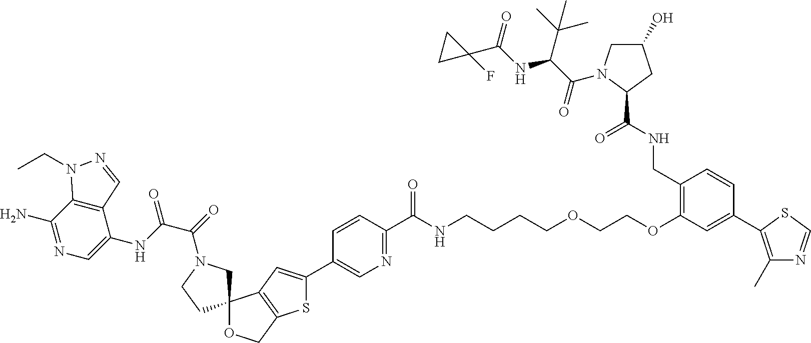 [SMILES: CCn1ncc2c(NC(=O)C(=O)N3CC[C@@]4(C3)OCc3sc(-c5ccc(C(=O)NCCCCOCCOc6cc(-c7scnc7C)ccc6CNC(=O)[C@@H]6C[C@@H](O)CN6C(=O)[C@@H](NC(=O)C6(F)CC6)C(C)(C)C)nc5)cc34)cnc(N)c21] |
| B24 | 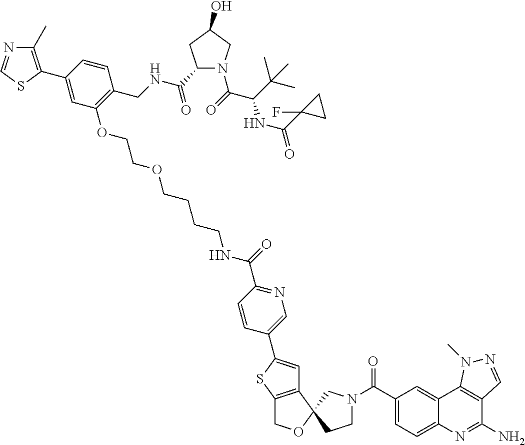 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3cc4c(s3)CO[C@]43CCN(C(=O)c4ccc5nc(N)c6cnn(C)c6c5c4)C3)cn2)c1] |
| B25 | 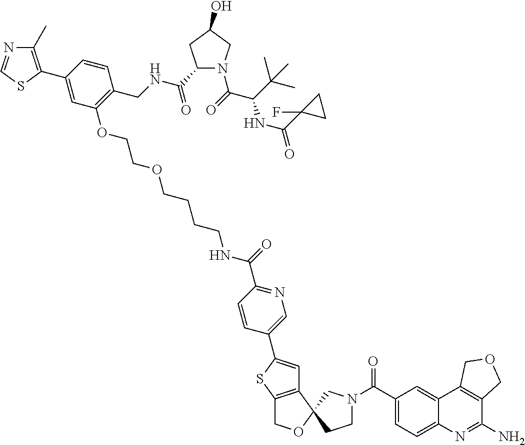 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3cc4c(s3)CO[C@]43CCN(C(=O)c4ccc5nc(N)c6c(c5c4)COC6)C3)cn2)c1] |
| B26 | 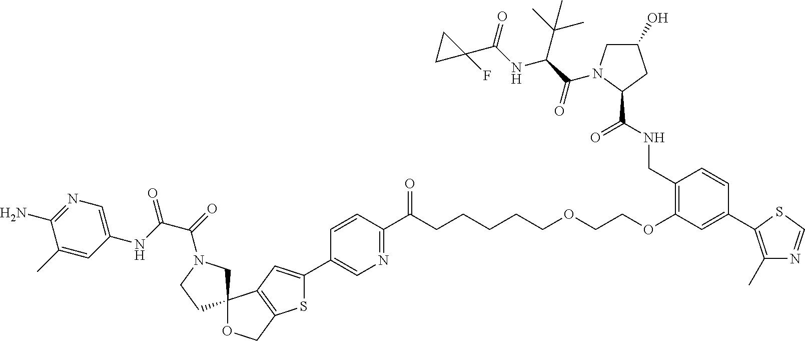 [SMILES: Cc1cc(NC(=O)C(=O)N2CC[C@@]3(C2)OCc2sc(-c4ccc(C(=O)CCCCCOCCOc5cc(-c6scnc6C)ccc5CNC(=O)[C@@H]5C[C@@H](O)CN5C(=O)[C@@H](NC(=O)C5(F)CC5)C(C)(C)C)nc4)cc23)cnc1N] |
| B27 | 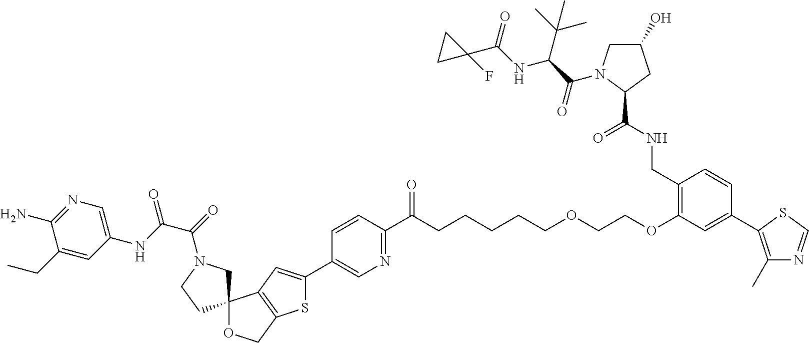 [SMILES: CCc1cc(NC(=O)C(=O)N2CC[C@@]3(C2)OCc2sc(-c4ccc(C(=O)CCCCCOCCOc5cc(-c6scnc6C)ccc5CNC(=O)[C@@H]5C[C@@H](O)CN5C(=O)[C@@H](NC(=O)C5(F)CC5)C(C)(C)C)nc4)cc23)cnc1N] |
| B28 | 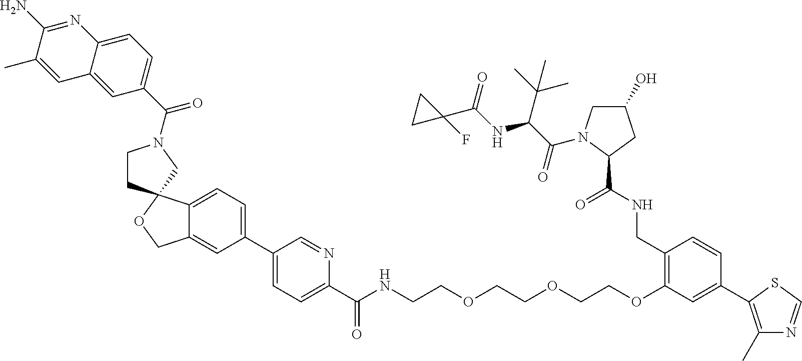 [SMILES: Cc1cc2cc(C(=O)N3CC[C@]4(C3)OCc3cc(-c5ccc(C(=O)NCCOCCOCCOc6cc(-c7scnc7C)ccc6CNC(=O)[C@@H]6C[C@@H](O)CN6C(=O)[C@@H](NC(=O)C6(F)CC6)C(C)(C)C)nc5)ccc34)ccc2nc1N] |
| B29 | 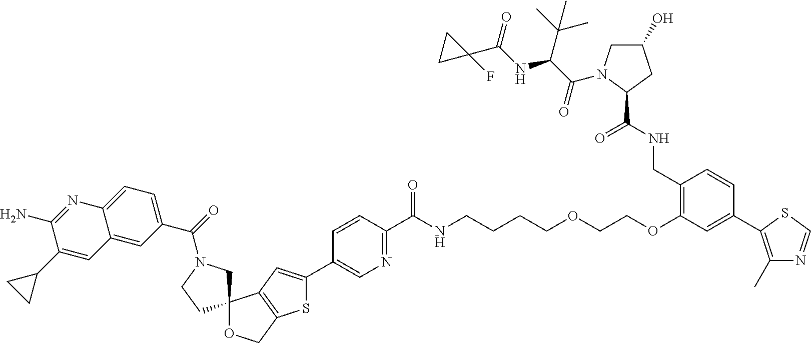 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3cc4c(s3)CO[C@]43CCN(C(=O)c4ccc5nc(N)c(C6CC6)cc5c4)C3)cn2)c1] |
| B30 | 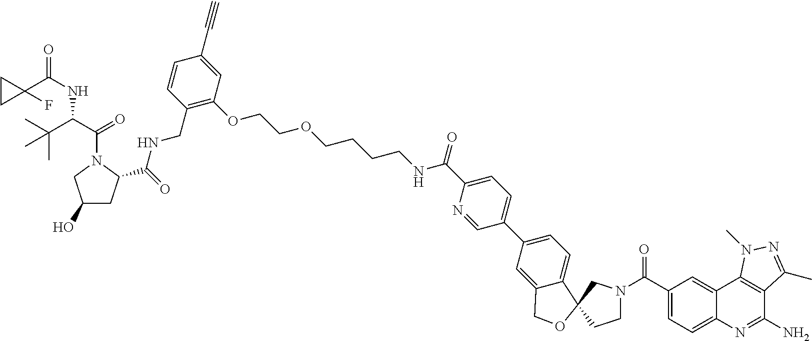 [SMILES: C#Cc1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)CO[C@@]43CCN(C(=O)c4ccc5nc(N)c6c(C)nn(C)c6c5c4)C3)cn2)c1] |
| B31 | 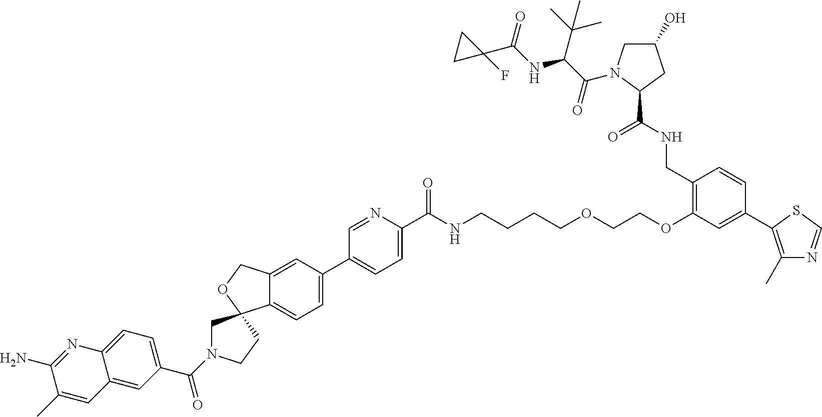 [SMILES: Cc1cc2cc(C(=O)N3CC[C@]4(C3)OCc3cc(-c5ccc(C(=O)NCCCCOCCOc6cc(-c7scnc7C)ccc6CNC(=O)[C@@H]6C[C@@H](O)CN6C(=O)[C@@H](NC(=O)C6(F)CC6)C(C)(C)C)nc5)ccc34)ccc2nc1N] |
| B32 | 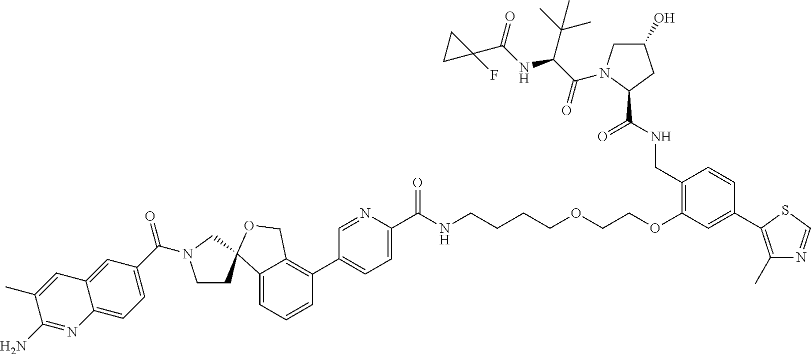 [SMILES: Cc1cc2cc(C(=O)N3CC[C@@]4(C3)OCc3c(-c5ccc(C(=O)NCCCCOCCOc6cc(-c7scnc7C)ccc6CNC(=O)[C@@H]6C[C@@H](O)CN6C(=O)[C@@H](NC(=O)C6(F)CC6)C(C)(C)C)nc5)cccc34)ccc2nc1N] |
| B33 | 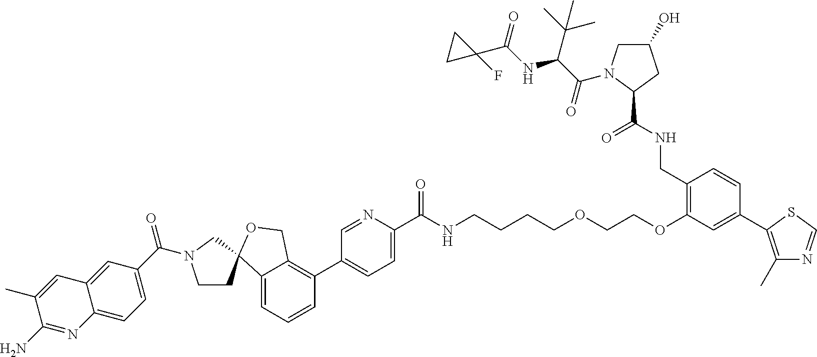 [SMILES: Cc1cc2cc(C(=O)N3CC[C@@]4(C3)OCc3c(-c5ccc(C(=O)NCCCCOCCOc6cc(-c7scnc7C)ccc6CNC(=O)[C@@H]6C[C@@H](O)CN6C(=O)[C@@H](NC(=O)C6(F)CC6)C(C)(C)C)nc5)cccc34)ccc2nc1N] |
| B34 | 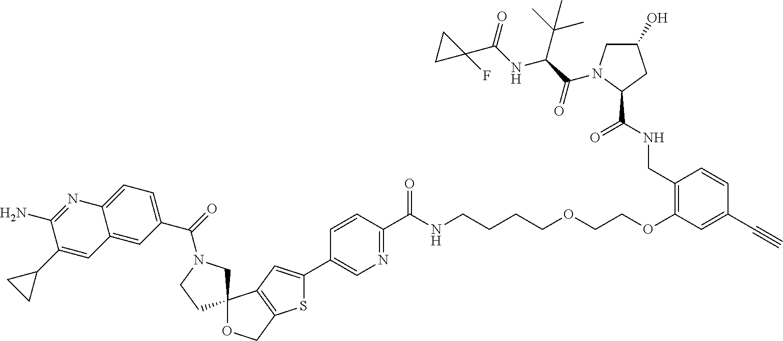 [SMILES: C#Cc1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3cc4c(s3)CO[C@]43CCN(C(=O)c4ccc5nc(N)c(C6CC6)cc5c4)C3)cn2)c1] |
| B35 | 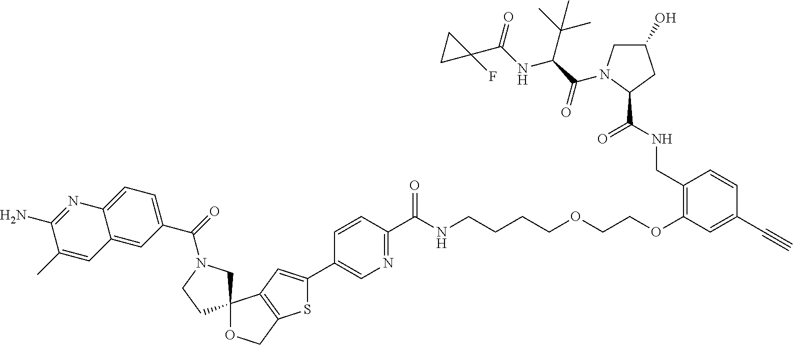 [SMILES: C#Cc1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3cc4c(s3)CO[C@]43CCN(C(=O)c4ccc5nc(N)c(C)cc5c4)C3)cn2)c1] |
| B36 | 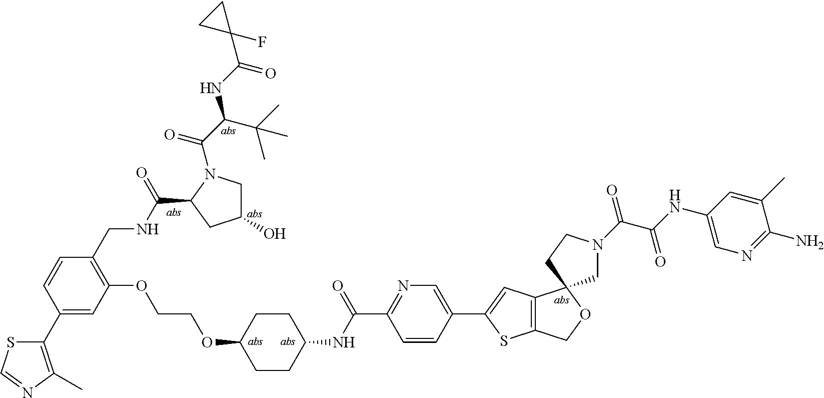 [SMILES: Cc1cc(NC(=O)C(=O)N2CC[C@@]3(C2)OCc2sc(-c4ccc(C(=O)N[C@H]5CC[C@H](OCCOc6cc(-c7scnc7C)ccc6CNC(=O)[C@@H]6C[C@@H](O)CN6C(=O)[C@@H](NC(=O)C6(F)CC6)C(C)(C)C)CC5)nc4)cc23)cnc1N] |
| B37 | 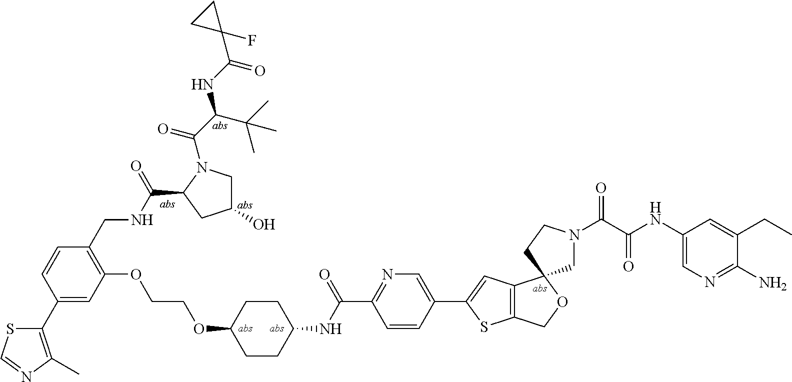 [SMILES: CCc1cc(NC(=O)C(=O)N2CC[C@@]3(C2)OCc2sc(-c4ccc(C(=O)N[C@H]5CC[C@H](OCCOc6cc(-c7scnc7C)ccc6CNC(=O)[C@@H]6C[C@@H](O)CN6C(=O)[C@@H](NC(=O)C6(F)CC6)C(C)(C)C)CC5)nc4)cc23)cnc1N] |
| B38 | 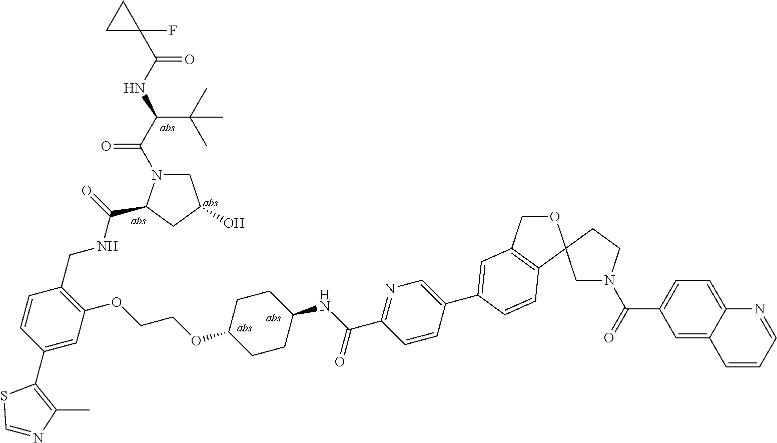 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)COC54CCN(C(=O)c5ccc6ncccc6c5)C4)cn3)CC2)c1] |
| B39 | 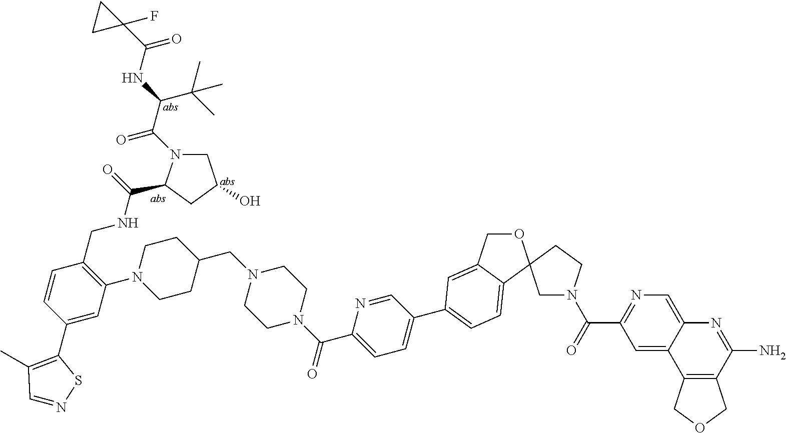 [SMILES: Cc1cnsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)COC65CCN(C(=O)c6cc7c8c(c(N)nc7cn6)COC8)C5)cn4)CC3)CC2)c1] |
| B40 | 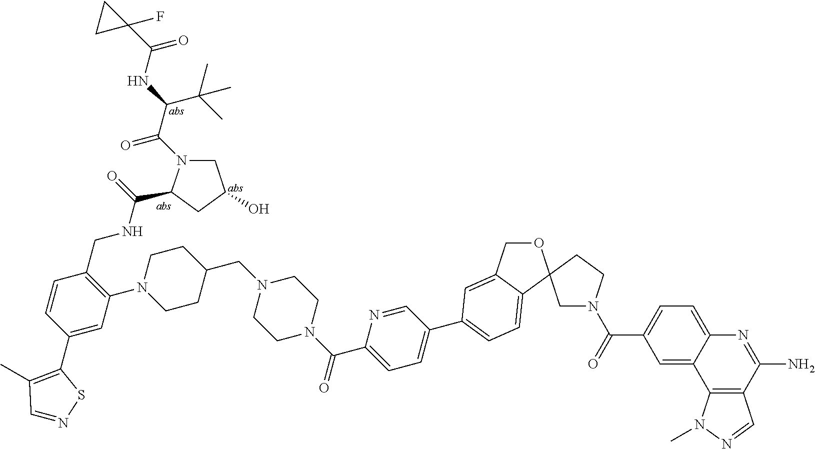 [SMILES: Cc1cnsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)COC65CCN(C(=O)c6ccc7nc(N)c8cnn(C)c8c7c6)C5)cn4)CC3)CC2)c1] |
| B41 | 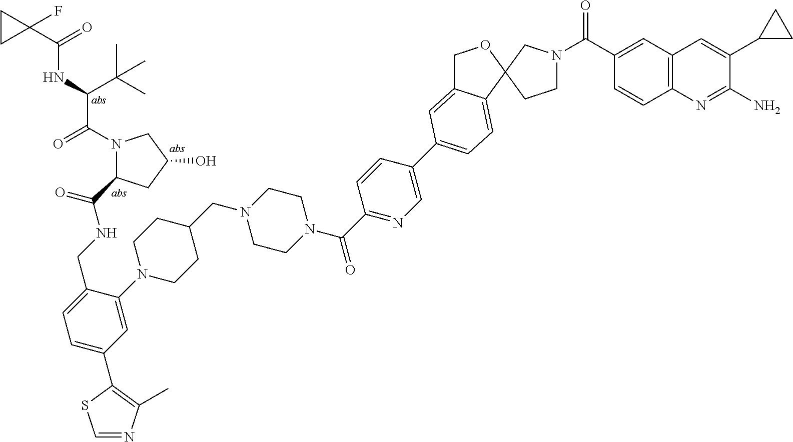 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)COC65CCN(C(=O)c6ccc7nc(N)c(C8CC8)cc7c6)C5)cn4)CC3)CC2)c1] |
| B42 | 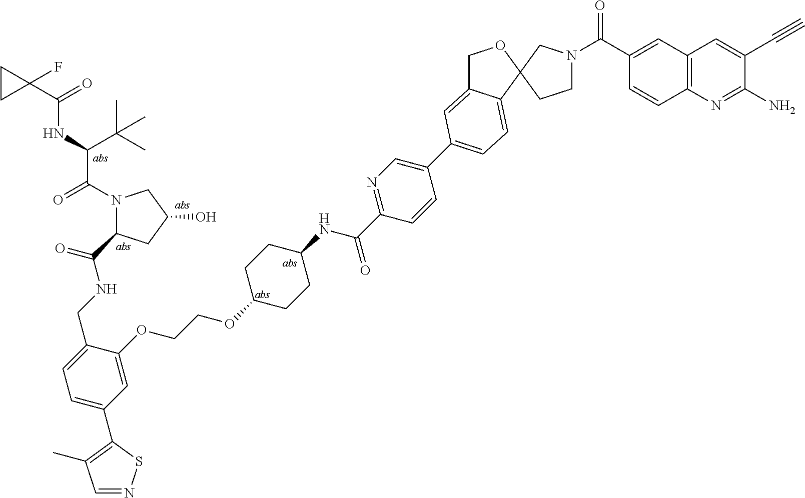 [SMILES: C#Cc1cc2cc(C(=O)N3CCC4(C3)OCc3cc(-c5ccc(C(=O)N[C@H]6CC[C@H](OCCOc7cc(-c8sncc8C)ccc7CNC(=O)[C@@H]7C[C@@H](O)CN7C(=O)[C@@H](NC(=O)C7(F)CC7)C(C)(C)C)CC6)nc5)ccc34)ccc2nc1N] |
| B43 | 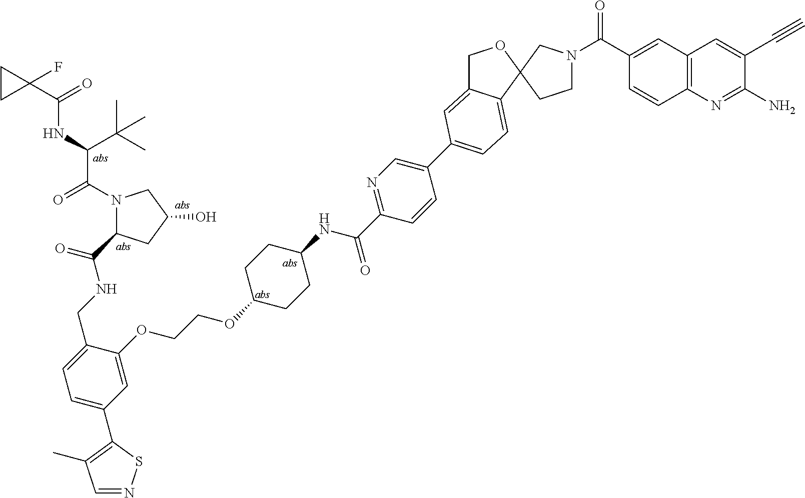 [SMILES: C#Cc1cc2cc(C(=O)N3CCC4(C3)OCc3cc(-c5ccc(C(=O)N[C@H]6CC[C@H](OCCOc7cc(-c8sncc8C)ccc7CNC(=O)[C@@H]7C[C@@H](O)CN7C(=O)[C@@H](NC(=O)C7(F)CC7)C(C)(C)C)CC6)nc5)ccc34)ccc2nc1N] |
| B44 | 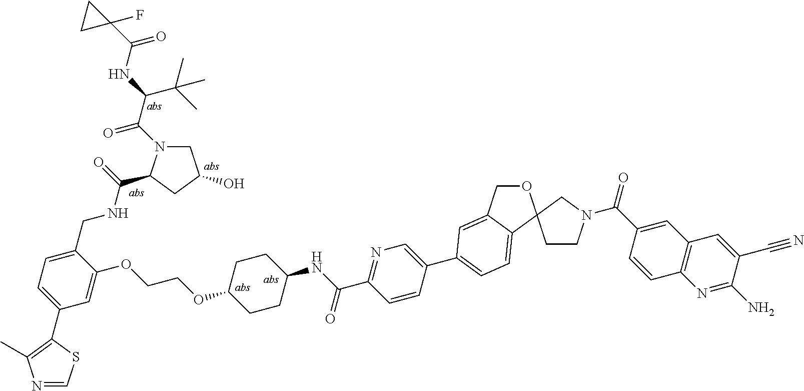 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)COC54CCN(C(=O)c5ccc6nc(N)c(C#N)cc6c5)C4)cn3)CC2)c1] |
| B45 | 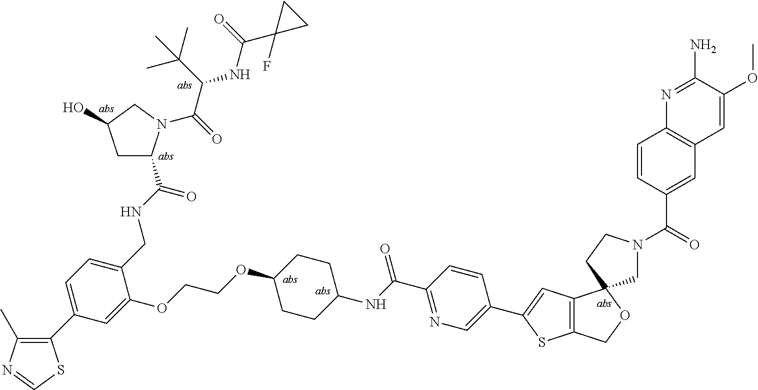 |
| B46 | 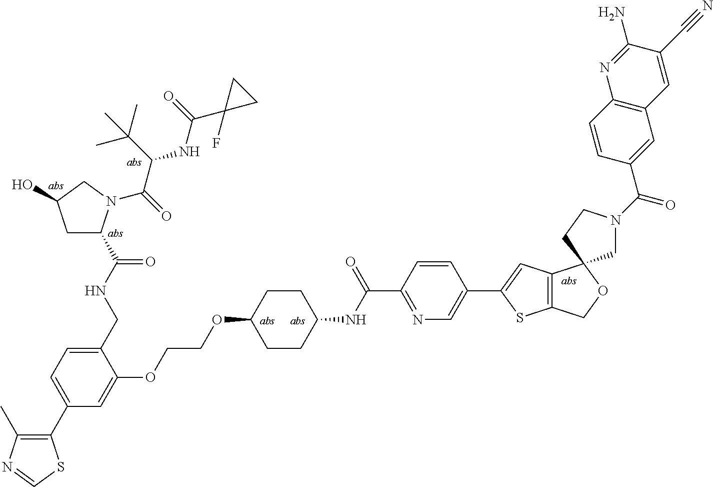 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4cc5c(s4)CO[C@]54CCN(C(=O)c5ccc6nc(N)c(C#N)cc6c5)C4)cn3)CC2)c1] |

| Example # | Chemical Name |
|---|---|
| B1 | 1′-(4-amino-1-methyl-1H-pyrazolo[4,3- |
| | c]quinoline-8-carbonyl)-N-(17-(2-(((2S,4R)-1- |
| | ((S)-2-(1-fluorocyclopropane-1-carboxamido)- |
| | 3,3-dimethylbutanoyl)-4-hydroxypyrrolidine- |
| | 2-carboxamido)methyl)-5-(4-methylthiazol-5- |
| | yl)phenoxy)-3,6,9,12,15-pentaoxaheptadecyl)- |
| | 3H-spiro[isobenzofuran-1,3′-pyrrolidine]-5- |
| | carboxamide |
| B2 | 1′-(4-amino-1-methyl-1H-pyrazolo[4,3- |
| | c]quinoline-8-carbonyl)-N-(14-(2-(((2S,4R)-1- |
| | ((S)-2-(1-fluorocyclopropane-1-carboxamido)- |
| | 3,3-dimethylbutanoyl)-4-hydroxypyrrolidine- |
| | 2-carboxamido)methyl)-5-(4-methylthiazol-5- |
| | yl)phenoxy)-3,6,9,12-tetraoxatetradecyl)-3H- |
| | spiro[isobenzofuran-1,3′-pyrrolidine]-5- |
| | carboxamide |
| B3 | 1′-(4-amino-1-methyl-1H-pyrazolo[4,3- |
| | c]quinoline-8-carbonyl)-N-(14-(2-(((2S,4R)-1- |
| | ((S)-2-(1-fluorocyclopropane-1-carboxamido)- |
| | 3,3-dimethylbutanoyl)-4-hydroxypyrrolidine- |
| | 2-carboxamido)methyl)-5-(4-methylthiazol-5- |
| | yl)phenoxy)tetradecyl)-3H- |
| | spiro[isobenzofuran-1,3′-pyrrolidine]-5- |
| | carboxamide |
| B4 | 5-(1′-(2-amino-3-methylquinoline-6-carbonyl)- |
| | 3H-spiro[isobenzofuran-1,3′-pyrrolidin]-5-yl)- |
| | N-(2-(2-(2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)ethoxy)ethyl)picolinamide |
| B5 | 5-(1′-(2-amino-3-methylquinoline-6-carbonyl)- |
| | 6′H-spiro[pyrrolidine-3,4′-thieno[2,3-c]furan]- |
| | 2′-yl)-N-(2-(2-(2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)ethoxy)ethyl)picolinamide |
| B6 | 5-(1′-(4-amino-1-methyl-1H-pyrazolo[4,3- |
| | c]quinoline-8-carbonyl)-3H- |
| | spiro[isobenzofuran-1,3′-pyrrolidin]-5-yl)-N- |
| | (2-(2-(2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)ethoxy)ethyl)picolinamide |
| B7 | 5-(1-(2-((7-amino-1-methyl-1H-pyrazolo[3,4- |
| | c]pyridin-4-yl)amino)-2-oxoacetyl)-6′H- |
| | spiro[pyrrolidine-3,4′-thieno[2,3-c]furan]-2′- |
| | yl)-N-(2-(2-(2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)ethoxy)ethyl)picolinamide |
| B8 | 5-(1′-(4-amino-1,3-dihydrofuro[3,4- |
| | c][1,7]naphthyridine-8-carbonyl)-3H- |
| | spiro[isobenzofuran-1,3′-pyrrolidin]-5-yl)-N- |
| | (2-(2-(2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)ethoxy)ethyl)picolinamide |
| B9 | 5-(1-(4-amino-1-methyl-1H-pyrazolo[4,3- |
| | c]quinoline-8-carbonyl)-6′H-spiro[pyrrolidine- |
| | 3,4′thieno[2,3-c]furan]-2′-yl)-N-(2-(2-(2-(2- |
| | (((2S,4R)-1-((S)-2-(1-fluorocyclopropane-1- |
| | carboxamido)-3,3-dimethylbutanoyl)-4- |
| | hydroxypyrrolidine-2-carboxamido)methyl)-5- |
| | (4-methylthiazol-5- |
| | yl)phenoxy)ethoxy)ethoxy)ethyl)picolinamide |
| B10 | 5-(1′-(2-((7-amino-1-methyl-1H-pyrazolo[3,4- |
| | c]pyridin-4-yl)amino)-2-oxoacetyl)-3H- |
| | spiro[isobenzofuran-1,3′-pyrrolidin]-5-yl)-N- |
| | (2-(2-(2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)ethoxy)ethyl)picolinamide |
| B11 | 5-((R)-1-(2-amino-3-methylquinoline-6- |
| | carbonyl)-6′H-spiro[pyrrolidine-3,4′- |
| | thieno[2,3-c]furan]-2′-yl)-N-((1S,4R)-4-(2-(2- |
| | (((2S,4R)-1-((S)-2-(1-fluorocyclopropane-1- |
| | carboxamido)-3,3-dimethylbutanoyl)-4- |
| | hydroxypyrrolidine-2-carboxamido)methyl)-5- |
| | (4-methylthiazol-5- |
| | yl)phenoxy)ethoxy)cyclohexyl)picolinamide |
| B12 | 5-((S)-1-(2-amino-3-methylquinoline-6- |
| | carbonyl)-6′H-spiro[pyrrolidine-3,4′- |
| | thieno[2,3-c]furan]-2′-yl)-N-((1S,4S)-4-(2-(2- |
| | (((2S,4R)-1-((S)-2-(1-fluorocyclopropane-1- |
| | carboxamido)-3,3-dimethylbutanoyl)-4- |
| | hydroxypyrrolidine-2-carboxamido)methyl)-5- |
| | (4-methylthiazol-5- |
| | yl)phenoxy)ethoxy)cyclohexyl)picolinamide |
| B13 | 5-((S)-1′-(2-amino-3-cyclopropylquinoline-6- |
| | carbonyl)-3H-spiro[isobenzofuran-1,3′- |
| | pyrrolidin]-5-yl)-N-((1S,4S)-4-(2-(5-ethynyl- |
| | 2-(((2S,4R)-1-((S)-2-(1-fluorocyclopropane-1- |
| | carboxamido)-3,3-dimethylbutanoyl)-4- |
| | hydroxypyrrolidine-2- |
| | carboxamido)methyl)phenoxy)ethoxy) |
| | cyclohexyl)picolinamide |
| B14 | 5-(1′-(2-amino-3-methylquinoline-6-carbonyl)- |
| | 3H-spiro[isobenzofuran-1,3′-pyrrolidin]-5-yl)- |
| | N-((1S,4r)-4-(2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)cyclohexyl)picolinamide |
| B15 | 5-((R)-1-(4-amino-1-methyl-1H-pyrazolo[4,3- |
| | c]quinoline-8-carbonyl)-6′H-spiro[pyrrolidine- |
| | 3,4′-thieno[2,3-c]furan]-2′-yl)-N-((1S,4R)-4- |
| | (2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)cyclohexyl)picolinamide |
| B16 | 5-((S)-1-(4-amino-1-methyl-1H-pyrazolo[4,3- |
| | c]quinoline-8-carbonyl)-6′H-spiro[pyrrolidine- |
| | 3,4′-thieno[2,3-c]furan]-2′-yl)-N-((1S,4S)-4- |
| | (2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)cyclohexyl)picolinamide |
| B17 | 5-((R)-1′-(2-amino-3-cyclopropylquinoline-6- |
| | carbonyl)-3H-spiro[isobenzofuran-1,3′- |
| | pyrrolidin]-5-yl)-N-((1S,4R)-4-(2-(5-ethynyl- |
| | 2-(((2S,4R)-1-((S)-2-(1-fluorocyclopropane-1- |
| | carboxamido)-3,3-dimethylbutanoyl)-4- |
| | hydroxypyrrolidine-2- |
| | carboxamido)methyl)phenoxy)ethoxy) |
| | cyclohexyl)picolinamide |
| B18 | 5-((R)-1-(2-amino-3-cyclopropylquinoline-6- |
| | carbonyl)-6′H-spiro[pyrrolidine-3,4′- |
| | thieno[2,3-c]furan]-2′-yl)-N-((1S,4R)-4-(2-(2- |
| | (((2S,4R)-1-((S)-2-(1-fluorocyclopropane-1- |
| | carboxamido)-3,3-dimethylbutanoyl)-4- |
| | hydroxypyrrolidine-2-carboxamido)methyl)-5- |
| | (4-methylthiazol-5- |
| | yl)phenoxy)ethoxy)cyclohexyl)picolinamide |
| B19 | 5-((S)-1′-(4-amino-1,3-dimethyl-1H- |
| | pyrazolo[4,3-c]quinoline-8-carbonyl)-3H- |
| | spiro[isobenzofuran-1,3′-pyrrolidin]-5-yl)-N- |
| | ((1S,4S)-4-(2-(5-ethynyl-2-(((2S,4R)-1-((S)-2- |
| | (1-fluorocyclopropane-1-carboxamido)-3,3- |
| | dimethylbutanoyl)-4-hydroxypyrrolidine-2- |
| | carboxamido)methyl)phenoxy)ethoxy) |
| | cyclohexyl)picolinamide |
| B20 | 5-((R)-1′-(4-amino-1,3-dimethyl-1H- |
| | pyrazolo[4,3-c]quinoline-8-carbonyl)-3H- |
| | spiro[isobenzofuran-1,3′-pyrrolidin]-5-yl)-N- |
| | ((1S,4R)-4-(2-(5-ethynyl-2-(((2S,4R)-1-((S)-2- |
| | (1-fluorocyclopropane-1-carboxamido)-3,3- |
| | dimethylbutanoyl)-4-hydroxypyrrolidine-2- |
| | carboxamido)methyl)phenoxy)ethoxy)cyclohexyl) |
| | picolinamide |
| B21 | 5-(1′-(4-amino-1-methyl-1H-pyrazolo[4,3- |
| | c]quinoline-8-carbonyl)-3H- |
| | spiro[isobenzofuran-1,3′-pyrrolidin]-4-yl)-N- |
| | ((1S,4r)-4-(2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)cyclohexyl)picolinamide |
| B22 | 5-((S)-1-(2-amino-3-methylquinoline-6- |
| | carbonyl)-6′H-spiro[pyrrolidine-3,4′- |
| | thieno[2,3-c]furan]-2′-yl)-N-(4-(2-(2- |
| | (((2S,4R)-1-((S)-2-(1-fluorocyclopropane-1- |
| | carboxamido)-3,3-dimethylbutanoyl)-4- |
| | hydroxypyrrolidine-2-carboxamido)methyl)-5- |
| | (4-methylthiazol-5- |
| | yl)phenoxy)ethoxy)butyl)picolinamide |
| B23 | 5-((R)-1-(2-((7-amino-1-ethyl-1H- |
| | pyrazolo[3,4-c]pyridin-4-yl)amino)-2- |
| | oxoacetyl)-6′H-spiro[pyrrolidine-3,4′- |
| | thieno[2,3-c]furan]-2′-yl)-N-(4-(2-(2- |
| | (((2S,4R)-1-((S)-2-(1-fluorocyclopropane-1- |
| | carboxamido)-3,3-dimethylbutanoyl)-4- |
| | hydroxypyrrolidine-2-carboxamido)methyl)-5- |
| | (4-methylthiazol-5- |
| | yl)phenoxy)ethoxy)butyl)picolinamide |
| B24 | 5-((R)-1-(4-amino-1-methyl-1H-pyrazolo[4,3- |
| | c]quinoline-8-carbonyl)-6′H-spiro[pyrrolidine- |
| | 3,4′-thieno[2,3-c]furan]-2′-yl)-N-(4-(2-(2- |
| | (((2S,4R)-1-((S)-2-(1-fluorocyclopropane-1- |
| | carboxamido)-3,3-dimethylbutanoyl)-4- |
| | hydroxypyrrolidine-2-carboxamido)methyl)-5- |
| | (4-methylthiazol-5- |
| | yl)phenoxy)ethoxy)butyl)picolinamide |
| B25 | 5-((R)-1-(4-amino-1,3-dihydrofuro[3,4- |
| | c][1,7]naphthyridine-8-carbonyl)-6′H- |
| | spiro[pyrrolidine-3,4′-thieno[2,3-c]furan]-2′- |
| | yl)-N-(4-(2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)butyl)picolinamide |
| B26 | 5-((R)-1-(2-((6-amino-5-methylpyridin-3- |
| | yl)amino)-2-oxoacetyl)-6′H-spiro[pyrrolidine- |
| | 3,4′-thieno[2,3-c]furan]-2′-yl)-N-(4-(2-(2- |
| | (((2S,4R)-1-((S)-2-(1-fluorocyclopropane-1- |
| | carboxamido)-3,3-dimethylbutanoyl)-4- |
| | hydroxypyrrolidine-2-carboxamido)methyl)-5- |
| | (4-methylthiazol-5- |
| | yl)phenoxy)ethoxy)butyl)picolinamide |
| B27 | 5-((R)-1-(2-((6-amino-5-ethylpyridin-3- |
| | yl)amino)-2-oxoacetyl)-6′H-spiro[pyrrolidine- |
| | 3,4′-thieno[2,3-c]furan]-2′-yl)-N-(4-(2-(2- |
| | (((2S,4R)-1-((S)-2-(1-fluorocyclopropane-1- |
| | carboxamido)-3,3-dimethylbutanoyl)-4- |
| | hydroxypyrrolidine-2-carboxamido)methyl)-5- |
| | (4-methylthiazol-5- |
| | yl)phenoxy)ethoxy)butyl)picolinamide |
| B28 | 5-((S)-1′-(2-amino-3-methylquinoline-6- |
| | carbonyl)-3H-spiro[isobenzofuran-1,3′- |
| | pyrrolidin]-5-yl)-N-(2-(2-(2-(2-(((2S,4R)-1- |
| | ((S)-2-(1-fluorocyclopropane-1-carboxamido)- |
| | 3,3-dimethylbutanoyl)-4-hydroxypyrrolidine- |
| | 2-carboxamido)methyl)-5-(4-methylthiazol-5- |
| | yl)phenoxy)ethoxy)ethoxy)ethyl)picolinamide |
| B29 | 5-((R)-1-(2-amino-3-cyclopropylquinoline-6- |
| | carbonyl)-6′H-spiro[pyrrolidine-3,4′- |
| | thieno[2,3-c]furan]-2′-yl)-N-(4-(2-(2- |
| | (((2S,4R)-1-((S)-2-(1-fluorocyclopropane-1- |
| | carboxamido)-3,3-dimethylbutanoyl)-4- |
| | hydroxypyrrolidine-2-carboxamido)methyl)-5- |
| | (4-methylthiazol-5- |
| | yl)phenoxy)ethoxy)butyl)picolinamide |
| B30 | 5-((S)-1′-(4-amino-1,3-dimethyl-1H- |
| | pyrazolo[4,3-c]quinoline-8-carbonyl)-3H- |
| | spiro[isobenzofuran-1,3′-pyrrolidin]-5-yl)-N- |
| | (4-(2-(5-ethynyl-2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)phenoxy)ethoxy)butyl) |
| | picolinamide |
| B31 | 5-((S)-1′-(2-amino-3-methylquinoline-6- |
| | carbonyl)-3H-spiro[isobenzofuran-1,3′- |
| | pyrrolidin]-5-yl)-N-(4-(2-(2-(((2S,4R)-1-((S)- |
| | 2-(1-fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)butyl)picolinamide |
| B32 | 5-((R)-1′-(2-amino-3-methylquinoline-6- |
| | carbonyl)-3H-spiro[isobenzofuran-1,3′- |
| | pyrrolidin]-4-yl)-N-(4-(2-(2-(((2S,4R)-1-((S)- |
| | 2-(1-fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)butyl)picolinamide |
| B33 | 5-((S)-1′-(2-amino-3-methylquinoline-6- |
| | carbonyl)-3H-spiro[isobenzofuran-1,3′- |
| | pyrrolidin]-4-yl)-N-(4-(2-(2-(((2S,4R)-1-((S)- |
| | 2-(1-fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)butyl)picolinamide |
| B34 | 5-((R)-1-(2-amino-3-cyclopropylquinoline-6- |
| | carbonyl)-6′H-spiro[pyrrolidine-3,4′- |
| | thieno[2,3-c]furan]-2′-yl)-N-(4-(2-(5-ethynyl- |
| | 2-(((2S,4R)-1-((S)-2-(1-fluorocyclopropane-1- |
| | carboxamido)-3,3-dimethylbutanoyl)-4- |
| | hydroxypyrrolidine-2- |
| | carboxamido)methyl)phenoxy)ethoxy)butyl) |
| | picolinamide |
| B35 | 5-((R)-1-(2-amino-3-methylquinoline-6- |
| | carbonyl)-6′H-spiro[pyrrolidine-3,4′- |
| | thieno[2,3-c]furan]-2′-yl)-N-(4-(2-(5-ethynyl- |
| | 2-(((2S,4R)-1-((S)-2-(1-fluorocyclopropane-1- |
| | carboxamido)-3,3-dimethylbutanoyl)-4- |
| | hydroxypyrrolidine-2- |
| | carboxamido)methyl)phenoxy)ethoxy)butyl) |
| | picolinamide |
| B36 | 5-((R)-1-(2-((6-amino-5-methylpyridin-3- |
| | yl)amino)-2-oxoacetyl)-6′H-spiro[pyrrolidine- |
| | 3,4′-thieno[2,3-c]furan]-2′-yl)-N-((1S,4R)-4- |
| | (2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)cyclohexyl)picolinamide |
| B37 | 5-((R)-1-(2-((6-amino-5-ethylpyridin-3- |
| | yl)amino)-2-oxoacetyl)-6′H-spiro[pyrrolidine- |
| | 3,4′-thieno[2,3-c]furan]-2′-yl)-N-((1S,4R)-4- |
| | (2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)cyclohexyl)picolinamide |
| B38 | N-((1S,4r)-4-(2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)cyclohexyl)-5-(1′- |
| | (quinoline-6-carbonyl)-3H- |
| | spiro[isobenzofuran-1,3′-pyrrolidin]-5- |
| | yl)picolinamide |
| B39 | (2S,4R)-N-(2-(4-((4-(5-(1′-(4-amino-1,3- |
| | dihydrofuro[3,4-c][1,7]naphthyridine-8- |
| | carbonyl)-3H-spiro[isobenzofuran-1,3′- |
| | pyrrolidin]-5-yl)picolinoyl)piperazin-1- |
| | yl)methyl)piperidin-1-yl)-4-(4-methylthiazol- |
| | 5-yl)benzyl)-1-((S)-2-(1-fluorocyclopropane- |
| | 1-carboxamido)-3,3-dimethylbutanoyl)-4- |
| | hydroxypyrrolidine-2-carboxamide |
| B40 | (2S,4R)-N-(2-(4-((4-(5-(1′-(4-amino-1-methyl- |
| | 1H-pyrazolo[4,3-c]quinoline-8-carbonyl)-3H- |
| | spiro[isobenzofuran-1,3′-pyrrolidin]-5- |
| | yl)picolinoyl)piperazin-1-yl)methyl)piperidin- |
| | 1-yl)-4-(4-methylthiazol-5-yl)benzyl)-1-((S)- |
| | 2-(1-fluorocyclopropane-1-carboxamido)-3,3- |
| | dimethylbutanoyl)-4-hydroxypyrrolidine-2- |
| | carboxamide |
| B41 | (2S,4R)-N-(2-(4-((4-(5-(1′-(2-amino-3- |
| | cyclopropylquinoline-6-carbonyl)-3H- |
| | spiro[isobenzofuran-1,3′-pyrrolidin]-5- |
| | yl)picolinoyl)piperazin-1-yl)methyl)piperidin- |
| | 1-yl)-4-(4-methylthiazol-5-yl)benzyl)-1-((S)- |
| | 2-(1-fluorocyclopropane-1-carboxamido)-3,3- |
| | dimethylbutanoyl)-4-hydroxypyrrolidine-2- |
| | carboxamide |
| B42 | 5-(1′-(2-amino-3-ethynylquinoline-6- |
| | carbonyl)-3H-spiro[isobenzofuran-1,3′- |
| | pyrrolidin]-5-yl)-N-((1S,4r)-4-(2-(2-(((2S,4R)- |
| | 1-((S)-2-(1-fluorocyclopropane-1- |
| | carboxamido)-3,3-dimethylbutanoyl)-4- |
| | hydroxypyrrolidine-2-carboxamido)methyl)-5- |
| | (4-methylthiazol-5- |
| | yl)phenoxy)ethoxy)cyclohexyl)picolinamide |
| B43 | 5-(1′-(2-amino-3-cyanoquinoline-6-carbonyl)- |
| | 3H-spiro[isobenzofuran-1,3′-pyrrolidin]-5-yl)- |
| | N-((1S,4r)-4-(2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)cyclohexyl)picolinamide |
| B44 | 5-(1′-(2-amino-3-cyanoquinoline-6-carbonyl)- |
| | 3H-spiro[isobenzofuran-1,3′-pyrrolidin]-5-yl)- |
| | N-((1S,4r)-4-(2-(2-(((2S,4R)-1-((S)-2-(1- |
| | fluorocyclopropane-1-carboxamido)-3,3- |
| | dimethylbutanoyl)-4-hydroxypyrrolidine-2- |
| | carboxamido)methyl)-5-(4-methylthiazol-5- |
| | yl)phenoxy)ethoxy)cyclohexyl)picolinamide |
| B45 | 5-((R)-1-(2-amino-3-methoxyquinoline-6- |
| | carbonyl)-6′H-spiro[pyrrolidine-3,4′- |
| | thieno[2,3-c]furan]-2′-yl)-N-((1S,4R)-4-(2-(2- |
| | (((2S,4R)-1-((S)-2-(1-fluorocyclopropane-1- |
| | carboxamido)-3,3-dimethylbutanoyl)-4- |
| | hydroxypyrrolidine-2-carboxamido)methyl)-5- |
| | (4-methylthiazol-5- |
| | yl)phenoxy)ethoxy)cyclohexyl)picolinamide |
| B46 | 5-((R)-1-(2-amino-3-cyanoquinoline-6- |
| | carbonyl)-6′H-spiro[pyrrolidine-3,4′- |
| | thieno[2,3-c]furan]-2′-yl)-N-((1S,4R)-4-(2-(2- |
| | (((2S,4R)-1-((S)-2-(1-fluorocyclopropane-1- |
| | carboxamido)-3,3-dimethylbutanoyl)-4- |
| | hydroxypyrrolidine-2-carboxamido)methyl)-5- |
| | (4-methylthiazol-5- |
| | yl)phenoxy)ethoxy)cyclohexyl)picolinamide |

The compounds of the present disclosure may possess advantageous characteristics, as compared to known compounds, such as known PRMT5 degraders. For example, the compounds of the present disclosure may display more PRMT5 activity, more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction). These beneficial properties of the compounds of the present disclosure may be measured according to methods commonly available in the art, such as methods exemplified herein.

Due to the existence of double bonds, the compounds of the present disclosure may be in cis or trans, or Z or E, configuration. It is understood that although one configuration may be depicted in the structure of the compounds or formulae of the present disclosure, the present disclosure also encompasses the other configuration. For example, the compounds or formulae of the present disclosure may be depicted in cis or trans, or Z or E, configuration.

In one embodiment, a compound of the present disclosure (e.g., a compound of any of the formulae or any individual compounds disclosed herein) is a pharmaceutically acceptable salt. In another embodiment, a compound of the present disclosure (e.g., a compound of any of the formulae or any individual compounds disclosed herein) is a solvate. In another embodiment, a compound of the present disclosure (e.g., a compound of any of the formulae or any individual compounds disclosed herein) is a hydrate.

### Pharmaceutically Acceptable Salts

In some embodiments, the compounds disclosed herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the disclosure may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates are within the scope of the disclosure.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof are contemplated and are within the scope of the present disclosure.

In some embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Isomers (Stereoisomers, Geometric Isomer, Tautomer, Etc.)

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is termed a “racemic mixture” or “racemate”.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In some embodiments, the weights are based upon total weight of all enantiomers or stereoisomers of the compound.

As used herein and unless otherwise indicated, the term “enantiomerically pure (R)-compound” refers to at least about 95% by weight (R)-compound and at most about 5% by weight (S)-compound, at least about 99% by weight (R)-compound and at most about 1% by weight (S)-compound, or at least about 99.9% by weight (R)-compound and at most about 0.1% by weight (S)-compound. In some embodiments, the weights are based upon total weight of compound.

As used herein and unless otherwise indicated, the term “enantiomerically pure (S)-compound” refers to at least about 95% by weight (S)-compound and at most about 5% by weight (R)-compound, at least about 99% by weight (S)-compound and at most about 1% by weight (R)-compound or at least about 99.9% by weight (S)-compound and at most about 0.1% by weight (R)-compound. In some embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure (R)-compound can comprise, for example, about 90% excipient and about 10% enantiomerically pure (R)-compound. In some embodiments, the enantiomerically pure (R)-compound in such compositions can, for example, comprise, at least about 95% by weight (R)-compound and at most about 5% by weight (S)-compound, by total weight of the compound. For example, a pharmaceutical composition comprising enantiomerically pure (S)-compound can comprise, for example, about 90% excipient and about 10% enantiomerically pure (S)-compound. In some embodiments, the enantiomerically pure (S)-compound in such compositions can, for example, comprise, at least about 95% by weight (S)-compound and at most about 5% by weight (R)-compound, by total weight of the compound. In some embodiments, the active ingredient can be formulated with little or no excipient or carrier.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

In some embodiments, the compounds described herein exist as geometric isomers. In some embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the disclosure.

In some embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the disclosure.

In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Tautomers

In some embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest. All tautomeric forms of the compounds disclosed herein are contemplated and are within the scope of the disclosure. The exact ratio of the tautomers depends on several factors, including temperature, solvent, and pH.

### Conjugates

In some aspects, the present disclosure provides conjugates comprising the compound described herein, wherein the compound is directly or indirectly attached to a conjugate partner.

In some aspects, the present disclosure provides a compound described herein, wherein the compound is conjugated to a conjugate partner.

In some embodiments, the conjugate comprises a compound described herein directly attached (e.g., via a bond) to a conjugate partner.

In some embodiments, the conjugate comprises a compound described herein indirectly attached (e.g., via a linker moiety) to a conjugate partner.

In some embodiments, the conjugate comprises one or more compound described herein.

In some embodiments, the conjugate partner is an antibody or fragment thereof.

In some embodiments, the conjugate partner is an antibody.

In some embodiments, the conjugate partner is an antibody fragment.

In some embodiments, the conjugate partner is a half-life extending moiety (i.e., a moiety that increases the circulatory half-life of the conjugate in vivo, compared to the unconjugated compound).

In some embodiments, the compound described herein is conjugated to a conjugate partner.

In some embodiments, the compound described herein is conjugated to an antibody or fragment thereof.

In some embodiments, the compound described herein is conjugated to a half-life extending moiety.

In some embodiments, the present disclosure provides a compound for conjugating with a conjugate partner.

In some embodiments, the present disclosure provides a compound for conjugating with an antibody or fragment thereof.

In some embodiments, the present disclosure provides a compound for conjugating with a half-life extending moiety.

In some embodiments, the antibody and/or linker moiety of the conjugate is described in PCT Application No. PCT/US2023/085698; Hong, B. K. et al. J. Med. Chem. 2023, 66 (1), 140-148; Dragovich, P. S. Chem. Soc. Rev. 2022, 51 (10), 3886-3897; or Poudel, Y. B. et al. J. Med. Chem. 2024, 67 (18), 15996-16001 (each incorporated herein by reference).

In some embodiments, the half-life extending moiety comprises one or more moiety each independently selected from albumin, albumin binding domain, XTEN polypeptide, and polyalkylene glycol (PAO).

In some embodiments, the half-life extending moiety is selected from albumin, albumin binding domain, XTEN polypeptide, and polyalkylene glycol (PAO).

In some embodiments, the half-life extending moiety is albumin

In some embodiments, the half-life extending moiety is albumin binding domain.

In some embodiments, the half-life extending moiety is XTEN polypeptide.

In some embodiments, the half-life extending moiety is polyalkylene glycol (PAO).

In some embodiments, the PAO comprises a polymer of alkylene oxide.

In some embodiments, the PAO comprises a polymer of ethylene oxide.

In some embodiments, the PAO comprises a polymer of propylene oxide.

In some embodiments, the PAO is polypropylene glycol, polyethylene glycol (PEG), polyethylene glycol methyl ether (mPEG), or polyoxyethylenated polyol, or a copolymer (e.g., block copolymer) thereof.

In some embodiments, the PAO is polyethylene glycol (PEG) or polyethylene glycol methyl ether (mPEG).

In some embodiments, the PAO is polyethylene glycol (PEG).

In some embodiments, the PAO is polyethylene glycol methyl ether (mPEG).

In some embodiments, the PEG is polydisperse PEG, monodisperse PEG, or discrete PEG. In some embodiments, polydisperse PEGs are heterogeneous mixtures of sizes and molecular weights, whereas monodisperse PEGs are purified from heterogeneous mixtures and provide a single chain length and molecular weight. In some embodiments, the PEG units are discrete PEGs. In some embodiments, the discrete PEGs provide a single molecule with defined and specified chain length.

In some embodiments, the half-life extending moiety is described in Binder, U. et al. Expert Opinion on Biological Therapy, 2024, 25, 93-118, or Zaman, R. et al. Journal of Controlled Release, 2019, 301 (10), 176-189 (each incorporated herein by reference).

### Pharmaceutical Compositions

In some embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, the present disclosure provides pharmaceutical compositions comprising a compound described herein, or a pharmaceutically acceptable salt, solvate, or stereoisomer thereof, and one or more pharmaceutically acceptable excipient.

In some embodiments, the compound provided herein is substantially pure, in that it contains less than about 5%, less than about 1%, or less than about 0.1% of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

### Preparation and Characterization of the Compounds

The compounds of the present disclosure can be prepared in a number of ways well known to those skilled in the art of organic synthesis. By way of example, the compounds of the present disclosure can be synthesized using the methods described below, together with synthetic methods known in the art of synthetic organic chemistry, or variations thereon as appreciated by those skilled in the art. The compounds of the present disclosure (i.e., a compound of the present application (e.g., a compound of any of the formulae or any individual compounds disclosed herein)) can be synthesized by following the general synthetic scheme below as well as the steps outlined in the examples, schemes, procedures, and/or synthesis described herein (e.g., Examples).

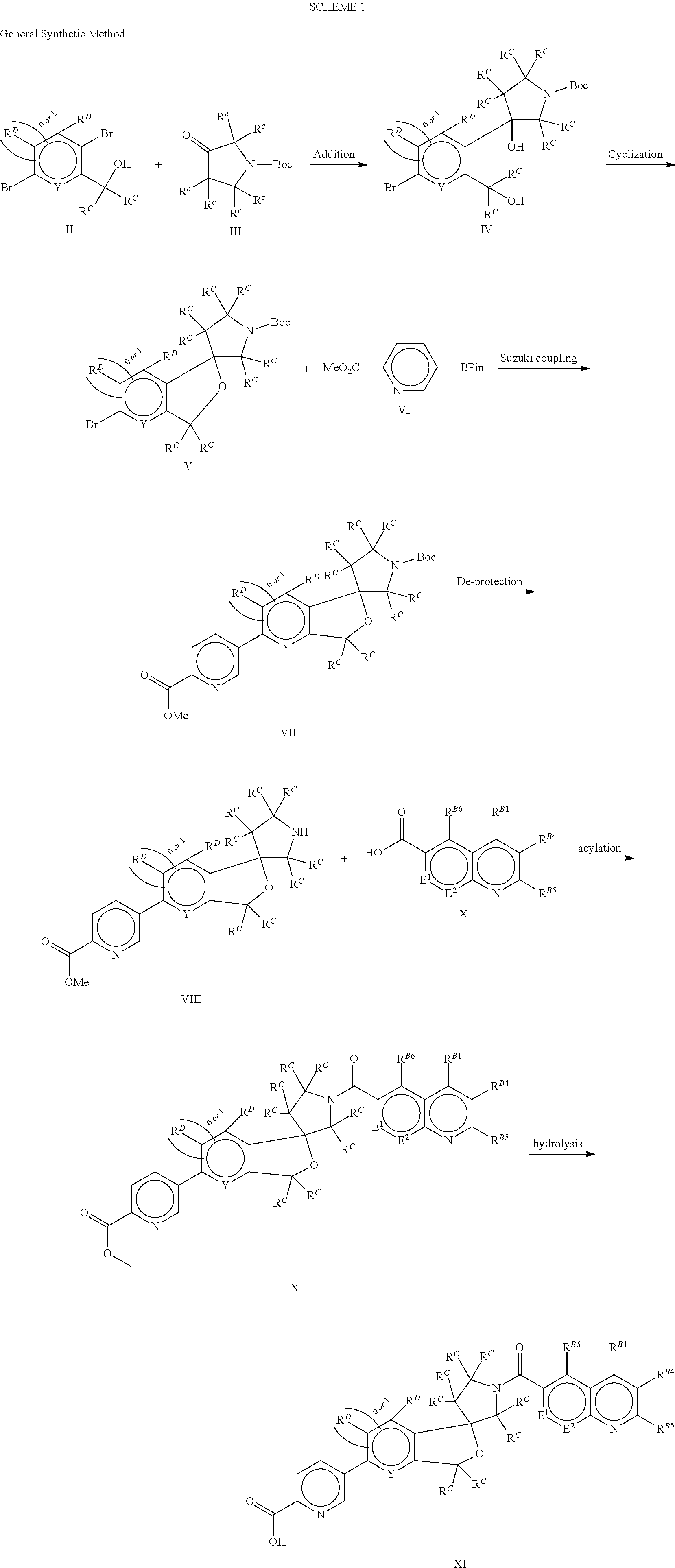

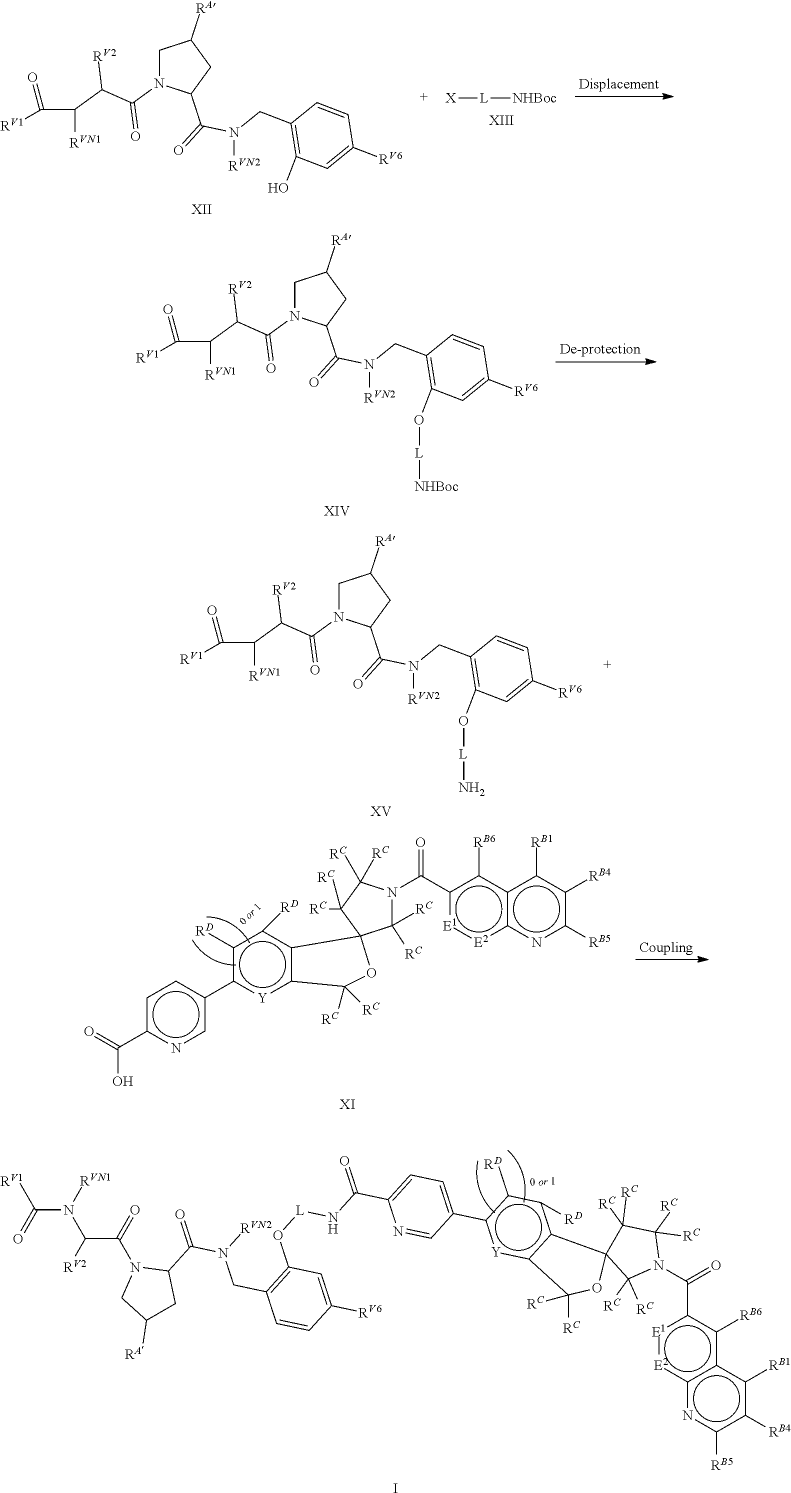

A compound of Formula I may be prepared according to the procedures shown in SCHEME 1.

According to SCHEME 1, commercially available bromo-benzyl alcohol compound of formula II is reacted with a metal reagent such as n-BuLi, sec-BuLi or i-PrMgBr and the like; in a suitable solvent such as THE, dioxane or hexanes and the like; at temperatures ranging from −78° C. to 0° C.; and reacted with commercially available ketone compound of formula III to give a diol compound of formula IV. A diol compound of formula IV is treated with a suitable dehydrate reagent such as (cyanomethylene)tributylphosphorane and the like; at temperatures ranging from 80° C. to 120° C., preferably 100° C.; in a suitable solvent such as toluene, dioxane or xylene and the like to provide a cyclized compound of formula V. A cyclized compound of formula V is coupled with a commercially available pinacol borate ester of formula VI; with a suitable catalyst such as PdCl₂(dppf), Pd(Ph₃P)₄ and the like; with a suitable base such as potassium acetate, sodium carbonate, and the like; in a suitable solvent such as 1,4-dioxane, DMSO and the like; at temperatures ranging from 60° C. to 120° C.; to provide coupled compound of formula VII. A coupled compound of formula VII is treated with an acidic solvent such as TFA, 6 N HCl and the like in a suitable solvent such as 1,4-dioxane, DCM and the like; at temperatures ranging from 0° C. to 40° C.; to provide a secondary amine compound of formula VIII. An amine compound of formula VIII is coupled with commercially available or prepared by known methods of formula IX; in a suitable solvent such as DMF, DCM and the like; with a suitable coupling agent such as HATU, T3P and the like; at temperatures ranging from 0° C. to 60° C.; to provide the coupled compound of formula X. A coupled compound of formula X is hydrolyzed with a suitable base such as LiOH, NaOH and the like; in a suitable solvent such as MeOH, THE, water and the like; at temperatures ranging from 25° C. to 80° C.; to provide hydrolyzed compound of formula XI.

A compound of formula XII (commercially available or prepared by known methods) is reacted with a compound of formula XIII (commercially available or prepared by known methods); under a suitable base such as Cs₂CO₃, K₂CO₃ and the like; in a suitable solvent such as DMF, DMSO and the like; at temperatures ranging from 60° C. to 120° C.; to provide a coupled compound of formula XIV. A coupled compound of formula XIV is treated with an acidic solvent such as TFA, 6 N HCl and the like in a suitable solvent such as 1,4-dioxane, DCM and the like; at temperatures ranging from 0° C. to 40° C.; to provide a primary amine compound of formula XV. An primary amine compound of formula XV is reacted with a compound of formula XI; in a suitable solvent such as DMF, DCM and the like; with a suitable coupling agent such as HATU, T3P and the like; at temperatures ranging from 0° C. to 60° C.; to provide the coupled compound of formula I.

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

In some embodiments, each of the intermediates prepared in the Schemes herein are considered embodiments of the present disclosure.

In some embodiments, each of the intermediates prepared in the Examples herein are considered embodiments of the present disclosure.

In some embodiments, each synthetic step as disclosed in the Schemes herein is separately considered as part of the present disclosure.

In some embodiments, each synthetic step as disclosed in the Examples herein is separately considered as part of the present disclosure.

### Analytical Methods, Materials, and Instrumentation

Unless otherwise noted, reagents and solvents were used as received from commercial suppliers. Proton nuclear magnetic resonance (NMR) spectra were obtained on either Bruker or Varian spectrometers at 400 MHz. Spectra are given in ppm (δ) and coupling constants, J, are reported in Hertz. Tetramethylsilane (TMS) was used as an internal standard. Liquid chromatography-mass spectrometry (LC/MS) were collected using a SHIMADZU LCMS-2020EV or Agilent 1260-6125B LCMS. Purity and low-resolution mass spectral data were measured using Agilent 1260-6125B LCMS system (with Diode Array Detector, and Agilent G6125BA Mass spectrometer) or using Waters Acquity UPLC system (with Diode Array Detector, and Waters 3100 Mass Detector). The purity was characterized by UV wavelength 214 nm, 220 nm, 254 nm and ESI. Column: poroshell 120 EC-C18 2.7 μm 4.6×100 mm; Flow rate 0.8 mL/min; Solvent A (100/0.1 water/formic acid), Solvent B (100 acetonitrile); gradient: hold 5% B to 0.3 min, 5-95% B from 0.3 to 2 min, hold 95% B to 4.8 min, 95-5% B from 4.8 to 5.4 min, then hold 5% B to 6.5 min. Or, column: Acquity UPLC BEH C18 1.7 μm 2.1×50 mm; Flow rate 0.5 mL/min; Solvent A (0.1% formic acid water), Solvent B (acetonitrile); gradient: hold 5% B for 0.2 min, 5-95% B from 0.2 to 2.0 min, hold 95% B to 3.1 min, then 5% B at 3.5 min.

### Biological Assays

The biological activities of the compounds of the present application can be assessed with methods and assays known in the art.

In some embodiments, PRMT5 binding potencies of compounds of the present application is measured using homogeneous time resolved fluorescence (HTRF) assay technology. In some embodiments, the PRMT5 binding potencies are determined in the presence of PRMT5 alone. In some embodiments, the PRMT5 binding potencies are determined in the presence of PRMT5 and methylthioadenosine (MTA). In some embodiments, the PRMT5 binding potencies are determined in the presence of PRMT5 and S-adenosyl-l-methionine (SAM).

In some embodiments, protein degradation is measured using HiBiT Assay. For example, PRMT5 degradation potency and Dₘₐₓ were determined using in HiBit technology in MTAP KO and MTAP WT cells. HCT116 cells +/−MTAP (Creative Biogene (CSC-RT2701) were genetically modified via CRISPR/Cas9 to fuse HiBiT to the carboxy terminus of PRMT5. Cells were cultured in McCoy's 5A containing 10% FBS and 1% (Vol:Vol) penicillin-streptomycin. At the time of experiment, cells were seeded at a density of 1250/25 μL/well in a 384-well plate (Corning; cat #3571) 24 hrs prior to compound treatment. Investigational compounds were prepared at 3 mM top concentration in 100% DMSO and an 11-point 3-fold serial dilution done in DMSO. 25 nL of serial diluted compounds were transferred to wells containing cell in 25 μL of media for a final top concentration of 3 μM. An equal volume of DMSO was added to control wells. Each treatment was performed in duplicate. Cells were incubated at 37° C.±5% CO₂ in a cell culture incubator for 18 hours. At the end of experiment, levels of PRMT5 were evaluated with Nano-Glo® HiBiT Lytic Detection Assay (Promega N3050). Assay reagent was prepared according to the manual provided with the Promega kit. 25 μL of this assay solution was added to each well. Luminescence from each well was measured with Envision plate reader (Perkin Elmer). Dose response curves were fit using Perkin Elmer Signals Lead Discovery software.

In some embodiments, the biological assay is described in the Examples herein.

### Methods of Use

In some aspects, the present disclosure provides methods of degrading a PRMT5 protein in a subject, comprising administering to the subject a compound disclosed herein.

In some aspects, the present disclosure provides uses of a compound disclosed herein in the manufacture of a medicament for degrading a PRMT5 protein in a subject.

In some aspects, the present disclosure provides compounds disclosed herein for use in degrading a PRMT5 protein in a subject.

In some aspects, the present disclosure provides methods of reducing the amount of a PRMT5 protein in a subject (e.g., in a biological sample (e.g., a cell or a tissue) obtained from the subject), comprising administering to the subject a compound disclosed herein.

In some aspects, the present disclosure provides uses of a compound disclosed herein in the manufacture of a medicament for reducing the amount of a PRMT5 protein in a subject (e.g., in a biological sample (e.g., a cell or a tissue) obtained from the subject).

In some aspects, the present disclosure provides compounds disclosed herein for use in reducing the amount of a PRMT5 protein in a subject (e.g., in a biological sample (e.g., a cell or a tissue) obtained from the subject).

In some aspects, the present disclosure provides methods of treating or preventing a disease or disorder in a subject in need thereof, comprising administering to the subject a compound disclosed herein (e.g., in a therapeutically effective amount).

In some aspects, the present disclosure provides methods of treating a disease or disorder in a subject in need thereof, comprising administering to the subject a compound disclosed herein (e.g., in a therapeutically effective amount).

In some aspects, the present disclosure provides uses of a compound disclosed herein in the manufacture of a medicament for treating or preventing a disease or disorder in a subject in need thereof.

In some aspects, the present disclosure provides uses of a compound disclosed herein in the manufacture of a medicament for treating a disease or disorder in a subject in need thereof.

In some aspects, the present disclosure provides compounds disclosed herein for use in treating or preventing a disease or disorder in a subject in need thereof.

In some aspects, the present disclosure provides compounds disclosed herein for use in treating a disease or disorder in a subject in need thereof.

In some embodiments, the disease or disorder is a PRMT5 protein-mediated disease or disorder, such as breast, lung, colorectal, and pancreatic cancers, as well as glioblastoma, neuroblastoma, and hematologic malignancies.

In some embodiments, the disease or disorder is cancer.

In some embodiments, the cancer is non-small cell lung cancer, small-cell lung cancer, colorectal cancer, bladder cancer, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

In some embodiments, the cancer is selected from NSCLC adenocarcinoma (LUAD), NSCL squamous cell carcinoma (LUSC), liver hepatocellular carcinoma (LIHC), uterine corpus endometrial carcinoma (UCEC), esophageal carcinoma (ESCA), skin cutaneous melanoma (SKCM), stomach adenocarcinoma (STAD), colon adenocarcinoma (COAD), bladder urothelial carcinoma (BLCA), and uterine carcinosarcoma (UCS).

In some embodiments, the cancer is selected from NSCLC adenocarcinoma (LUAD), NSCL squamous cell carcinoma (LUSC), liver hepatocellular carcinoma (LIHC), and uterine corpus endometrial carcinoma (UCEC).

In some embodiments, the cancer includes, but is not limited to, one or more of the cancers of Table A.

TABLE A

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic fibroma |
| lymphoma | rhabdomyosarcoma | sarcoma | |
| anaplastic large cell | anaplastic thyroid | angioimmunoblastic | angiomyolipoma |
| lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial tumor | |
| embryonal carcinoma | endocrine gland | endodermal sinus | enteropathy- |
| | neoplasm | tumor | associated T-cell |
| | | | lymphoma |
| esophageal cancer | fetus in fetu | fibroma | fibrosarcoma |
| follicular lymphoma | follicular thyroid | ganglioneuroma | gastrointestinal |
| | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck cancer | hemangiopericytoma | hematological cancer | hepatoblastoma |
| hepatosplenic T-cell | Hodgkin's lymphoma | non-Hodgkin's | invasive lobular |
| lymphoma | | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic lymphocytic |
| | leukemia | leukemia | leukemia |
| liver cancer | small cell lung cancer | non-small cell lung | MALT lymphoma |
| | | cancer | |
| malignant fibrous | malignant peripheral | malignant triton | mantle cell |
| histiocytoma | nerve sheath tumor | tumor | lymphoma |
| marginal zone B-cell | mast cell leukemia | mediastinal germ cell | medullary carcinoma |
| lymphoma | | tumor | of the breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid liposarcoma | myxoma | myxosarcoma | nasopharyngeal |
| | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | primary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell carcinoma | renal medullary | retinoblastoma | rhabdomyoma |
| | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell carcinoma |
| carcinoma | | tumors | |
| soft tissue sarcoma | somatostatinoma | soot wart | spinal tumor |
| splenic marginal zone | squamous cell | synovial sarcoma | Sezary's disease |
| lymphoma | carcinoma | | |
| small intestine cancer | squamous carcinoma | stomach cancer | T-cell lymphoma |
| testicular cancer | thecoma | thyroid cancer | transitional cell |
| | | | carcinoma |
| throat cancer | urachal cancer | urogenital cancer | urothelial carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

In some embodiments, the cancer is a solid tumor. In some embodiments, the cancer is a hematological cancer. Exemplary hematological cancers include, but are not limited to, the cancers listed in Table B. In some embodiments, the hematological cancer is acute lymphocytic leukemia, chronic lymphocytic leukemia (including B-cell chronic lymphocytic leukemia), or acute myeloid leukemia.

TABLE B

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic lymphoma |
| Waldenstrom's macroglobulinemia (WM) | T-cell lymphoma |
| diffuse large B-cell lymphoma (DLBCL) | mast cell leukemia |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell lymphoma |
| Richter's transformation | |

In some embodiments, the subject is a mammal.

In some embodiments, the subject is a human.

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.F. Eliel, Ed., Univ. of Notre Dame Press, Notre Dame, IN 1972).

The invention additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇alkyl”). In some embodiments, an alkyl group has 1 to 6 carbon atoms (“C₁₋₆ alkyl”, which is also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In some embodiments, the alkyl group is unsubstituted C₁₋₁₀ alkyl (e.g., —CH₃). In some embodiments, the alkyl group is substituted C₁₋₁₀ alkyl. Common alkyl abbreviations include Me (—CH₃), Et (—CH₂CH₃), i-Pr (—CH(CH₃)₂), n-Pr (—CH₂CH₂CH₃), n-Bu (—CH₂CH₂CH₂CH₃), or i-Bu (—CH₂CH(CH₃)₂).

“Alkylene” as used herein, refers to an alkyl group wherein two hydrogens are removed to provide a divalent radical. When a range or number of carbons is provided for a particular “alkylene” group, it is understood that the range or number refers to the range or number of carbons in the linear carbon divalent chain. An “alkylene” group may be substituted or unsubstituted with one or more substituents as described herein. Exemplary unsubstituted divalent alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted divalent alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” as used herein, refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In some embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In some embodiments, the alkenyl group is unsubstituted C₂₋₁₀ alkenyl. In some embodiments, the alkenyl group is substituted C₂₋₁₀ alkenyl.

“Alkenylene” as used herein, refers to an alkenyl group wherein two hydrogens are removed to provide a divalent radical. When a range or number of carbons is provided for a particular “alkenylene” group, it is understood that the range or number refers to the range or number of carbons in the linear carbon divalent chain. An “alkenylene” group may be substituted or unsubstituted with one or more substituents as described herein. Exemplary unsubstituted divalent alkenylene groups include, but are not limited to, ethenylene (—CH═CH—) and propenylene (e.g., —CH═CHCH₂—, —CH₂—CH═CH—). Exemplary substituted divalent alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“Alkynyl” as used herein, refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In some embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In some embodiments, the alkynyl group is unsubstituted C₂₋₁₀ alkynyl. In some embodiments, the alkynyl group is substituted C₂₋₁₀ alkynyl.

“Alkynylene” as used herein, refers to a linear alkynyl group wherein two hydrogens are removed to provide a divalent radical. When a range or number of carbons is provided for a particular “alkynylene” group, it is understood that the range or number refers to the range or number of carbons in the linear carbon divalent chain. An “alkynylene” group may be substituted or unsubstituted with one or more substituents as described herein. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In some embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and/or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In some embodiments, the heteroalkyl group is an unsubstituted heteroC₁₋₁₀ alkyl. In some embodiments, the heteroalkyl group is a substituted heteroC₁₋₁₀ alkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In some embodiments, a heteroalkenyl group refers to a group having from 2 to 10 carbon atoms, at least one double bond, and 1, 2, 3, or 4 heteroatoms (“heteroC₂₋₁₀ alkenyl”). In some embodiments, a heteroalkenyl group has 2 to 9 carbon atoms at least one double bond, and 1, 2, 3, or 4 heteroatoms (“heteroC₂₋₉ alkenyl”). In some embodiments, a heteroalkenyl group has 2 to 8 carbon atoms, at least one double bond, and 1, 2, 3, or 4 heteroatoms (“heteroC₂₋₈ alkenyl”). In some embodiments, a heteroalkenyl group has 2 to 7 carbon atoms, at least one double bond, and 1, 2, 3, or 4 heteroatoms (“heteroC₂₋₇ alkenyl”). In some embodiments, a heteroalkenyl group has 2 to 6 carbon atoms, at least one double bond, and 1, 2, or 3 heteroatoms (“heteroC₂₋₆ alkenyl”). In some embodiments, a heteroalkenyl group has 2 to 5 carbon atoms, at least one double bond, and 1 or 2 heteroatoms (“heteroC₂₋₅ alkenyl”). In some embodiments, a heteroalkenyl group has 2 to 4 carbon atoms, at least one double bond, and lor 2 heteroatoms (“heteroC₂₋₄ alkenyl”). In some embodiments, a heteroalkenyl group has 2 to 3 carbon atoms, at least one double bond, and 1 heteroatom (“heteroC₂₋₃ alkenyl”). In some embodiments, a heteroalkenyl group has 2 to 6 carbon atoms, at least one double bond, and 1 or 2 heteroatoms (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In some embodiments, the heteroalkenyl group is an unsubstituted heteroC₂₋₁₀ alkenyl. In some embodiments, the heteroalkenyl group is a substituted heteroC₂₋₁₀ alkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms are inserted between a carbon atom and the parent molecule, i.e., between the point of attachment. In some embodiments, a heteroalkynyl group refers to a group having from 2 to 10 carbon atoms, at least one triple bond, and 1, 2, 3, or 4 heteroatoms (“heteroC₂₋₁₀ alkynyl”). In some embodiments, a heteroalkynyl group has 2 to 9 carbon atoms, at least one triple bond, and 1, 2, 3, or 4 heteroatoms (“heteroC₂₋₉ alkynyl”). In some embodiments, a heteroalkynyl group has 2 to 8 carbon atoms, at least one triple bond, and 1, 2, 3, or 4 heteroatoms (“heteroC₂₋₈ alkynyl”). In some embodiments, a heteroalkynyl group has 2 to 7 carbon atoms, at least one triple bond, and 1, 2, 3, or 4 heteroatoms (“heteroC₂₋₇ alkynyl”). In some embodiments, a heteroalkynyl group has 2 to 6 carbon atoms, at least one triple bond, and 1, 2, or 3 heteroatoms (“heteroC₂₋₆ alkynyl”). In some embodiments, a heteroalkynyl group has 2 to 5 carbon atoms, at least one triple bond, and 1 or 2 heteroatoms (“heteroC₂₋₅ alkynyl”). In some embodiments, a heteroalkynyl group has 2 to 4 carbon atoms, at least one triple bond, and 1 or 2 heteroatoms (“heteroC₂₋₄ alkynyl”). In some embodiments, a heteroalkynyl group has 2 to 3 carbon atoms, at least one triple bond, and 1 heteroatom (“heteroC₂₋₃ alkynyl”). In some embodiments, a heteroalkynyl group has 2 to 6 carbon atoms, at least one triple bond, and 1 or 2 heteroatoms (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In some embodiments, the heteroalkynyl group is an unsubstituted heteroC₂₋₁₀ alkynyl. In some embodiments, the heteroalkynyl group is a substituted heteroC₂₋₁₀ alkynyl.

Analogous to “alkylene,” “alkenylene,” and “alkynylene” as defined above, “heteroalkylene,” “heteroalkenylene,” and “heteroalkynylene,” as used herein, refer to a divalent radical of heteroalkyl, heteroalkenyl, and heteroalkynyl group respectively. When a range or number of carbons is provided for a particular “heteroalkylene,” “heteroalkenylene,” or “heteroalkynylene,” group, it is understood that the range or number refers to the range or number of carbons in the linear divalent chain, “Heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl).

Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In some embodiments, the aryl group is unsubstituted C₆₋₁₄ aryl. In some embodiments, the aryl group is substituted C₆₋₁₄ aryl.

“Arylene” as used herein, refers to an aryl group wherein two hydrogens are removed to provide a divalent radical. When a range or number of carbons is provided for a particular “arylene” group, it is understood that the range or number refers to the range or number of carbons in the aryl group. An “arylene” group may be substituted or unsubstituted with one or more substituents as described herein.

“Heteroaryl” refers to a radical of a 5- to 14-membered monocyclic or polycyclic 4n+2 aromatic ring system (e.g., having 6, 10, or 14 π electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl group, as defined above, is fused with one or more aryl groups wherein the point of attachment is either on the heteroaryl or the one or more aryl groups, and in such instances, the number of ring members designates the total number of ring members in the fused (aryl/heteroaryl) ring system. When substitution is indicated in such instances, unless otherwise specified, substitution can occur on either the heteroaryl or the one or more aryl groups. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In some embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In some embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In some embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In some embodiments, the 5- to 6-membered heteroaryl has 1-3 ring heteroatoms independently selected from nitrogen, oxygen, and sulfur. In some embodiments, the 5- to 6-membered heteroaryl has 1-2 ring heteroatoms independently selected from nitrogen, oxygen, and sulfur. In some embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In some embodiments, the heteroaryl group is unsubstituted 5- to 14-membered heteroaryl. In some embodiments, the heteroaryl group is substituted 5- to 14-membered heteroaryl.

Exemplary 5-membered heteroaryl containing one heteroatom include, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Heteroarylene” as used herein, refers to a heteroaryl group wherein two hydrogens are removed to provide a divalent radical. When a range or number of ring members is provided for a particular “heteroarylene” group, it is understood that the range or number refers to the number of ring members in the heteroaryl group. A “heteroarylene” group may be substituted or unsubstituted with one or more substituents as described herein.

“Carbocyclyl” refers to a radical of a non-aromatic cyclic hydrocarbon group having from 3 to 12 ring carbon atoms (“C₃₋₁₂ carbocyclyl”) and zero heteroatoms in the nonaromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 5 to 12 ring carbon atoms (“C₅₋₁₂ carbocyclyl”). In some embodiments, a carbocyclyl group has 5 to 10 ring carbon atoms (“C₅₋₁₀ carbocyclyl”). In some embodiments, a carbocyclyl group has 5 to 8 ring carbon atoms (“C₅₋₈ carbocyclyl”). In some embodiments, a carbocyclyl group has 5 or 6 ring carbon atoms (“C₅₋₆ carbocyclyl”). Exemplary C₃₋₆ carbocyclyl include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

In some embodiments, “carbocyclyl” is a monocyclic, saturated carbocyclyl group having from 3 to 12 ring carbon atoms (“C₃₋₁₂ carbocyclyl”). In some embodiments, “carbocyclyl” is a monocyclic, saturated carbocyclyl group having from 3 to 10 ring carbon atoms (“C₃₋₁₀ carbocyclyl”). In some embodiments, “carbocyclyl” is a monocyclic, saturated carbocyclyl group having from 3 to 8 ring carbon atoms (“C₃₋₈ carbocyclyl”). In some embodiments, “carbocyclyl” is a monocyclic, saturated carbocyclyl group having from 3 to 6 ring carbon atoms (“C₃₋₆ carbocyclyl”). In some embodiments, “carbocyclyl” is a monocyclic, saturated carbocyclyl group having from 5 to 12 ring carbon atoms (“C₅₋₁₂ carbocyclyl”). In some embodiments, a carbocyclyl group has 5 to 10 ring carbon atoms (“C₅₋₁₀ carbocyclyl”). In some embodiments, a carbocyclyl group has 5 to 8 ring carbon atoms (“C₅₋₈ carbocyclyl”). In some embodiments, “carbocyclyl” is a monocyclic, saturated carbocyclyl group having 5 or 6 ring carbon atoms (“C₅₋₆ carbocyclyl”). Examples of C₅₋₆ carbocyclyl include cyclopentyl (C₅) and cyclohexyl (C₅). Examples of C₃₋₆ carbocyclyl include the aforementioned C₅₋₆ carbocyclyl groups as well as cyclopropyl (C₃) and cyclobutyl (C₄). Examples of C₃₋₈ carbocyclyl include the aforementioned C₃₋₆ carbocyclyl groups as well as cycloheptyl (C₇) and cyclooctyl (C₈). Unless otherwise specified, each instance of a carbocyclyl group is independently unsubstituted (an “unsubstituted carbocyclyl”) or substituted (a “substituted carbocyclyl”) with one or more substituents. In some embodiments, the carbocyclyl group is unsubstituted C₃₋₁₂ carbocyclyl. In some embodiments, the carbocyclyl group is substituted C₃₋₁₂ carbocyclyl.

As the foregoing examples illustrate, in some embodiments, the carbocyclyl group is either monocyclic (“monocyclic carbocyclyl”) or polycyclic (“polycyclic carbocyclyl”) that contains a fused, bridged or spiro ring system and can be saturated or can be partially unsaturated. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In some embodiments, the carbocyclyl group is unsubstituted C₃₋₁₂ carbocyclyl. In some embodiments, the carbocyclyl group is a substituted C₃₋₁₂ carbocyclyl.

“Fused carbocyclyl” or “fused carbocycle” refers to ring systems wherein the carbocyclyl group, as defined above, is fused with, i.e., share two common atoms (as such, share one common bond), one or more carbocyclyl groups, as defined above, wherein the point of attachment is on any of the fused rings. In such instances, the number of carbons designates the total number of carbons in the fused ring system. When substitution is indicated, unless otherwise specified, substitution can occur on any of the fused rings.

“Spiro carbocyclyl” or “spiro carbocycle” refers to ring systems wherein the carbocyclyl group, as defined above, form spiro structure with, i.e., share one common atom with, one or more carbocyclyl groups, as defined above, wherein the point of attachment is on the carbocyclyl rings in which the spiro structure is embedded. In such instances, the number of carbons designates the total number of carbons of the carbocyclyl rings in which the spiro structure is embedded. When substitution is indicated, unless otherwise specified, substitution can occur on the carbocyclyl rings in which the spiro structure is embedded.

“Bridged carbocyclyl” or “bridged carbocycle” refers to ring systems wherein the carbocyclyl group, as defined above, form bridged structure with, i.e., share more than two atoms (as such, share more than one bonds) with, one or more carbocyclyl groups, as defined above, wherein the point of attachment is on any of the carbocyclyl rings in which the bridged structure is embedded. In such instances, the number of carbons designates the total number of carbons of the carbocyclyl rings in which the bridged structure is embedded. When substitution is indicated, unless otherwise specified, substitution can occur on any of the carbocyclyl rings in which the bridged structure is embedded.

“Carbocyclylene” as used herein, refers to a carbocyclyl group wherein two hydrogens are removed to provide a divalent radical. The divalent radical may be present on different atoms or the same atom of the carbocycle group. When a range or number of carbons is provided for a particular “carbocyclyl” group, it is understood that the range or number refers to the range or number of carbons in the carbocyclyl group. A “carbocyclyl” group may be substituted or unsubstituted with one or more substituents as described herein.

“Heterocyclyl” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”). In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

In some embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In some embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In some embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In some embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In some embodiments, the 5- to 6-membered heterocyclyl has 1-3 ring heteroatoms selected from nitrogen, oxygen, and sulfur. In some embodiments, the 5- to 6-membered heterocyclyl has 1-2 ring heteroatoms selected from nitrogen, oxygen, and sulfur. In some embodiments, the 5- to 6-membered heterocyclyl has one ring heteroatom selected from nitrogen, oxygen, and sulfur.

As the foregoing examples illustrate, in some embodiments, a heterocyclyl group can either be monocyclic (“monocyclic heterocyclyl”) or polycyclic (“polycyclic heterocyclyl”) that contains a fused, bridged or spiro ring system, and can be saturated or can be partially unsaturated. Heterocyclyl polycyclic ring systems can include one or more heteroatoms in one or both rings. “Heterocyclyl” also includes ring systems wherein the heterocyclyl group, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In some embodiments, the heterocyclyl group is unsubstituted 3- to 12-membered heterocyclyl. In some embodiments, the heterocyclyl group is substituted 3- to 12-membered heterocyclyl.

“Fused heterocyclyl” or “fused heterocycle” refers to ring systems wherein the heterocyclyl group, as defined above, is fused with, i.e., share two common atoms (as such, share one common bond) with, one or more heterocyclyl or carbocyclyl groups, as defined above, wherein the point of attachment is on any of the fused rings. In such instances, the number of ring members designates the total number of ring members in the fused ring system. When substitution is indicated, unless otherwise specified, substitution can occur on any of the fused rings.

“Spiro heterocyclyl” or “spiro heterocycle” refers to ring systems wherein the heterocyclyl group, as defined above, form spiro structure with, i.e., share one common atom with, one or more heterocyclyl or carbocyclyl groups, as defined above, wherein the point of attachment is on the heterocyclyl or carbocyclyl rings in which the spiro structure is embedded. In such instances, the number of ring members designates the total number of ring members of the heterocyclyl or carbocyclyl rings in which the spiro structure is embedded. When substitution is indicated, unless otherwise specified, substitution can occur on any of the heterocyclyl or carbocyclyl rings in which the spiro structure is embedded.

“Bridged heterocyclyl” or “bridged heterocycle” refers to ring systems wherein the heterocyclyl group, as defined above, form bridged structure with, i.e., share more than two atoms (as such, share more than one bonds) with, one or more heterocyclyl or carbocyclyl groups, as defined above, wherein the point of attachment is on the heterocyclyl or carbocyclyl rings in which the bridged structure is embedded. In such instances, the number of ring members designates the total number of ring members of the heterocyclyl or carbocyclyl rings in which the bridged structure is embedded. When substitution is indicated, unless otherwise specified, substitution can occur on any of the heterocyclyl or carbocyclyl rings in which the bridged structure is embedded.

“Heterocyclylene” as used herein, refers to a heterocyclyl group wherein two hydrogens are removed to provide a divalent radical. The divalent radical may be present on different atoms or the same atom of the heterocycle group. When a range or number of ring members is provided for a particular “heterocyclylene” group, it is understood that the range or number refers to the number of ring members in the heterocyclylene group. A “heterocyclylene” group may be substituted or unsubstituted with one or more substituents as described herein.

“Alkoxy” as used herein, refers to the group —OR, wherein R is alkyl as defined herein. C₁₋₆ alkoxy refers to the group —OR, wherein each R is C₁₋₆ alkyl, as defined herein. Exemplary C₁₋₆ alkyl is set forth above.

“Alkylamino” as used herein, refers to the group —NHR or —NR₂, wherein each R is independently alkyl, as defined herein. C₁₋₆ alkylamino refers to the group —NHR or —NR₂, wherein each R is independently C₁₋₆ alkyl, as defined herein. Exemplary C₁₋₆ alkyl is set forth above.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance structure/tautomer involving a heteroatom provides a carbon atom that is able to form two geminal radicals, which form a double bond with an oxygen radical.

“Halo” or “halogen” refers to fluoro (F), chloro (Cl), bromo (Br), and iodo (I). In some embodiments, the halo group is either fluoro or chloro.

“Protecting group” as used herein is art-recognized and refers to a chemical moiety introduced into a molecule by chemical modification of a functional group (e.g., hydroxyl, amino, thio, and carboxylic acid) to obtain chemoselectivity in a subsequent chemical reaction, during which the unmodified functional group may not survive or may interfere with the chemical reaction. Common functional groups that need to be protected include but not limited to hydroxyl, amino, thiol, and carboxylic acid. Accordingly, the protecting groups are termed hydroxyl-protecting groups, amino-protecting groups, thiol-protecting groups, and carboxylic acid-protecting groups, respectively.

Common types of hydroxyl-protecting groups include but not limited to ethers (e.g., methoxymethyl (MOM), β-Methoxyethoxymethyl (MEM), tetrahydropyranyl (THP), p-methoxyphenyl (PMP), t-butyl, triphenylmethyl (Trityl), allyl, and benzyl ether (Bn)), silyl ethers (e.g., t-butyldiphenylsilyl (TBDPS), trimethylsilyl (TMS), triisopropylsilyl (TIPS), tri-iso-propylsilyloxymethyl (TOM), and t-butyldimethylsilyl (TBDMS)), and esters (e.g., pivalic acid ester (Piv) and benzoic acid ester (benzoate; Bz)).

Common types of amino-protecting groups include but not limited to carbamates (e.g., t-butyloxycarbonyl (Boc), 9-fluorenylmethyloxycarbonyl (Fmoc), p-methoxybenzyl carbonyl (Moz or MeOZ), 2,2,2-trichloroehtoxycarbonyl (Troc), and benzyl carbamate (Cbz)), esters (e.g., acetyl (Ac); benzoyl (Bz), trifluoroacetyl, and phthalimide), amines (e.g., benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)).

Common types of thiol-protecting groups include but not limited to sulfide (e.g., p-methylbenzyl (Meb), t-butyl, acetamidomethyl (Acm), and triphenylmethyl (Trityl)).

Common types of carboxylic acid-protecting groups include but not limited to esters (e.g., methyl ester, triphenylmethyl (Trityl), t-butyl ester, benzyl ester (Bn), S-t-butyl ester, silyl esters, and orthoesters) and oxazoline.

These and other exemplary substituents are described in more detail in the Detailed Description, Examples, and claims. The invention is not intended to be limited in any manner by the above exemplary listing of substituents.

### Other Definitions

“Antibody” is used in the broadest sense and specifically encompasses monoclonal antibodies (including full length monoclonal antibodies), polyclonal antibodies, multispecific antibodies (e.g., bispecific antibodies), and antibody fragments so long as they exhibit the desired biological activity. “Antibody fragment” and all grammatical variants thereof as used herein are defined as a portion of an intact antibody comprising the antigen binding site or variable region of the intact antibody, wherein the portion is free of the constant heavy chain domains (i.e., CH₂, CH₃, and CH₄, depending on antibody isotype) of the Fc region of the intact antibody. Examples of antibody fragments include Fab, Fab, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; (2) single chain polypeptides containing only one light chain variable domain, or a fragment thereof that contains the three CDRs of the light chain variable domain, without an associated heavy chain moiety; (3) single chain polypeptides containing only one heavy chain variable region, or a fragment thereof containing the three CDRs of the heavy chain variable region, without an associated light chain moiety; (4) nanobodies comprising single Ig domains from non-human species or other specific single-domain binding modules; and (5) multispecific or multivalent structures formed from antibody fragments. In an antibody fragment comprising one or more heavy chains, the heavy chain(s) can contain any constant domain sequence (e.g., CHI in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). “Antibody” refers to a polypeptide comprising an antigen binding region (including the complementarity determining region (CDRs)) from an immunoglobulin gene or fragments thereof. The term “antibody” specifically encompasses monoclonal antibodies (including full length monoclonal antibodies), polyclonal antibodies, multispecific antibodies (e.g., bispecific antibodies), and antibody fragments that exhibit the desired biological activity. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (VL and VH, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class γ heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant). Typically, the antigen binding domain of an antibody will be most critical in specificity and affinity of binding to cancer cells. An antibody that targets a particular antigen includes a bispecific or multispecific antibody with at least one antigen binding region that targets the particular antigen. In some embodiments, the targeted monoclonal antibody is a bispecific antibody with at least one antigen binding region that targets tumor cells. “Antibody construct” refers to an antibody or a fusion protein comprising (i) an antigen binding domain and (ii) an Fc domain. In some embodiments, the binding agent is an antigen-binding antibody “fragment,” which is a construct that comprises at least an antigen-binding region of an antibody, alone or with other components that together constitute the antigen-binding construct. Many different types of antibody “fragments” are known in the art, including, for instance, (i) a Fab fragment, which is a monovalent fragment consisting of the VL, VH, C_{L}, and CH₁ domains, (ii) a F(ab′)₂ fragment, which is a bivalent fragment comprising two Fab fragments linked by a disulfide bridge at the hinge region, (iii) a Fv fragment consisting of the VL and VH domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a single chain Fv (scFv), which is a monovalent molecule consisting of the two domains of the Fv fragment (i.e., VL and VH) joined by a synthetic linker which enables the two domains to be synthesized as a single polypeptide chain. The antibody or antibody fragments can be part of a larger construct, for example, a conjugate or fusion construct of the antibody fragment to additional regions. For instance, in some embodiments, the antibody fragment can be fused to an Fc region as described herein. In other embodiments, the antibody fragment (e.g., a Fab or scFv) can be part of a chimeric antigen receptor or chimeric T-cell receptor, for instance, by fusing to a transmembrane domain (optionally with an intervening linker or “stalk” (e.g., hinge region)) and optional intercellular signaling domain. For instance, the antibody fragment can be fused to the gamma and/or delta chains of a T-cell receptor, so as to provide a T-cell receptor like construct that binds TROP2. In yet another embodiment, the antibody fragment is part of a bispecific T-cell engager (BiTEs) comprising a CD1 or CD3 binding domain and linker. “Epitope” means any antigenic determinant or epitopic determinant of an antigen to which an antigen binding domain binds (i.e., at the paratope of the antigen binding domain). Antigenic determinants usually consist of chemically active surface groupings of molecules, such as amino acids or sugar side chains, and usually have specific three dimensional structural characteristics, as well as specific charge characteristics. The terms “Fc receptor” or “FcR” refer to a receptor that binds to the Fc region of an antibody. There are three main classes of Fc receptors: (1) FcγR which bind to IgG, (2) FcαR which binds to IgA, and (3) FcεR which binds to IgE. The FcγR family includes several members, such as FcγI (CD64), FcγRIIA (CD32A), FcγRIIB (CD32B), FcγRIIIA (CD16A), and FcγRIIIB (CD16B). The Fcγ receptors differ in their affinity for IgG and also have different affinities for the IgG subclasses (e.g., IgG1, IgG2, IgG3, and IgG4).

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

A “subject” to which administration is contemplated includes, but is not limited to, humans (i.e., a male or female of any age group, e.g., a pediatric subject (e.g, infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In some embodiments, the subject is a human. In some embodiments, the subject is a non-human animal.

An “effective amount” means the amount of a compound that, when administered to a subject for treating or preventing a disease, is sufficient to affect such treatment or prevention. The “effective amount” can vary depending on the compound, the disease and its severity, and the age, weight, etc., of the subject to be treated. A “therapeutically effective amount” refers to the effective amount for therapeutic treatment. A “prophylactically effective amount” refers to the effective amount for prophylactic treatment.

“Preventing”, “prevention” or “prophylactic treatment” refers to a reduction in risk of acquiring or developing a disease or disorder (i.e., causing at least one of the clinical symptoms of the disease not to develop in a subject not yet exposed to a disease-causing agent, or in a subject who is predisposed to the disease in advance of disease onset).

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

The term “about” when referring to a number or a numerical range means that the number or numerical range referred to is an approximation within experimental variability or within statistical experimental error, and thus the number or numerical range, in some instances, will vary between 1% and 15% of the stated number or numerical range. In some embodiments, the number or numerical range vary by 1%, 2%, 3%, 4%, 5%, 6%, 7%, 8%, 9%, 10%, 11%, 12%, 13%, 14%, or 15% of the stated number or numerical range. In some embodiments, the number or numerical range vary by 1%, 2%, 3%, 4%, or 5% of the stated number or numerical range. In some embodiments, the number or numerical range vary by 1%, 2%, or 3% of the stated number or numerical range.

The term “comprising” (and related terms such as “comprise” or “comprises” or “having” or “including”) is not intended to exclude that in other some embodiments, for example, an embodiment of any composition of matter, composition, method, or process, or the like, described herein, “consist of” or “consist essentially of” the described features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, when a moiety is “optionally substituted with one or more A, B, C, D, or E,” it is understood that the moiety is optionally substituted with one more substituent, wherein each substituent is independently selected from A, B, C, D, and E.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### Exemplary Embodiments

Exemplary Embodiment No. 1. A compound of Formula I

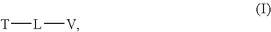

or a pharmaceutically acceptable salt, solvate, or stereoisomer thereof, wherein:
T is of Formula I-1

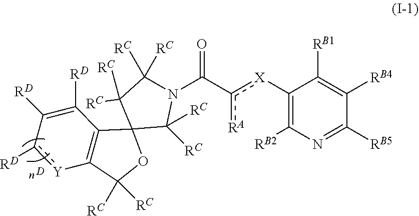

wherein:
each [symbol] is independently a single bond or a double bond, as valency permits;
X is N(R^{X1}), and R^{A} is oxo; or
X is C(R^{X2}), and R^{A} and R^{B2}, together with the intervening atoms to which they are attached, form Ce aryl or 6-membered heteroaryl, wherein the Co aryl or 6-membered heteroaryl is optionally substituted with one or more halogen, —CN, —NO₂, —OH, or —NH₂;
R^{X1} is hydrogen or C₁₋₆ alkyl, wherein the C₁₋₆ alkyl is optionally substituted with one or more halogen, —CN, —NO₂, —OH, or —NH₂;
R^{X2} is hydrogen, halogen, —CN, —NO₂, —OH, —NH₂, or C₁₋₆ alkyl, wherein the C₁₋₆ alkyl is optionally substituted with one or more halogen, —CN, —NO₂, —OH, or —NH₂;
R^{B1}, R^{B2}, R^{B4}, and R^{B5} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, or —NH₂; or
R^{B1} and R^{B4}, together with the intervening atoms to which they are attached, form 3- to 8-membered heterocyclyl or 5- to 6-membered heteroaryl, wherein the 3- to 8-membered heterocyclyl or 5- to 6-membered heteroaryl is optionally substituted with one or more halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl;
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
n^{D} is 0 or 1;
Y is O, S, or C(R^{D}); and
each R^{D} is independently

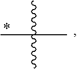

hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂, wherein * denotes attachment to L, and one R^{D} is

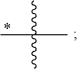

L is of Formula I-2

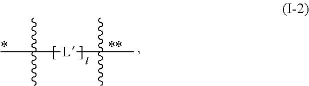

wherein:
* denotes attachment to T, and ** denotes attachment to V;
each L′ is independently —C(═O)—, —C(═O)N(R^{L′})—, —N(R^{L′})C(═O)—, —C(═O)O—, —OC(═O)—, —N(R^{L′})—, —O—, —O—(C₁₋₆ alkylene)-, —(C₁₋₆ alkylene)-O—, —S—, —S(═O)₂—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene, wherein the —O—(C₁₋₆ alkylene)-, —(C₁₋₆ alkylene)-O—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene is optionally substituted with one or more halogen, —CN, —NO₂, —OH, or —NH₂;
each occurrence of R^{L′} is independently hydrogen, C₁₋₆ alkyl, C₂₋₆ alkenyl, C₂₋₆ alkynyl, C₃₋₈ carbocyclyl, 3- to 8-membered heterocyclyl, —S(═O)₂R^{a}, —S(═O)₂OR^{a}, —S(—O)₂N(R^{a})₂, —C(═O)R^{a}, —C(═O)OR^{a}, or —C(═O)N(R^{a})₂, wherein the C₁₋₆ alkyl, C₂₋₆ alkenyl, C₂₋₆ alkynyl, C₃₋₈ carbocyclyl, or 3- to 8-membered heterocyclyl is optionally substituted with one or more halogen, —CN, —NO₂, —OH, or —NH₂; and
each R^{a} independently is hydrogen, C₁₋₆ alkyl, C₂₋₆ alkenyl, or C₂₋₆ alkynyl, wherein the C₁₋₆ alkyl, C₂₋₆ alkenyl, or C₂₋₆ alkynyl is optionally substituted with one or more halogen, —CN, —NO₂, —OH, or —NH₂;
l is an integer selected from 0 to 10;
V is of Formula 1-3

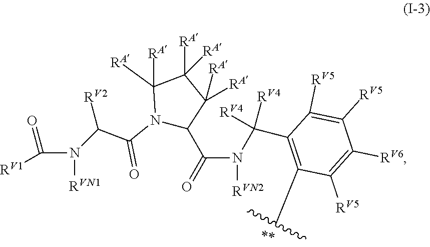

wherein:
* denotes attachment to L;
R^{V1} is hydrogen, C₁₋₆ alkyl, C₃₋₆ carbocyclyl, or 3- to 6-membered heterocyclyl, wherein the C₁₋₆ alkyl, C₃₋₆ carbocyclyl, or 3- to 6-membered heterocyclyl is optionally substituted with one or more halogen, —CN, —NO₂, —OH, or —NH₂;
R^{VN1} is hydrogen or C₁₋₆ alkyl, wherein the C₁₋₆ alkyl is optionally substituted with one or more halogen, —CN, —NO₂, —OH, or —NH₂;
R^{V2} is hydrogen or C₁₋₆ alkyl, wherein the C₁₋₆ alkyl is optionally substituted with one or more halogen, —CN, —NO₂, —OH, or —NH₂;
each R^{A′} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
R^{VN2} is hydrogen or C₁₋₆ alkyl, wherein the C₁₋₆ alkyl is optionally substituted with one or more halogen, —CN, —NO₂, —OH, or —NH₂;
each R^{V4} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
each R^{V5} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂; and
R^{V6} is C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, —NH₂, C₁₋₆ alkyl, C₂₋₆ alkenyl, or C₂₋₆ alkynyl.

Exemplary Embodiment No. 2. The compound of Exemplary Embodiment No. 1, wherein X is N(R^{X1}), and R^{A} is oxo.

Exemplary Embodiment No. 3. The compound of Exemplary Embodiment No. 1, wherein X is C(R^{X2}), and R^{A} and R^{B2}, together with the intervening atoms to which they are attached, form C₆ aryl or 6-membered heteroaryl, wherein the Co aryl or 6-membered heteroaryl is optionally substituted with one or more halogen, —CN, —NO₂, —OH, or —NH₂.

Exemplary Embodiment No. 4. The compound of Exemplary Embodiment No. 3, wherein R^{A} and R^{B2}, together with the intervening atoms to which they are attached, form C₆ aryl or pyridinyl.

Exemplary Embodiment No. 5. The compound of any one of Exemplary Embodiment Nos. 1-4, wherein R^{B1} and R^{B4}, together with the intervening atoms to which they are attached, form 3- to 8-membered heterocyclyl or 5- to 6-membered heteroaryl, wherein the 3- to 8-membered heterocyclyl or 5- to 6-membered heteroaryl is optionally substituted with one or more halogen, —CN, —NO₂, —OH, —NH₂, C₁₋₆ alkyl, C₁₋₆ alkoxy, C₁₋₆ alkylamino, C₂₋₆ alkenyl, or C₂-6 alkynyl.

Exemplary Embodiment No. 6. The compound of Exemplary Embodiment No. 5, wherein R^{B1} and R^{B4}, together with the intervening atoms to which they are attached, form dihydrofuran or pyrazole optionally substituted with one or more C₁₋₆ alkyl.

Exemplary Embodiment No. 7. The compound of any one of Exemplary Embodiment Nos. 1-4, wherein R^{B1} is hydrogen, and R^{B4} is C₁₋₆ alkyl or C₃₋₁₂ carbocyclyl.

Exemplary Embodiment No. 8. The compound of any one of Exemplary Embodiment Nos. 1-7, wherein R^{B5} is —NH₂.

Exemplary Embodiment No. 9. The compound of any one of Exemplary Embodiment Nos. 1-8, wherein each R^{C} is independently hydrogen.

Exemplary Embodiment No. 10. The compound of any one of Exemplary Embodiment Nos. 1-9, wherein n^{D} is 0, and Y is S.

Exemplary Embodiment No. 11. The compound of any one of Exemplary Embodiment Nos. 1-9, wherein n^{D} is 1, and Y is C(R^{D}).

Exemplary Embodiment No. 12. The compound of any one of Exemplary Embodiment Nos. 1-11, wherein one R^{D} is

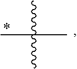

and each of the remaining R^{D} is independently hydrogen.

Exemplary Embodiment No. 13. The compound of any one of Exemplary Embodiment Nos. 1-12, wherein Tis of Formula I-1-a, I-1-b, I-1-c, or I-1-d

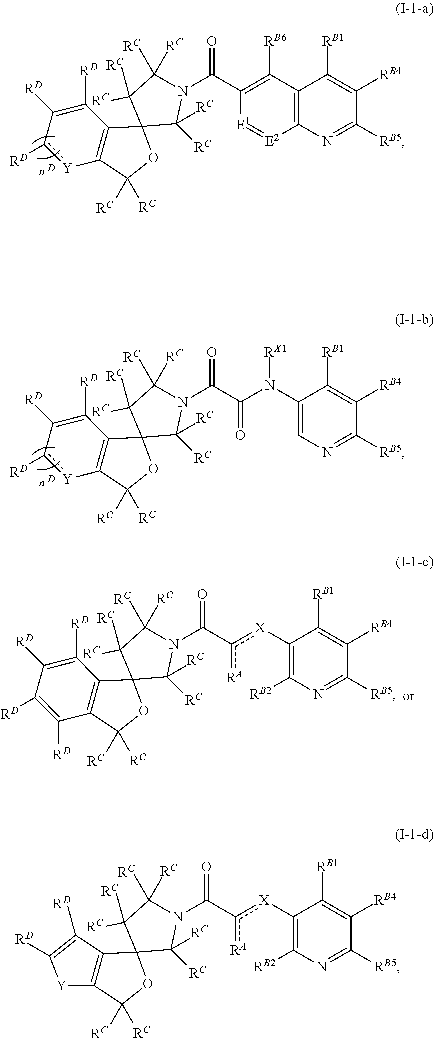

wherein:
E¹ is N or CR^{B7};
E² is N or CR^{B8};
R^{B6}, R^{B7}, and R^{B8} are independently hydrogen, halogen, —CN, —NO₂, —OH, or —NH₂; and
when T is of Formula I-1-d, Y is O or S.

Exemplary Embodiment No. 14. The compound of any one of Exemplary Embodiment Nos. 1-13, wherein each L′ is independently selected from —C(═O)—, —C(═O)N(R^{L′})—, —N(R^{L′})C(═O)—, —N(R^{L′})—, —O—, —O—(C₁₋₆ alkylene)-, —(C₁₋₆ alkylene)-O—, C₁₋₆ alkylene, C₃₋₁₂ carbocyclylene, and 5- to 10-membered heteroarylene.

Exemplary Embodiment No. 15. The compound of any one of Exemplary Embodiment Nos. 1-14, wherein L is **—[O—(C₁₋₆ alkylene)]₁₋₆-N(R^{L′})C(═O)—*, **—O—[(C₁₋₆ alkylene)]₁₋₃—N(R^{L′})C(═O)—*, *-(5- to 6-membered heteroarylene)-C(═O)NH—[(C₁₋₆ alkylene)-O]₁₋₆—**, *-(5- to 6-membered heteroarylene)-C(═O)N(R^{L′})—(C₃₋₁₂ carbocyclylene)-O—[(C₁₋₆ alkylene)-O]₁₋₃—**, or *-(5- to 6-membered heteroarylene)-C(═O)N(R^{L′})—[(C₁₋₆ alkylene)-O]₁₋₃—**.

Exemplary Embodiment No. 16. The compound of Exemplary Embodiment No. 15, wherein L is **—[O—(C₁₋₆ alkylene)]₁₋₆-N(H)C(═O)—*, *—O—[(C₁₋₆ alkylene)]₁₋₃—N(H)C(═O)—*, *-(pyridinylene)-C(═O)NH—[(C₁₋₆ alkylene)-O]₁₋₆—**-(pyridinylene)-C(═O)NH—(C₃₋₁₂ carbocyclylene)-O—[(C₁₋₆ alkylene)-O]₁₋₃—**, or *-(pyridinylene)-C(═O)NH—[(C₁₋₆ alkylene)-O]₁₋₃—**.

Exemplary Embodiment No. 17. The compound of any one of Exemplary Embodiment Nos. 1-16, wherein R^{V1} is C₃₋₆ carbocyclyl optionally substituted with one or more halogen.

Exemplary Embodiment No. 18. The compound of any one of Exemplary Embodiment Nos. 1-17, wherein R^{VN1} and R^{VN2} are each independently hydrogen.

Exemplary Embodiment No. 19. The compound of any one of Exemplary Embodiment Nos. 1-18, wherein R^{V2} is C₁₋₆ alkyl.

Exemplary Embodiment No. 20. The compound of any one of Exemplary Embodiment Nos. 1-19, wherein one R^{A′} is —OH, and each of the remaining R^{A′} is independently hydrogen.

Exemplary Embodiment No. 21. The compound of any one of Exemplary Embodiment Nos. 1-20, wherein each R^{V4} is independently hydrogen.

Exemplary Embodiment No. 22. The compound of any one of Exemplary Embodiment Nos. 1-21, wherein each R^{V5} is independently hydrogen.

Exemplary Embodiment No. 23. The compound of any one of Exemplary Embodiment Nos. 1-22, wherein R^{V6} is C₂₋₆ alkynyl or 5- to 10-membered heteroaryl optionally substituted with one or more C₁₋₆ alkyl.

Exemplary Embodiment No. 24. The compound of any one of Exemplary Embodiment Nos. 1-23, wherein V is of Formula I-3-a

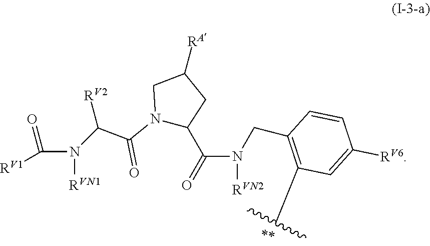

Exemplary Embodiment No. 25. A compound selected from the compounds in Table 1, or a pharmaceutically acceptable salt thereof.

Exemplary Embodiment No. 26. A pharmaceutical composition comprising the compound of any one of Exemplary Embodiment Nos. 1-25, and a pharmaceutically acceptable excipient.

Exemplary Embodiment No. 27. A method of treating a disease or disorder comprising administering to a patient in need thereof a compound of any one of Exemplary Embodiment Nos. 1-25.

Exemplary Embodiment No. 28. Use of a compound of any one of Exemplary Embodiment Nos. 1-25 in the manufacture of a medicament for treating a disease or disorder.

Exemplary Embodiment No. 29. A compound of any one of Exemplary Embodiment Nos. 1-25 for use in treating a disease or disorder.

Exemplary Embodiment No. 30. The method, use, or compound for use of any one of Exemplary Embodiment Nos. 27-29, wherein the disease or disorder is a PRMT5 protein-mediated disease or disorder.

Exemplary Embodiment No. 31. The method, use, or compound for use of any one of Exemplary Embodiment Nos. 27-30, wherein the disease or disorder is cancer.

### Examples

In order that the invention described herein may be more fully understood, the following examples are set forth. The examples described in this application are offered to illustrate the compounds, pharmaceutical compositions, and methods provided herein and are not to be construed in any way as limiting their scope.

### I. Synthesis and Characterization of Compounds

In the following examples, the chemical reagents were purchased from commercial sources (such as Alfa, Acros, Sigma Aldrich, TCI and Shanghai Chemical Reagent Company), and used without further purification.

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure. Compound purification was carried out as needed using a variety of traditional methods including, but not limited to, preparative chromatography under acidic, neutral, or basic conditions using either normal phase or reverse phase HPLC or flash columns or Prep-TLC plates.

Flash chromatography was performed on a Biotage Isolera One via column with silica gel particles of 100-200 mesh or 200-300 mesh. Analytical and preparative thin-layer chromatography was performed using silica gel 60 GF254 plates. Normal-phase silica gel chromatography (FCC) was also performed on silica gel (SiO₂) using prepacked cartridges.

Preparative reverse-phase high performance liquid chromatography (RP HPLC) was performed on either:

### Method A

Prep-HPLC with YMC-Actus Triart 18C (5 μm, 20×250 mm), and mobile phase of 5-99% ACN in water (0.1% HCOOH) over 10 min and then hold at 100% ACN for 2 min, at a flow rate of 25 mL/min; or

### Method B

Preparative supercritical fluid high performance liquid chromatography (SFC) was performed either on a Thar 80 Prep-SFC system, or Waters 80Q Prep-SFC system from Waters. The ABPR was set to 100 bar to keep the CO₂ in SF conditions, and the flow rate may verify according to the compound characteristics, with a flow rate ranging from 50 g/min to 70 g/min. The column temperature was ambient temperature.

Nuclear magnetic resonance (NMR) spectra were recorded using Brucker AVANCE NEO 400 MHz at around 20-30° C. unless otherwise specified. The following abbreviations are used: s, singlet; d, doublet; t, triplet; q, quartet; m, multiplet; dd, doublet of doublets; ddd, doublet of doublet of doublets; dt, doublet of triplets; bs, broad signal. Chemical shifts were reported in parts per million (ppm, δ) downfield from tetramethylsilane. It will be understood that for compounds comprising an exchangeable proton, said proton may or may not be visible on an NMR spectrum depending on the choice of solvent used for running the NMR spectrum and the concentration of the compound in the solution.

Mass spectra (MS) were obtained on a SHIMADZU LCMS-2020 MSD using electrospray ionization (ESI) in positive mode unless otherwise indicated. Calculated (calcd.) mass corresponds to the exact mass.

Chemical names were generated using ChemDraw Ultra 12.0, ChemDraw Ultra 14.0, ChemDraw Ultra 20.0 (CambridgeSoft Corp., Cambridge, MA) or ACD/Name Version 10.01 (Advanced Chemistry).

Compounds designated as R or S are enantiopure compounds where the absolute configuration was determined.

Intermediate 1. Ethyl 1′-(4-((tert-butoxycarbonyl)amino)-1-methyl-1H-pyrazolo[4,3-c]quinoline-8-carbonyl)-3H-spiro[isobenzofuran-1,3′-pyrrolidine]-5-carboxylate

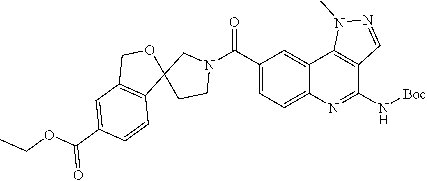

Step A: Ethyl 3H-spiro[isobenzofuran-1,3′-pyrrolidine]-5-carboxylate

1′-(tert-Butyl) 5-ethyl 3H-spiro[isobenzofuran-1,3′-pyrrolidine]-1′,5-dicarboxylate (prepared by analogous synthesis according to WO2005110992 A1, 0.10 g, 1 eq, 0.20 mmol) was dissolved in 4 N HCl-dioxane (1.00 mL) at 20° C. Then the mixture was stirred at 20° C. for 2 hours. The reaction was concentrated to ethyl 3H-spiro[isobenzofuran-1,3′-pyrrolidine]-5-carboxylate as a white solid.

LC-MS (ESI): mass calcd, for C₁₄H₁₇NO₃, 247.1; m/z found, 248.2 [M+H]⁺.

Step B: Ethyl 1′-(4-((tert-butoxycarbonyl)amino)-1-methyl-1H-pyrazolo[4,3-c]quinoline-8-carbonyl)-3H-spiro[isobenzofuran-1,3′-pyrrolidine]-5-carboxylate

To a mixture ethyl 3H-spiro[isobenzofuran-1,3′-pyrrolidine]-5-carboxylate (0.08 g, 1 eq, 0.2 mmol) in DMA (2.00 mL) was added DIEA (0.08 g, 0.1 mL, 3 eq, 0.6 mmol), 4-((tert-butoxycarbonyl)amino)-1-methyl-1H-pyrazolo[4,3-c]quinoline-8-carboxylic acid (prepared according to WO2022115377A1, 0.08 g, 1.1 eq, 0.2 mmol) and HATU (0.1 g, 1.5 eq, 0.3 mmol), then the mixture was stirred at 20° C. for 2 hours. The reaction was purified by Prep-HPLC (MeCN, H2O, FA(0.1%)) to Ethyl 1′-(4-((tert-butoxycarbonyl)amino)-1-methyl-1H-pyrazolo[4,3-c]quinoline-8-carbonyl)-3H-spiro[isobenzofuran-1,3′-pyrrolidine]-5-carboxylate (0.13 g, 0.18 mmol, 90%) as a yellow solid.

LC-MS (ESI): mass calcd, for C₃₁H₃₃N₅O₆, 571.2; m/z found, 572.2 [M+H]⁺.

Intermediate 2. (2S,4R)—N-(2-((17-amino-3,6,9,12,15-pentaoxaheptadecyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

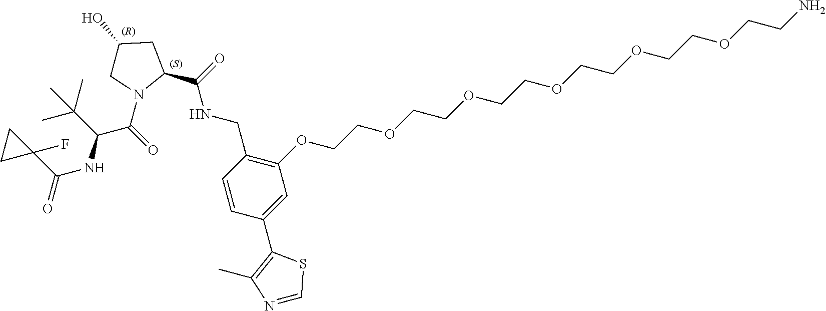

Step A: tert-butyl (17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12,15-pentaoxaheptadecyl)carbamate

To a solution of tert-butyl (17-bromo-3,6,9,12,15-pentaoxaheptadecyl)carbamate (0.11 mg, 1.0 mmol, 1.0 eq.), (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (50 mg, 0.1 mmol, 1.0 eq.) in DMF (3 mL) was added K₂CO₃ (25 mg, 0.18 mmol, 3.0 eq.). The mixture was stirred at 60° C. for 16 hours under N₂ atmosphere. The mixture was diluted with water (30 mL) and extracted with DCM (20 mL×3). The combined organic layers were washed with brine, dried over anhydrous Na₂SO₄ and concentrated under reduced pressure. The residue was purified by flash chromatographer (eluent, 0~10% MeOH in DCM) to give tert-butyl (17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12,15-pentaoxaheptadecyl)carbamate (55 mg, 65% yield) as a white solid.

LC-MS (ESI): mass calcd, for C₄₃H₆₆FN₅O₁₂S, 895.4; m/z found, 896.4 [M+H]⁺.

Step B: (2S,4R)—N-(2-((17-amino-3,6,9,12,15-pentaoxaheptadecyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

To a solution of tert-butyl (17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12,15-pentaoxaheptadecyl)carbamate (55 mg, 0.6 mmol) in Dioxane (2 mL) was added HCl in Dioxane (2 mL, 4 mol/L) at 0° C. The reaction mixture was stirred for 1 hour at room temperature under N₂ atmosphere. The mixture was concentrated by vacuum to give (2S,4R)—N-(2-((17-amino-3,6,9,12,15-pentaoxaheptadecyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (35 mg, 71.6% yield) as a white solid.

LC-MS (ESI): mass calcd, for C₃₈H₅₈FN₅O₁₀S, 795.4; m/z found, 796.4 [M+H]⁺.

Intermediate 3. (2S,4R)—N-(2-((14-amino-3,6,9,12-tetraoxatetradecyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

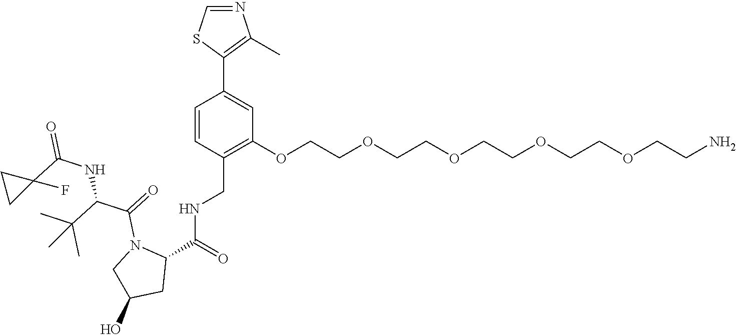 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCN)c1]

Prepared in an analogous synthesis as Intermediate 2. LC-MS (ESI): mass calcd. for C₃₆H₅₄FN₅O₉S, 751.4; m/z found, 752.2 [M+H]⁺.

Intermediate 4. (2S,4R)—N-(2-((14-aminotetradecyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

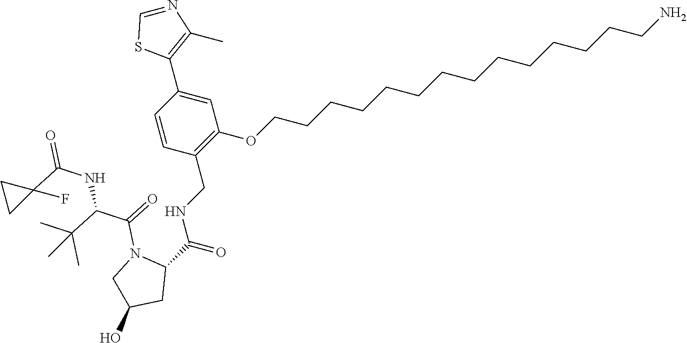 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCCCCN)c1]

Prepared in an analogous synthesis as Intermediate 2. LC-MS (ESI): mass calcd. for C₄₀H₆₂FN₅O₅S, 743.5; m/z found, 744.4 [M+H]⁺.

Intermediate 5. (2S,4R)—N-(2-(2-(2-(2-aminoethoxy)ethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

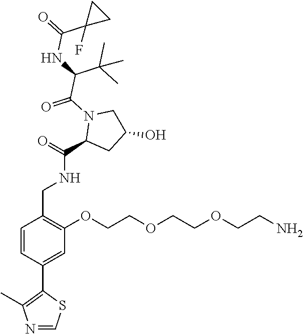 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCN)c1]

Prepared in analogous synthesis as Intermediate 2. LC-MS (ESI): mass calcd. for C₃₂H₄₆FN₅O₇S, 663.3; m/z found, 664.1 [M+H]⁺.

Intermediate 6. (2S,4R)—N-(2-(2-(((1r,4S)-4-aminocyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

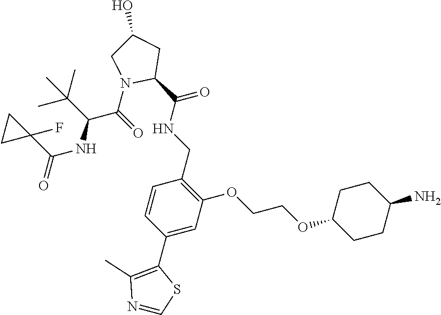 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](N)CC2)c1]

Step A: tert-butyl ((1r,4r)-4-(2-(benzyloxy)ethoxy)cyclohexyl)carbamate

To a solution of tert-butyl ((1r,4r)-4-hydroxycyclohexyl)carbamate (1.0 g, 4.6 mmol, 1.0 eq.) in THF (10 mL) was added 2-methylpropan-2-olate potassium (625 mg, 0.64 mL, 5.6 mmol, 1.2 eq.) at 0° C. and stirred for 30 min under 0° C. Then ((2-bromoethoxy)methyl)benzene (1 g, 4.64 mmol, 1.0 eq.) was added at 0° C. and the mixture stirred at room temperature for 16 hours. The mixture was poured into water and extracted with EtOAc (30 mL×3). The combined organic layers were washed with brine, dried over anhydrous Na₂SO₄ and concentrated under reduced pressure. The residue was purified by flash chromatographer (eluent, 0~30% EtOAc in PE) to give the tert-butyl ((1r,4r)-4-(2-(benzyloxy)ethoxy)cyclohexyl)carbamate (100 mg, 6% yield) as a colorless oil.

LC-MS (ESI): mass calcd, for C₂₀H₃₁NO₄, 349.26; m/z found, 366.8 [M+H₂O]⁺.

Step B: tert-butyl ((1r,4r)-4-(2-hydroxyethoxy)cyclohexyl)carbamate

To a solution of tert-butyl ((1r,4r)-4-(2-(benzyloxy)ethoxy)cyclohexyl)carbamate (100 mg, 286 μmol, 1.0 eq.) in MeOH (2.0 mL) was added Pd/C (20 mg, 10% Wt) at room temperature under nitrogen atmosphere. Then was stirred at room temperature for 5 hours under H₂ balloon. The mixture was filtered and concentrated under reduced pressure to dryness to give the tert-butyl ((1r,4r)-4-(2-hydroxyethoxy)cyclohexyl)carbamate (60 mg, 81% yield) as a yellow oil. LC-MS (ESI): mass calcd, for C₁₃H₂₅NO₄, 259.18; m/z found, 204.2 [M+H−56]⁺.

Step C: 2-(((1r,4r)-4-((tert-butoxycarbonyl)amino)cyclohexyl)oxy)ethyl4-methylbenzenesulfonate

To a mixture of tert-butyl ((1r,4r)-4-(2-hydroxyethoxy)cyclohexyl)carbamate (30.0 mg, 116 μmol, 1.0 eq.), TEA (35.1 mg, 48.4 μL, 347 μmol, 3.0 eq.) and DMAP (14.0 mg, 116 μmol, 1.0 eq.) in DCM (2 mL) was added TsCl (33 mg, 26.4 μL, 174 μmol, 1.5 eq.) at 0° C. Then the mixture was stirred at room temperature for 1 hour. The mixture was poured into water and extracted with EtOAc (20 mL×3). The combined organic layers were washed with brine, dried over anhydrous Na₂SO₄ and concentrated under reduced pressure to give the 2-(((1r,4r)-4-((tert-butoxycarbonyl)amino)cyclohexyl)oxy)ethyl 4-methylbenzenesulfonate (35 mg, 73% yield) as a yellow oil. The mixture was used for next step without further purification.

LC-MS (ESI): mass calcd, for C₂₀H₃₁NO₆S, 413.19; m/z found, 414.3 [M+H]⁺.

Step D: tert-butyl ((1S,4r)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamate

To a mixture of 2-(((1r,4r)-4-((tert-butoxycarbonyl)amino)cyclohexyl)oxy)ethyl 4-methylbenzenesulfonate (23.0 mg, 56.3 μmol, 1.0 eq.) and (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (30.0 mg, 56.3 μmol, 1.0 eq.) in DMF (2 mL) was added K₂CO₃ (23.4 mg, 169 μmol, 3.0 eq.) at room temperature. Then the mixture was stirred at 55° C. for 5 hours. The mixture was poured into water and extracted with EtOAc (30 mL×3). The combined organic layers were washed with brine, dried over anhydrous Na₂SO₄ and concentrated under reduced pressure. The residue was purified by flash chromatographer (eluent, 0~5% MeOH in DCM) to give the tert-butyl (1S,4r)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamate (30 mg, 69% yield) as a colorless oil.

LC-MS (ESI): mass calcd, for C₂₀H₃₁NO₄, 773.38; m/z found, 774.6 [M+H]⁺.

Step E: (2S,4R)—N-(2-(2-(((1r,4S)-4-aminocyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

Treatment with HCl in dioxane deprotection on tert-butyl ((1S,4r)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamate gave the title product.

LC-MS (ESI): mass calcd, for C₃₄H₄₈FN₅O₆S, 673.3; m/z found, 674.2 [M+H]⁺.

Intermediate 7. (2S,4R)—N-(2-(2-(((1r,4S)-4-aminocyclohexyl)oxy)ethoxy)-4-ethynylbenzyl)-1-((S)-2-(1-fluorocyclopropane-1-carboxamido)-3,3-dimethylbutanoyl)-4-hydroxypyrrolidine-2-carboxamide

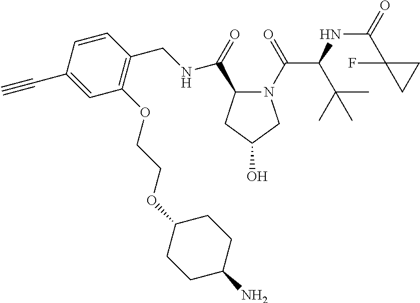 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](N)CC2)c1]

Step A: (2S,4R)—N-(4-ethynyl-2-hydroxybenzyl)-1-((S)-2-(1-fluorocyclopropane-1-carboxamido)-3,3-dimethylbutanoyl)-4-hydroxypyrrolidine-2-carboxamide

To a solution of 2-(aminomethyl)-5-ethynylphenol (90 mg, 0.61 mmol, 1.2 eq., WO2023278402) and (2S,4R)-1-((S)-2-(1-fluorocyclopropane-1-carboxamido)-3,3-dimethylbutanoyl)-4-hydroxypyrrolidine-2-carboxylic acid (170 mg, 0.51 mmol, 1.0 eq.) in DMF (15 mL) was added HATU (293 mg, 0.77 mmol, 1.5 eq.) and DIPEA (25 μL, 1.55 mmol, 3.0 eq.). Then the mixture was stirred at room temperature for 1 hour. The resulting solution was diluted with 30 mL of H₂O and extracted with EtOAc (20 mL×3). The combined organic layers were washed with brine, dried over Na₂SO₄ and concentrated under reduced pressure. The residue was purified by prep-HPLC (5~55% MeOH in water) to afford (2S,4R)—N-(4-ethynyl-2-hydroxybenzyl)-1-((S)-2-(1-fluorocyclopropane-1-carboxamido)-3,3-dimethylbutanoyl)-4-hydroxypyrrolidine-2-carboxamide (80 mg, 34% yield) as a yellow solid.

LC-MS (ESI): mass calcd, for C₂₄H₃₀FN₃O₅, 459.2; m/z found, 460.3 [M+H]⁺.

Steps B-C: (2S,4R)—N-(2-(2-(((1r,4S)-4-aminocyclohexyl)oxy)ethoxy)-4-ethynylbenzyl)-1-((S)-2-(1-fluorocyclopropane-1-carboxamido)-3,3-dimethylbutanoyl)-4-hydroxypyrrolidine-2-carboxamide

Prepared in an analogous synthesis to Intermediate 6. LC-MS (ESI): mass calcd. for C₃₂H₄₅FN₄O₆, 600.3; m/z found, 601.3 [M+H]⁺.

Intermediate 8. (2S,4R)—N-(2-(2-(4-aminobutoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

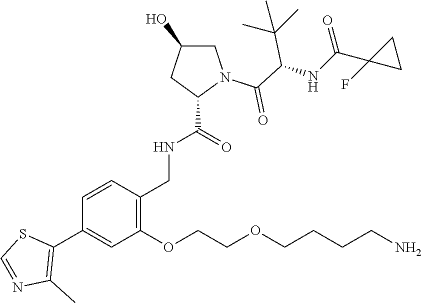 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCCN)c1]

Prepared in an analogous synthesis as Intermediate 2. LC-MS (ESI): mass calcd. for C₃₂H₄₆FN₅O₅S, 647.3; m/z found, 648.1 [M+H]⁺.

Intermediate 9. tert-Butyl 6′H-spiro[pyrrolidine-3,4′-thieno[2,3-c]furan]-1-carboxylate

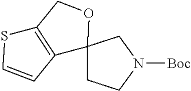 [SMILES: CC(C)(C)OC(=O)N1CCC2(C1)OCc1sccc12]

Step A: tert-Butyl 3-(4-bromo-2-(hydroxymethyl)phenyl)-3-hydroxypyrrolidine-1-carboxylate

To a mixture of 3-bromo-2-(hydroxymethyl)thiophene (3.00 g, 1 eq, 15.5 mmol) in THE (15.00 mL), was added with n-butyllithium (2.5M in hexanes 2.49 g, 3.49 mL, 2.5 eq, 38.8 mmol) at −70° C. Then the mixture was stirred at −70° C. for 1 hour. It was then added by N—BOC protected pyrrolidin-3-one, (3.17 g, 1.1 eq, 17.1 mmol) in THF (10 mL) at −50° C. and the mixture was stirred for 0.5 hour before warmed up to 20° C. overnight. The reaction was poured to water (30 mL), extracted with EtOAc (20 mL×3), the organic layers were washed with brine (30 mL), dried over Na₂SO₄, filtered, concentrated and purified by Prep-HPLC (MeCN, H₂O, FA (0.1%)) to give tert-butyl 3-hydroxy-3-(2-(hydroxymethyl)thiophen-3-yl)pyrrolidine-1-carboxylate (1.70 g, 5.68 mmol, 36.5%) as a yellow solid.

MS Calcd.: 299.1; MS Found: 322.0 [M+23]⁺.

Step B: tert-Butyl 6′H-spiro[pyrrolidine-3,4′-thieno[2,3-c]furan]-1-carboxylate

To a mixture of tert-butyl 3-hydroxy-3-(2-(hydroxymethyl)thiophen-3-yl)pyrrolidine-1-carboxylate (1.00 g, 1 eq, 3.34 mmol) in THF (1.50 mL) at 20° C., was added with LiHMDS (1.40 g, 2.5 eq, 8.35 mmol) at 0° C. Then the mixture was stirred at 0° C. for 0.5 hour followed by addition of p-toluenesulfonyl chloride (764 mg, 610 μL, 1.2 eq, 4.01 mmol) at 0° C. Then the mixture was stirred at 20° C. for 12 hours. The reaction was poured to water (30 mL), extracted with EtOAc (20 mL×3). The organic layers were washed with brine (30 mL), dried over Na₂SO₄, filtered, concentrated and purified by Prep-HPLC (MeCN, H₂O, FA (0.1%)) to give tert-butyl 6′H-spiro[pyrrolidine-3,4′-thieno[2,3-c]furan]-1-carboxylate (0.3 g, 1 mmol, 31.9%) as a yellow solid.

MS Calcd.: 281.1; MS Found: 226.0 [M+H−56]⁺.

Example B1. 1′-(4-amino-1-methyl-1H-pyrazolo[4,3-c]quinoline-8-carbonyl)-N-(17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12,15-pentaoxaheptadecyl)-3H-spiro[isobenzofuran-1,3′-pyrrolidine]-5-carboxamide

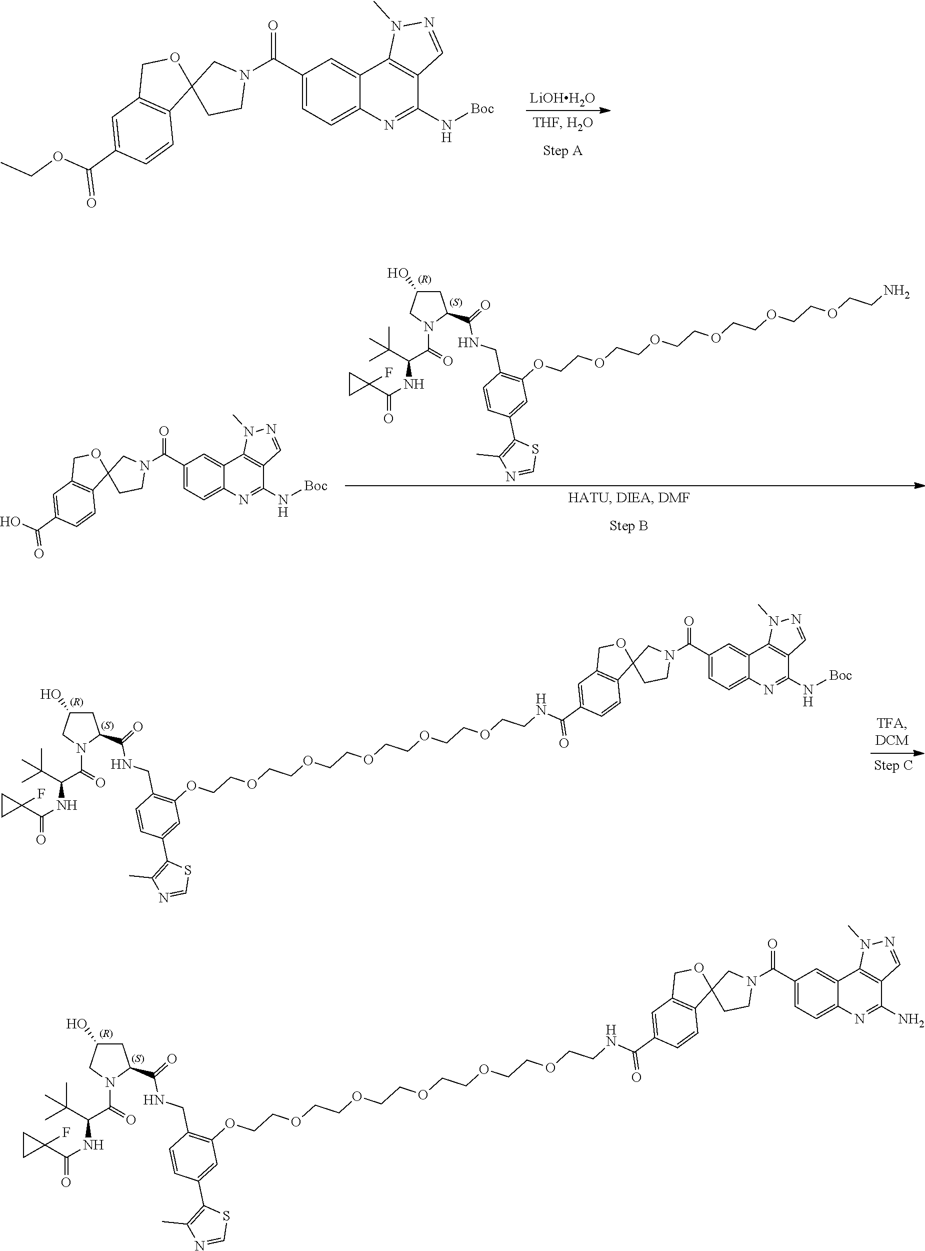

Step A: 1′-(4-((tert-butoxycarbonyl)amino)-1-methyl-1H-pyrazolo[4,3-c]quinoline-8-carbonyl)-3H-spiro[isobenzofuran-1,3′-pyrrolidine]-5-carboxylic acid

To a mixture of ethyl 1′-(4-((tert-butoxycarbonyl)amino)-1-methyl-1H-pyrazolo[4,3-c]quinoline-8-carbonyl)-3H-spiro[isobenzofuran-1,3′-pyrrolidine]-5-carboxylate (130.0 mg, 227 μmol, 1.0 eq.) in THF (4.0 mL) was added H₂O (1.0 mL) and LiOH·H₂O (8.2 mg, 341 μmol, 1.5 eq.) at room temperature. Then the mixture was stirred at room temperature for 2 hours. The mixture was concentrated under reduced pressure to dryness to give the 1′-(4-((tert-butoxycarbonyl)amino)-1-methyl-1H-pyrazolo[4,3-c]quinoline-8-carbonyl)-3H-spiro[isobenzofuran-1,3′-pyrrolidine]-5-carboxylic acid (120 mg, 97% yield) as a white solid.

LC-MS (ESI): mass calcd, for C₂₉H₂₉N₅O₆, 543.2; m/z found, 544.2 [M+H]⁺.

Step B: tert-butyl (8-(5-((17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12,15-pentaoxaheptadecyl)carbamoyl)-3H-spiro[isobenzofuran-1,3′-pyrrolidine]-1′-carbonyl)-1-methyl-1H-pyrazolo[4,3-c]quinolin-4-yl)carbamate

To a solution of 1′-(4-((tert-butoxycarbonyl)amino)-1-methyl-1H-pyrazolo[4,3-c]quinoline-8-carbonyl)-3H-spiro[isobenzofuran-1,3′-pyrrolidine]-5-carboxylic acid (6.8 mg, 12.6 μmol, 1.0 eq.), (2S,4R)—N-(2-((17-amino-3,6,9,12,15-pentaoxaheptadecyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (10 mg, 12.6 μmol, 1.0 eq.), HATU (7.2 mg, 18.8 μmol, 1.5 eq.) in DMF (1 mL) was added DIPEA (4.9 mg, 37.7 μmol, 3.0 eq.) at room temperature. The reaction mixture was stirred at room temperature for 30 mins under N₂ atmosphere. The mixture was diluted with water (10 mL) and extracted with EtOAc (10 mL×3). The combined organic layers were washed with brine, dried over anhydrous Na₂SO₄ and concentrated under reduced pressure to dryness to give tert-butyl (8-(5-((17-(2-(((2S,4R)-1-((S)-2-(1-fluorocyclopropane-1-carboxamido)-3,3-dimethyl butanoyl)-4-hydroxypyrrolidine-2-carboxamido)methyl)-5-(4-methylthiazol-5-yl)phenoxy)-3,6,9,12,15-pentaoxaheptadecyl)carbamoyl)-3H-spiro[isobenzofuran-1,3′-pyrrolidine]-1′-carbonyl)-1-methyl-1H-pyrazolo[4,3-c]quinolin-4-yl)carbamate (10 mg, 60% yield) as yellow oil.

LC-MS (ESI): mass calcd, for C₆₇H₈₅FN₁₀O₁₅S, 1320.6; m/z found, 1321.1 [M+H]⁺.

Step C: 1′-(4-amino-1-methyl-1H-pyrazolo[4,3-c]quinoline-8-carbonyl)-N-(17-(2-(((2S,4R)-1-((S)-2-(1-fluorocyclopropane-1-carboxamido)-3,3-dimethylbutanoyl)-4-hydroxypyrrolidi-ne-2-carboxamido)methyl)-5-(4-methylthiazol-5-yl)phenoxy)-3,6,9,12,15-pentaoxaheptade-cyl)-3H-spiro[isobenzofuran-1,3′-pyrrolidine]-5-carboxamide

To a solution of tert-butyl (8-(5-((17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methyl thiazol-5-yl)phenoxy)-3,6,9,12,15-pentaoxaheptadecyl)carbamoyl)-3H-spiro[isobenzofuran-1,3′-pyrrolidine]-1′-carbonyl)-1-methyl-1H-pyrazolo[4,3-c]quinolin-4-yl)carbamate (10 mg, 7.6 μmol, 1.0 eq.) in DCM (2.0 mL) was added TFA (0.7 mL) under N₂ atmosphere. The mixture was concentrated under reduced pressure to dryness. The residue was purified by prep-HPLC (C18, 20~95%, MeCN in H₂O with 0.1% HCOOH) to give 1′-(4-amino-1-methyl-1H-pyrazolo[4,3-c]quinoline-8-carbonyl)-N-(17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12,15-pentaoxaheptadecyl)-3H-spiro[isobenzofuran-1,3′-pyrrolidine]-5-carboxamide (1.5 mg, 13% yield) as a white solid.

LC-MS (ESI): mass calcd, for C₆₂H₇₇FN₁₀O₁₃S, 1220.5; m/z found, 1221.6 [M+H]⁺.

Example B2. 1′-(4-amino-1-methyl-1H-pyrazolo[4,3-c]quinoline-8-carbonyl)-N-(14-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12-tetraoxatetradecyl)-3H-spiro[isobenzofuran-1,3′-pyrrolidine]-5-carboxamide

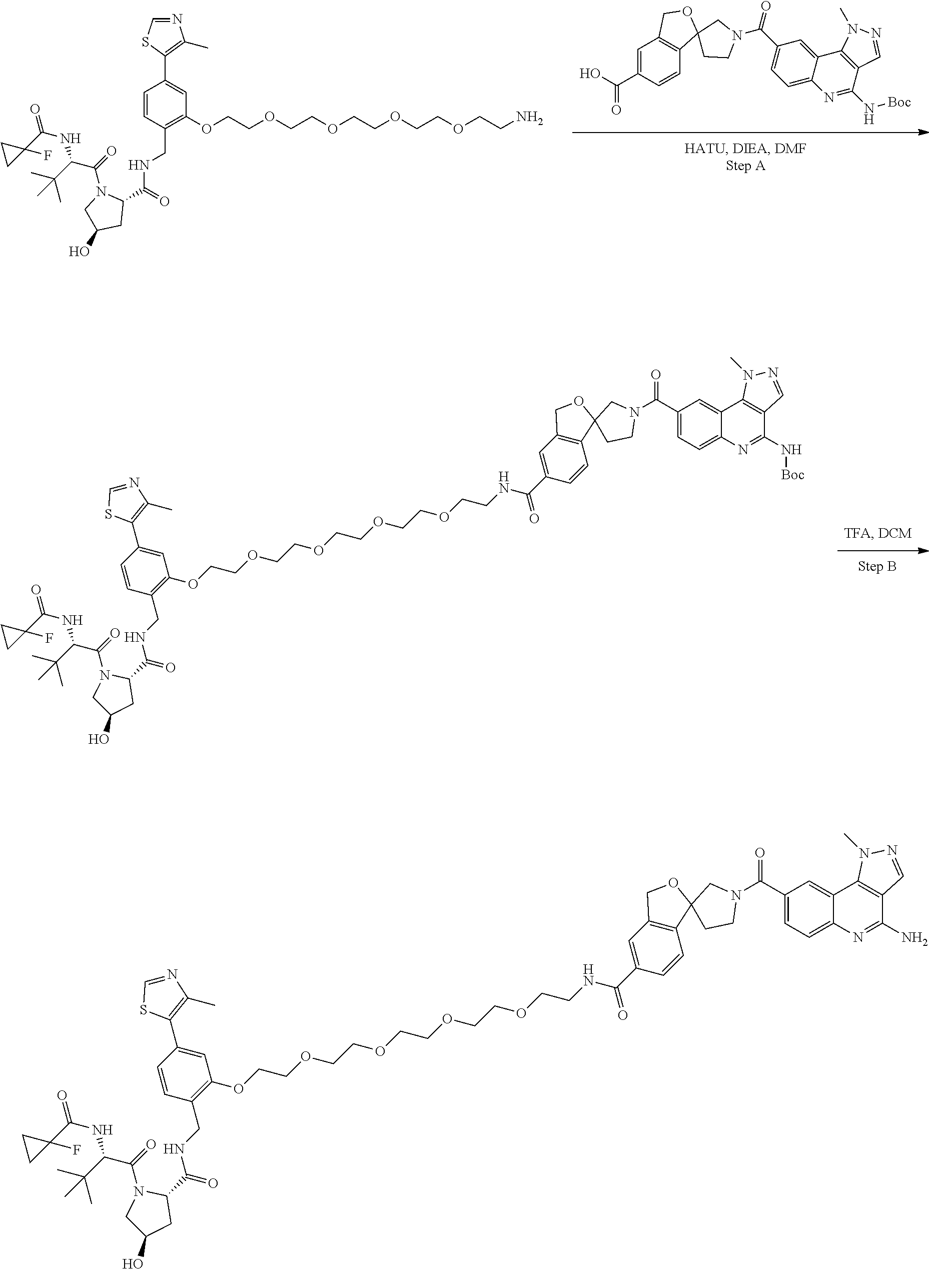

Example B2 was synthesized following the same procedures as described in Example B1 (1.5 mg, 13% yield over 2 steps) as a white solid. LC-MS (ESI): mass calcd, for C₆₀H₇₃FN₁₀O₁₂S, 1176.5; m/z found, 1177.2 [M+H]⁺.

Example B3. 1′-(4-amino-1-methyl-1H-pyrazolo[4,3-c]quinoline-8-carbonyl)-N-(14-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)tetradecyl)-3H-spiro[isobenzofuran-1,3′-pyrrolidine]-5-carboxamide

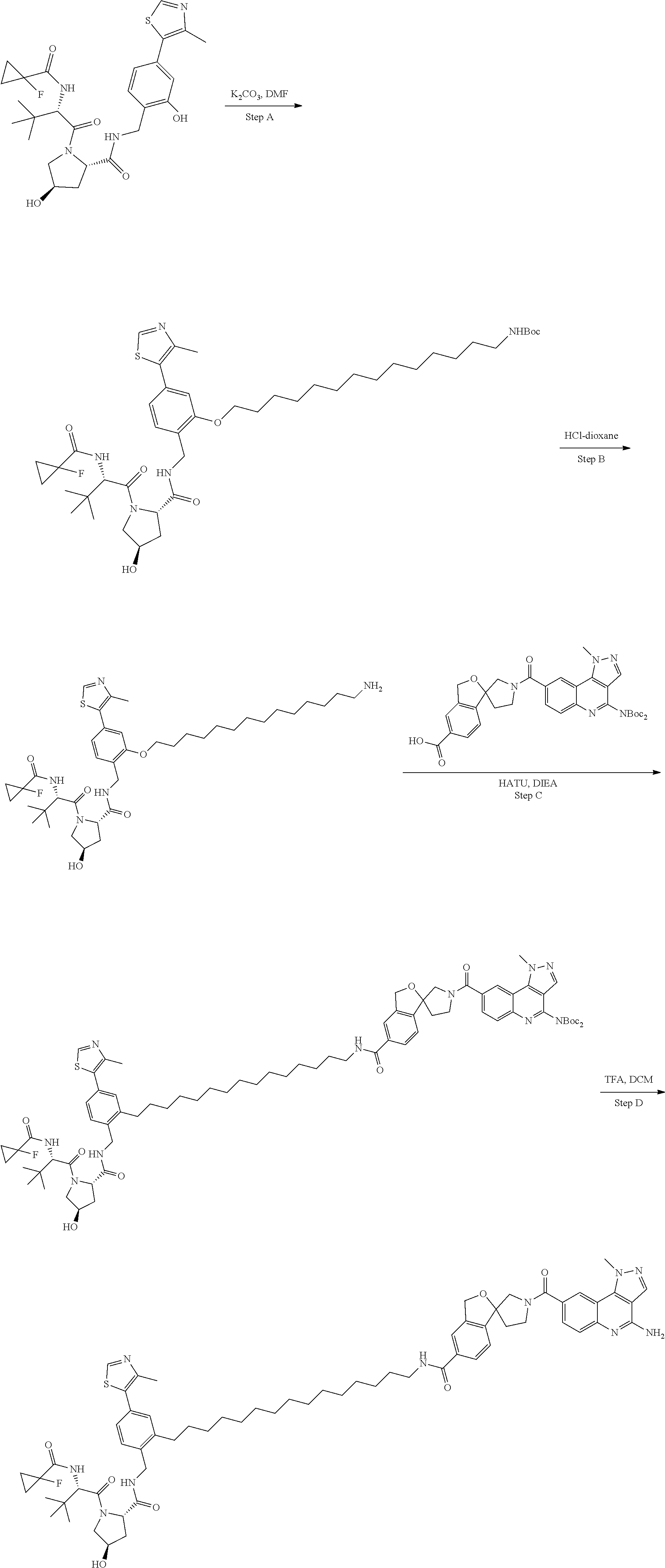

Example B3 was synthesized following the same procedures as described in Example B1 (3.1 mg, 9% yield over 4 steps) as a white solid. LC-MS (ESI): mass calcd, for C₆₆H₈₂FN₉O₈S, 1168.6; m/z found, 1170.1 [M+H]⁺.

Example B4. 5-(1′-(2-amino-3-methylquinoline-6-carbonyl)-3H-spiro[isobenzofuran-1,3′-pyrrolidin]-5-yl)-N-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)picolinamide

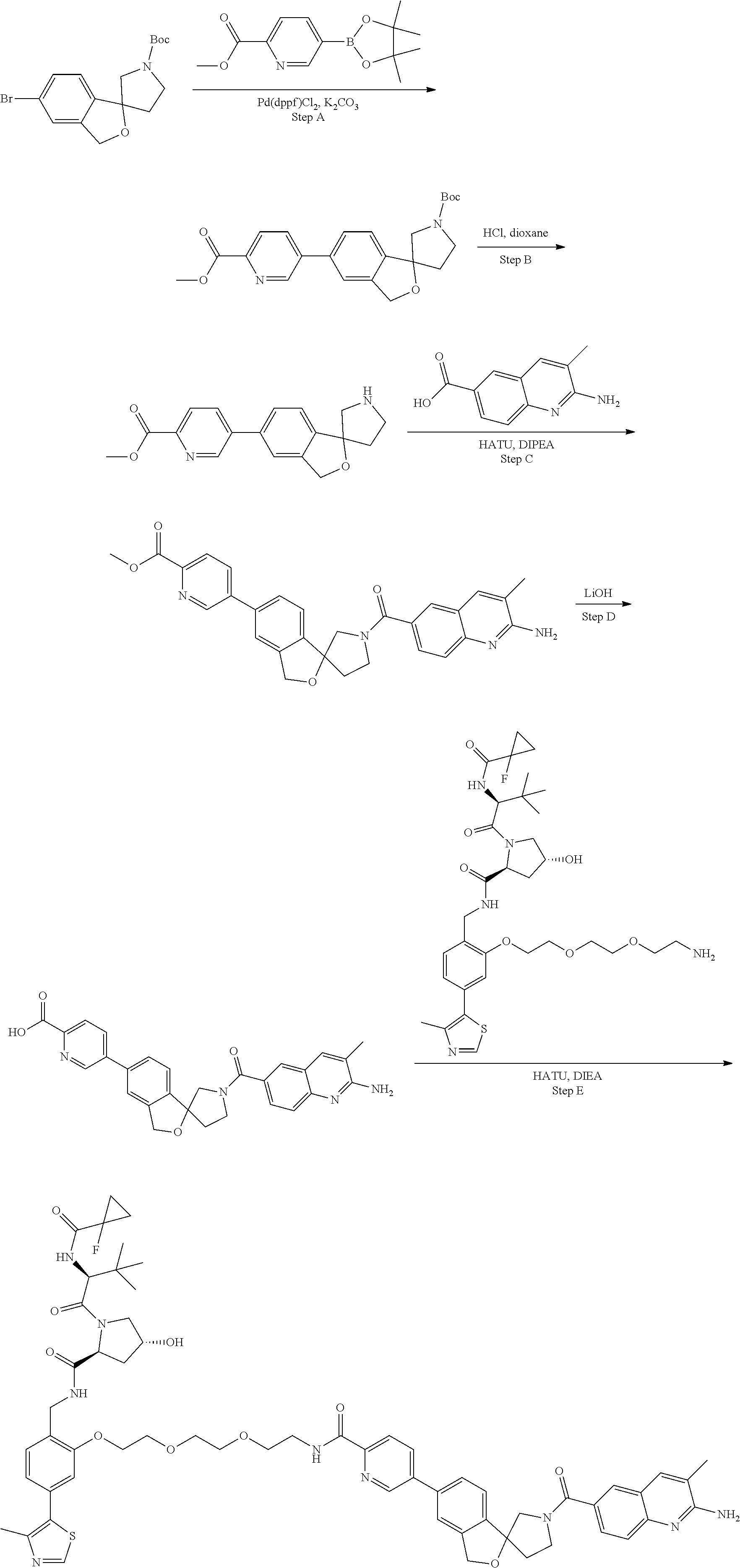

Step A: tert-butyl 5-(6-(methoxycarbonyl)pyridin-3-yl)-3H-spiro[isobenzofuran-1,3′-pyrrolidine]-1′-carboxylate

To a mixture of tert-butyl 5-bromo-3H-spiro[isobenzofuran-1,3′-pyrrolidine]-1′-carboxylate (prepared according to the procedure in WO2005110992 A1, 300 mg, 850 μmol, 1.0 eq.), methyl 5-(4,4,5,5-tetramethyl-1,3,2-dioxaborolan-2-yl)picolinate (300 mg, 1.1 mol, 1.3 eq.) and K₂CO₃ (350 mg, 2.5 mmol, 3.0 eq.) in 1,4-dioxane (4.0 mL) and H₂O (0.5 mL) was added Pd(dppf)Cl₂ (123 mg, 170 μmol, 0.2 eq.) under nitrogen atmosphere. Then the mixture was stirred at 90° C. for 5 hours. The mixture was poured into water and extracted with EtOAc (20 mL×3). The combined organic layers were washed with brine, dried over anhydrous Na₂SO₄ and concentrated under reduced pressure. The residue was purified by flash chromatographer (eluent, 0~50% EtOAc in PE) to give tert-butyl 5-(6-(methoxycarbonyl)pyridin-3-yl)-3H-spiro[isobenzofuran-1,3′-pyrrolidine]-1′-carboxylate (210 mg, 60% yield) as a white solid.

LC-MS (ESI): mass calcd, for C₂₃H₂₆N₂O₅, 410.4; m/z found, 411.1 [M+H]⁺.

Step B: methyl 5-(3H-spiro[isobenzofuran-1,3′-pyrrolidin]-5-yl)picolinate

To a solution of tert-butyl 5-(6-(methoxycarbonyl)pyridin-3-yl)-3H-spiro[isobenzofuran-1,3′-pyrrolidine]-1′-carboxylate (210 mg, 510 μmol, 1.0 eq.) in dioxane (2 mL) was added HCl in dioxane (2 mL, 4 mol/L) at 0° C. The reaction mixture was stirred for 1 hour at room temperature under N₂ atmosphere. The mixture was concentrated by vacuum to give methyl 5-(3H-spiro[isobenzofuran-1,3′-pyrrolidin]-5-yl)picolinate (190 mg HCl salt, 99% yield) as a white solid.

LC-MS (ESI): mass calcd, for C₁₈H₁₈N₂O₃, 310.1; m/z found, 311.1 [M+H]⁺.

Step C: methyl 5-(1′-(2-amino-3-methylquinoline-6-carbonyl)-3H-spiro[isobenzofuran-1,3′-pyrrolidin]-5-yl)picolinate

To a solution of methyl 5-(3H-spiro[isobenzofuran-1,3′-pyrrolidin]-5-yl)picolinate (40 mg, 0.13 mmol, 1.0 eq.), 2-amino-3-methylquinoline-6-carboxylic acid (30 mg, 0.15 mmol, 1.2 eq.), HATU (75 mg, 0.2 mmol, 1.5 eq.) in DMF (2 mL) was added DIPEA (50 mg, 0.4 mmol, 3.0 eq.) at 0° C. The reaction mixture was stirred at 0° C. for 0.5 h under N₂ atmosphere. The mixture was diluted with water (30 mL) and extracted with EtOAc (20 mL×3). The combined organic layers were washed with brine, dried over anhydrous Na₂SO₄ and concentrated under reduced pressure. The residue was purified by flash chromatographer (eluent, 0~10% MeOH in DCM) to give methyl 5-(1′-(2-amino-3-methylquinoline-6-carbonyl)-3H-spiro[isobenzofuran-1,3′-pyrrolidin]-5-yl)picolinate (45 mg, 70% yield) as a solid.

LC-MS (ESI): mass calcd, for C₂₉H₂₆N₄O₄, 494.2; m/z found, 495.2 [M+H]⁺.

Step D: 5-(1′-(2-amino-3-methylquinoline-6-carbonyl)-3H-spiro[isobenzofuran-1,3′-pyrrolidin]-5-yl)picolinic acid

To a mixture of methyl 5-(1′-(2-amino-3-methylquinoline-6-carbonyl)-3H-spiro[isobenzofuran-1,3′-pyrrolidin]-5-yl)picolinate (45.0 mg, 0.1 mmol, 1.0 eq.) in THF (2.0 mL)/H₂O (0.4 mL) was added LiOH·H₂O (10 mg, 0.3 mol, 3.0 eq.) at room temperature. Then the mixture was stirred at room temperature for 2 hours. The mixture was concentrated under reduced pressure to dryness to give 5-(1′-(2-amino-3-methylquinoline-6-carbonyl)-3H-spiro[isobenzofuran-1,3′-pyrrolidin]-5-yl)picolinic acid (45 mg lithium salt, 95% yield) as a white solid.

LC-MS (ESI): mass calcd, for C₂₈H₂₄N₄O₄, 480.2; m/z found, 481.2 [M+H]⁺.

Step E: 5-(1′-(2-amino-3-methylquinoline-6-carbonyl)-3H-spiro[isobenzofuran-1,3′-pyrrolidin]-5-yl)-N-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)picolinamide

To a solution of (2S,4R)—N-(2-(2-(2-(2-aminoethoxy)ethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (30 mg, 0.05 mmol, 1.0 eq.), 5-(1′-(2-amino-3-methylquinoline-6-carbonyl)-3H-spiro[isobenzofuran-1,3′-pyrrolidin]-5-yl)picolinic acid (20 mg, 0.05 mmol, 1.0 eq.), HATU (75 mg, 0.2 mmol, 1.5 eq.) in DMF (2 mL) was added DIPEA (30 mg, 0.2 mmol, 3.0 eq.) at 0° C. The reaction mixture was stirred at 0° C. for 30 mins under N₂ atmosphere. The mixture was diluted with water (30 mL) and extracted with EtOAc (20 mL×3). The combined organic layers were washed with brine, dried over anhydrous Na₂SO₄ and concentrated under reduced pressure. The residue was purified by prep-HPLC (C18, 20~95%, MeCN in H₂O with 0.1% HCOOH) to give 5-(1′-(2-amino-3-methylquinoline-6-carbonyl)-3H-spiro[isobenzofuran-1,3′-pyrrolidin]-5-yl)-N-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)picolinamide (5 mg, 10% yield) as a white solid.

LC-MS (ESI): mass calcd, for C₆₀H₆₈FN₉O₁₀S, 1125.5; m/z found, 1126.3 [M+H]⁺.

Example B5. 5-(1-(2-amino-3-methylquinoline-6-carbonyl)-6′H-spiro[pyrrolidine-3,4′-thieno[2,3-c]furan]-2′-yl)-N-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)picolinamide

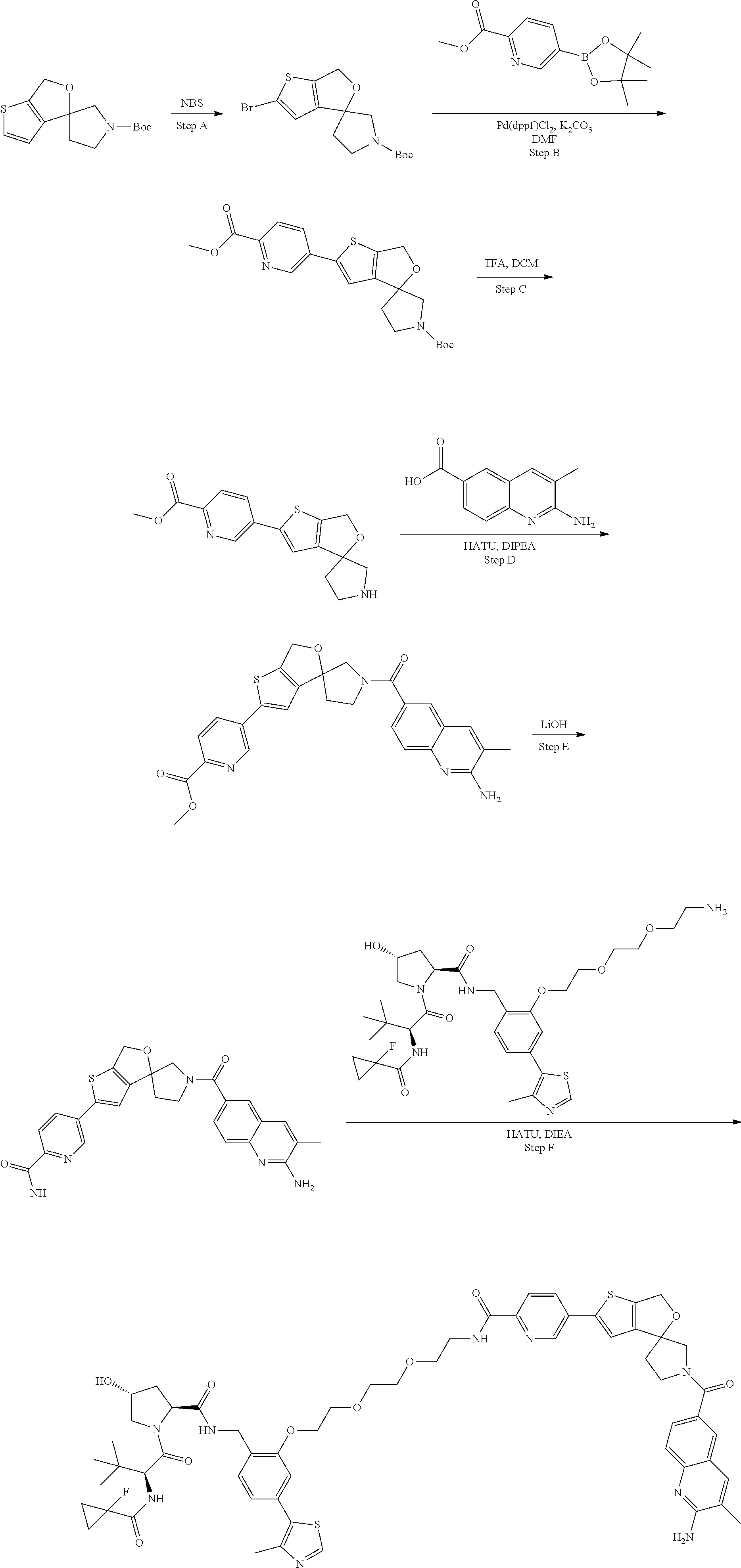

Step A: tert-butyl 2′-bromo-6′H-spiro[pyrrolidine-3,4′-thieno[2,3-c]furan]-1-carboxylate

To a solution of tert-butyl 6′H-spiro[pyrrolidine-3,4′-thieno[2,3-c]furan]-1-carboxylate (400 mg, 1.4 mmol, 1.0 eq.) in MeCN (8 mL) was added NBS (254 mg, 1.4 mmol, 1.0 eq.). The mixture was stirred at 0° C. for 1 hour under N₂ atmosphere. The mixture was diluted with water (30 mL) and extracted with EtOAc (20 mL×3). The combined organic layers were washed with brine, dried over anhydrous Na₂SO₄ and concentrated under reduced pressure. The residue was purified by flash chromatographer (eluent, 0~30% EtOAc in PE) to give tert-butyl 2′-bromo-6′H-spiro[pyrrolidine-3,4′-thieno[2,3-c]furan]-1-carboxylate (350 mg, 72% yield) as a white solid.

LC-MS (ESI): mass calcd, for C₁₄H₁₈BrNO₃S, 359, 361; m/z found, 360,362 [M+H]⁺.

Step B: tert-butyl 2′-(6-(methoxycarbonyl)pyridin-3-yl)-6′H-spiro[pyrrolidine-3,4′-thieno[2,3-c]furan]-1-carboxylate

To a solution of tert-butyl 2′-bromo-6′H-spiro[pyrrolidine-3,4′-thieno[2,3-c]furan]-1-carboxylate (400 mg, 1.11 mmol, 1.0 eq.), methyl 5-(4,4,5,5-tetramethyl-1,3,2-dioxaborolan-2-yl)picolinate (436 mg, 1.7 mmol, 1.5 eq.) and Pd(dppf)Cl₂ (160 mg, 0.25 mmol, 0.1 eq.) in DMF (30 mL) was added K₂CO₃ (300 mg, 2.2 mmol, 2.0 eq.), the mixture was stirred for 10 min at 100° C. The resulting solution was diluted with H₂O (50 mL), extracted with EtOAc (20 mL×3). The combined organic layers were washed with brine (20 mL×2), dried over Na₂SO₄ and concentrated under reduced pressure, the residue was purified by flash column chromatography on silica gel using 1:1~0:1 of hexanes ethyl acetate as eluent to give tert-butyl 2′-(6-(methoxycarbonyl)pyridin-3-yl)-6′H-spiro[pyrrolidine-3,4′-thieno[2,3-c]furan]-1-carboxylate (320 mg, 69% yield) as a white solid.

LC-MS (ESI): mass calcd, for C₂₁H₂₄N₂O₅S, 416.1; m/z found, 417.1 [M+H]⁺.

Step C: methyl 5-(6′H-spiro[pyrrolidine-3,4′-thieno[2,3-c]furan]-2′-yl)picolinate

To a solution of tert-butyl 2′-(6-(methoxycarbonyl)pyridin-3-yl)-6′H-spiro[pyrrolidine-3,4′-thieno[2,3-c]furan]-1-carboxylate (100 mg, 240 μmol, 1.0 eq.) in DCM (3.0 mL) was added TFA (1 mL), the mixture was stirred for 1 hour at room temperature. The mixture was concentrated by vacuum to afford methyl 5-(6′H-spiro[pyrrolidine-3,4′-thieno[2,3-c]furan]-2′-yl)picolinate (100 mg, TFA salt) as a yellow oil.

LC-MS (ESI): mass calcd, for C₁₆H₁₆N₂O₃S, 316.1; m/z found, 317.1 [M+H]⁺.

Step D: methyl 5-(1-(2-amino-3-methylquinoline-6-carbonyl)-6′H-spiro[pyrrolidine-3,4′-thieno[2,3-c]furan]-2′-yl)picolinate

To a solution of methyl 5-(6′H-spiro[pyrrolidine-3,4′-thieno[2,3-c]furan]-2′-yl)picolinate (35 mg, 111 μmol, 1.0 eq.), HATU (84 mg, 221 μmol, 2.0 eq.), 2-amino-3-methylquinoline-6-carboxylic acid (27 mg, 111 μmol, 1.0 eq.) in DMF (4 mL) was added Et₃N (47 μL, 332 μmol, 3.0 eq.), the mixture was stirred for 1 hour. The resulting solution was diluted with H₂O (20 mL), extracted with EtOAc (20 mL×3). The combined organic layers were washed with brine (20 mL×2), dried over Na₂SO₄ and concentrated under reduced pressure, the residue was purified by flash column chromatography on silica gel using 3:1~1:1 hexanes and ethyl acetate as eluent to give methyl 5-(1-(2-amino-3-methylquinoline-6-carbonyl)-6′H-spiro[pyrrolidine-3,4′-thieno[2,3-c]furan]-2′-yl)picolinate (20 mg, 33% yield) as a yellow solid.

LC-MS (ESI): mass calcd, for C₂₇H₂₄N₄O₄S, 500.1; m/z found, 501.1 [M+H]⁺.

Step E: 5-(1-(2-amino-3-methylquinoline-6-carbonyl)-6′H-spiro[pyrrolidine-3,4′-thieno[2,3-c]furan]-2′-yl)picolinic acid

To a solution of methyl 5-(1-(2-amino-3-methylquinoline-6-carbonyl)-6′H-spiro[pyrrolidine-3,4′-thieno[2,3-c]furan]-2′-yl)picolinate (23 mg, 46 μmol, 1.0 eq.) in THF (2 mL) and water (2 mL) was added LiOH (2.2 mg, 92 μmol, 2.0 eq.), the mixture was stirred for 2 hours. The mixture was concentrated by vacuum to afford 5-(1-(2-amino-3-methylquinoline-6-carbonyl)-6′H-spiro[pyrrolidine-3,4′-thieno[2,3-c]furan]-2′-yl)picolinic acid (20 mg, lithium salt, 90% yield) as a white solid.

LC-MS (ESI): mass calcd, for C₂₆H₂₂N₄O₄S, 486.1; m/z found, 487.2 [M+H]⁺.

Step F: 5-(1-(2-amino-3-methylquinoline-6-carbonyl)-6′H-spiro[pyrrolidine-3,4′-thieno[2,3-c]furan]-2′-yl)-N-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)picolinamide

To a solution of 5-(1-(2-amino-3-methylquinoline-6-carbonyl)-6′H-spiro[pyrrolidine-3,4′-thieno[2,3-c]furan]-2′-yl)picolinic acid (20 mg, 41 μmol, 1.0 eq.), (2S,4R)—N-(2-(2-(2-(2-aminoethoxy)ethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (27 mg, 41 μmol, 1.0 eq.), HATU (32 mg, 83 μmol, 2.0 eq.) in DMF (5.0 mL) was added Et₃N (46 μL, 82 μmol, 4.0 eq.), the mixture was stirred for 1 hour. The resulting solution was diluted with H₂O (20 mL), extracted with EtOAc (20 mL×3). The combined organic layers were washed with brine (20 mL×2), dried over Na₂SO₄ and concentrated under reduced pressure, the residue was purified by prep-HPLC (C18, 20~95%, MeCN in H₂O with 0.1% HCOOH) to give 5-(1-(2-amino-3-methylquinoline-6-carbonyl)-6′H-spiro[pyrrolidine-3,4′-thieno[2,3-c]furan]-2′-yl)-N-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)picolinamide (8 mg, 16% yield) as a white solid.

LC-MS (ESI): mass calcd, for C₅₈H₆₆FN₉O₁₀S₂, 1131.4; m/z found, 1132.1 [M+H]⁺. ¹HNMR (400 MHz, DMSO-d₆) δ 8.96 (s, 1H), 8.85 (d, J=11.9 Hz, 1H), 8.69-8.64 (m, 1H), 8.48 (t, J=5.9 Hz, 1H), 8.16-8.10 (m, 1H), 8.05 (d, J=7.8 Hz, 1H), 7.89-7.77 (m, 2H), 7.71-7.57 (m, 2H), 7.50-7.43 (m, 1H), 7.39 (d, J=7.8 Hz, 1H), 7.28 (d, J=7.0 Hz, 1H), 7.01 (s, 1H), 6.95 (d, J=8.0 Hz, 1H), 6.54-6.44 (m, 2H), 5.18-4.97 (m, 3H), 4.59 (d, J=9.1 Hz, 1H), 4.51 (t, J=8.3 Hz, 1H), 4.37-4.33 (m, 1H), 4.29-4.22 (m, 1H), 4.19-4.15 (m, 2H), 4.04-3.73 (m, 6H), 3.68-3.56 (m, 9H), 3.50-3.47 (m, 2H), 2.44 (s, 3H), 2.25-2.16 (m, 4H), 2.11-2.05 (m, 1H), 1.95-1.87 (m, 1H), 1.41-1.32 (m, 2H), 1.26-1.20 (m, 3H), 0.95 (s, 9H).

Example B6. 5-(1′-(4-amino-1-methyl-1H-pyrazolo[4,3-c]quinoline-8-carbonyl)-3H-spiro[isobenzofuran-1,3′-pyrrolidin]-5-yl)-N-(2-(2-(2-(2-(((2S,4R)-1-((S)-2-(1-fluorocyclo propane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)picolinamide

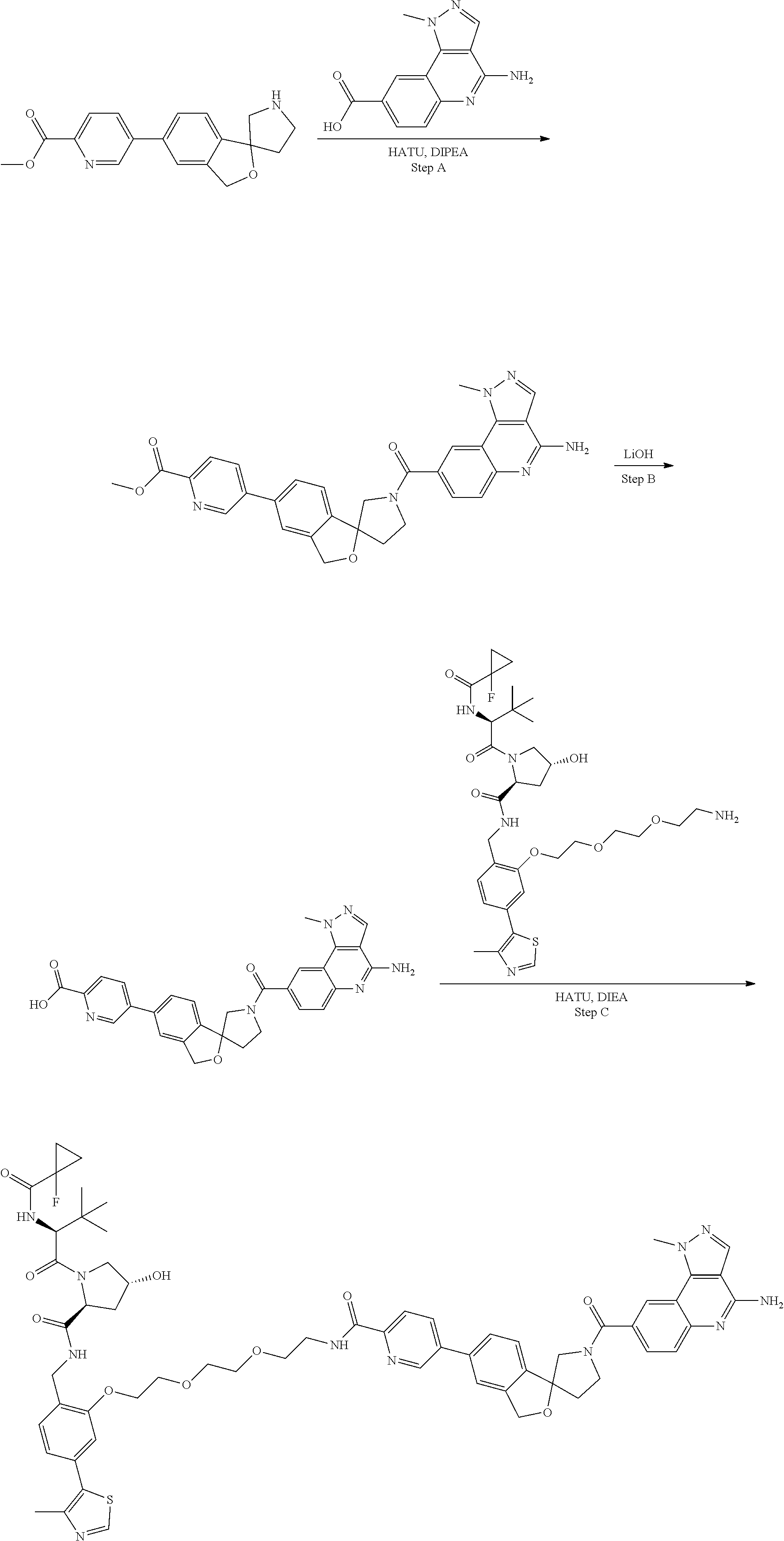

Example B6 was synthesized following the same procedures as described in Example B4 (4.5 mg, 7% yield over 3 steps) as a white solid. LC-MS (ESI): mass calcd, for C₆₁H₆₈FN₁₁O₁₀S, 1165.5; m/z found, 1166.3 [M+H]⁺. ¹H NMR (400 MHz, DMSO-d₆) δ 8.99-8.83 (m, 2H), 8.76-8.67 (m, 1H), 8.50-8.34 (m, 3H), 8.26-8.19 (m, 1H), 8.09 (t, J=9.7 Hz, 1H), 7.86-7.79 (m, 1H), 7.76-7.69 (m, 2H), 7.66-7.56 (m, 2H), 7.39 (d, J=7.2 Hz, 1H), 7.28 (d, J=8.9 Hz, 1H), 7.02-6.98 (m, 1H), 6.97-6.89 (m, 1H), 5.20-5.16 (m, 1H), 5.15-4.96 (m, 2H), 4.59 (d, J=8.9 Hz, 1H), 4.53-4.49 (m, 1H), 4.44 (s, 3H), 4.34 (s, 1H), 4.25 (d, J=23.7 Hz, 2H), 4.16 (s, 2H), 4.07 (d, J=11.4 Hz, 1H), 3.88-3.86 (m, 1H), 3.80 (s, 2H), 3.64-3.59 (m, 6H), 3.51-3.47 (m, 4H), 2.96-2.90 (m, 1H), 2.58-2.54 (m, 1H), 2.43-2.40 (m, 3H), 2.31-2.13 (m, 2H), 2.12-2.05 (m, 1H), 1.95-1.89 (m, 1H), 1.40-1.32 (m, 2H), 1.27-1.13 (m, 4H), 0.94 (s, 9H).

Example B7. 5-(1-(2-((7-amino-1-methyl-1H-pyrazolo[3,4-c]pyridin-4-yl)amino)-2-oxoacetyl)-6′H-spiro[pyrrolidine-3,4′-thieno[2,3-c]furan]-2′-yl)-N-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl) picolinamide

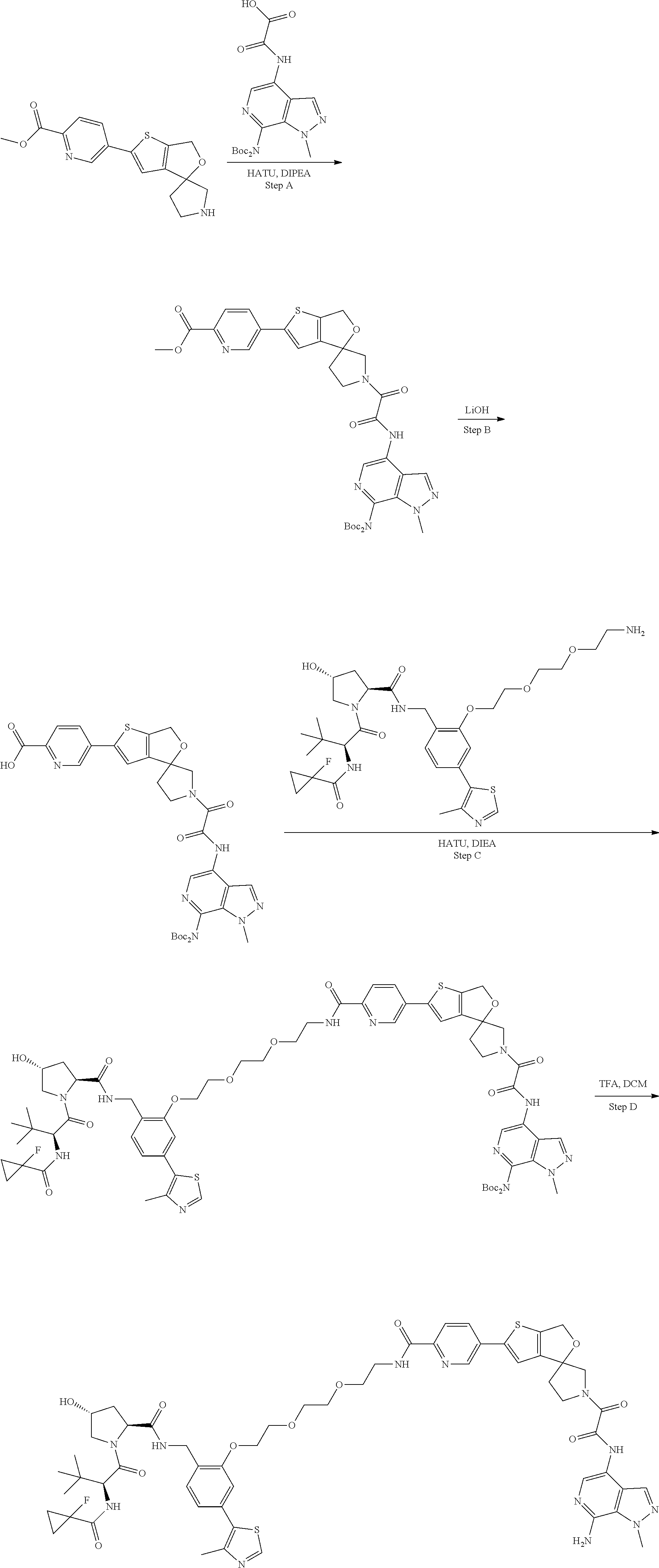

Step A: methyl 5-(1-(2-((7-(bis(tert-butoxycarbonyl)amino)-1-methyl-1H-pyrazolo[3,4-c]pyridin-4-yl)amino)-2-oxoacetyl)-6′H-spiro[pyrrolidine-3,4′-thieno[2,3-c]furan]-2′-yl)picolinate

To a solution of methyl 5-(6′H-spiro[pyrrolidine-3,4′-thieno[2,3-c]furan]-2′-yl)picolinate (25 mg, 79 μmol, 1.0 eq.), 2-((7-(bis(tert-butoxycarbonyl)amino)-1-methyl-1H-pyrazolo[3,4-c]pyridin-4-yl)amino)-2-oxoacetic acid (30 mg, 79 μmol, 1.0 eq.) and HATU (60 mg, 160 μmol, 2.0 eq.) in DMF (5.0 mL) was added Et₃N (0.1 mL, 160 μmol, 2.0 eq.), the mixture was stirred at room temperature for 1 hour. The mixture was diluted with water (30 mL) and extracted with EtOAc (20 mL×3). The combined organic layers were washed with brine, dried over anhydrous Na₂SO₄ and concentrated under reduced pressure. The residue was purified by flash chromatography (eluent, 0~4% MeOH in DCM) to give methyl 5-(1-(2-((7-(bis(tert-butoxycarbonyl)amino)-1-methyl-1H-pyrazolo[3,4-c]pyridin-4-yl)amino)-2-oxoacetyl)-6′H-spiro[pyrrolidine-3,4′-thieno[2,3-c]furan]-2′-yl)picolinate (10 mg, 50% yield) as a white solid.

LC-MS (ESI): mass calcd, for C₃₅H₃₉N₇O₉S, 733.2; m/z found, 734.2 [M+H]⁺.

### Steps B-C

Example B7 was synthesized following the same procedures as described in Example B1 (2.2 mg, 10% yield over 2 steps) as a white solid. LC-MS (ESI): mass calcd, for C₅₆H₆₅FN₁₂O₁₁S₂, 1164.4; m/z found, 1165.3.

Example B8. 5-(1′-(4-amino-1,3-dihydrofuro[3,4-c][1,7]naphthyridine-8-carbonyl)-3H-spiro[isobenzofuran-1,3′-pyrrolidin]-5-yl)-N-(2-(2-(2-(2-(((2S,4R)-1-((S)-2-(1-fluorocyclopro pane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)picolinamide

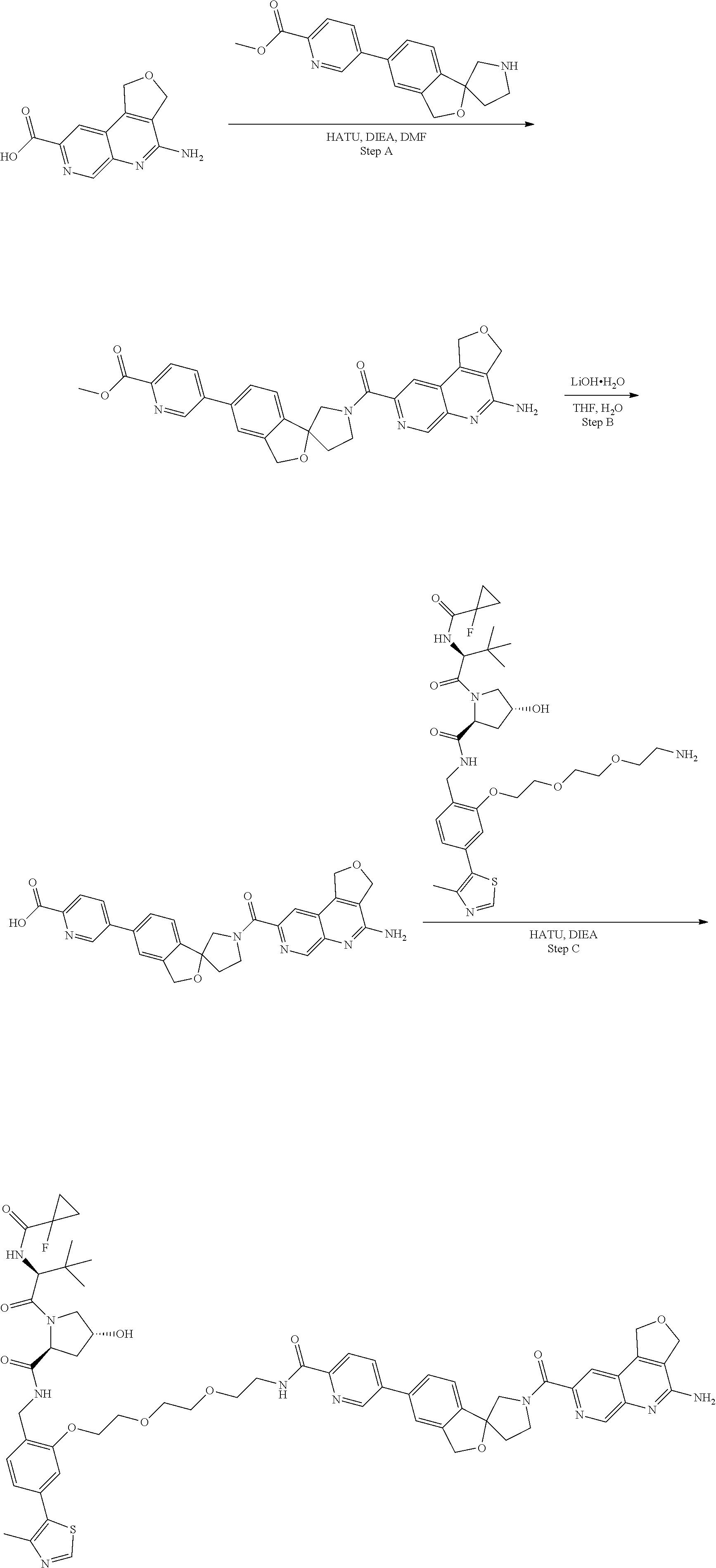

Example B8 was synthesized following the same procedures as described in Example B4 (4 mg, 4% yield over 3 steps) as a white solid. LC-MS (ESI): mass calcd, for C₆₀H₆₇FN₁₀O₁₁S, 1154.5; m/z found, 1155.3 [M+H]⁺.

Example B9. 5-(1-(4-amino-1-methyl-1H-pyrazolo[4,3-c]quinoline-8-carbonyl)-6′H-spiro[pyrrolidine-3,4′-thieno[2,3-c]furan]-2′-yl)-N-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)picolinamide

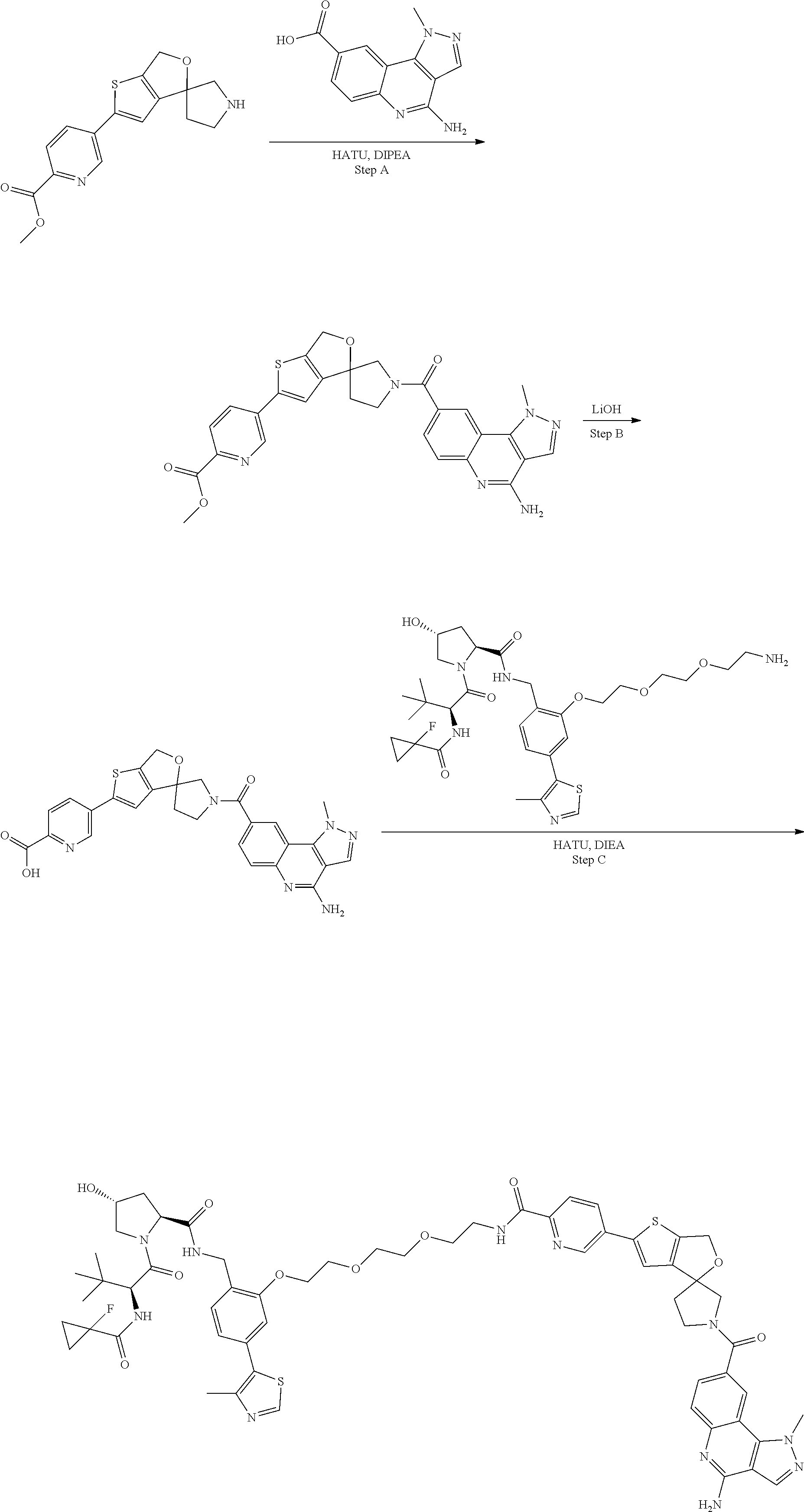

Example B9 was synthesized following the same procedures as described in Example B4 (2.2 mg, 2% yield over 3 steps) as a white solid. LC-MS (ESI): mass calcd, for C₅₉H₆₆FN₁₁O₁₀S₂, 1171.4; m/z found, 1172.4 [M+H]⁺.

Example B10. 5-(1′-(2-((7-amino-1-methyl-1H-pyrazolo[3,4-c]pyridin-4-yl)amino)-2-oxoacetyl)-3H-spiro[isobenzofuran-1,3′-pyrrolidin]-5-yl)-N-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)picolinamide

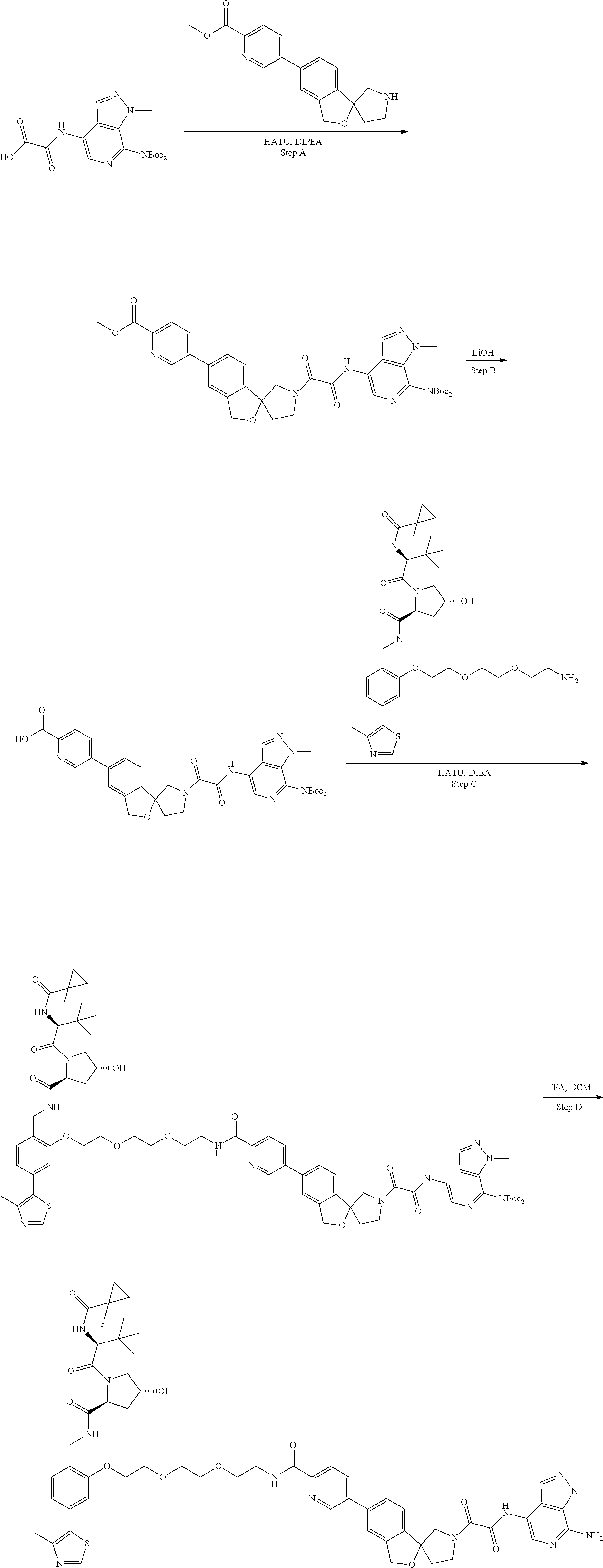

Example B10 was synthesized following the same procedures as described in Example B4 (5.4 mg, 7% yield over 4 steps) as a white solid. LC-MS (ESI): mass calcd. for C₅₈H₆₇FN₁₂O₁₁S, 1158.5; m/z found, 1159.3 [M+H]⁺. ¹H NMR (400 MHz, DMSO-d₆) δ 10.58 (s, 1H), 8.96 (d, J=3.9 Hz, 1H), 8.90 (d, J=6.3 Hz, 1H), 8.76-8.69 (m, 1H), 8.53-8.42 (m, 1H), 8.29-8.22 (m, 1H), 8.13-8.05 (m, 1H), 7.92 (d, J=19.1 Hz, 1H), 7.81-7.71 (m, 3H), 7.62 (d, J=7.6 Hz, 1H), 7.40 (d, J=7.9 Hz, 1H), 7.29 (d, J=7.0 Hz, 1H), 7.01 (s, 1H), 6.95 (d, J=7.8 Hz, 1H), 6.27-6.18 (m, 2H), 5.20-5.16 (m, 1H), 5.13-5.11 (m, 1H), 4.59 (d, J=9.2 Hz, 1H), 4.52 (t, J=8.2 Hz, 1H), 4.37-4.33 (m, 1H), 4.29-4.25 (m, 3H), 4.24-4.19 (m, 1H), 4.18-4.15 (m, 2H), 4.10-4.01 (m, 1H), 3.95-3.87 (m, 1H), 3.82-3.79 (m, 2H), 3.67-3.63 (m, 3H), 3.61-3.58 (m, 4H), 3.53-3.48 (m, 2H), 3.42-3.36 (m, 2H), 3.31-3.28 (m, 2H), 2.43 (s, 3H), 2.40-2.33 (m, 1H), 2.24-2.17 (m, 1H), 2.12-2.06 (m, 1H), 1.95-1.89 (m, 1H), 1.40-1.32 (m, 2H), 1.24-1.20 (m, 2H), 0.95 (s, 9H).

Example B11. 5-((R)-1-(2-amino-3-methylquinoline-6-carbonyl)-6′H-spiro[pyrrolidine-3,4′-thieno[2,3-c]furan]-2′-yl)-N-((1S,4R)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)picolinamide

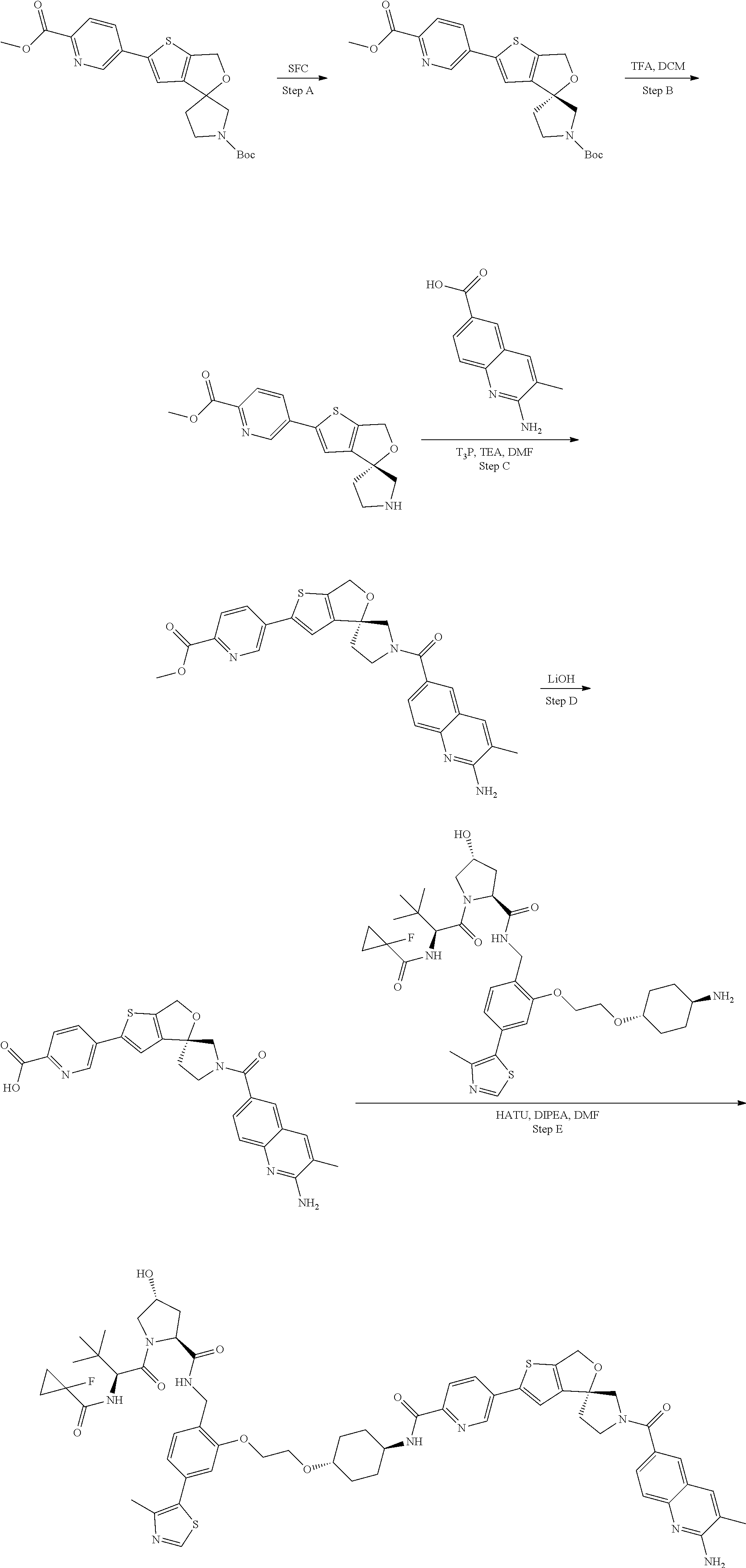

Step A: tert-butyl (R)-2′-(6-(methoxycarbonyl)pyridin-3-yl)-6′H-spiro[pyrrolidine-3,4′-thieno[2,3-c]furan]-1-carboxylate

The tert-butyl (R)-2′-(6-(methoxycarbonyl)pyridin-3-yl)-6′H-spiro[pyrrolidine-3,4′-thieno[2,3-c]furan]-1-carboxylate (80 mg) was separated by SFC [Instrument: SHIMADZU PREP SOLUTION SFC; Column: (R,R)-WHELK, 250×20 mm I.D., 5 μm; Column temperature: 35° C.; Mobile phase: A for CO₂ and B for MeOH; Gradient: B 50%; Flow rate: 40 mL/min] to give the title compound as a yellow solid. LC-MS (ESI): mass calcd, for C₂₁H₂₄N₂O₅S, 416.1; m/z found, 417.1 [M+H]⁺.

### Steps B-E

Example B11 was synthesized following the same procedures as described in Example B5 (9.6 mg, 13% yield over 4 steps) as a white solid. LC-MS (ESI): mass calcd. for C₆₀H₆₈FN₉O₉S₂, 1141.5; m/z found, 1142.5 [M+H]⁺. ¹H NMR (400 MHz, DMSO-d₆) δ 8.99 (s, 1H), 8.90-8.82 (m, 1H), 8.56-8.48 (m, 1H), 8.47-8.42 (m, 1H), 8.18-8.13 (m, 1H), 8.07-8.03 (m, 1H), 7.93-7.83 (m, 2H), 7.75-7.63 (m, 2H), 7.55-7.46 (m, 1H), 7.43-7.39 (m, 1H), 7.31-7.26 (m, 1H), 7.05 (s, 1H), 6.98-6.95 (m, 1H), 5.17-4.98 (m, 2H), 4.63-4.57 (m, 1H), 4.56-4.50 (m, 1H), 4.37-4.34 (m, 1H), 4.33-4.28 (m, 1H), 4.27-4.21 (m, 1H), 4.19-4.16 (m, 2H), 4.04-3.90 (m, 1H), 3.84-3.74 (m, 5H), 3.72-3.56 (m, 4H), 2.51 (s, 3H), 2.47 (s, 3H), 2.22-2.18 (m, 2H), 2.13-2.01 (m, 4H), 1.88-1.82 (m, 2H), 1.59-1.41 (m, 4H), 1.35-1.31 (m, 2H), 1.23-1.20 (m, 2H), 0.96 (s, 9H).

Example B12. 5-((S)-1-(2-amino-3-methylquinoline-6-carbonyl)-6′H-spiro[pyrrolidine-3,4′-thieno[2,3-c]furan]-2′-yl)-N-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)picolinamide

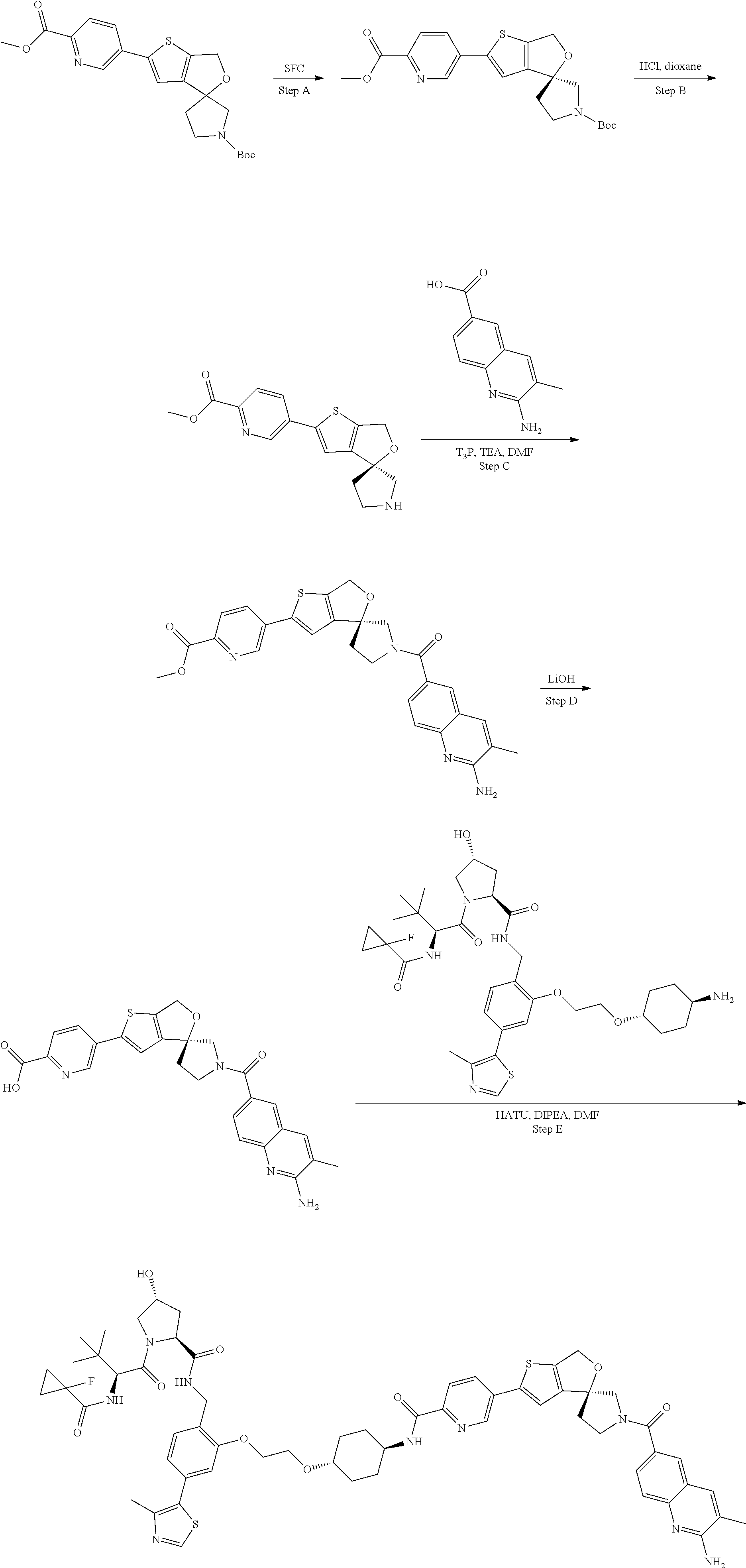

Step A: tert-butyl(S)-2′-(6-(methoxycarbonyl)pyridin-3-yl)-6′H-spiro[pyrrolidine-3,4′-thieno[2,3-c]furan]-1-carboxylate

The tert-butyl(S)-2′-(6-(methoxycarbonyl)pyridin-3-yl)-6′H-spiro[pyrrolidine-3,4′-thieno[2,3-c]furan]-1-carboxylate (100 mg) was separated by SFC [Instrument: SHIMADZU PREP SOLUTION SFC; Column: (R,R)-WHELK, 250×20 mm I.D., 5 μm; Column temperature: 35° C.; Mobile phase: A for CO₂ and B for MeOH; Gradient: B 50%; Flow rate: 40 mL/min] to give the title compound as a yellow solid from racemic tert-butyl 2′-(6-(methoxycarbonyl)pyridin-3-yl)-6′H-spiro[pyrrolidine-3,4′-thieno[2,3-c]furan]-1-carboxylate (180 mg). LC-MS (ESI): mass calcd, for C₂₁H₂₄N₂O₅S, 416.14; m/z found, 417.1 [M+H]⁺.

### Steps B-E

Example B12 was synthesized following the same procedures as described in Example B5 (2 mg, 2.6% yield over 4 steps) as a white solid. LC-MS (ESI): mass calcd, for C₆₀H₆₈FN₉O₉S₂, 1141.5; m/z found, 1142.3 [M+H]⁺. ¹H NMR (400 MHz, DMSO-d₆) δ 8.99 (s, 1H), 8.87 (d, J=14.5 Hz, 1H), 8.53-8.41 (m, 2H), 8.18-8.12 (m, 1H), 8.06 (d, J=7.2 Hz, 1H), 8.01-7.84 (m, 2H), 7.76-7.64 (m, 2H), 7.63-7.45 (m, 1H), 7.41 (d, J=7.9 Hz, 1H), 7.29 (d, J=6.9 Hz, 1H), 7.05 (s, 1H), 6.97 (d, J=7.8 Hz, 1H), 5.18-4.97 (m, 2H), 4.62-4.56 (m, 1H), 4.55-4.49 (m, 1H), 4.37-4.34 (m, 1H), 4.33-4.27 (m, 1H), 4.26-4.21 (m, 1H), 4.19-4.14 (m, 2H), 4.05-3.95 (m, 1H), 3.85-3.76 (m, 5H), 3.67-3.59 (m, 4H), 2.52 (s, 3H), 2.47 (s, 3H), 2.25-2.20 (m, 2H), 2.12-2.00 (m, 4H), 1.87-1.82 (m, 2H), 1.52-1.45 (m, 4H), 1.35-1.33 (m, 2H), 1.24-1.21 (m, 2H), 0.98-0.94 (m, 9H).

Example B13. 5-((S)-1′-(2-amino-3-cyclopropylquinoline-6-carbonyl)-3H-spiro[isobenzofuran-1,3′-pyrrolidin]-5-yl)-N-((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)picolinamide

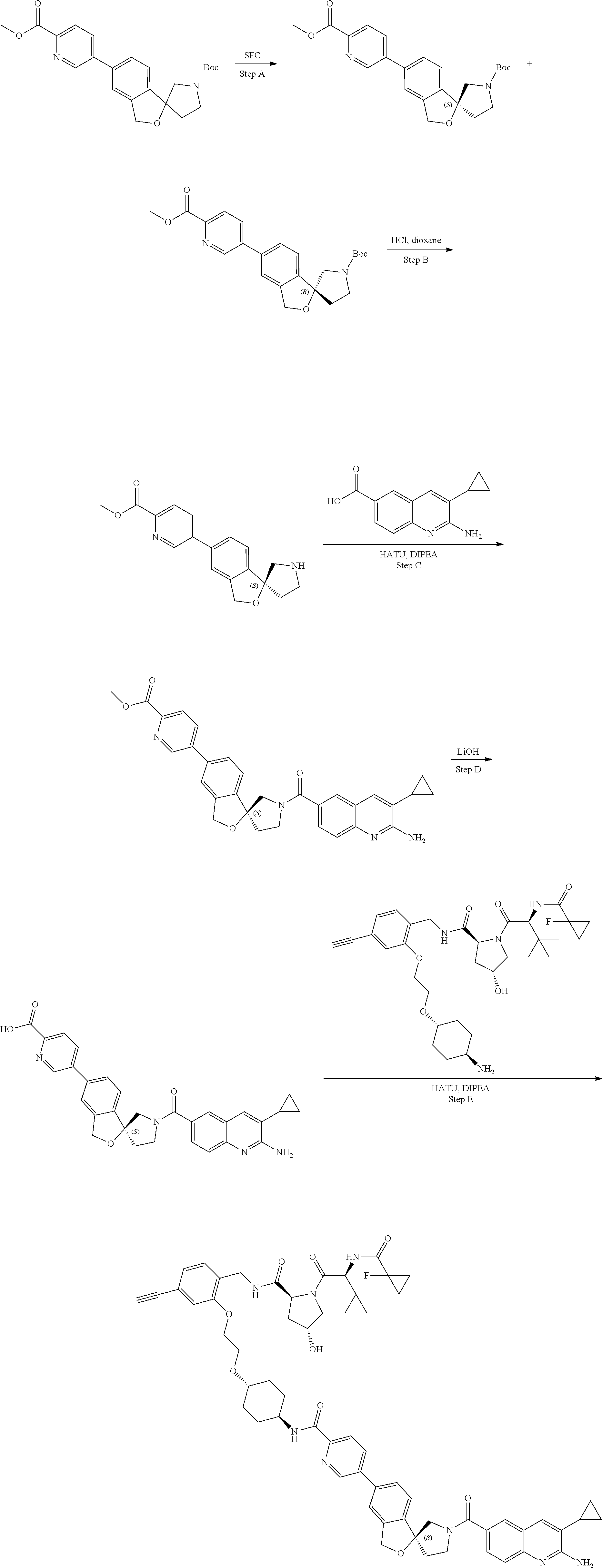

Step A: tert-butyl(S)-5-(6-(methoxycarbonyl)pyridin-3-yl)-3H-spiro[isobenzofuran-1,3′-pyrrolidine]-1′-carboxylate

The tert-butyl(S)-5-(6-(methoxycarbonyl)pyridin-3-yl)-3H-spiro[isobenzofuran-1,3′-pyrrolidine]-1′-carboxylate (100 mg) was separated as a yellow solid by SFC [Instrument: SHIMADZU PREP SOLUTION SFC; Column: (R,R)-WHELK, 250×20 mm I.D., 5 μm; Column temperature: 35° C.; Mobile phase: A for CO₂ and B for MeOH; Gradient: B 50%; Flow rate: 40 mL/min] to give the title compound as a yellow solid from racemic tert-butyl 5-(6-(methoxycarbonyl)pyridin-3-yl)-3H-spiro[isobenzofuran-1,3′-pyrrolidine]-1′-carboxylate (200 mg). LC-MS (ESI): mass calcd, for C₂₃H₂₆N₂O₅, 410.2; m/z found, 411.2 [M+H]⁺.

### Steps B-E

Example B13 was synthesized following the same procedures as described in Example B11 (6.6 mg, 5% yield over 5 steps) as a white solid. LC-MS (ESI): mass calcd, for C₆₂H₆₉FN₈O₉, 1088.5; m/z found, 1089.9 [M+H]⁺. ¹H NMR (400 MHz, DMSO-d₆) δ 9.10-8.68 (m, 3H), 8.58-8.39 (m, 2H), 8.26 (t, J=8.1 Hz, 1H), 8.18 (s, 1H), 8.12-8.09 (m, 1H), 7.98-7.86 (m, 1H), 7.82-7.72 (m, 2H), 7.69 (d, J=7.4 Hz, 1H), 7.61 (dd, J=25.0, 8.1 Hz, 1H), 7.37-7.21 (m, 2H), 7.07-7.03 (m, 1H), 6.98 (d, J=7.8 Hz, 1H), 5.27-5.08 (m, 1H), 5.10-4.97 (m, 1H), 4.59 (d, J=9.1 Hz, 1H), 4.49 (t, J=8.1 Hz, 1H), 4.35 (s, 1H), 4.32-4.18 (m, 2H), 4.15-4.09 (m, 3H), 3.92-3.75 (m, 6H), 3.66-3.62 (m, 2H), 3.38-3.32 (m, 2H), 2.47-2.29 (m, 1H), 2.25-2.13 (m, 1H), 2.12-2.00 (m, 3H), 1.96-1.81 (m, 4H), 1.55-1.45 (m, 2H), 1.40-1.28 (m, 4H), 1.24-1.21 (m, 2H), 1.11-1.02 (m, 2H), 0.95 (s, 9H), 0.86-0.77 (m, 2H).

Example B14. 5-(1′-(2-amino-3-methylquinoline-6-carbonyl)-3H-spiro[isobenzofuran-1,3′-pyrrolidin]-5-yl)-N-((1S,4r)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)picolinamide

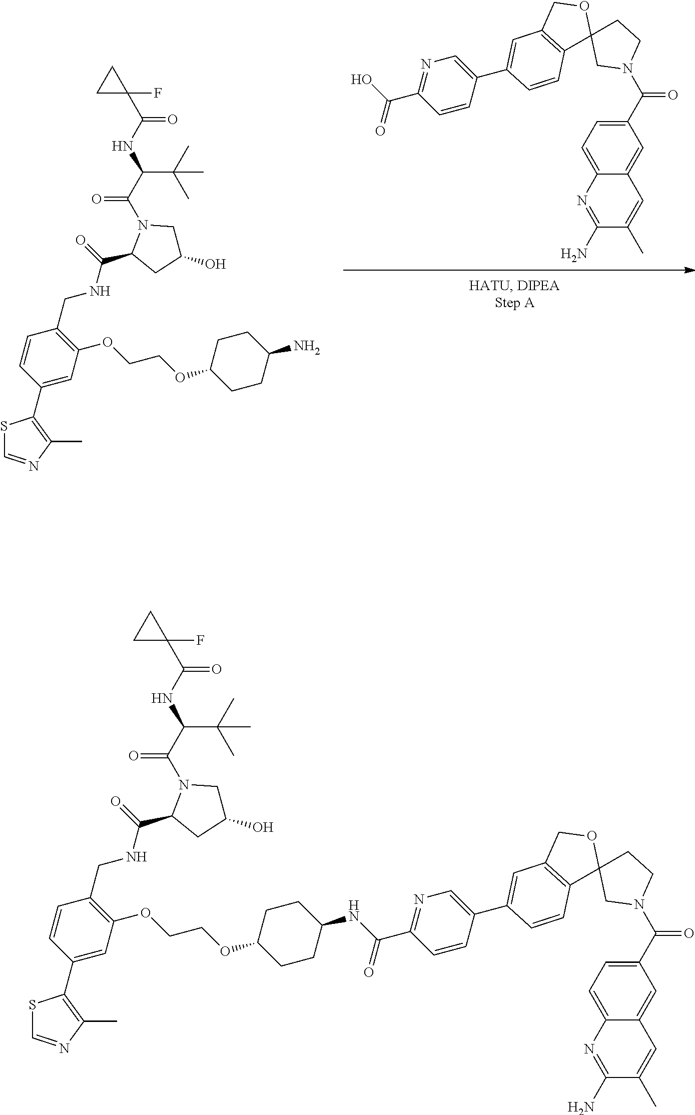 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](N)CC2)c1 ; Cc1cc2cc(C(=O)N3CCC4(C3)OCc3cc(-c5ccc(C(=O)O)nc5)ccc34)ccc2nc1N ; Cc1cc2cc(C(=O)N3CCC4(C3)OCc3cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc34)ccc2nc1N]

Example B14 was synthesized following the same procedures as described in Example B4 (8.0 mg, 13% yield) as a white solid. LC-MS (ESI): mass calcd, for C₆₂H₇₀FN₉O₉S, 1135.5; m/z found, 1136.4 [M+H]⁺. ¹H NMR (400 MHz, DMSO-d₆) δ 8.98 (s, 1H), 8.91 (d, J=19.1 Hz, 1H), 8.54-8.44 (m, 2H), 8.30-8.22 (m, 1H), 8.09 (t, J=8.5 Hz, 1H), 7.93 (s, 1H), 7.89-7.84 (m, 1H), 7.77-7.74 (m, 1H), 7.72-7.57 (m, 3H), 7.56-7.44 (m, 1H), 7.41 (d, J=7.9 Hz, 1H), 7.29 (dd, J=9.1, 2.4 Hz, 1H), 7.05 (s, 1H), 6.98-6.80 (m, 2H), 5.22-5.09 (m, 1H), 5.07-4.99 (m, 1H), 4.59 (d, J=9.3 Hz, 1H), 4.52 (t, J=8.1 Hz, 1H), 4.36 (s, 1H), 4.33-4.27 (m, 1H), 4.26-4.20 (m, 1H), 4.19-4.14 (m, 2H), 4.03 (d, J=11.3 Hz, 1H), 3.85-3.77 (m, 5H), 3.68-3.57 (m, 4H), 2.46 (s, 3H), 2.27-2.17 (m, 4H), 2.10-2.01 (m, 3H), 1.88-1.82 (m, 2H), 1.53-1.47 (m, 2H), 1.39-1.30 (m, 4H), 1.26-1.17 (m, 4H), 0.95 (s, 9H).

Example B15. 5-((R)-1-(4-amino-1-methyl-1H-pyrazolo[4,3-c]quinoline-8-carbonyl)-6′H-spiro[pyrrolidine-3,4′-thieno[2,3-c]furan]-2′-yl)-N-((1S,4R)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)picolinamide

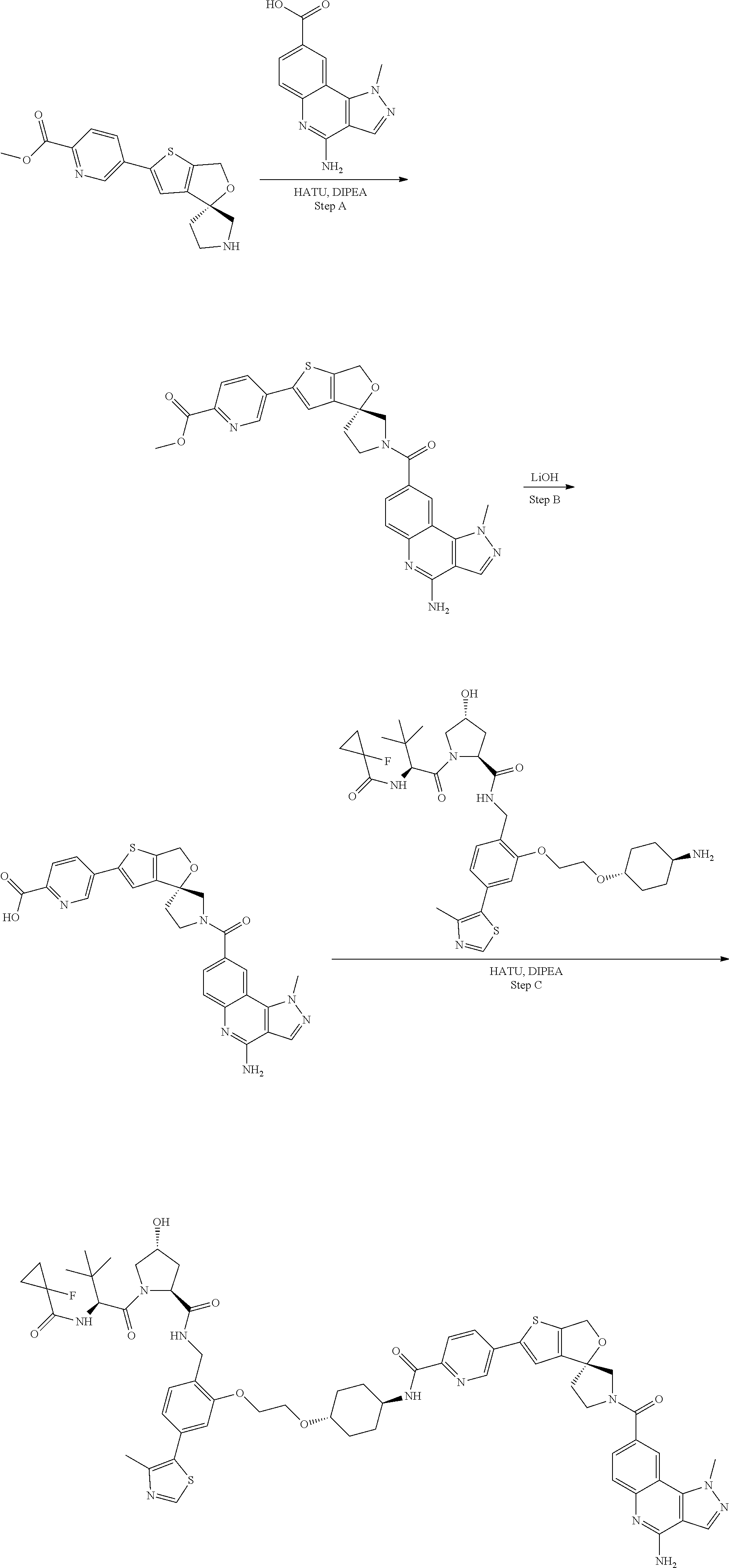

Example B15 was synthesized following the same procedures as described in Example B5 (5.6 mg, 7% yield over 3 steps) as a white solid. LC-MS (ESI): mass calcd. for C₆₁H₆₈FN₁₁O₉S₂, 1181.5; m/z found, 1182.5 [M+H]⁺. ¹H NMR (400 MHz, DMSO-d₆) δ 8.99 (s, 1H), 8.87 (d, J=16.2 Hz, 1H), 8.53-8.47 (m, 1H), 8.46-8.36 (m, 2H), 8.26 (d, J=13.3 Hz, 1H), 8.20 (s, 1H), 8.17-8.03 (m, 2H), 7.75 (s, 1H), 7.69-7.56 (m, 2H), 7.41 (d, J=7.9 Hz, 1H), 7.29 (d, J=6.4 Hz, 1H), 7.20-7.13 (m, 2H), 7.05 (s, 1H), 6.97 (d, J=7.3 Hz, 1H), 5.17-5.00 (m, 2H), 4.62-4.57 (m, 1H), 4.52 (t, J=8.1 Hz, 1H), 4.44-4.36 (m, 4H), 4.27 (dd, J=20.3, 6.0 Hz, 2H), 4.19-4.15 (m, 2H), 4.05-3.87 (m, 2H), 3.87-3.77 (m, 5H), 3.68-3.57 (m, 3H), 2.47 (s, 3H), 2.23-2.16 (m, 1H), 2.15-2.01 (m, 4H), 1.96-1.91 (m, 1H), 1.87-1.82 (m, 2H), 1.54-1.47 (m, 2H), 1.39-1.29 (m, 4H), 1.23-1.20 (m, 2H), 0.96 (s, 9H).

Example B16. 5-((S)-1-(4-amino-1-methyl-1H-pyrazolo[4,3-c]quinoline-8-carbonyl)-6′H-spiro[pyrrolidine-3,4′-thieno[2,3-c]furan]-2′-yl)-N-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)picolinamide

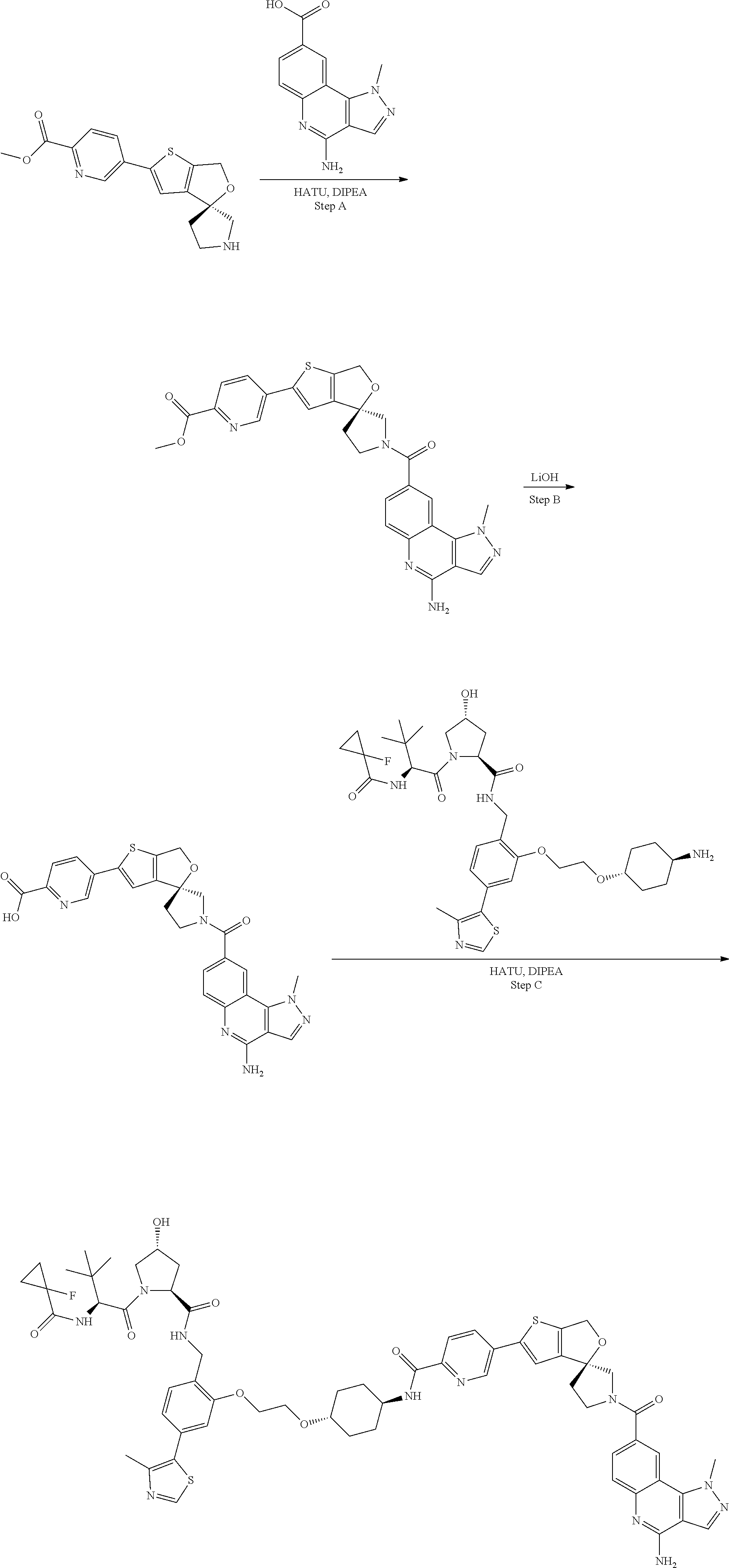

Example B16 was synthesized following the same procedures as described in Example B5

(1.2 mg, 2% yield over 3 steps) as a white solid. LC-MS (ESI): mass calcd. for C₆₁H₆₈FN₁₁O₉S₂, 1181.5; m/z found, 1182.5 [M+H]⁺. ¹H NMR (400 MHz, DMSO-d₆) δ 8.99 (s, 1H), 8.87 (d, J=16.2 Hz, 1H), 8.53-8.47 (m, 1H), 8.46-8.36 (m, 2H), 8.26 (d, J=13.3 Hz, 1H), 8.20 (s, 1H), 8.17-8.03 (m, 2H), 7.75 (s, 1H), 7.69-7.56 (m, 2H), 7.41 (d, J=7.9 Hz, 1H), 7.29 (d, J=6.4 Hz, 1H), 7.20-7.13 (m, 2H), 7.05 (s, 1H), 6.97 (d, J=7.3 Hz, 1H), 5.17-5.00 (m, 2H), 4.62-4.57 (m, 1H), 4.52 (t, J=8.1 Hz, 1H), 4.44-4.36 (m, 4H), 4.27 (dd, J=20.3, 6.0 Hz, 2H), 4.19-4.15 (m, 2H), 4.05-3.87 (m, 2H), 3.87-3.77 (m, 5H), 3.68-3.57 (m, 3H), 2.47 (s, 3H), 2.23-2.16 (m, 1H), 2.15-2.01 (m, 4H), 1.96-1.91 (m, 1H), 1.87-1.82 (m, 2H), 1.54-1.47 (m, 2H), 1.39-1.29 (m, 4H), 1.23-1.20 (m, 2H), 0.96 (s, 9H).

Example B17. 5-((R)-1′-(2-amino-3-cyclopropylquinoline-6-carbonyl)-3H-spiro[isobenzofuran-1,3′-pyrrolidin]-5-yl)-N-((1S,4R)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)picolinamide

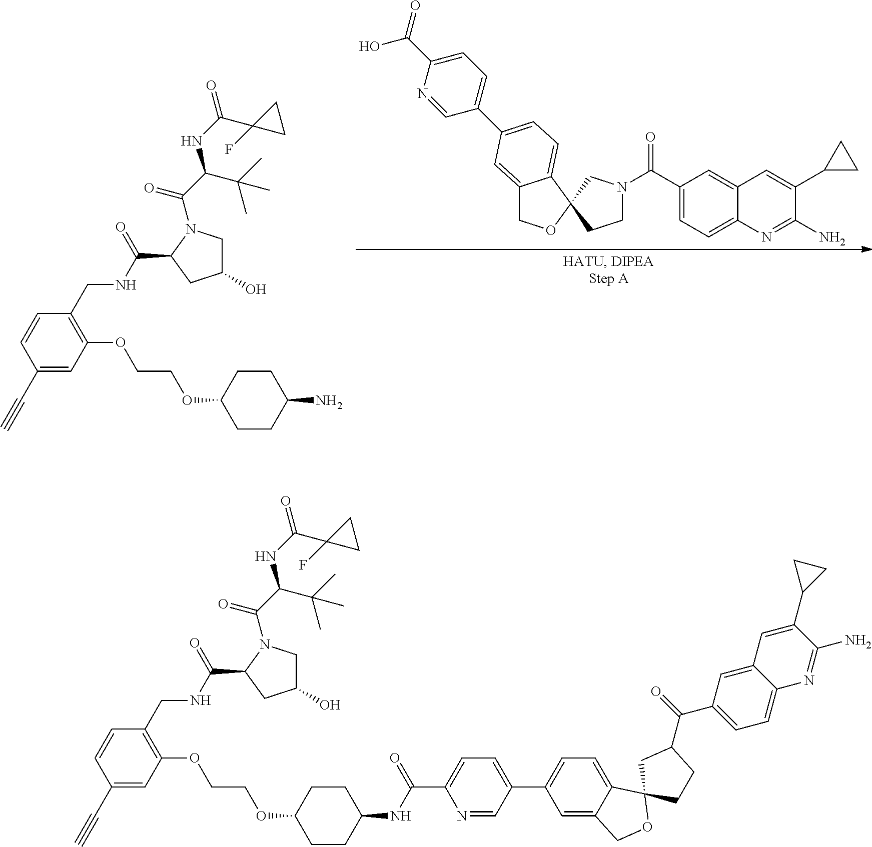

Example B17 was synthesized following the same procedures as described in Example B4 (3 mg, 8% yield) as a white solid. LC-MS (ESI): mass calcd, for C₆₂H₆₉FN₈O₉, 1088.5; m/z found, 1089.3 [M+H]⁺. ¹H NMR (400 MHz, DMSO-d₆) δ 8.97-8.74 (m, 3H), 8.52-8.45 (m, 2H), 8.29-8.09 (m, 4H), 7.95-7.91 (m, 1H), 7.78-7.73 (m, 2H), 7.71-7.68 (m, 1H), 7.66-7.57 (m, 1H), 7.34-7.27 (m, 2H), 7.06 (s, 1H), 6.98 (d, J=7.7 Hz, 1H), 5.19-5.00 (m, 2H), 4.60 (d, J=9.2 Hz, 1H), 4.53-4.48 (m, 1H), 4.37-4.34 (m, 1H), 4.30-4.18 (m, 2H), 4.15-4.11 (m, 3H), 3.92-3.76 (m, 7H), 3.39-3.31 (m, 2H), 2.46-2.35 (m, 1H), 2.27-1.99 (m, 5H), 1.94-1.83 (m, 4H), 1.56-1.46 (m, 2H), 1.40-1.20 (m, 7H), 1.11-1.04 (m, 2H), 0.96 (s, 9H), 0.85-0.76 (m, 2H).

Example B18. 5-((R)-1-(2-amino-3-cyclopropylquinoline-6-carbonyl)-6′H-spiro[pyrrolidine-3,4′-thieno[2,3-c]furan]-2′-yl)-N-((1S,4R)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)picolinamide

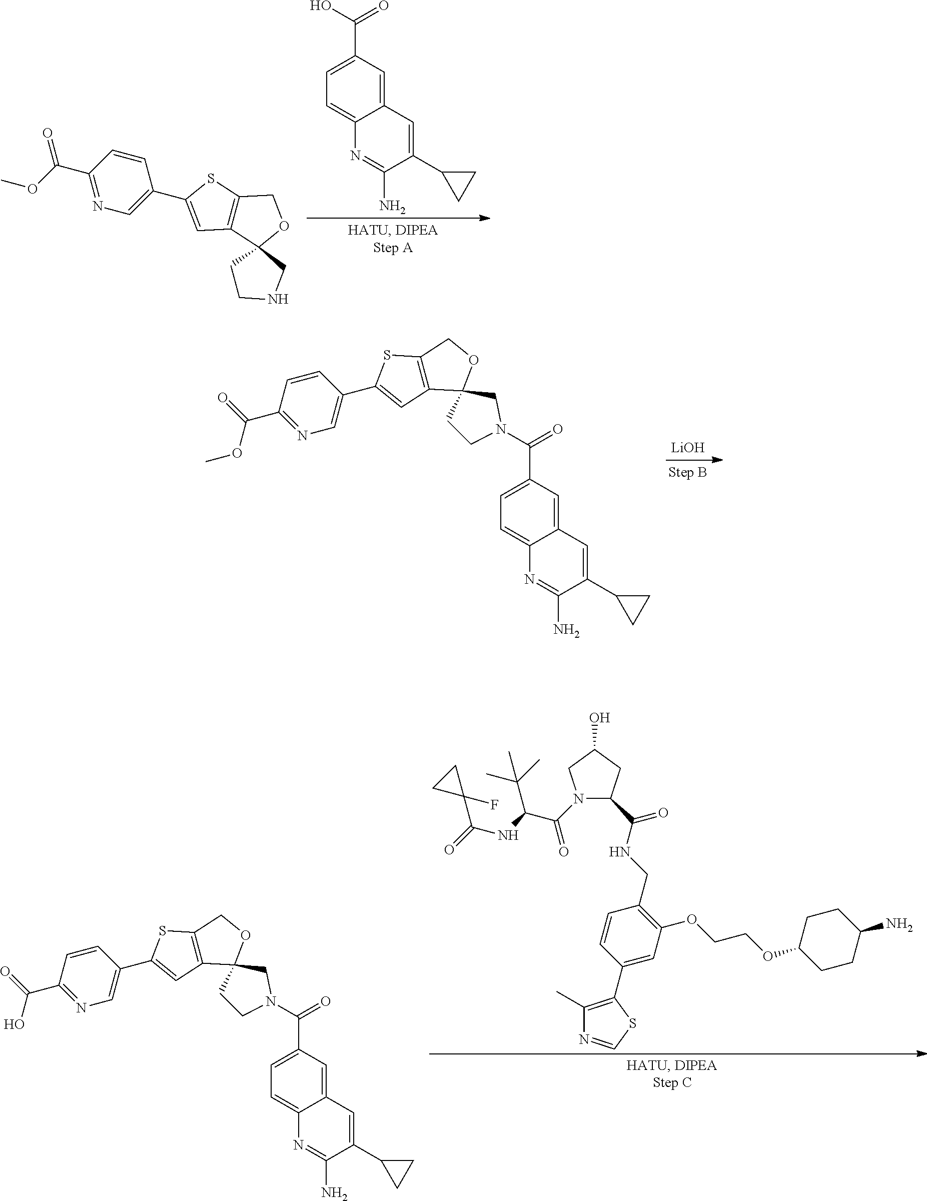 [SMILES: COC(=O)c1ccc(-c2cc3c(s2)CO[C@]32CCNC2)cn1 ; Nc1nc2ccc(C(=O)O)cc2cc1C1CC1 ; COC(=O)c1ccc(-c2cc3c(s2)CO[C@]32CCN(C(=O)c3ccc4nc(N)c(C5CC5)cc4c3)C2)cn1 ; "LiOH" ; Nc1nc2ccc(C(=O)N3CC[C@@]4(C3)OCc3sc(-c5ccc(C(=O)O)nc5)cc34)cc2cc1C1CC1 ; Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](N)CC2)c1]

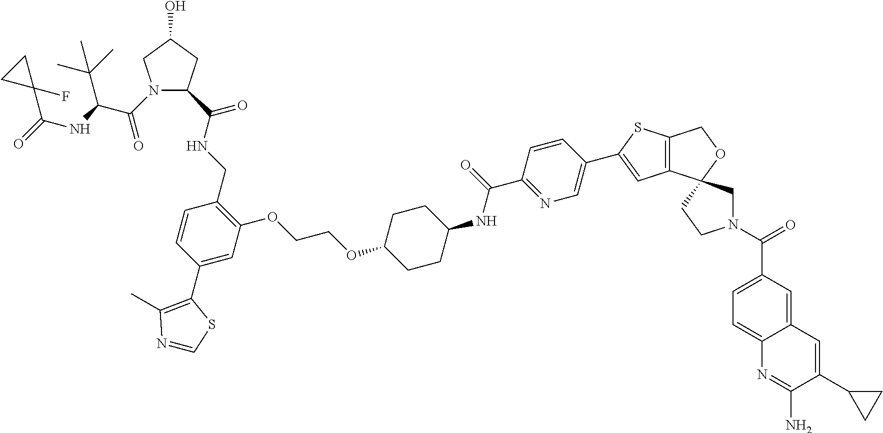 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4cc5c(s4)CO[C@]54CCN(C(=O)c5ccc6nc(N)c(C7CC7)cc6c5)C4)cn3)CC2)c1]

Example B18 was synthesized following the same procedures as described in Example B5 (20 mg, 27% yield over 3 steps) as a white solid. LC-MS (ESI): mass calcd. for C₆₂H₇₀FN₉O₉S₂, 1167.5; m/z found, 1168.5 [M+H]⁺. ¹H NMR (400 MHz, DMSO-d₆) δ 8.99 (s, 1H), 8.90-8.72 (m, 3H), 8.53-8.47 (m, 1H), 8.46-8.41 (m, 1H), 8.28-8.18 (m, 1H), 8.18-8.15 (m, 1H), 8.14-8.08 (m, 1H), 8.08-8.04 (m, 1H), 7.94-7.88 (m, 1H), 7.76-7.64 (m, 2H), 7.44-7.38 (m, 1H), 7.32-7.26 (m, 1H), 7.05 (s, 1H), 6.99-6.96 (m, 1H), 5.17-4.99 (m, 2H), 4.61-4.57 (m, 1H), 4.55-4.50 (m, 1H), 4.37-4.34 (m, 1H), 4.33-4.23 (m, 2H), 4.22-4.12 (m, 3H), 4.19-4.15 (m, 2H), 4.00-3.83 (m, 6H), 4.01-3.85 (m, 5H), 3.62-3.60 (m, 2H), 3.62-3.58 (m, 3H), 3.43-3.30 (m, 2H), 2.47 (s, 3H), 2.38-2.29 (m, 1H), 2.25-2.15 (m, 1H), 2.11-2.02 (m, 3H), 1.96-1.83 (m, 4H), 1.54-1.46 (m, 2H), 1.40-1.29 (m, 4H), 1.24-1.20 (m, 2H), 1.10-1.01 (m, 2H), 0.96 (s, 9H), 0.83-0.76 (m, 2H).

Example B19. 5-((S)-1′-(4-amino-1,3-dimethyl-1H-pyrazolo[4,3-c]quinoline-8-carbonyl)-3H-spiro[isobenzofuran-1,3′-pyrrolidin]-5-yl)-N-((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)picolinamide

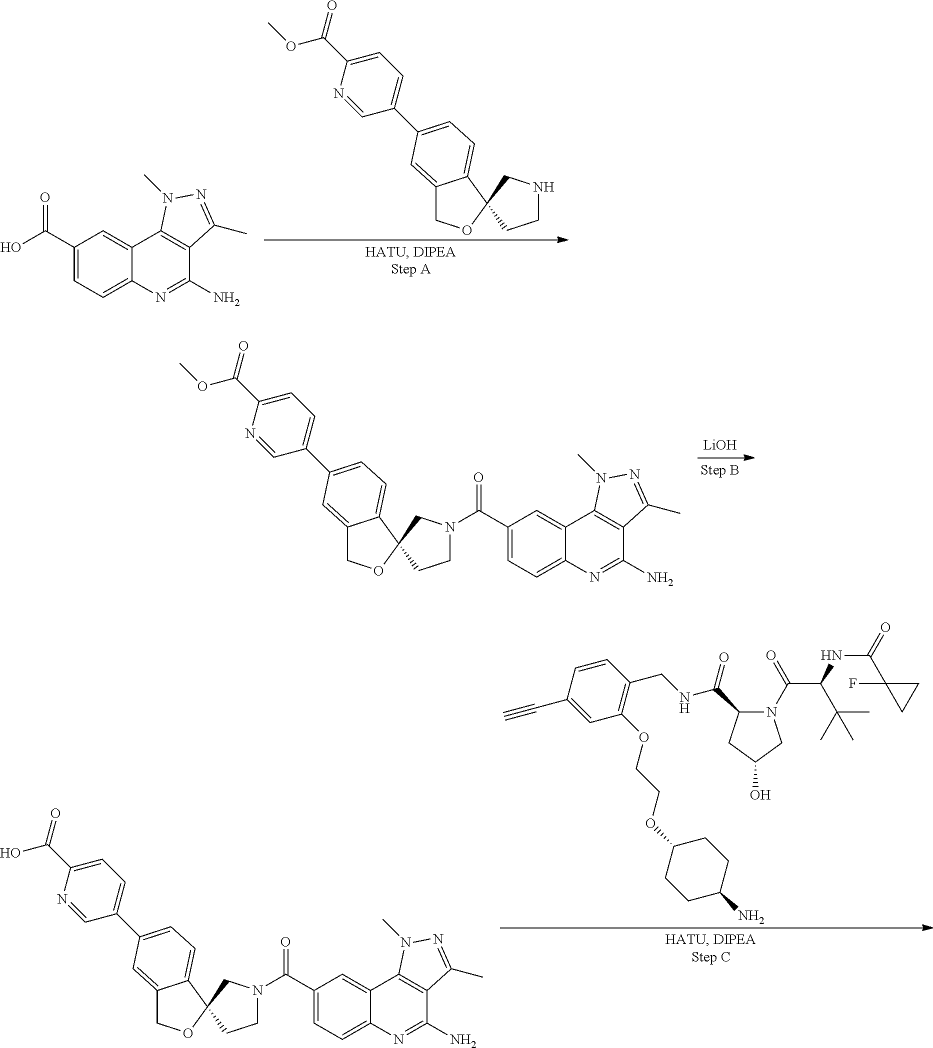

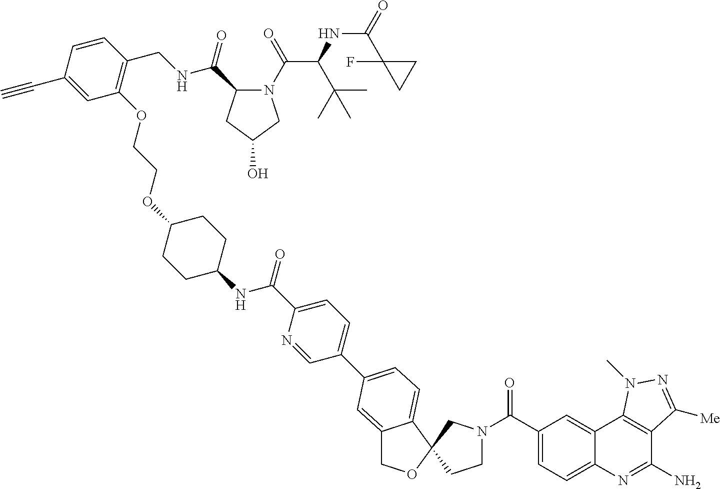 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)CO[C@@]54CCN(C(=O)c5ccc6nc(N)c7c(C)nn(C)c7c6c5)C4)cn3)CC2)c1]

Example B19 was synthesized following the same procedures as described in Example B4 (4.5 mg, 14% yield over 3 steps) as a white solid. LC-MS (ESI): mass calcd, for C₆₂H₆₉FN₁₀O₉, 1116.5; m/z found, 1117.1 [M+H]⁺. ¹H NMR (400 MHz, DMSO-d₆) δ 8.90 (d, J=22.3 Hz, 1H), 8.85-8.62 (m, 1H), 8.61-8.37 (m, 3H), 8.26 (dd, J=20.9, 8.1 Hz, 1H), 8.16-8.05 (m, 1H), 8.05-7.87 (m, 1H), 7.87-7.68 (m, 3H), 7.68-7.48 (m, 1H), 7.35-7.22 (m, 2H), 7.07-7.02 (m, 1H), 6.98 (d, J=7.7 Hz, 1H), 5.21-4.99 (m, 2H), 4.59 (d, J=9.3 Hz, 1H), 4.50 (t, J=8.1 Hz, 1H), 4.43 (s, 1H), 4.38 (s, 1H), 4.37-4.32 (m, 1H), 4.31-4.18 (m, 2H), 4.18-4.03 (m, 4H), 3.97-3.85 (m, 2H), 3.84-3.74 (m, 4H), 3.69-3.57 (m, 4H), 2.74-2.58 (m, 3H), 2.47-2.32 (m, 1H), 2.28-2.16 (m, 1H), 2.10-2.00 (m, 3H), 1.94-1.82 (m, 3H), 1.53-1.44 (m, 2H), 1.38-1.23 (m, 6H), 0.95 (s, 9H).

Example B20. 5-((R)-1′-(4-amino-1,3-dimethyl-1H-pyrazolo[4,3-c]quinoline-8-carbonyl)-3H-spiro[isobenzofuran-1,3′-pyrrolidin]-5-yl)-N-((1S,4R)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)picolinamide

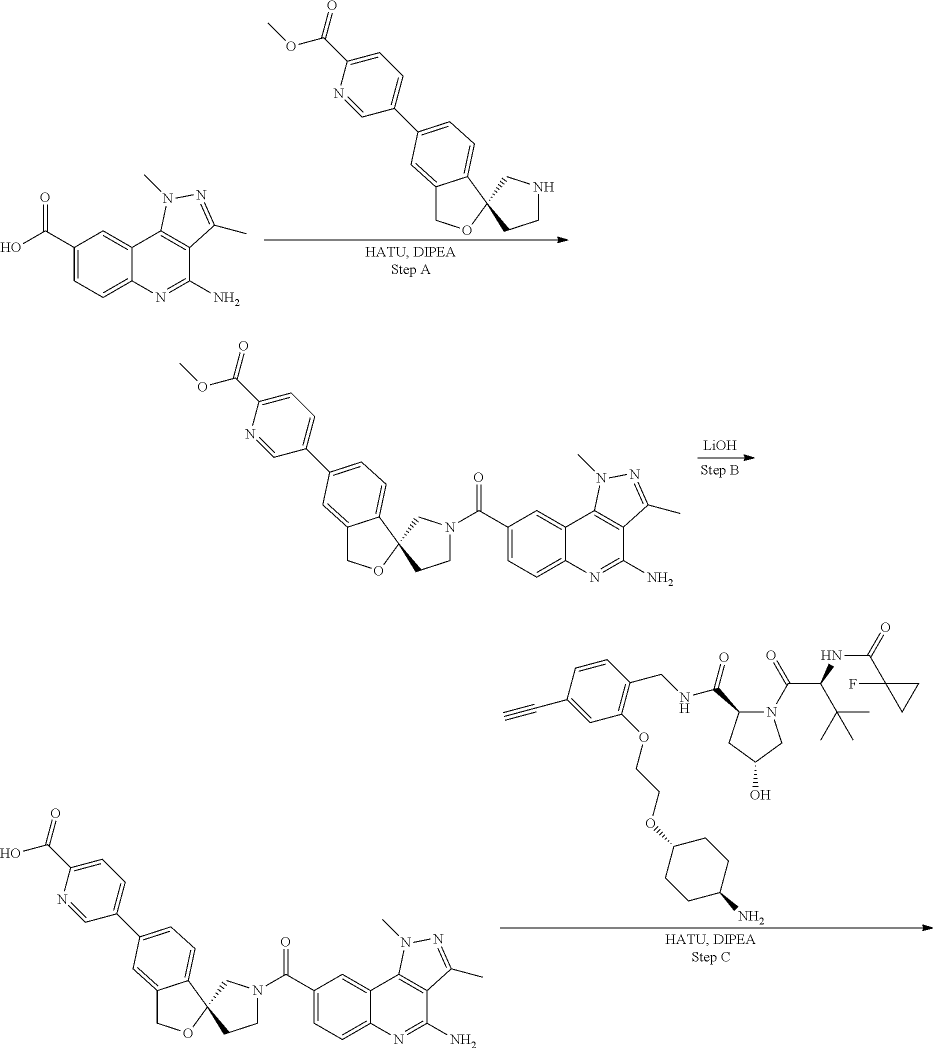

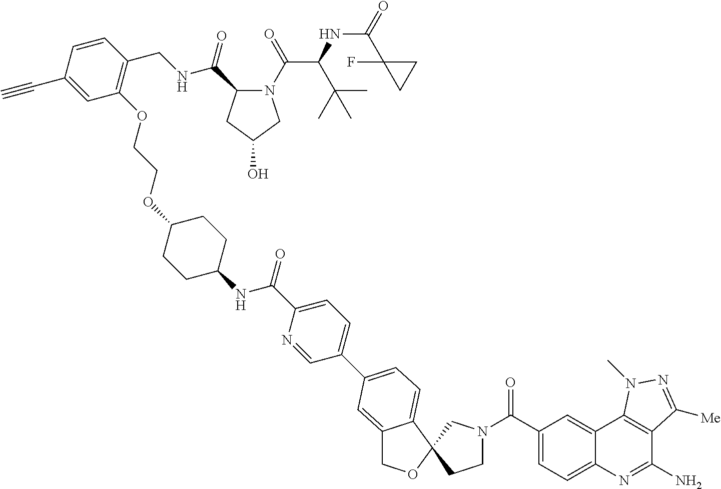 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)CO[C@]54CCN(C(=O)c5ccc6nc(N)c7c(C)nn(C)c7c6c5)C4)cn3)CC2)c1]

Example B20 was synthesized following the same procedures (with starting material prepared according to WO2022115377 A1) as described in Example B4 (4 mg, 8% yield over 3 steps) as a white solid. LC-MS (ESI): mass calcd, for C₆₂H₆₉FN₁₀O₉, 1116.52; m/z found, 1117.1 [M+H]⁺. ¹H NMR (400 MHz, DMSO-d₆) δ 8.91 (d, J=22.1 Hz, 1H), 8.67 (s, 2H), 8.48 (dd, J=19.6, 10.6 Hz, 3H), 8.26 (dd, J=19.9, 9.2 Hz, 1H), 8.10 (dd, J=11.1, 8.4 Hz, 1H), 7.99 (dd, J=19.9, 8.3 Hz, 1H), 7.88-7.69 (m, 3H), 7.62 (dd, J=22.0, 8.2 Hz, 1H), 7.31 (t, J=9.4 Hz, 2H), 7.07 (s, 1H), 6.98 (d, J=7.8 Hz, 1H), 5.20-5.02 (m, 2H), 4.60 (d, J=9.1 Hz, 1H), 4.50 (t, J=7.9 Hz, 1H), 4.42 (d, J=18.5 Hz, 3H), 4.42 (d, J=18.5 Hz, 3H), 4.35 (s, 1H), 4.29-4.18 (m, 2H), 4.13 (s, 3H), 3.90 (d, J=12.8 Hz, 2H), 3.79 (s, 4H), 3.63 (dd, J=22.9, 10.8 Hz, 4H), 2.67 (d, J=13.0 Hz, 3H), 2.45-2.36 (m, 1H), 2.22 (d, J=26.5 Hz, 1H), 2.09 (d, J=21.3 Hz, 3H), 1.89 (d, J=18.9 Hz, 3H), 1.51 (s, 2H), 1.41-1.19 (m, 7H), 0.96 (s, 9H).

Example B21. 5-(1′-(4-amino-1-methyl-1H-pyrazolo[4,3-c]quinoline-8-carbonyl)-3H-spiro[isobenzofuran-1,3′-pyrrolidin]-4-yl)-N-((1S,4r)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)picolinamide

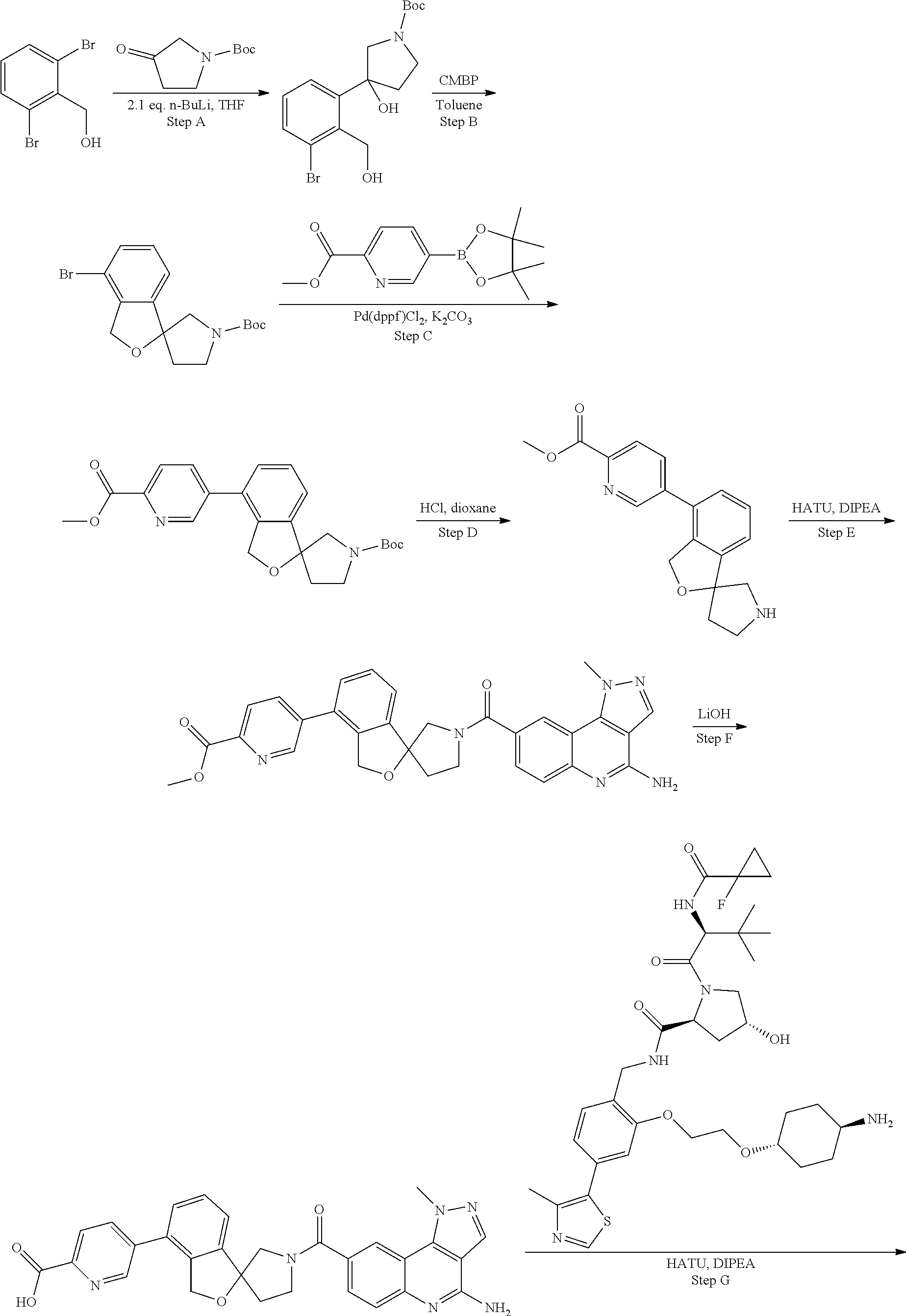

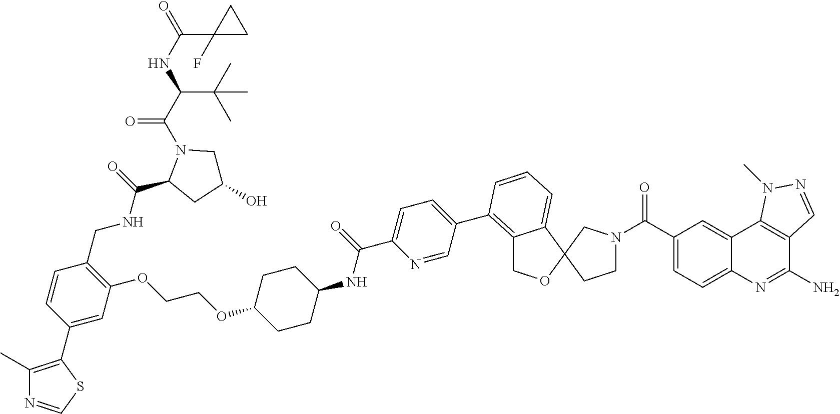 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4cccc5c4COC54CCN(C(=O)c5ccc6nc(N)c7cnn(C)c7c6c5)C4)cn3)CC2)c1]

Step A: tert-butyl 3-(3-bromo-2-(hydroxymethyl)phenyl)-3-hydroxypyrrolidine-1-carboxylate

To a solution of (2,6-dibromophenyl) methanol (2.0 g, 7.52 mmol, 1.0 eq.) in THF (20 mL) was added n-BuLi (9.87 mL, 1.6 M, 15.8 mmol, 2.1 eq.) under N₂ at −78° C. Then the mixture was stirred at −78° C. for 1 hour. A solution of tert-butyl 3-oxopyrrolidine-1-carboxylate (1.53 g, 8.27 mmol, 1.1 eq.) in THF at −78° C. The mixture was stirred at −78° C. for 2 hours under N₂ atmosphere. The mixture was quenched by addition to ice-water and (50 mL) and extracted with EtOAc (20 mL×3). The combined organic layers were washed with brine, dried over anhydrous Na₂SO₄ and concentrated under reduced pressure. The residue was purified by flash chromatographer (eluent, 0~30% EtOAc in PE) to give tert-butyl 3-(3-bromo-2-(hydroxymethyl)phenyl)-3-hydroxypyrrolidine-1-carboxylate (900 mg, 32% yield) as a white solid.

LC-MS (ESI): mass calcd, for C₁₆H₂₂BrNO₄, 371.1, 373.1; m/z found, 297.8, 299.8 [M−56+18+H]⁺.

Step B: tert-butyl 4-bromo-3H-spiro[isobenzofuran-1,3′-pyrrolidine]-1′-carboxylate

To a solution of tert-butyl 3-(3-bromo-2-(hydroxymethyl)phenyl)-3-hydroxypyrrolidine-1-carboxylate (200 mg, 537.3 μmol, 1.0 eq.) in toluene (5 mL) was added (cyanomethylene)tributylphosphorane (CMBP, 259.3 mg, 1.075 mmol, 2.1 eq.) under N₂. Then was stirred at 110° C. for 16 hours. The mixture was concentrated under reduced pressure to dryness. The residue was purified by flash chromatographer (eluent, 0~10% EtOAc in PE) to give tert-butyl 4-bromo-3H-spiro[isobenzofuran-1,3′-pyrrolidine]-1′-carboxylate (70 mg, yield 37%) as a yellow oil.

LC-MS (ESI): mass calcd, for C₁₆H₂₀BrNO₃, 353.1, 355.1; m/z found, 297.8, 299.8 [M−56+H]⁺.

### Steps C-G

Example B21 was synthesized following the same procedures as described in Example B4 (5.1 mg, 5% yield over 5 steps) as a white solid. LC-MS (ESI): mass calcd, for C₆₃H₇₀FN₁₁O₉S, 1175.5; m/z found, 1176.5 [M+H]⁺. ¹H NMR (400 MHz, DMSO-d₆) δ 9.95-9.44 (m, 1H), 8.98 (s, 1H), 8.78-8.68 (m, 1H), 8.62 (d, J=13.7 Hz, 1H), 8.55-8.44 (m, 3H), 8.19-8.08 (m, 1H), 8.08-8.03 (m, 1H), 8.03-7.94 (m, 1H), 7.88-7.78 (m, 1H), 7.61-7.57 (m, 1H), 7.56-7.48 (m, 2H), 7.44-7.37 (m, 1H), 7.29 (d, J=9.0 Hz, 1H), 7.05 (d, J=1.8 Hz, 1H), 6.97 (d, J=7.8 Hz, 1H), 5.32-5.19 (m, 1H), 5.16-5.09 (m, 1H), 4.59 (d, J=8.8 Hz, 1H), 4.54-4.50 (m, 2H), 4.50-4.45 (m, 2H), 4.37-4.34 (m, 1H), 4.32-4.27 (m, 1H), 4.27-4.20 (m, 1H), 4.19-4.14 (m, 2H), 4.13-3.97 (m, 1H), 3.97-3.86 (m, 2H), 3.84-3.77 (m, 4H), 3.70-3.57 (m, 4H), 2.46 (d, J=2.3 Hz, 3H), 2.45-2.33 (m, 1H), 2.29-2.21 (m, 1H), 2.18-1.97 (m, 4H), 1.87-1.79 (m, 2H), 1.54-1.44 (m, 2H), 1.39-1.22 (m, 6H), 0.97-0.92 (m, 9H).

Example B22. 5-((R)-1-(2-amino-3-methylquinoline-6-carbonyl)-6′H-spiro[pyrrolidine-3,4′-thieno[2,3-c]furan]-2′-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide

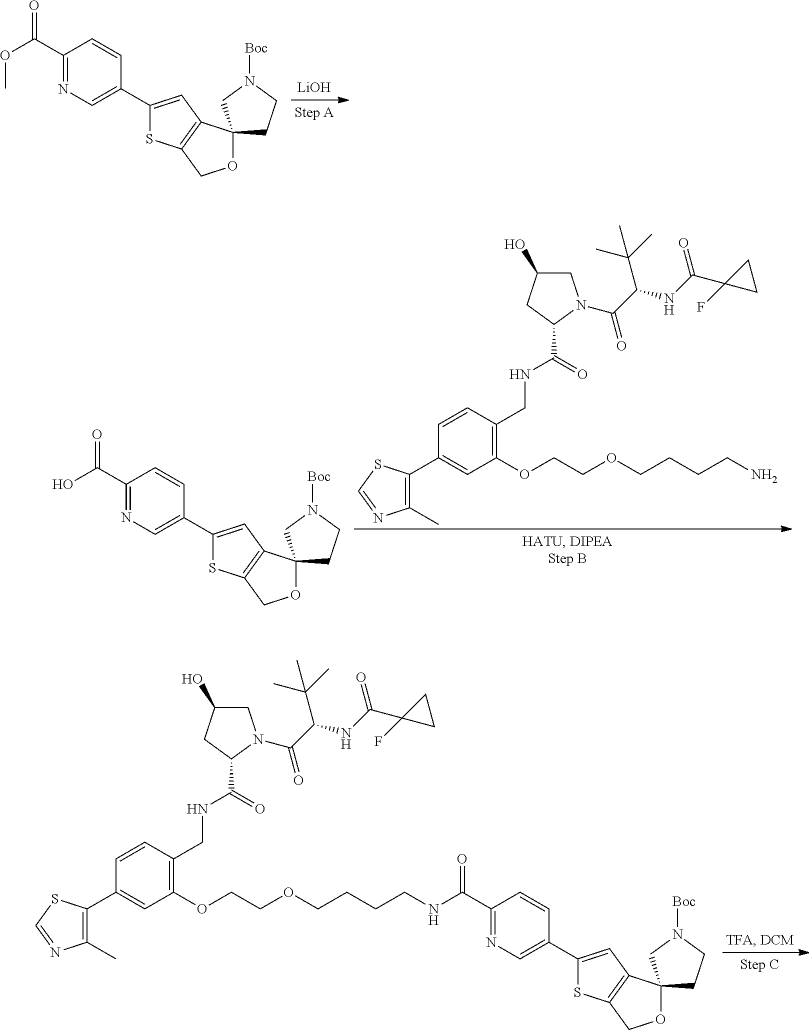

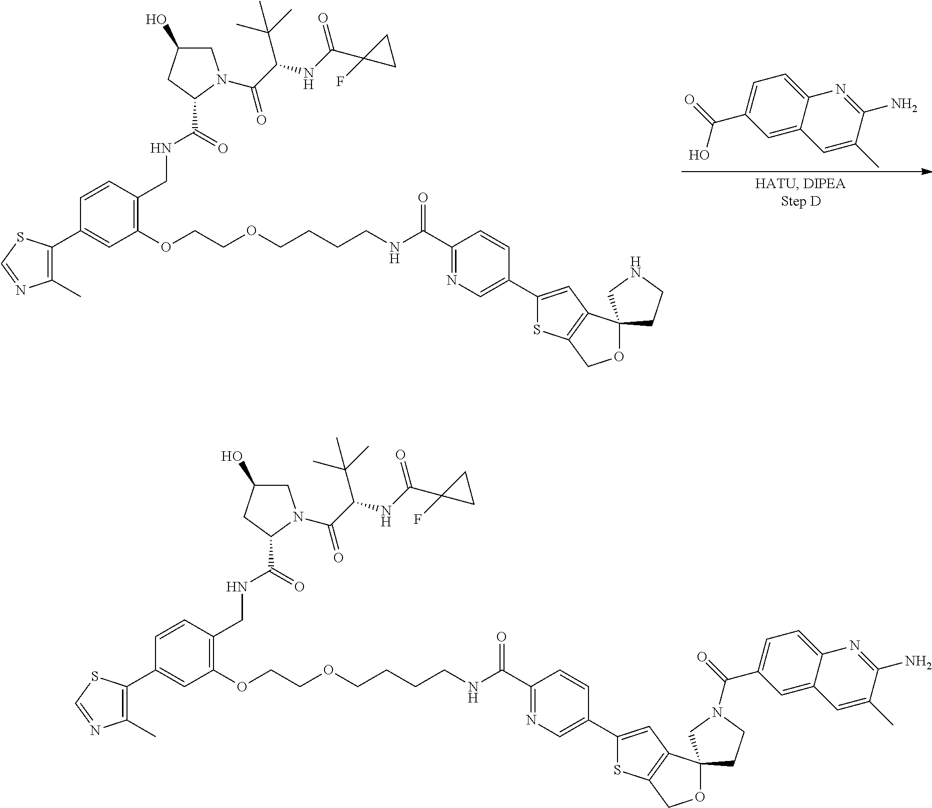 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3cc4c(s3)CO[C@]43CCNC3)cn2)c1 ; Cc1cc2cc(C(=O)O)ccc2nc1N ; Cc1cc2cc(C(=O)N3CC[C@@]4(C3)OCc3sc(-c5ccc(C(=O)NCCCCOCCOc6cc(-c7scnc7C)ccc6CNC(=O)[C@@H]6C[C@@H](O)CN6C(=O)[C@@H](NC(=O)C6(F)CC6)C(C)(C)C)nc5)cc34)ccc2nc1N]

Step A: (R)-5-(1-(tert-butoxycarbonyl)-6′H-spiro[pyrrolidine-3,4′-thieno[2,3-c]furan]-2′-yl)picolinic acid

To a solution of tert-butyl (R)-2′-(6-(methoxycarbonyl)pyridin-3-yl)-6′H-spiro[pyrrolidine-3,4′-thieno[2,3-c]furan]-1-carboxylate (80 mg, 192 μmol, 1.0 eq.) in THF:H₂O=4:1 (4.00 mL) was added LiOH (9 mg, 384 μmol, 2.0 eq.) at room temperature. The mixture was stirred at room temperature for 1 hour under N₂ atmosphere. The resulting solution was concentrated under reduced pressure to afford (R)-5-(1-(tert-butoxycarbonyl)-6′H-spiro[pyrrolidine-3,4′-thieno[2,3-c]furan]-2′-yl)picolinic acid (lithium salt, 77 mg, 99.6% yield) as a yellow solid, which was used directly without further purification. LC-MS (ESI): mass calcd. for C₂₀H₂₂N₂O₅S, 402.1; m/z found, 403.1 [M+H]⁺.

Step B: ter-butyl (R)-2′-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-6′H-spiro[pyrrolidine-3,4′-thieno[2,3-c]furan]-1-carboxylate

To a solution of (R)-5-(1-(tert-butoxycarbonyl)-6′H-spiro[pyrrolidine-3,4′-thieno[2,3-c]furan]-2′-yl)picolinic acid (77 mg, 191 μmol, 1.0 eq) in DMF (3.00 mL) was added (2S,4R)—N-(2-(2-(4-aminobutoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (124 mg, 191 μmol, 1.0 eq.), HATU (145 mg, 383 μmol, 2.0 eq.) and DIPEA (141 μL, 99 mg, 765 μmol, 4.0 eq.) at room temperature. The mixture was then stirred at room temperature for 1 hour under N₂ atmosphere. The resulting solution was diluted with 10 mL of water and extracted with EtOAc (10 mL×3). The combined organic layers were washed with brine, dried over anhydrous Na₂SO₄ and concentrated under reduced pressure. The residue was purified by flash column chromatography on silica gel (100-200 mesh silica gel, 0-10% MeOH in DCM) to afford tert-butyl (R)-2′-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl) carbamoyl)pyridin-3-yl)-6′H-spiro[pyrrolidine-3,4′-thieno[2,3-c]furan]-1-carboxylate (150 mg, 76% yield) as a white solid.

LC-MS (ESI): mass calcd, for C₅₂H₆₆FN₇O₁₀S₂, 1031.4; m/z found, 1032.4 [M+H]⁺.

Step C: N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)-5-((R)-6′H-spiro[pyrrolidine-3,4′-thieno[2,3-c]furan]-2′-yl)picolinamide

To a solution of tert-butyl (R)-2′-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methyl thiazol-5-yl)phenoxy)ethoxy)butyl)carbamoyl)pyridin-3-yl)-6′H-spiro[pyrrolidine-3,4′-thieno[2,3-c]furan]-1-carboxylate (150 mg, 145 μmol, 1.0 eq.) in DCM (3.00 mL) was added TFA (1 mL). The mixture was stirred at room temperature for 1 hours under N₂ atmosphere. The resulting solution was concentrated under reduced pressure to afford N-(4-(2-(2-(((2S,4R)-1-((S)-2-(1-fluorocyclopropane-1-carboxamido)-3,3-dimethylbutanoyl)-4-hydroxypyrrolidine-2-carboxami-mido)methyl)-5-(4-methylthiazol-5-yl)phenoxy)ethoxy)butyl)-5-((R)-6′H-spiro[pyrrolidine-3,4′-thieno[2,3-c]furan]-2′-yl)picolinamide (TFA salt, 135 mg, 100% yield) as a brown oil, which was used directly without further purification.

LC-MS (ESI): mass calcd, for C₄₇H₅₈FN₇O₈S₂, 931.4; m/z found, 932.4 [M+H]⁺.

Step D: 5-((R)-1-(2-amino-3-methylquinoline-6-carbonyl)-6′H-spiro[pyrrolidine-3,4′-thieno[2,3-c]furan]-2′-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide

To a mixture of 2-amino-3-methylquinoline-6-carboxylic acid (20.0 mg, 1 Eq, 41.1 μmol), HATU (31.3 mg, 2 Eq, 82.2 μmol) and N-(4-(2-(2-(((2S,4R)-1-((S)-2-(1-fluorocyclopropane-1-carboxamido)-3,3-dimethylbutanoyl)-4-hydroxypyrrolidine-2-carboxami-mido)methyl)-5-(4-methylthiazol-5-yl)phenoxy)ethoxy)butyl)-5-((R)-6′H-spiro[pyrrolidine-3,4′-thieno[2,3-c]furan]-2′-yl)picolinamide (32.0 mg, 1.2 Eq, 49.3 μmol) in DMF (2.00 mL) was added DIEA (21.3 mg, 28.6 μL, 4 Eq, 164 μmol). The reaction mixture was purged with nitrogen three times, then stirred at 0° C. for 30 min under nitrogen. The reaction mixture was purified by Prep-HPLC to afford 5-((R)-1-(2-amino-3-methylquinoline-6-carbonyl)-6′H-spiro[pyrrolidine-3,4′-thieno[2,3-c]furan]-2′-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide (1.50 mg, 1.34 μmol, 3.27%) as a yellow solid.

LC-MS (ESI): mass calcd, for C₅₈H₆₆FN₉O₉S₂, 1115.4; m/z found, 1116.4 [M+H]⁺. ¹H NMR (400 MHz, DMSO-d₆) δ 8.97 (s, 1H), 8.88-8.83 (m, 1H), 8.77 (s, 1H), 8.70-8.53 (m, 1H), 8.49 (s, 1H), 8.34-8.26 (m, 1H), 8.16-8.03 (m, 3H), 7.94-7.87 (m, 1H), 7.75-7.63 (m, 2H), 7.42-7.34 (m, 1H), 7.31-7.25 (m, 1H), 7.04 (s, 1H), 6.97-6.93 (m, 1H), 5.16-4.99 (m, 2H), 4.63-4.55 (m, 1H), 4.55-4.46 (m, 1H), 4.36-4.33 (m, 1H), 4.32-4.27 (m, 1H), 4.27-4.21 (m, 1H), 4.21-4.17 (m, 2H), 3.91-3.81 (m, 2H), 3.76 (s, 3H), 3.63-3.60 (m, 2H), 3.56-3.50 (m, 6H), 2.45 (s, 3H), 2.34-2.28 (m, 4H), 1.62-1.55 (m, 4H), 1.38-1.32 (m, 2H), 1.27-1.19 (m, 4H), 0.95 (s, 9H).

Example B23. 5-((R)-1-(2-((7-amino-1-ethyl-1H-pyrazolo[3,4-c]pyridin-4-yl)amino)-2-oxoacetyl)-6′H-spiro[pyrrolidine-3,4′-thieno[2,3-c]furan]-2′-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide

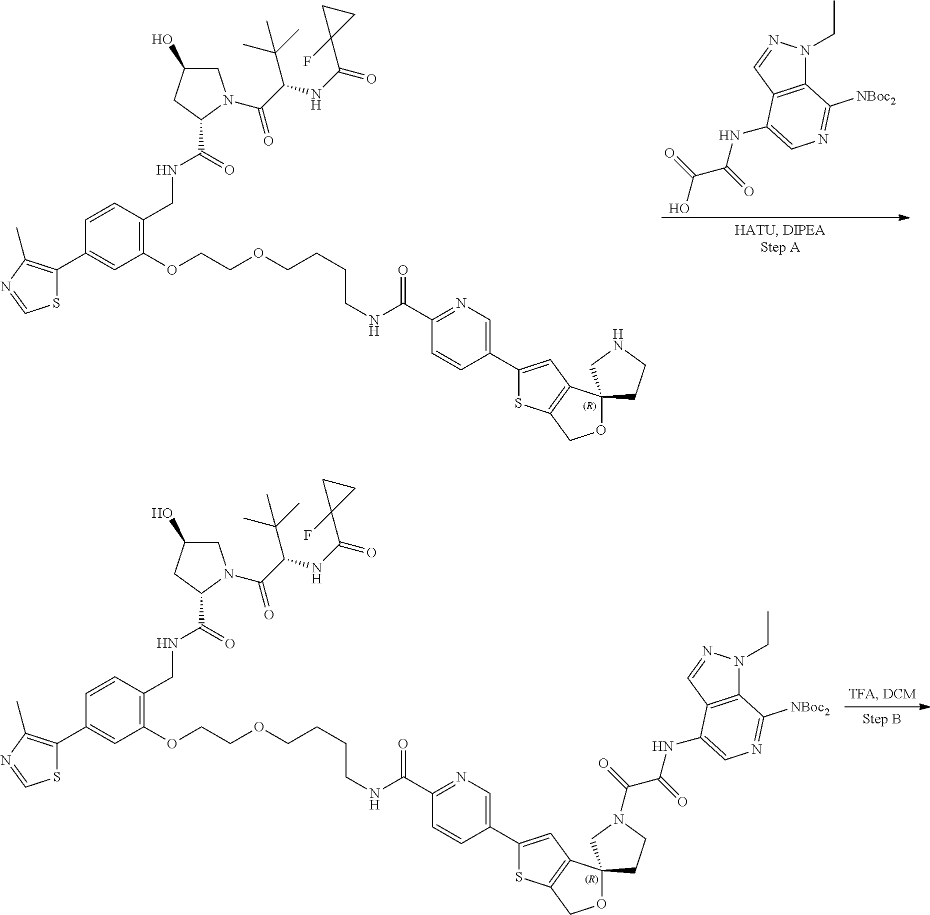

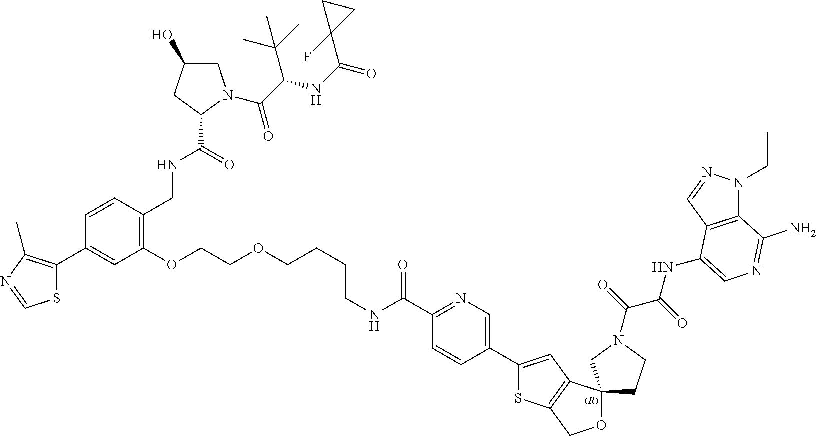

Step A: tert-butyl (tert-butoxycarbonyl) (1-ethyl-4-(2-((R)-2′-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-6′H-spiro[pyrrolidine-3,4′-thieno[2,3-c]furan]-1-yl)-2-oxoacetamido)-1H-pyrazolo[3,4-c]pyridin-7-yl)carbamate

To a solution of N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)-5-((R)-6′H-spiro[pyrrolidine-3,4′-thieno[2,3-c]furan]-2′-yl)picolinamide (20 mg, 22 μmol, 1.0 eq.) in DMF (3 mL) was added 2-((7-(bis(tert-butoxycarbonyl)amino)-1-ethyl-1H-pyrazolo[3,4-c]pyridin-4-yl)amino)-2-oxoacetic acid (WO2022026892 A1, 10 mg, 22 μmol, 1.0 eq.), HATU (16 mg, 43 μmol, 2.0 eq.) and DIPEA (11 mg, 86 μmol, 4.0 eq.) at room temperature. The mixture was then stirred at room temperature for 1 hour under N₂ atmosphere. The resulting solution was diluted with 30 mL of H₂O and extracted with EtOAc (30 mL×3). The combined organic layers were washed with brine, dried over Na₂SO₄. The reaction was concentrated under reduced pressure to give tert-butyl (tert-butoxycarbonyl) (1-ethyl-4-(2-((R)-2′-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-6′H-spiro[pyrrolidine-3,4′-thieno[2,3-c]furan]-1-yl)-2-oxoacetamido)-1H-pyrazolo[3,4-c]pyridin-7-yl)carbamate (20 mg, crude). The product was used for next step directly without further purification.

LC-MS (ESI): mass calcd, for C₆₇H₈₃FN₁₂O₁₄S₂, 1362.6; m/z found, 1363.6 [M+H]⁺.

Step B: 5-((R)-1-(2-((7-amino-1-ethyl-1H-pyrazolo[3,4-c]pyridin-4-yl)amino)-2-oxoacetyl)-6′H-spiro[pyrrolidine-3,4′-thieno[2,3-c]furan]-2′-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide

To a solution of tert-butyl (tert-butoxycarbonyl) (1-ethyl-4-(2-((R)-2′-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-6′H-spiro[pyrrolidine-3,4′-thieno[2,3-c]furan]-1-yl)-2-oxoacetamido)-1H-pyrazolo[3,4-c]pyridin-7-yl)carbamate (20 mg, 15 μmol, 1.0 eq.) in DCM:TFA (4 mL, 3:1) was added at room temperature. The mixture was then stirred at room temperature for 1 hour under N₂ atmosphere. The residue was purified by Pre-HPLC(C18, 20~95%, MeCN in H₂O with 0.1% TFA) to afford 5-((R)-1-(2-((7-amino-1-ethyl-1H-pyrazolo[3,4-c]pyridin-4-yl)amino)-2-oxoacetyl)-6′H-spiro[pyrrolidine-3,4′-thieno[2,3-c]furan]-2′-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide (3.3 mg, 19% yield) as a white solid.

LC-MS (ESI): mass calcd, for C₅₇H₆₇FN₁₂O₁₀S₂, 1162.5; m/z found, 1163.8 [M+H]⁺. ¹H NMR (400 MHz, DMSO-d₆) δ 11.04 (d, J=7.5 Hz, 1H), 8.97 (d, J=1.6 Hz, 1H), 8.89-8.82 (m, 1H), 8.79-8.72 (m, 1H), 8.52-8.44 (m, 1H), 8.41 (d, 1H), 8.35 (s, 1H), 8.18-8.11 (m, 1H), 8.09-8.03 (m, 1H), 7.98 (d, 1H), 7.69 (s, 1H), 7.39 (d, J=7.7 Hz, 1H), 7.28 (d, 1H), 7.04 (s, 1H), 6.95 (d, 1H), 5.16-5.08 (m, 2H), 4.77-4.69 (m, 2H), 4.59 (d, J=9.2 Hz, 1H), 4.54-4.47 (m, 1H), 4.36-4.33 (m, 1H), 4.28 (d, 1H), 4.24-4.17 (m, 3H), 4.02 (s, 1H), 3.92-3.82 (m, 2H), 3.78-3.74 (m, 3H), 3.64-3.62 (m, 2H), 3.52-3.52 (m, 2H), 3.34-3.30 (m, 2H), 2.45 (d, J=1.4 Hz, 3H), 2.39-2.33 (m, 1H), 2.26-2.18 (m, 1H), 2.12-2.04 (m, 1H), 1.95-1.86 (m, 1H), 1.63-1.53 (m, 4H), 1.43-1.36 (m, 4H), 1.36-1.31 (m, 1H), 1.24-1.19 (m, 2H), 0.94 (s, 9H).

Example B24. 5-((R)-1-(4-amino-1-methyl-1H-pyrazolo[4,3-c]quinoline-8-carbonyl)-6′H-spiro[pyrrolidine-3,4′-thieno[2,3-c]furan]-2′-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide

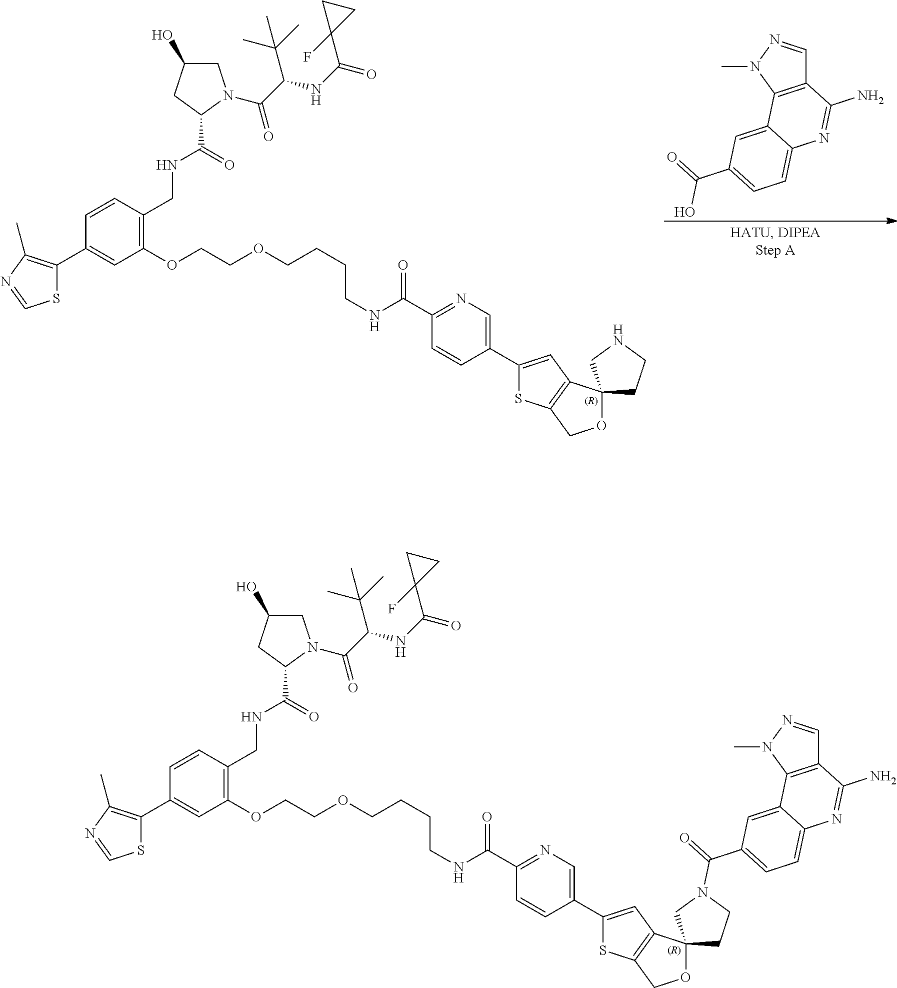

Example B24 was synthesized following the same procedures as described in Example B22 (9 mg, 30% yield) as a white solid. LC-MS (ESI): mass calcd, for C₅₉H₆₆FN₁₁O₉S₂, 1155.4; m/z found, 1156.4 [M+H]⁺. ¹H NMR (400 MHz, DMSO-d₆) δ 13.45 (s, 1H), 9.64 (s, 1H), 8.97 (d, J=2.2 Hz, 1H), 8.89-8.82 (m, 1H), 8.78-8.73 (m, 1H), 8.66-8.58 (m, 1H), 8.54-8.41 (m, 2H), 8.18-8.10 (m, 1H), 8.08-7.91 (m, 2H), 7.89-7.78 (m, 1H), 7.73-7.61 (m, 1H), 7.40 (d, J=7.7 Hz, 1H), 7.32-7.23 (m, 1H), 7.04 (s, 1H), 6.95 (d, J=6.9 Hz, 1H), 5.19-5.09 (m, 1H), 5.06-4.96 (m, 1H), 4.59 (d, J=9.4 Hz, 1H), 4.54-4.45 (m, 4H), 4.35-4.27 (m, 2H), 4.24-4.17 (m, 3H), 4.01-3.96 (m, 1H), 3.90-3.87 (m, 1H), 3.85-3.83 (m, 1H), 3.76-3.75 (m, 2H), 3.63-3.61 (m, 3H), 3.54-3.51 (m, 2H), 3.34-3.29 (m, 2H), 2.45 (s, 3H), 2.37-2.31 (m, 1H), 2.26-2.16 (m, 1H), 2.11-2.04 (m, 1H), 1.96-1.87 (m, 1H), 1.58 (s, 4H), 1.41-1.31 (m, 2H), 1.24-1.18 (m, 2H), 0.94 (s, 9H).

Example B25. 5-((R)-1-(4-amino-1,3-dihydrofuro[3,4-c][1,7]naphthyridine-8-carbonyl)-6′H-spiro[pyrrolidine-3,4′-thieno[2,3-c]furan]-2′-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide

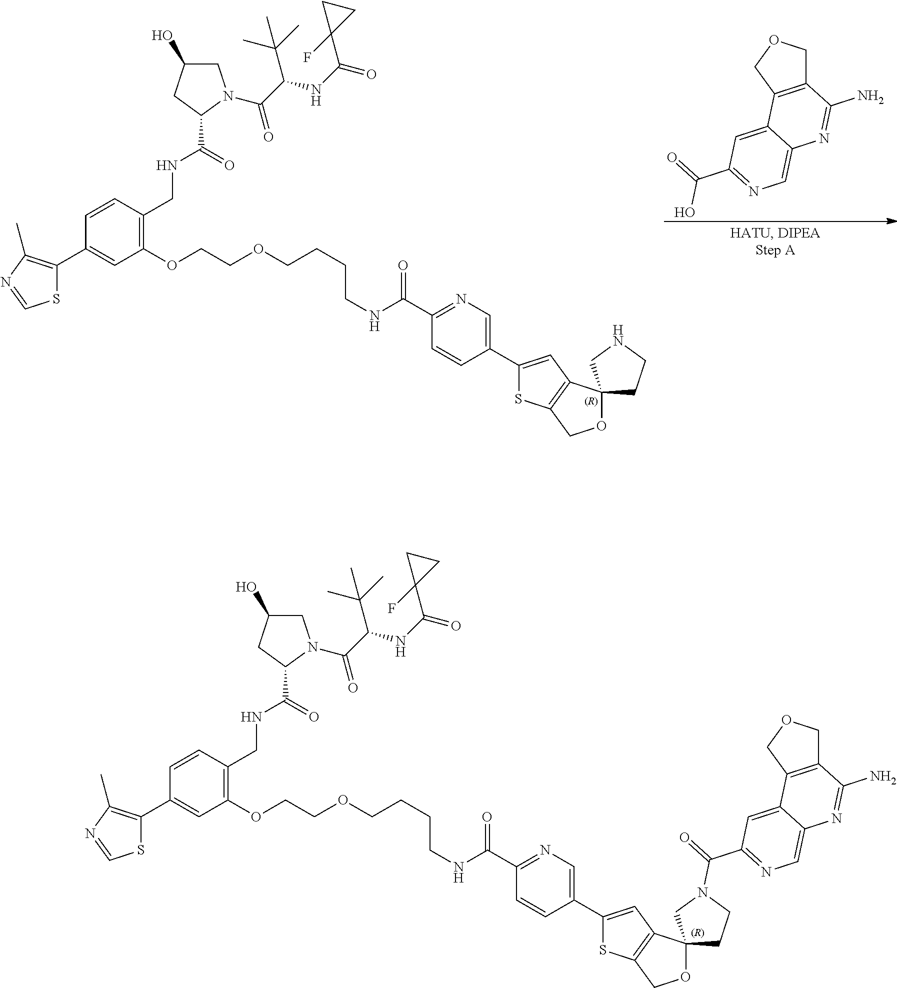

Example B25 was synthesized following the same procedures as described in Example B22 (10.4 mg, 40% yield) as a white solid. LC-MS (ESI): mass calcd, for C₅₈H₆₅FN₁₀O₁₀S₂, 1144.4; m/z found, 1145.2 [M+H]⁺. ¹H NMR (400 MHz, DMSO-d₆) δ 9.05-8.91 (m, 2H), 8.90-8.82 (m, 1H), 8.80-8.70 (m, 1H), 8.53-8.43 (m, 1H), 8.16-8.12 (m, 1H), 8.08-8.03 (m, 2H), 7.72 (d, J=7.6 Hz, 1H), 7.40 (d, J=7.8 Hz, 1H), 7.32-7.23 (m, 1H), 7.05-7.01 (m, 1H), 6.98-6.93 (m, 1H), 5.48-5.42 (m, 2H), 5.13-5.06 (m, 3H), 5.05-5.02 (m, 1H), 4.60-4.58 (m, 1H), 4.53-4.49 (m, 2H), 4.35-4.34 (m, 1H), 4.30-4.28 (m, 1H), 4.24-4.22 (m, 1H), 4.20-4.18 (m, 2H), 3.99-3.91 (m, 2H), 3.89-3.84 (m, 1H), 3.77-3.74 (m, 2H), 3.68-3.59 (m, 2H), 3.55-3.50 (m, 2H), 3.36-3.29 (m, 2H), 2.45 (s, 3H), 2.40-2.31 (m, 1H), 2.23-2.15 (m, 1H), 2.12-2.04 (m, 1H), 1.96-1.87 (m, 1H), 1.63-1.52 (m, 4H), 1.41-1.30 (m, 2H), 1.24-1.19 (m, 2H), 0.95 (s, 9H).

Example B26. 5-((R)-1-(2-((6-amino-5-methylpyridin-3-yl)amino)-2-oxoacetyl)-6′H-spiro[pyrrolidine-3,4′-thieno[2,3-c]furan]-2′-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide

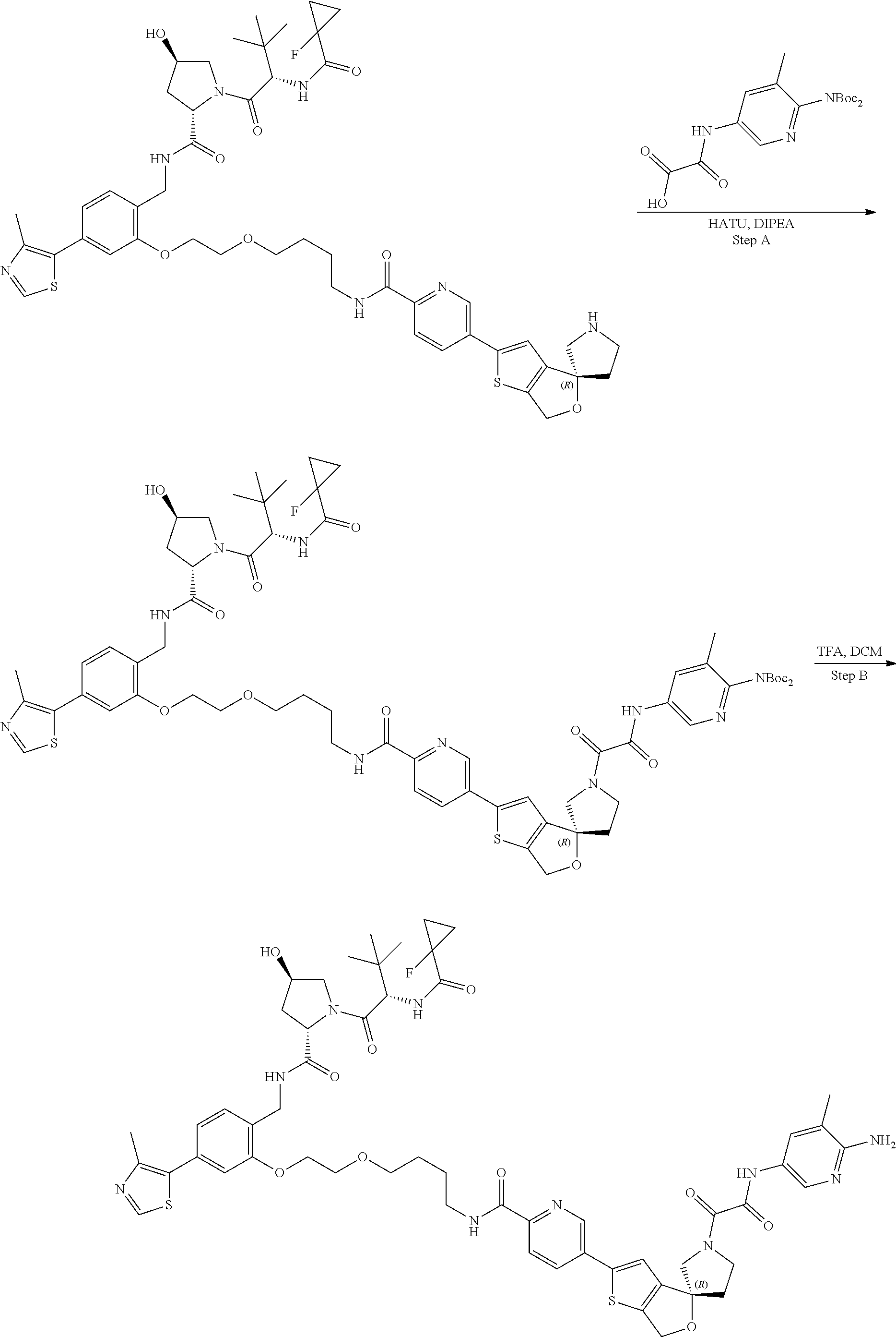

Example B26 was synthesized following the same procedures as described in Example B23 (3.5 mg, 15% yield over 2 steps) as a white solid. LC-MS (ESI): mass calcd. For C₅₅H₆₅FN₁₀O₁₀S₂, 1108.4; m/z found, 1109.4 [M+H]⁺. ¹H NMR (400 MHz, DMSO-d₆) δ 10.98 (s, 1H), 8.97 (s, 1H), 8.85 (s, 1H), 8.79-8.72 (m, 1H), 8.52-8.43 (m, 1H), 8.39-8.31 (m, 1H), 8.17-8.11 (m, 1H), 8.06-8.01 (m, 2H), 7.82-7.78 (m, 1H), 7.68 (s, 1H), 7.40 (d, J=7.8 Hz, 1H), 7.31-7.24 (m, 1H), 7.06-7.02 (m, 1H), 6.97-6.94 (m, 1H), 5.12-5.07 (m, 2H), 4.59 (d, J=8.9 Hz, 1H), 4.53-4.49 (m, 1H), 4.35-4.33 (m, 1H), 4.31-4.27 (m, 1H), 4.24-4.21 (m, 1H), 4.20-4.18 (m, 2H), 4.07-4.05 (m, 1H), 3.90-3.88 (m, 1H), 3.86-3.83 (m, 2H), 3.76-3.75 (m, 2H), 3.64-3.62 (m, 1H), 3.62-3.60 (m, 1H), 3.54-3.51 (m, 2H), 3.34-3.30 (m, 2H), 2.45 (s, 3H), 2.36-2.29 (m, 1H), 2.22-2.16 (m, 4H), 2.11-2.05 (m, 1H), 1.95-1.87 (m, 1H), 1.61-1.54 (m, 4H), 1.40-1.32 (m, 2H), 1.23-1.18 (m, 2H), 0.95 (s, 9H).

Example B27. 5-((R)-1-(2-((6-amino-5-ethylpyridin-3-yl)amino)-2-oxoacetyl)-6′H-spiro[pyrrolidine-3,4′-thieno[2,3-c]furan]-2′-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide

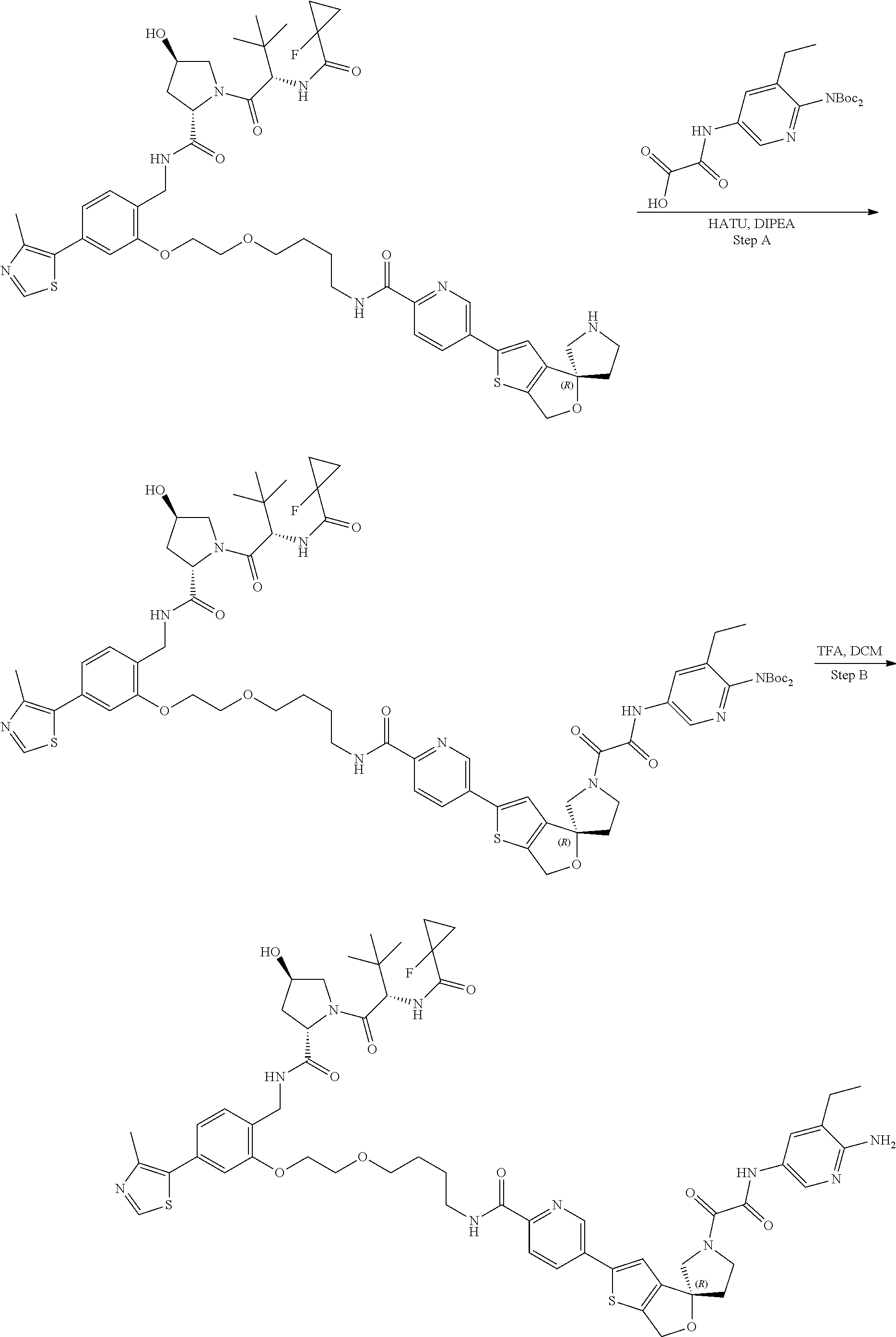

Example B27 was synthesized following the same procedures as described in Example B23 (4 mg, 17% yield over 2 steps) as a white solid. LC-MS (ESI): mass calcd. for C₅₆H₆₇FN₁₀O₁₀S₂, 1122.4; m/z found, 1123.4 [M+H]⁺. ¹H NMR (400 MHz, DMSO-d₆) δ 10.99 (s, 1H), 8.97 (s, 1H), 8.85 (s, 1H), 8.79-8.72 (m, 1H), 8.51-8.45 (m, 1H), 8.44-8.34 (m, 1H), 8.19-8.11 (m, 1H), 8.08-8.03 (m, 2H), 7.76 (s, 1H), 7.68 (s, 1H), 7.40 (d, J=7.8 Hz, 1H), 7.28 (d, J=6.6 Hz, 1H), 7.04 (s, 1H), 6.96 (d, J=7.7 Hz, 1H), 5.12-5.06 (m, 2H), 4.59 (d, J=9.1 Hz, 1H), 4.55-4.47 (m, 1H), 4.37-4.27 (m, 2H), 4.24-4.17 (m, 3H), 4.11-4.04 (m, 1H), 3.94-3.83 (m, 1H), 3.79-3.71 (m, 3H), 3.67-3.58 (m, 3H), 3.53-3.51 (m, 2H), 3.33-3.31 (m, 2H), 2.45 (s, 3H), 2.41-2.31 (m, 2H), 2.24-2.17 (m, 1H), 2.11-2.05 (m, 1H), 1.94-1.87 (m, 1H), 1.62-1.53 (m, 4H), 1.41-1.32 (m, 2H), 1.26-1.13 (m, 6H), 0.94 (s, 9H).

Example B28. 5-((S)-1′-(2-amino-3-methylquinoline-6-carbonyl)-3H-spiro[isobenzofuran-1,3′-pyrrolidin]-5-yl)-N-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)picolinamide

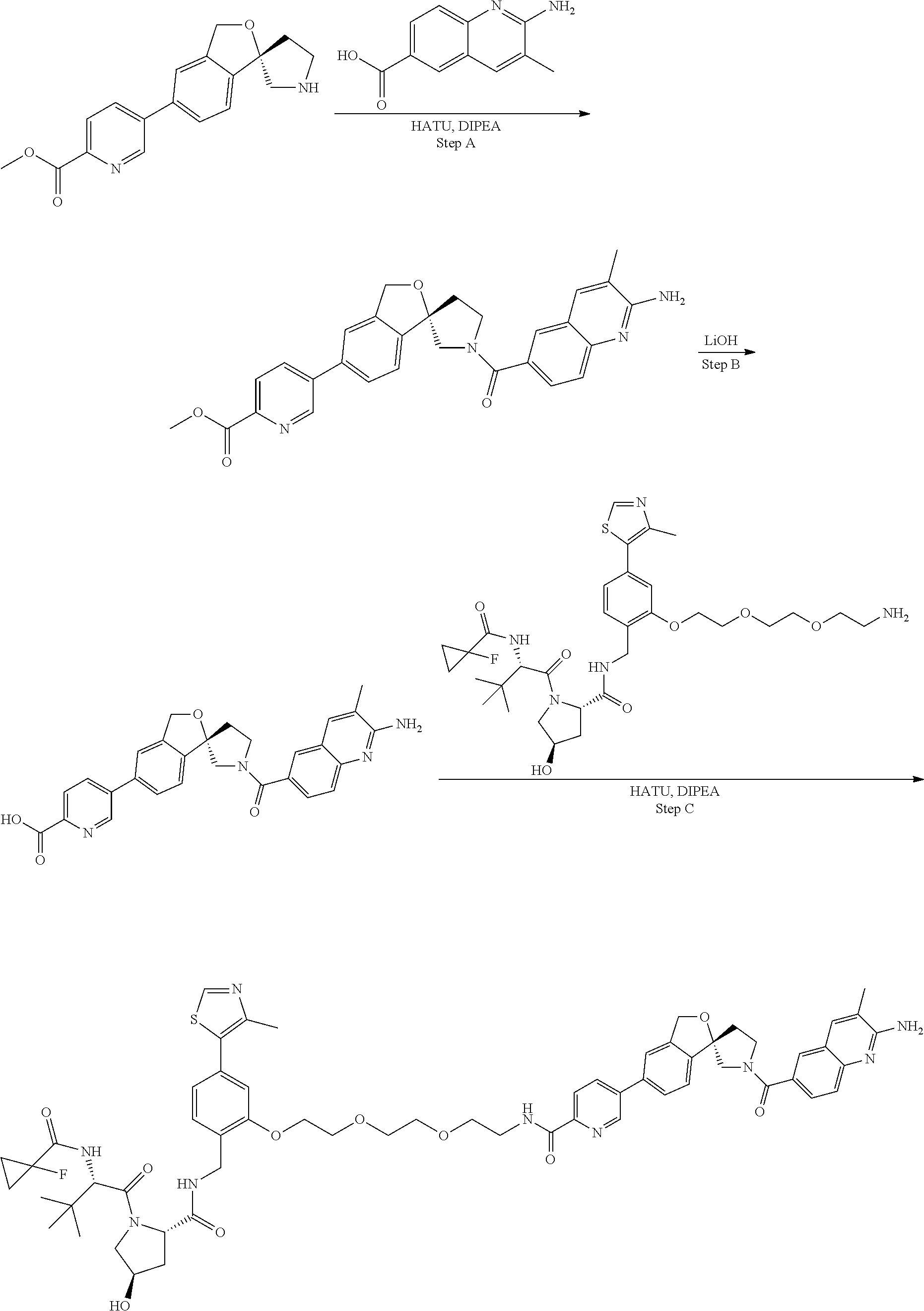

Example B28 was synthesized following the same procedures as described in Example B13 (4 mg, 11% yield over 3 steps) as a white solid. LC-MS (ESI): mass calcd. for C₆₀H₆₈FN₉O₁₀S, 1125.5; m/z found, 1126.4 [M+H]⁺. ¹H NMR (400 MHz, DMSO-d₆) δ 8.99-8.65 (m, 5H), 8.52-8.45 (m, 1H), 8.37-8.28 (m, 1H), 8.27-8.20 (m, 1H), 8.17-8.07 (m, 2H), 7.97-7.90 (m, 1H), 7.77-7.66 (m, 3H), 7.63-7.55 (m, 1H), 7.42-7.36 (m, 1H), 7.32-7.24 (m, 1H), 7.02-6.99 (m, 1H), 6.97-6.91 (m, 1H), 5.18-4.99 (m, 2H), 4.62-4.57 (m, 1H), 4.55-4.49 (m, 1H), 4.36-4.14 (m, 6H), 3.92-3.80 (m, 6H), 3.60-3.57 (m, 6H), 3.53-3.46 (m, 4H), 2.46-2.38 (m, 4H), 2.35-2.28 (m, 3H), 2.24-2.07 (m, 2H), 1.96-1.88 (m, 1H), 1.41-1.31 (m, 2H), 1.24-1.19 (m, 2H), 0.98-0.89 (m, 9H).

Example B29. 5-((R)-1-(2-amino-3-cyclopropylquinoline-6-carbonyl)-6′H-spiro[pyrrolidine-3,4′-thieno[2,3-c]furan]-2′-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide

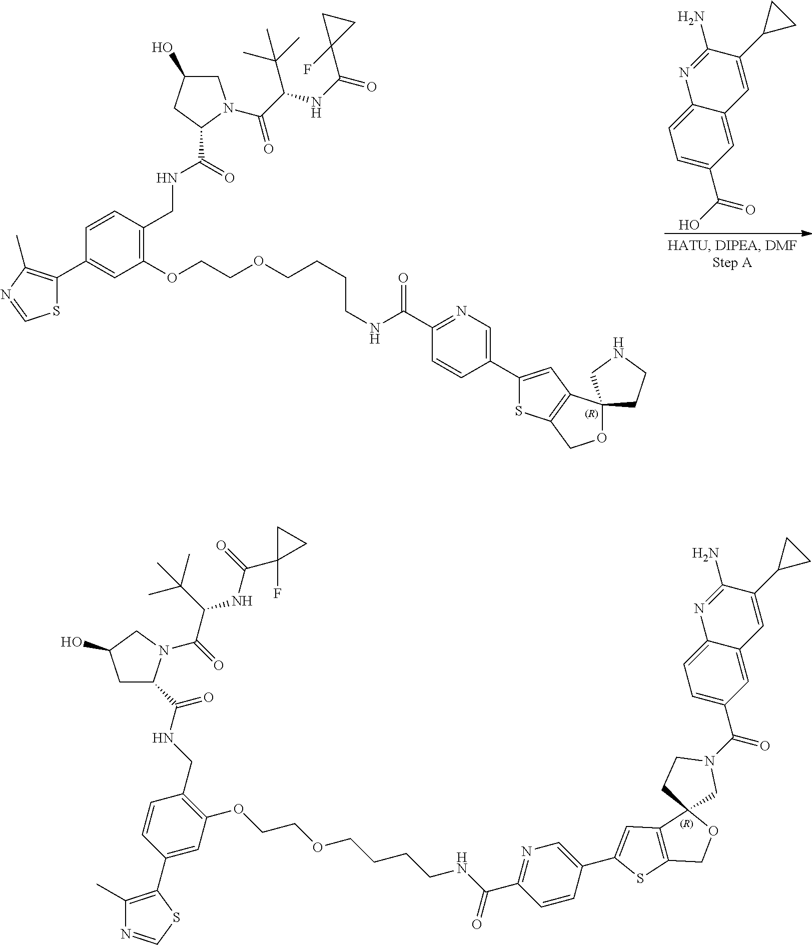

Example B29 was synthesized following the same procedures as described in Example B22 (1.8 mg, 3.5% yield) as a white solid. LC-MS (ESI): mass calcd, for C₆₀H₆₈FN₉O₉S₂, 1141.5; m/z found, 1142.3 [M+H]⁺. ¹H NMR (400 MHz, DMSO-d₆) δ 8.97 (s, 1H), 8.85 (d, J=11.2 Hz, 1H), 8.76 (s, 1H), 8.53-8.44 (m, 1H), 8.16-8.12 (m, 1H), 8.07-8.03 (m, 1H), 8.00-7.77 (m, 2H), 7.73-7.63 (m, 2H), 7.57-7.48 (m, 1H), 7.40 (d, J=7.9 Hz, 1H), 7.28 (d, J=9.0 Hz, 1H), 7.04 (s, 1H), 6.96 (d, J=7.7 Hz, 1H), 5.17 (d, J=3.3 Hz, 1H), 5.14-4.95 (m, 2H), 4.59 (d, J=9.4 Hz, 1H), 4.51 (t, J=8.1 Hz, 1H), 4.35 (s, 1H), 4.26 (dd, J=23.6, 5.8 Hz, 2H), 4.20-4.17 (m, 2H), 4.00-3.83 (m, 2H), 3.80-3.69 (m, 4H), 3.67-3.57 (m, 3H), 3.54-3.48 (m, 3H), 2.45 (s, 3H), 2.21-2.15 (m, 1H), 2.14-2.08 (m, 1H), 2.07-2.04 (m, 1H), 1.94-1.89 (m, 1H), 1.87-1.81 (m, 1H), 1.61-1.54 (m, 4H), 1.39-1.32 (m, 2H), 1.23-1.20 (m, 2H), 1.03-0.97 (m, 2H), 0.94 (s, 9H), 0.73-0.64 (m, 2H).

Example B30. 5-((S)-1′-(4-amino-1,3-dimethyl-1H-pyrazolo[4,3-c]quinoline-8-carbonyl)-3H-spiro[isobenzofuran-1,3′-pyrrolidin]-5-yl)-N-(4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)butyl)picolinamide

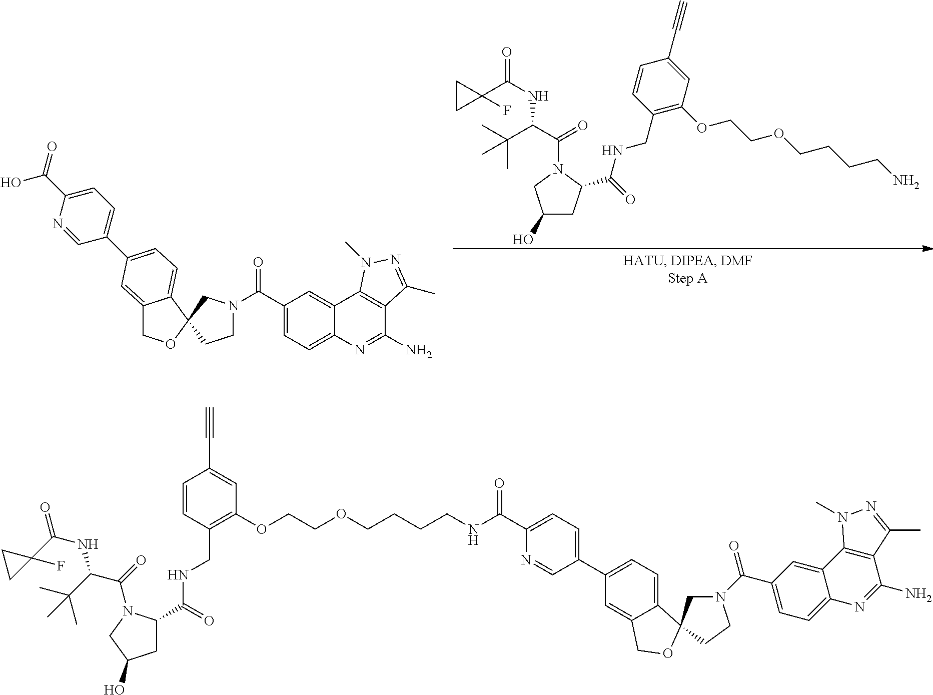 [SMILES: Cc1nn(C)c2c1c(N)nc1ccc(C(=O)N3CC[C@]4(C3)OCc3cc(-c5ccc(C(=O)O)nc5)ccc34)cc12 ; C#Cc1ccc(CNC(=O)[C@@H]2C[C@@H](O)CN2C(=O)[C@@H](NC(=O)C2(F)CC2)C(C)(C)C)c(OCCOCCCCN)c1 ; C#Cc1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)CO[C@@]43CCN(C(=O)c4ccc5nc(N)c6c(C)nn(C)c6c5c4)C3)cn2)c1]

Example B30 was synthesized following the same procedures as described in Example B19 (3.0 mg, 9% yield) as a white solid. LC-MS (ESI): mass calcd, for C₆₀H₆₇FN₁₀O₉, 1090.5/z found, 1091.5 [M+H]⁺. ¹H NMR (400 MHz, DMSO-d₆) δ 8.89 (d, J=21.7 Hz, 1H), 8.84-8.75 (m, 1H), 8.68-8.56 (m, 1H), 8.51-8.41 (m, 2H), 8.24 (dd, 1H), 8.09 (t, 1H), 7.98 (dd, 1H), 7.90-7.67 (m, 3H), 7.61 (dd, 1H), 7.36-7.20 (m, 2H), 7.04 (s, 1H), 6.96 (d, J=7.7 Hz, 1H), 5.23-5.11 (m, 1H), 5.10-4.98 (m, 1H), 4.63-4.53 (m, 1H), 4.52-4.45 (m, 1H), 4.41 (d, J=18.6 Hz, 3H), 4.36-4.30 (m, 1H), 4.29-4.21 (m, 1H), 4.20-4.09 (m, 4H), 3.97-3.85 (m, 2H), 3.84-3.78 (m, 1H), 3.75-3.70 (m, 2H), 3.66-3.55 (m, 3H), 3.55-3.47 (m, 4H), 2.66 (d, J=13.0 Hz, 3H), 2.45-2.38 (m, 1H), 2.27-2.17 (m, 1H), 2.10-2.03 (m, 1H), 2.03-1.95 (m, 1H), 1.92-1.83 (m, 1H), 1.63-1.52 (m, 4H), 1.39-1.31 (m, 2H), 1.22-1.17 (m, 2H), 0.93 (d, J=4.1 Hz, 9H).

Example B31. 5-((S)-1′-(2-amino-3-methylquinoline-6-carbonyl)-3H-spiro[isobenzofuran-1,3′-pyrrolidin]-5-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide

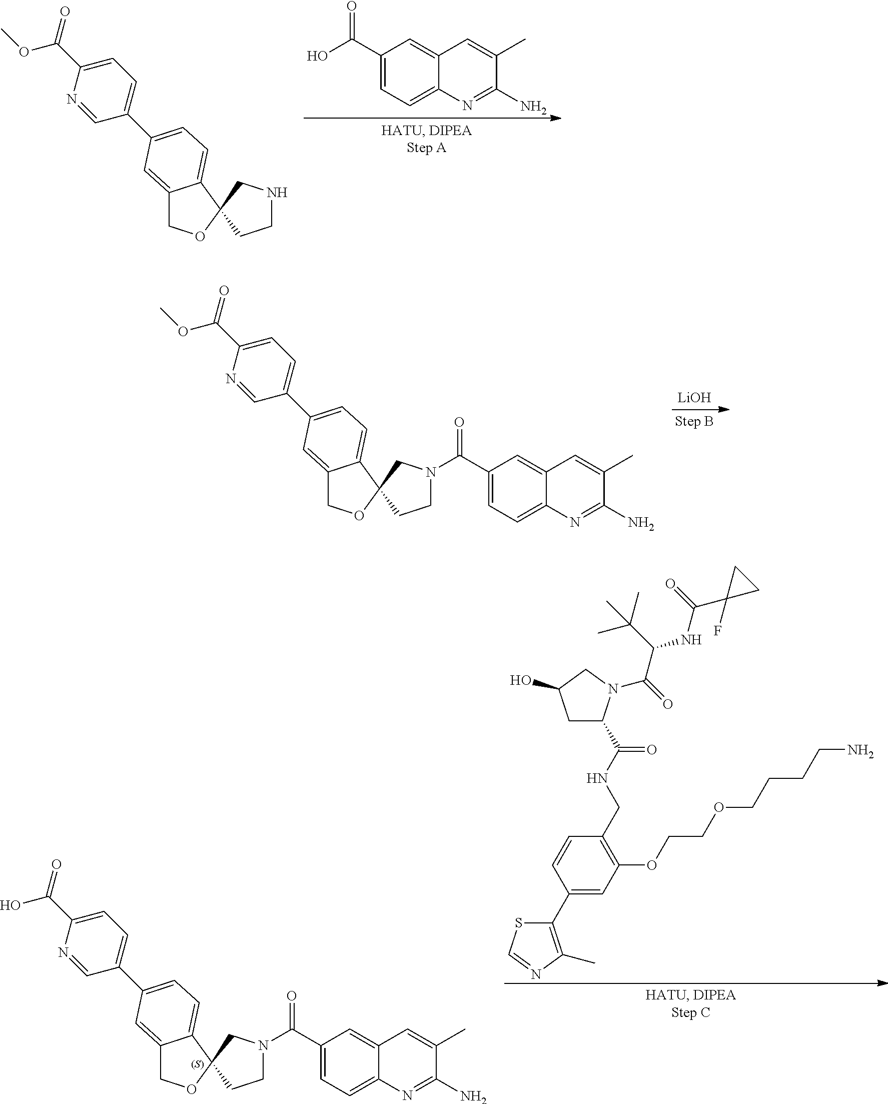

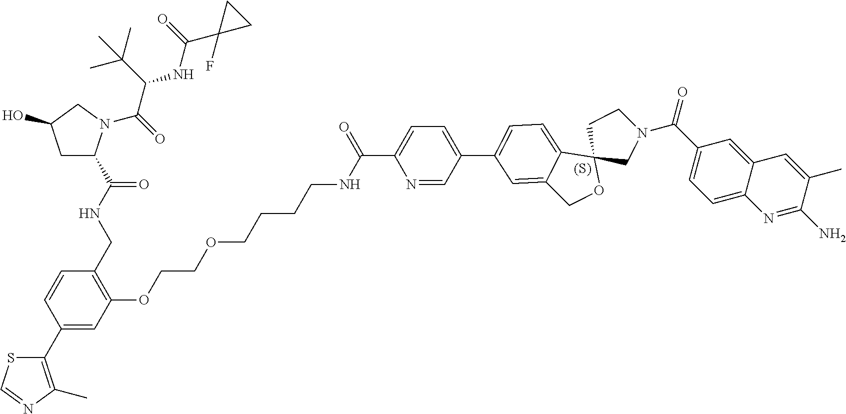

Example B31 was synthesized following the same procedures as described in Example B13 (6.2 mg, 16% yield over 3 steps) as a white solid. LC-MS (ESI): mass calcd. for C₆₀H₆₈FN₉O₉S, 1109.5 m/z found, 1110.5 [M+H]⁺. ¹H NMR (400 MHz, DMSO-d₆) δ 8.97 (d, J=2.4 Hz, 1H), 8.92-8.86 (m, 1H), 8.83-8.77 (m, 1H), 8.72-8.60 (m, 2H), 8.51-8.45 (m, 1H), 8.36-8.29 (m, 1H), 8.28-8.20 (m, 1H), 8.17-8.07 (m, 2H), 7.95-7.90 (m, 1H), 7.78-7.69 (m, 3H), 7.65-7.56 (m, 1H), 7.42-7.37 (m, 1H), 7.31-7.25 (m, 1H), 7.04 (s, 1H), 6.97-6.93 (m, 1H), 5.18-5.01 (m, 2H), 4.61-4.56 (m, 1H), 4.54-4.48 (m, 1H), 4.35-4.32 (m, 1H), 4.30-4.17 (m, 4H), 4.05-3.83 (m, 3H), 3.77-3.74 (m, 2H), 3.64-3.50 (m, 9H), 2.45-2.44 (m, 3H), 2.32 (d, J=16.8 Hz, 3H), 2.23-2.14 (m, 1H), 2.10-2.04 (m, 1H), 1.94-1.88 (m, 1H), 1.62-1.54 (m, 4H), 1.40-1.31 (m, 2H), 1.24-1.19 (m, 2H), 0.96-0.92 (m, 9H).

Example B32. 5-((R)-1′-(2-amino-3-methylquinoline-6-carbonyl)-3H-spiro[isobenzofuran-1,3′-pyrrolidin]-4-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide

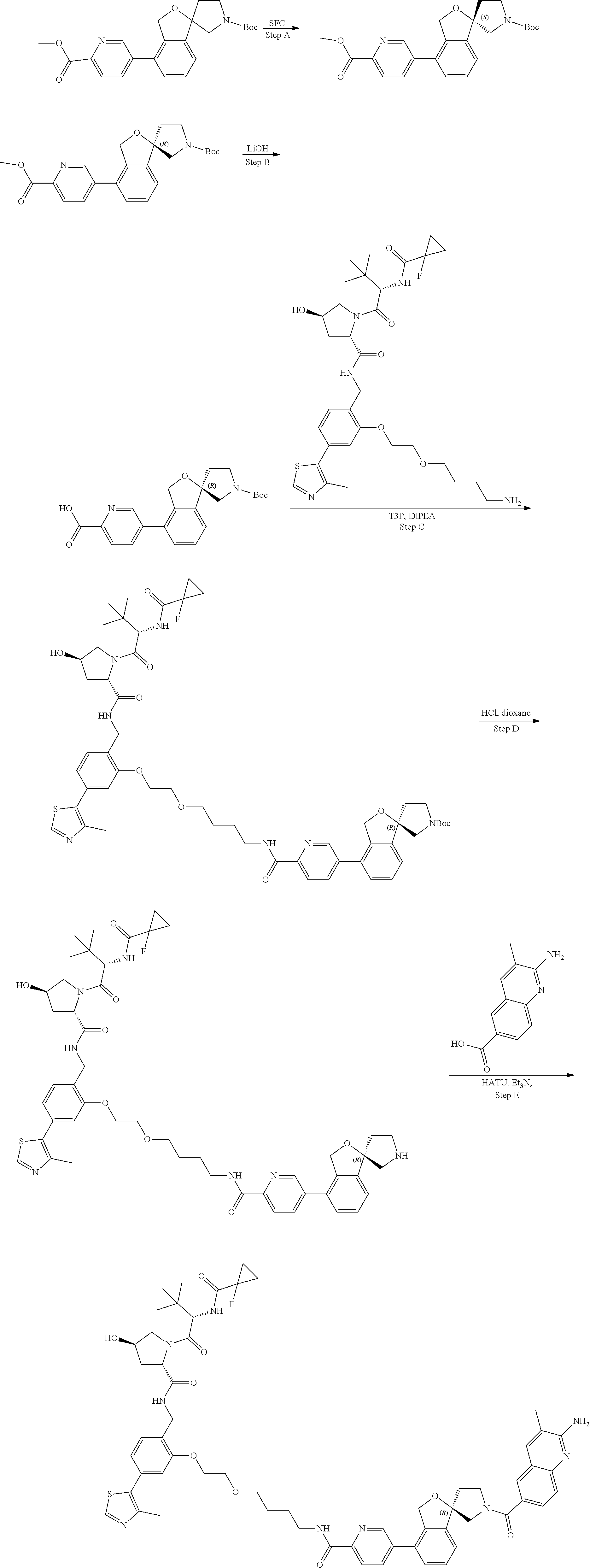

Step A: tert-butyl (R)-4-(6-(methoxycarbonyl)pyridin-3-yl)-3H-spiro[isobenzofuran-1,3′-pyrrolidine]-1′-carboxylate

The tert-butyl (R)-4-(6-(methoxycarbonyl)pyridin-3-yl)-3H-spiro[isobenzofuran-1,3′-pyrrolidine]-1′-carboxylate (85 mg) was separated by SFC [Instrument: SHIMADZU PREP SOLUTION SFC; Column: (R,R)-WHELK, 250×20 mm I.D., 5 μm; Column temperature: 35° C.; Mobile phase: A for CO₂ and B for MeOH; Gradient: B 50%; Flow rate: 40 mL/min] to give the title compound as a yellow solid from racemic tert-butyl 4-(6-(methoxycarbonyl)pyridin-3-yl)-3H-spiro[isobenzofuran-1,3′-pyrrolidine]-1′-carboxylate (180 mg).

LC-MS (ESI): mass calcd, for C₂₃H₂₆N₂O₅, 410.2; m/z found, 411.2 [M+H]⁺.

Step B: (R)-5-(1′-(tert-butoxycarbonyl)-3H-spiro[isobenzofuran-1,3′-pyrrolidin]-4-yl)picolinic acid

To mixture of tert-butyl (R)-4-(6-(methoxycarbonyl)pyridin-3-yl)-3H-spiro[isobenzofuran-1,3′-pyrrolidine]-1′-carboxylate (80.0 mg, 159 μmol, 1.0 eq.) in THF (2.0 mL) was added H₂O (0.4 mL) and LiOH H₂O (14 mg, 585 μmol, 3.0 eq.) at room temperature. Then the mixture was stirred at room temperature for 2 hours. The mixture was concentrated under reduced pressure to dryness to give the (R)-5-(1′-(tert-butoxycarbonyl)-3H-spiro[isobenzofuran-1,3′-pyrrolidin]-4-yl)picolinic acid (lithium salt, 75 mg, 97% yield) as a white solid.

LC-MS (ESI): mass calcd, for C₂₂H₂₄N₂O₅, 396.2; m/z found, 397.0 [M+H]⁺.

Step C: tert-butyl (R)-4-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-3H-spiro[isobenzofuran-1,3′-pyrrolidine]-1′-carboxylate

To a mixture of (2S,4R)—N-(2-(2-(4-aminobutoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (50 mg, 77.2 μmol, 1.0 eq.), (R)-5-(1′-(tert-butoxycarbonyl)-3H-spiro[isobenzofuran-1,3′-pyrrolidin]-4-yl)picolinic acid (30.6 mg, 77.2 μmol, 1.0 eq.) and DIPEA (23.4 mg, 232 μmol, 3.0 eq.) in DMF (1 mL) was added T3P (73.7 mg, 68.2 μL, 116 μmol, 1.5 eq., 50% in DMF) at room temperature. Then the mixture was stirred for 1 hour. The mixture was filtered and purified by flash chromatographer (eluent, 0~3% MeOH in DCM) to give the tert-butyl (R)-4-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-3H-spiro[isobenzofuran-1,3′-pyrrolidine]-1′-carboxylate (50 mg, 63% yield) as a white solid.

LC-MS (ESI): mass calcd, for C₅₄H₆₈FN₇O₁₀S, 1025.5; m/z found, 1026.4 [M+H]⁺.

Step D: N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)-5-((R)-3H-spiro[isobenzofuran-1,3′-pyrrolidin]-4-yl)picolinamide

To a solution of tert-butyl (R)-4-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-3H-spiro[isobenzofuran-1,3′-pyrrolidine]-1′-carboxylate (70 mg, 58.5 μmol, 1.0 eq.) in dioxane (2 mL) was added HCl in dioxane (2 mL, 4 mol/L) at 0° C. The reaction mixture was stirred for 1 hour at room temperature under N₂ atmosphere. The mixture was concentrated by vacuum to give N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)-5-((R)-3H-spiro[isobenzofuran-1,3′-pyrrolidin]-4-yl)picolinamide (HCl salt, 50 mg, 92% yield) as a colorless oil.

LC-MS (ESI): mass calcd, for C₄₉H₆₀FN₇O₈S, 925.4; m/z found, 926.4 [M+H]⁺.

Step E: 5-((R)-1′-(2-amino-3-methylquinoline-6-carbonyl)-3H-spiro[isobenzofuran-1,3′-pyrrolidin]-4-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide

To a solution of N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)-5-((R)-3H-spiro[isobenzofuran-1,3′-pyrrolidin]-4-yl)picolinamide (20 mg, 21.6 μmol, 1.0 eq.), 2-amino-3-methylquinoline-6-carboxylic acid (4.4 mg, 0.04 mmol, 1.0 eq.), HATU (30 mg, 0.08 mmol, 2.0 eq.) in DMF (5 mL) was added Et₃N (6.6 mg, 64.8 μmol, 3.0 eq.) at 0° C. The reaction mixture was stirred at 0° C. for 30 mins under N₂ atmosphere. The mixture was filtered and purified by prep-HPLC (C18, 20~95%, MeCN in H₂O with 0.1% HCOOH) to give 5-((R)-1′-(2-amino-3-methylquinoline-6-carbonyl)-3H-spiro[isobenzofuran-1,3′-pyrrolidin]-4-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide (5 mg, 18% yield) as a white solid.

LC-MS (ESI): mass calcd, for C₆₀H₆₈FN₉O₉S, 1109.5; m/z found, 1110.6 [M+H]⁺. ¹H NMR (400 MHz, DMSO-d₆) δ 8.99-8.95 (m, 1H), 8.87-8.67 (m, 4H), 8.51-8.44 (m, 1H), 8.33 (d, J=20.7 Hz, 1H), 8.18-8.09 (m, 2H), 8.07-8.03 (m, 1H), 7.95-7.90 (m, 1H), 7.77-7.69 (m, 1H), 7.60-7.52 (m, 3H), 7.42-7.38 (m, 1H), 7.31-7.25 (m, 1H), 7.06-7.03 (m, 1H), 6.97-6.93 (m, 1H), 5.31-5.19 (m, 1H), 5.12 (s, 1H), 4.61-4.56 (m, 1H), 4.54-4.48 (m, 1H), 4.35-4.18 (m, 5H), 4.04-3.95 (m, 1H), 3.90-3.85 (m, 2H), 3.79-3.72 (m, 4H), 3.53-3.50 (m, 4H), 3.35-3.30 (m, 2H), 2.46-2.44 (m, 3H), 2.40-2.17 (m, 5H), 2.11-2.04 (m, 1H), 1.95-1.87 (m, 1H), 1.62-1.53 (m, 4H), 1.40-1.31 (m, 2H), 1.24-1.19 (m, 2H), 0.96-0.92 (m, 9H).

Example B33. 5-((S)-1′-(2-amino-3-methylquinoline-6-carbonyl)-3H-spiro[isobenzofuran-1,3′-pyrrolidin]-4-yl)-N-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)picolinamide

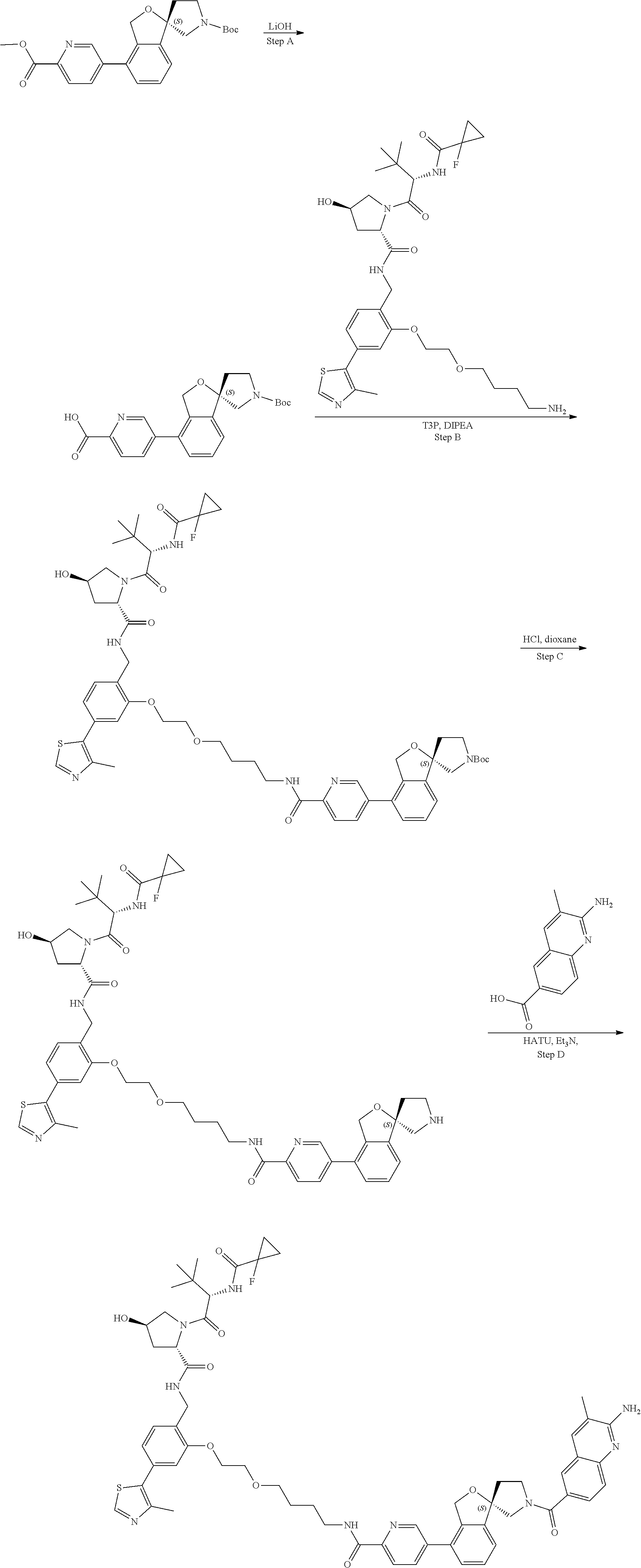

Example B33 was synthesized following the same procedures as described in Example B32 (5 mg, 6% yield over 4 steps) as a white solid. LC-MS (ESI): mass calcd, for C₆₀H₆₈FN₉O₉S, 1109.5; m/z found, 1110.6 [M+H]⁺. ¹H NMR (400 MHz, DMSO-d₆) δ 8.96 (d, J=4.5 Hz, 1H), 8.89-8.74 (m, 2H), 8.72-8.64 (m, 2H), 8.52-8.45 (m, 1H), 8.32 (d, J=20.7 Hz, 1H), 8.18-8.09 (m, 2H), 8.07-8.02 (m, 1H), 7.95-7.89 (m, 1H), 7.78-7.69 (m, 1H), 7.60-7.51 (m, 3H), 7.43-7.36 (m, 1H), 7.32-7.23 (m, 1H), 7.04 (d, J=3.9 Hz, 1H), 7.00-6.91 (m, 1H), 5.30-5.18 (m, 1H), 5.12 (s, 1H), 4.61-4.56 (m, 1H), 4.53-4.48 (m, 1H), 4.33-4.18 (m, 5H), 4.01-3.97 (m, 1H), 3.89-3.85 (m, 2H), 3.78-3.73 (m, 4H), 3.67-3.64 (m, 4H), 3.33-3.29 (m, 2H), 2.44 (d, J=4.7 Hz, 3H), 2.38-2.19 (m, 5H), 2.11-2.04 (m, 1H), 1.95-1.87 (m, 1H), 1.63-1.54 (m, 4H), 1.39-1.31 (m, 2H), 1.23-1.19 (m, 2H), 0.94 (d, J=4.1 Hz, 9H).

Example B34. 5-((R)-1-(2-amino-3-cyclopropylquinoline-6-carbonyl)-6′H-spiro[pyrrolidine-3,4′-thieno[2,3-c]furan]-2′-yl)-N-(4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)butyl)picolinamide

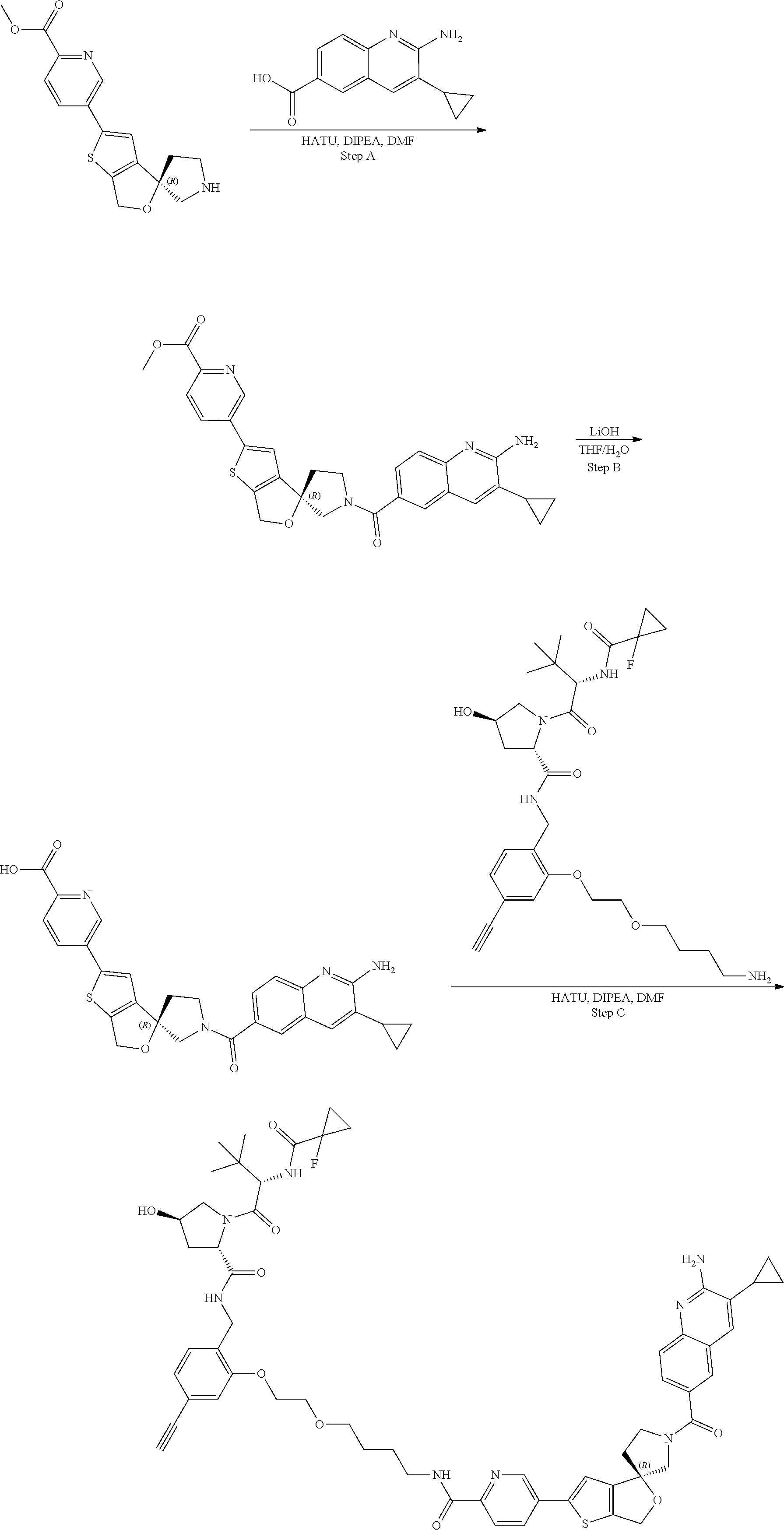

Example B34 was synthesized following the same procedures as described in Example B5 (2.2 mg, 3.3% yield over 3 steps) as a white solid. LC-MS (ESI): mass calcd. for C₅₈H₆₅FN₈O9s, 1068.5; m/z found, 1069.3 [M+H]⁺. ¹H NMR (400 MHz, DMSO-d₆) δ 8.86 (d, J=11.9 Hz, 1H), 8.81-8.71 (m, 1H), 8.47 (t, J=5.9 Hz, 1H), 8.20-8.12 (m, 1H), 8.09-8.01 (m, 1H), 7.95-7.82 (m, 1H), 7.78-7.59 (m, 3H), 7.52-7.42 (m, 1H), 7.33-7.25 (m, 2H), 7.05 (s, 1H), 6.99-6.94 (m, 1H), 6.83-6.47 (m, 2H), 5.19-5.16 (m, 1H), 5.16-4.95 (m, 2H), 4.62-4.56 (m, 1H), 4.52-4.45 (m, 1H), 4.36-4.31 (m, 1H), 4.30-4.17 (m, 2H), 4.16-4.11 (m, 3H), 4.00-3.82 (m, 2H), 3.78-3.69 (m, 4H), 3.66-3.55 (m, 4H), 3.52-3.50 (m, 2H), 2.47-2.35 (m, 1H), 2.23-2.13 (m, 1H), 2.09-2.02 (m, 1H), 1.92-1.78 (m, 2H), 1.62-1.53 (m, 4H), 1.40-1.32 (m, 2H), 1.24-1.20 (m, 2H), 0.99 (s, 2H), 0.96-0.93 (m, 9H), 0.70-0.58 (m, 2H).

Example B35. 5-((R)-1-(2-amino-3-methylquinoline-6-carbonyl)-6′H-spiro[pyrrolidine-3,4′-thieno[2,3-c]furan]-2′-yl)-N-(4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)butyl)picolinamide

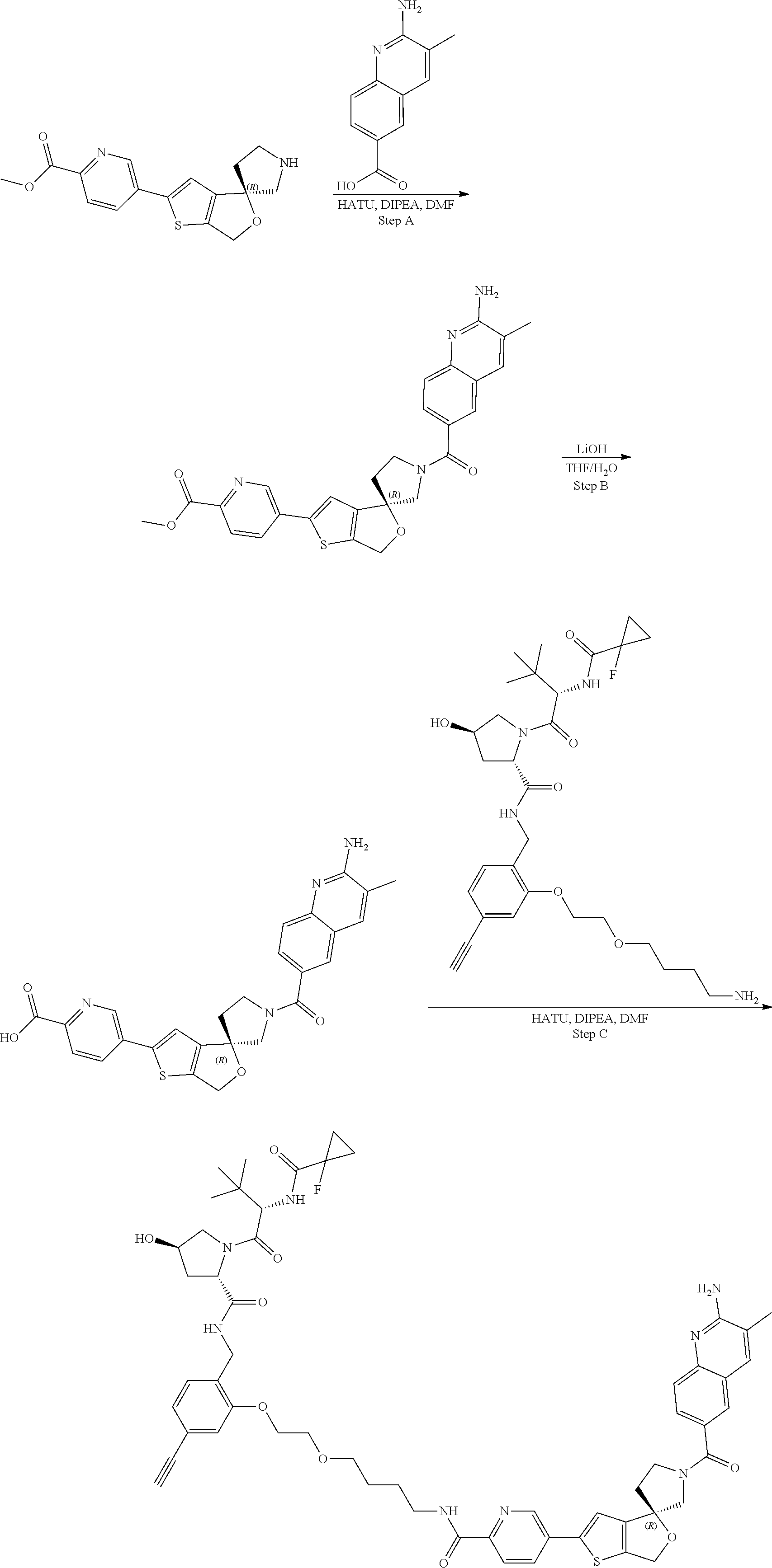

Example B35 was synthesized following the same procedures as described in Example B5 (11 mg, 15% yield over 3 steps) as a white solid. LC-MS (ESI): mass calcd, for C₅₆H₆₃FN₈O₉S, 1042.4; m/z found, 1043.0 [M+H]⁺. ¹H NMR (400 MHz, DMSO-d₆) δ 8.92-8.84 (m, 1H), 8.82-8.73 (m, 1H), 8.53-8.42 (m, 1H), 8.19-8.11 (m, 1H), 8.09-8.01 (m, 1H), 7.91-7.78 (m, 2H), 7.73-7.57 (m, 2H), 7.51-7.42 (m, 1H), 7.35-7.26 (m, 2H), 7.07-7.03 (m, 1H), 6.99-6.95 (m, 1H), 6.54-6.44 (m, 2H), 6.44-6.40 (m, 1H), 5.20-5.16 (m, 1H), 5.16-4.97 (m, 2H), 4.62-4.55 (m, 1H), 4.52-4.45 (m, 1H), 4.37-4.31 (m, 1H), 4.31-4.18 (m, 2H), 4.16-4.11 (m, 3H), 4.02-3.82 (m, 2H), 3.79-3.68 (m, 4H), 3.67-3.55 (m, 4H), 3.53-3.50 (m, 2H), 2.41-2.30 (m, 1H), 2.24-2.14 (m, 4H), 2.10-2.04 (m, 1H), 1.94-1.85 (m, 1H), 1.63-1.54 (m, 4H), 1.41-1.31 (m, 2H), 1.24-1.18 (m, 2H), 0.94 (s, 9H).

Example B36. 5-((R)-1-(2-((6-amino-5-methylpyridin-3-yl)amino)-2-oxoacetyl)-6′H-spiro[pyrrolidine-3,4′-thieno[2,3-c]furan]-2′-yl)-N-((1S,4R)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)picolinamide

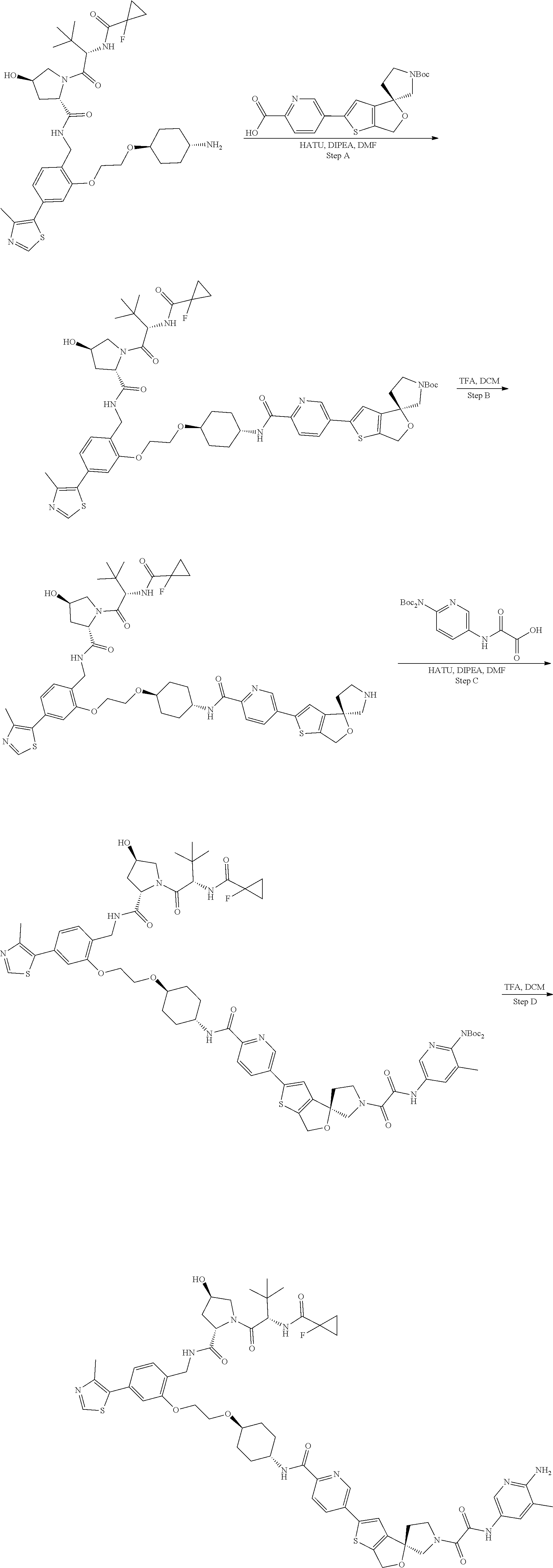

Example B36 was synthesized following the same procedures as described in Examples B11 and A31 (1.9 mg, 1% yield over 4 steps) as a white solid. LC-MS (ESI): mass calcd. for C₅₇H₆₇FN₁₀O₁₀S₂, 1134.45; m/z found, 1135.6 [M+H]⁺. ¹H NMR (400 MHz, DMSO-d₆) δ 10.40 (s, 1H), 8.99 (s, 1H), 8.87 (s, 1H), 8.52-8.43 (m, 2H), 8.18-8.10 (m, 2H), 8.08-8.04 (m, 1H), 7.69 (s, 1H), 7.62-7.56 (m, 1H), 7.41 (d, J=7.8 Hz, 1H), 7.29 (d, J=9.5 Hz, 1H), 7.05 (s, 1H), 6.97 (d, J=7.8 Hz, 1H), 5.68 (s, 2H), 5.19-5.15 (m, 1H), 5.11-5.05 (m, 2H), 4.62-4.57 (m, 1H), 4.54-4.49 (m, 1H), 4.38-4.34 (m, 1H), 4.32-4.22 (m, 2H), 4.19-4.16 (m, 2H), 4.04-3.89 (m, 2H), 3.81-3.79 (m, 2H), 3.63-3.60 (m, 2H), 3.40-3.39 (m, 2H), 3.31-3.31 (m, 2H), 2.47 (s, 3H), 2.24-2.18 (m, 1H), 2.13-2.08 (m, 1H), 2.07-2.01 (m, 6H), 1.96-1.91 (m, 1H), 1.87-1.82 (m, 2H), 1.54-1.47 (m, 2H), 1.39-1.28 (m, 4H), 1.24-1.21 (m, 2H), 0.96 (s, 9H).

Example B37. 5-((R)-1-(2-((6-amino-5-ethylpyridin-3-yl)amino)-2-oxoacetyl)-6′H-spiro[pyrrolidine-3,4′-thieno[2,3-c]furan]-2′-yl)-N-((1S,4R)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)picolinamide

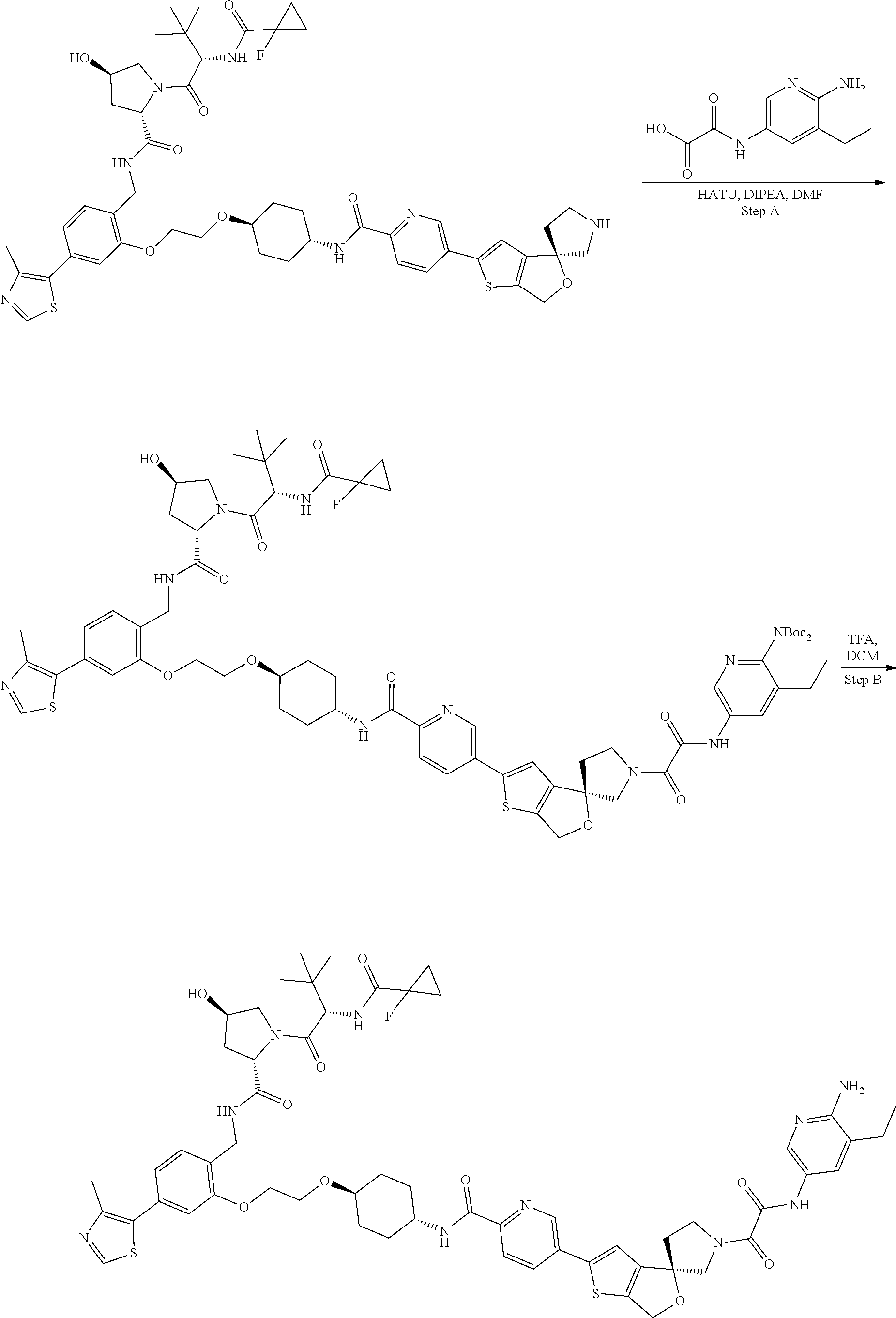

Example B37 was synthesized following the same procedures as described in Example B11 (1 mg, 4% yield over 2 steps) as a white solid. LC-MS (ESI): mass calcd, for C₅₈H₆₉FN₁₀O₁₀S₂, 1148.46; m/z found, 1149.6 [M+H]⁺. ¹H NMR (400 MHz, DMSO-d₆) δ 10.40 (d, J=3.3 Hz, 1H), 8.99 (s, 1H), 8.87 (s, 1H), 8.52-8.41 (m, 2H), 8.18-8.11 (m, 2H), 8.05 (d, J=8.2 Hz, 1H), 7.69 (s, 1H), 7.60 (d, J=8.7 Hz, 1H), 7.41 (d, J=7.8 Hz, 1H), 7.29 (d, J=6.9 Hz, 1H), 7.05 (s, 1H), 6.97 (d, J=7.6 Hz, 1H), 5.68-5.58 (m, 2H), 5.20-5.15 (m, 1H), 5.12-5.05 (m, 2H), 4.60 (d, J=8.9 Hz, 1H), 4.55-4.48 (m, 1H), 4.36 (s, 1H), 4.33-4.21 (m, 2H), 4.19-4.16 (m, 2H), 4.06-3.88 (m, 2H), 3.82-3.80 (m, 2H), 3.67-3.61 (m, 2H), 3.31-3.31 (m, 2H), 2.47 (s, 3H), 2.43-2.37 (m, 3H), 2.16-2.10 (m, 1H), 2.09-2.01 (m, 3H), 1.97-1.91 (m, 1H), 1.88-1.82 (m, 2H), 1.54-1.47 (m, 2H), 1.39-1.29 (m, 4H), 1.24-1.22 (m, 2H), 1.15-1.09 (m, 3H), 0.96 (s, 9H).

Example B38. N-((1S,4r)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-5-(1′-(quinoline-6-carbonyl)-3H-spiro[isobenzofuran-1,3′-pyrrolidin]-5-yl)picolinamide

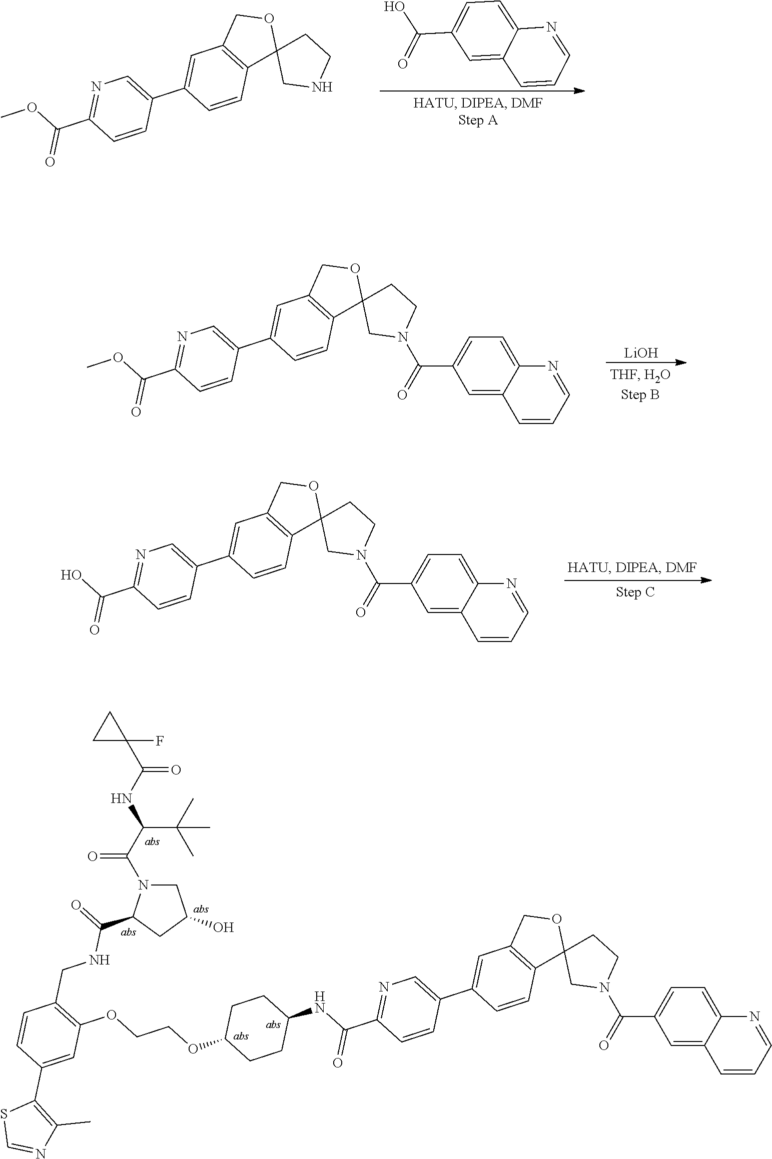 [SMILES: COC(=O)c1ccc(-c2ccc3c(c2)COC32CCNC2)cn1 ; O=C(O)c1ccc2ncccc2c1 ; COC(=O)c1ccc(-c2ccc3c(c2)COC32CCN(C(=O)c3ccc4ncccc4c3)C2)cn1 ; "LiOH" ; O=C(O)c1ccc(-c2ccc3c(c2)COC32CCN(C(=O)c3ccc4ncccc4c3)C2)cn1 ; Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)COC54CCN(C(=O)c5ccc6ncccc6c5)C4)cn3)CC2)c1]

Example B38 was synthesized following the same procedures as described in Example B4 (2.5 mg, 6% yield over 3 steps) as a white solid. LC-MS (ESI): mass calcd, for C₆₁H₆₇FN₈O₉S, 1106.47/z found, 1107.5 [M+H]⁺. ¹H NMR (400 MHz, DMSO-d₆) δ 9.01-8.86 (m, 3H), 8.55-8.42 (m, 3H), 8.33-8.26 (m, 1H), 8.25-8.22 (m, 1H), 8.14-8.04 (m, 2H), 7.95-7.90 (m, 1H), 7.79-7.68 (m, 2H), 7.67-7.55 (m, 2H), 7.41 (d, J=7.7 Hz, 1H), 7.30 (d, J=9.0 Hz, 1H), 7.05 (s, 1H), 6.97 (d, J=7.8 Hz, 1H), 5.23-5.08 (m, 2H), 5.07-4.99 (m, 1H), 4.60 (d, J=9.2 Hz, 1H), 4.56-4.48 (m, 1H), 4.39-4.29 (m, 2H), 4.26-4.14 (m, 3H), 3.95-3.85 (m, 2H), 3.84-3.74 (m, 4H), 3.70-3.56 (m, 3H), 2.47 (d, J=1.6 Hz, 3H), 2.07-2.01 (m, 2H), 1.97-1.82 (m, 4H), 1.56-1.47 (m, 2H), 1.41-1.29 (m, 4H), 1.27-1.17 (m, 4H), 0.95 (s, 9H).

Example B39. (2S,4R)—N-(2-(4-((4-(5-(1′-(4-amino-1,3-dihydrofuro[3,4-c][1,7]naphthyridine-8-carbonyl)-3H-spiro[isobenzofuran-1,3′-pyrrolidin]-5-yl)picolinoyl)piperazin-1-yl)methyl)piperidin-1-yl)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

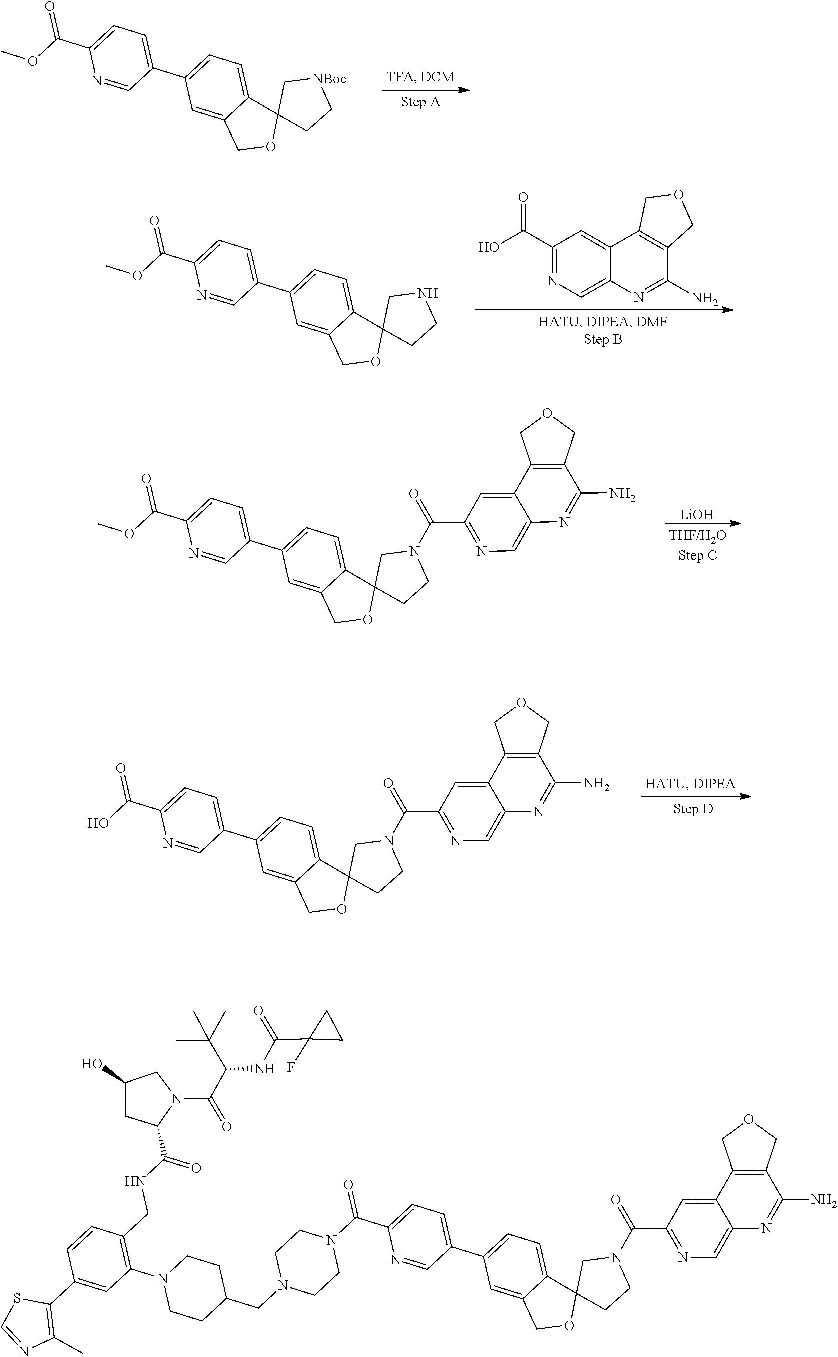

Example B39 was synthesized following the same procedures as described in Example B4 (1.6 mg, 1% yield over 4 steps) as a white solid. LC-MS (ESI): mass calcd, for C₆₄H₇₃FN₁₂O₈S, 1188.54; m/z found, 1189.6 [M+H]⁺. ¹H NMR (400 MHz, DMSO-d₆) δ 9.60 (s, 1H), 9.00-8.88 (m, 3H), 8.52 (s, 1H), 8.31-8.24 (m, 1H), 8.06-7.99 (m, 1H), 7.83-7.71 (m, 4H), 7.67-7.63 (m, 1H), 7.44 (d, J=8.3 Hz, 1H), 7.30-7.25 (m, 1H), 7.16-7.10 (m, 2H), 5.43 (s, 2H), 5.19-5.00 (m, 4H), 4.63-4.56 (m, 2H), 4.51-4.47 (m, 1H), 4.42-4.31 (m, 4H), 4.30-4.20 (m, 2H), 4.18-4.10 (m, 2H), 4.03-3.91 (m, 4H), 3.85-3.80 (m, 2H), 3.67-3.66 (m, 2H), 3.16-3.10 (m, 4H), 2.75-2.69 (m, 2H), 2.46 (s, 3H), 2.43-2.39 (m, 1H), 2.21-2.17 (m, 1H), 2.12-2.07 (m, 1H), 2.02-1.97 (m, 1H), 1.93-1.84 (m, 3H), 1.51-1.43 (m, 2H), 1.40-1.33 (m, 2H), 1.24-1.21 (m, 2H), 1.00-0.95 (m, 9H).

Example B40. (2S,4R)—N-(2-(4-((4-(5-(1′-(4-amino-1-methyl-1H-pyrazolo[4,3-c]quinoline-8-carbonyl)-3H-spiro[isobenzofuran-1,3′-pyrrolidin]-5-yl)picolinoyl)piperazin-1-yl)methyl)piperidin-1-yl)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

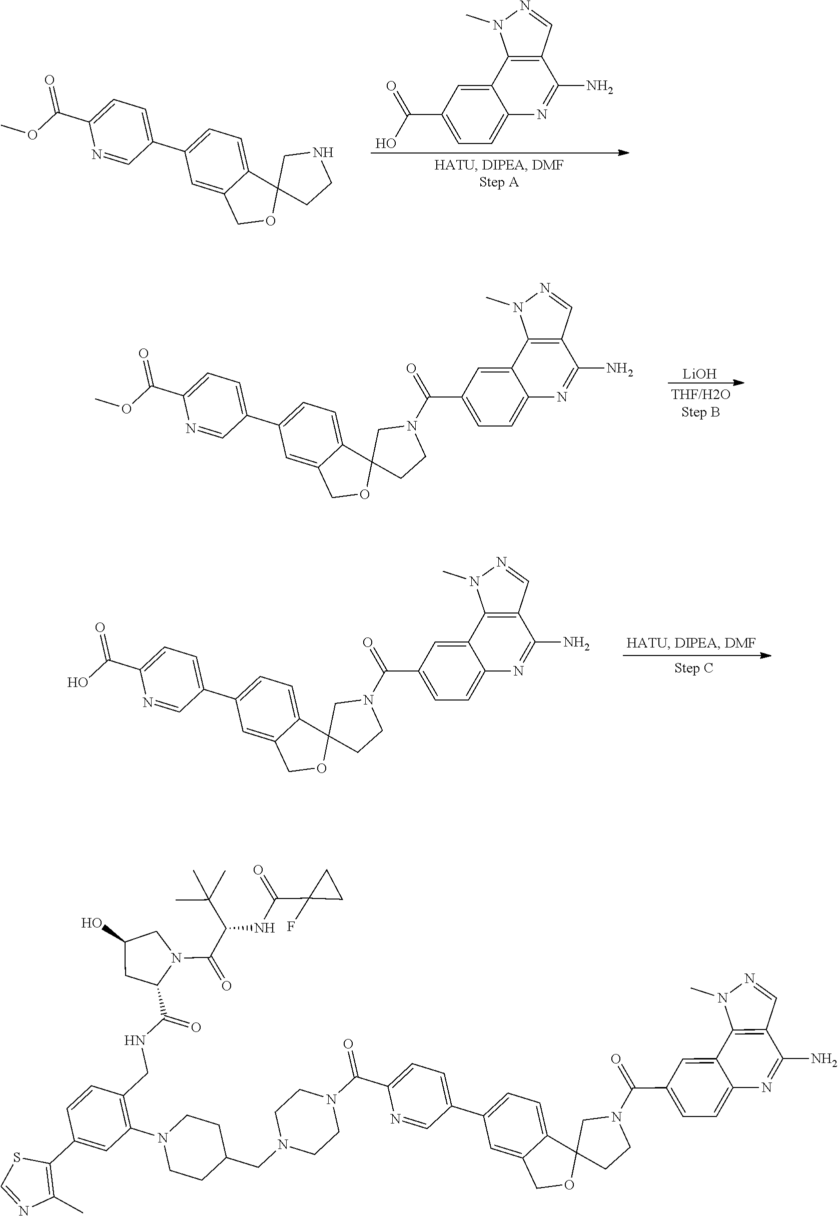 [SMILES: COC(=O)c1ccc(-c2ccc3c(c2)COC32CCNC2)cn1 ; Cn1ncc2c(N)nc3ccc(C(=O)O)cc3c21 ; COC(=O)c1ccc(-c2ccc3c(c2)COC32CCN(C(=O)c3ccc4nc(N)c5cnn(C)c5c4c3)C2)cn1 ; "LiOH" ; Cn1ncc2c(N)nc3ccc(C(=O)N4CCC5(C4)OCc4cc(-c6ccc(C(=O)O)nc6)ccc45)cc3c21 ; Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)COC65CCN(C(=O)c6ccc7nc(N)c8cnn(C)c8c7c6)C5)cn4)CC3)CC2)c1]

Example B40 was synthesized following the same procedures as described in Example B4 (2 mg, 5% yield over 3 steps) as a white solid. LC-MS (ESI): mass calcd, for C₆₅H₇₄FN₁₃O₇S, 1199.55; m/z found, 1200.6 [M+H]⁺. ¹H NMR (400 MHz, DMSO-d₆) δ 9.68 (s, 1H), 9.09 (s, 1H), 9.01-8.88 (m, 2H), 8.63 (d, J=15.9 Hz, 1H), 8.58-8.37 (m, 2H), 8.26 (dd, J=22.9, 8.2 Hz, 1H), 8.05-7.93 (m, 1H), 7.89-7.76 (m, 3H), 7.76-7.67 (m, 1H), 7.67-7.57 (m, 1H), 7.45 (d, J=8.1 Hz, 1H), 7.28 (d, J=8.6 Hz, 1H), 7.17-7.08 (m, 2H), 5.22-4.98 (m, 2H), 4.70-4.56 (m, 2H), 4.54-4.46 (m, 4H), 4.44-4.26 (m, 4H), 4.08-3.94 (m, 2H), 3.93-3.89 (m, 2H), 3.48-3.47 (m, 8H), 3.19-3.10 (m, 4H), 2.77-2.67 (m, 2H), 2.46 (s, 3H), 2.44-2.36 (m, 1H), 2.30-2.19 (m, 1H), 2.13-2.07 (m, 1H), 2.04-1.97 (m, 1H), 1.95-1.84 (m, 3H), 1.52-1.42 (m, 2H), 1.41-1.33 (m, 2H), 1.24-1.20 (m, 2H), 1.01-0.92 (m, 9H).

Example B41. (2S,4R)—N-(2-(4-((4-(5-(1′-(2-amino-3-cyclopropylquinoline-6-carbonyl)-3H-spiro[isobenzofuran-1,3′-pyrrolidin]-5-yl)picolinoyl)piperazin-1-yl)methyl)piperidin-1-yl)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

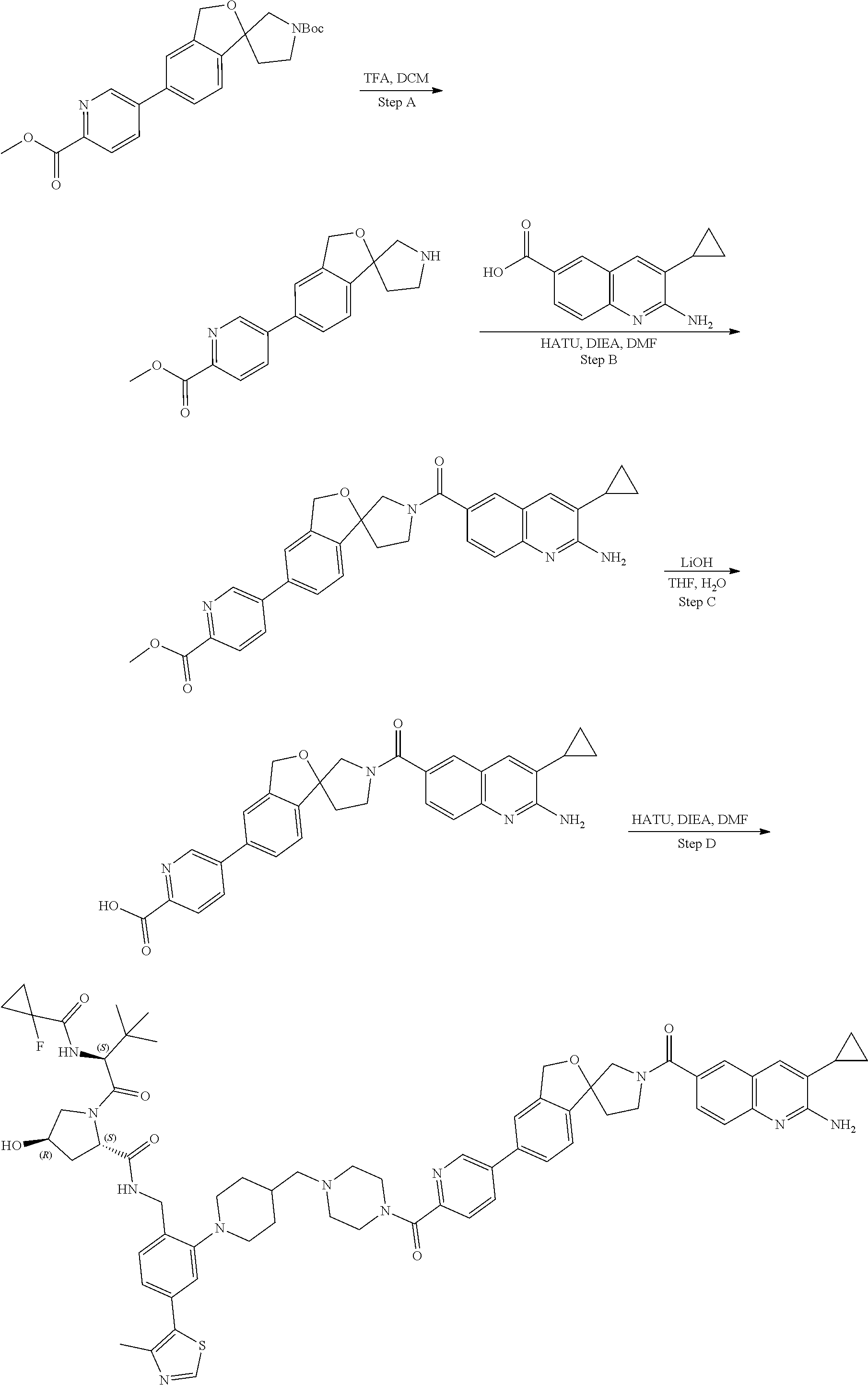

Example B41 was synthesized following the same procedures as described in Example B4 (3.5 mg, 4% yield over 4 steps) as a white solid. LC-MS (ESI): mass calcd, for C₆₆H₇₆FN₁₁O₇S, 1185.6/z found, 1186.6 [M+H]⁺. ¹H NMR (400 MHz, DMSO-d₆) δ 9.59 (s, 1H), 8.99 (s, 1H), 8.94-8.90 (m, 1H), 8.82 (s, 1H), 8.52 (s, 1H), 8.27 (t, J=12.6 Hz, 2H), 8.18-8.10 (m, 1H), 7.91 (s, 1H), 7.84-7.73 (m, 3H), 7.72-7.57 (m, 2H), 7.44 (d, J=8.2 Hz, 1H), 7.27 (d, J=6.9 Hz, 1H), 7.14 (s, 2H), 5.26-4.92 (m, 3H), 4.71-4.52 (m, 2H), 4.49 (t, J=8.2 Hz, 1H), 4.43-4.28 (m, 3H), 4.06-3.92 (m, 1H), 3.90-3.73 (m, 5H), 3.78-3.57 (m, 4H), 3.26-2.96 (m, 6H), 2.87-2.58 (m, 2H), 2.46 (s, 3H), 2.27-2.17 (m, 1H), 2.14-2.01 (m, 2H), 1.96-1.79 (m, 4H), 1.51-1.29 (m, 5H), 1.27-1.17 (m, 2H), 1.10-1.02 (m, 2H), 0.97 (s, 9H), 0.86-0.74 (m, 2H).

Example B42. 5-(1′-(2-amino-3-ethynylquinoline-6-carbonyl)-3H-spiro[isobenzofuran-1,3′-pyrrolidin]-5-yl)-N-((1S,4r)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)picolinamide

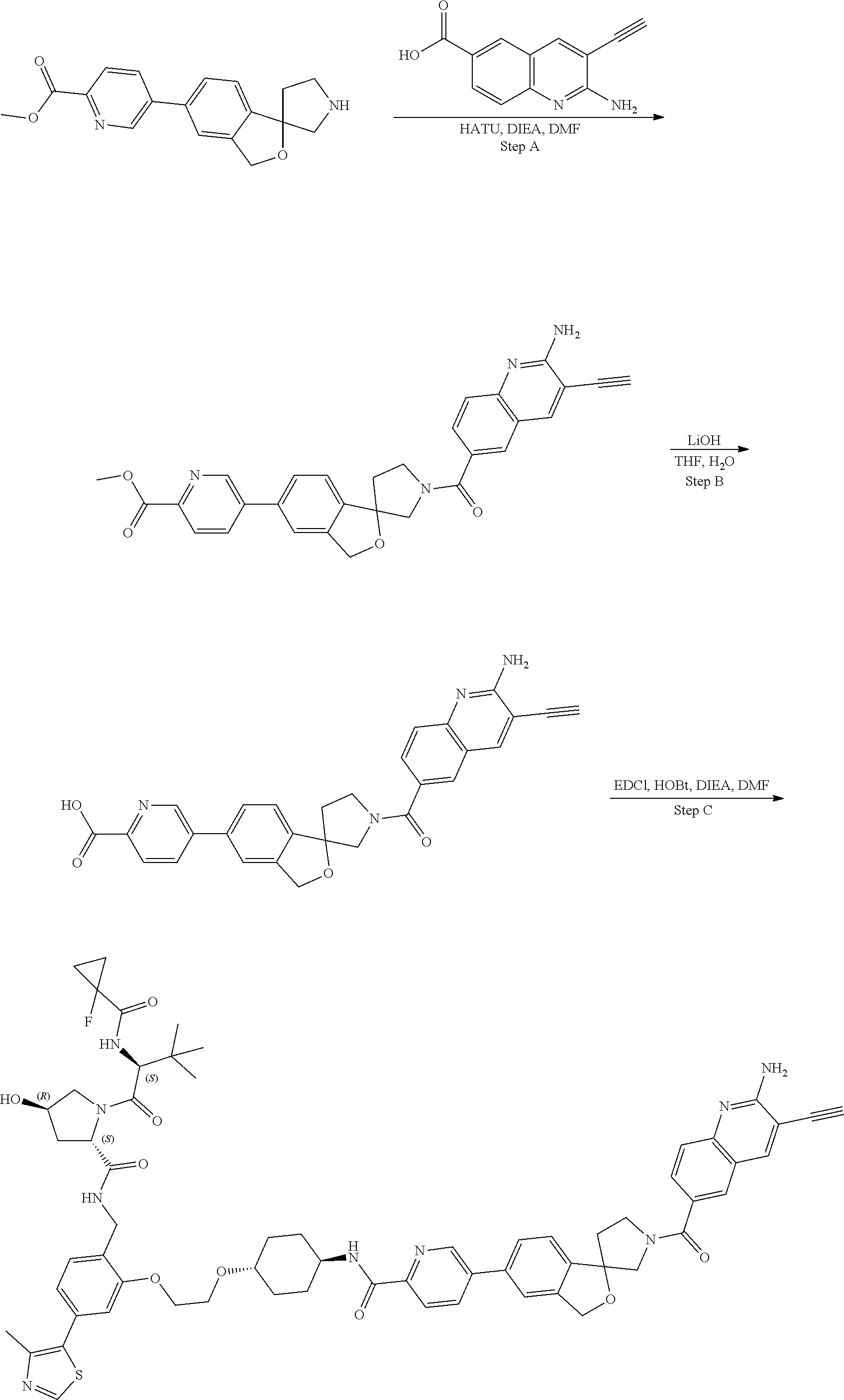

Example B42 was synthesized following the same procedures as described in Example B4 (2.3 mg, 9% yield over 3 steps) as a white solid. LC-MS (ESI): mass calcd, for C₆₃H₆₈FNO₉S, 1145.48/z found, 1146.7 [M+H]⁺. ¹H NMR (400 MHz, DMSO-d₆) δ 8.99 (s, 1H), 8.91 (d, J=19.9 Hz, 1H), 8.69-8.53 (m, 1H), 8.49 (s, 2H), 8.31-8.01 (m, 4H), 7.96-7.68 (m, 4H), 7.67-7.55 (m, 1H), 7.41 (d, J=7.8 Hz, 1H), 7.29 (d, J=9.3 Hz, 1H), 7.05 (s, 1H), 6.97 (d, J=7.7 Hz, 1H), 5.25-4.98 (m, 2H), 4.93-4.83 (m, 1H), 4.59 (d, J=9.3 Hz, 1H), 4.52 (t, J=8.5 Hz, 1H), 4.38-4.23 (m, 3H), 4.17 (s, 3H), 4.00 (d, J=11.5 Hz, 2H), 3.94-3.80 (m, 6H), 2.47 (s, 3H), 2.14-2.00 (m, 3H), 1.95-1.78 (m, 3H), 1.57-1.14 (m, 10H), 0.95 (s, 9H).

Example B43. 5-(1′-(2-amino-3-cyanoquinoline-6-carbonyl)-3H-spiro[isobenzofuran-1,3′-pyrrolidin]-5-yl)-N-((1S,4r)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)picolinamide

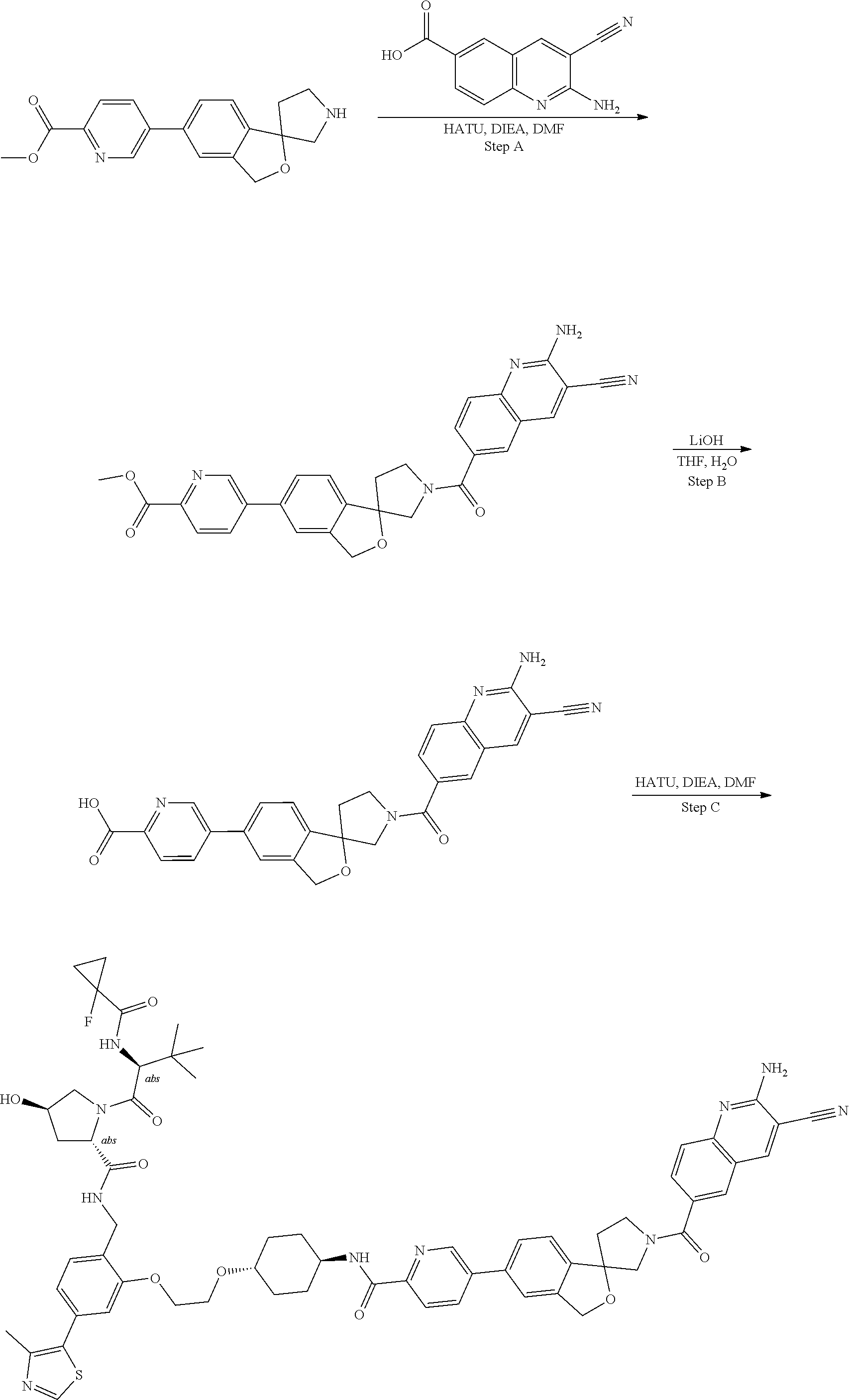 [SMILES: COC(=O)c1ccc(-c2ccc3c(c2)COC32CCNC2)cn1 ; N#Cc1cc2cc(C(=O)O)ccc2nc1N ; COC(=O)c1ccc(-c2ccc3c(c2)COC32CCN(C(=O)c3ccc4nc(N)c(C#N)cc4c3)C2)cn1 ; "LiOH" ; N#Cc1cc2cc(C(=O)N3CCC4(C3)OCc3cc(-c5ccc(C(=O)O)nc5)ccc34)ccc2nc1N ; Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)COC54CCN(C(=O)c5ccc6nc(N)c(C#N)cc6c5)C4)cn3)CC2)c1]

Example B43 was synthesized following the same procedures as described in Example B4 (1.0 mg, 4% yield over 3 steps) as a white solid. LC-MS (ESI): mass calcd, for C₆₂H₆₇FN₁₀O₉S, 1146.48/z found, 1147.6 [M+H]⁺. ¹H NMR (400 MHz, DMSO-d₆) δ 8.99 (s, 1H), 8.95-8.77 (m, 2H), 8.49 (s, 2H), 8.32-8.19 (m, 1H), 8.11-8.01 (m, 2H), 7.88 (s, 1H), 7.79-7.68 (m, 2H), 7.66-7.52 (m, 3H), 7.50-7.43 (m, 1H), 7.41 (d, J=7.5 Hz, 1H), 7.29 (d, J=8.0 Hz, 1H), 7.05 (s, 1H), 6.97 (d, J=7.6 Hz, 1H), 5.23-4.95 (m, 2H), 4.64-4.48 (m, 2H), 4.40-4.10 (m, 6H), 4.07-3.78 (m, 9H), 2.46 (s, 3H), 2.15-1.98 (m, 3H), 1.97-1.79 (m, 3H), 1.61-1.45 (m, 2H), 1.40-1.16 (m, 8H), 0.95 (s, 9H).

Example B44. 5-(1′-(2-amino-3-cyanoquinoline-6-carbonyl)-3H-spiro[isobenzofuran-1,3′-pyrrolidin]-5-yl)-N-((1S,4r)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)picolinamide

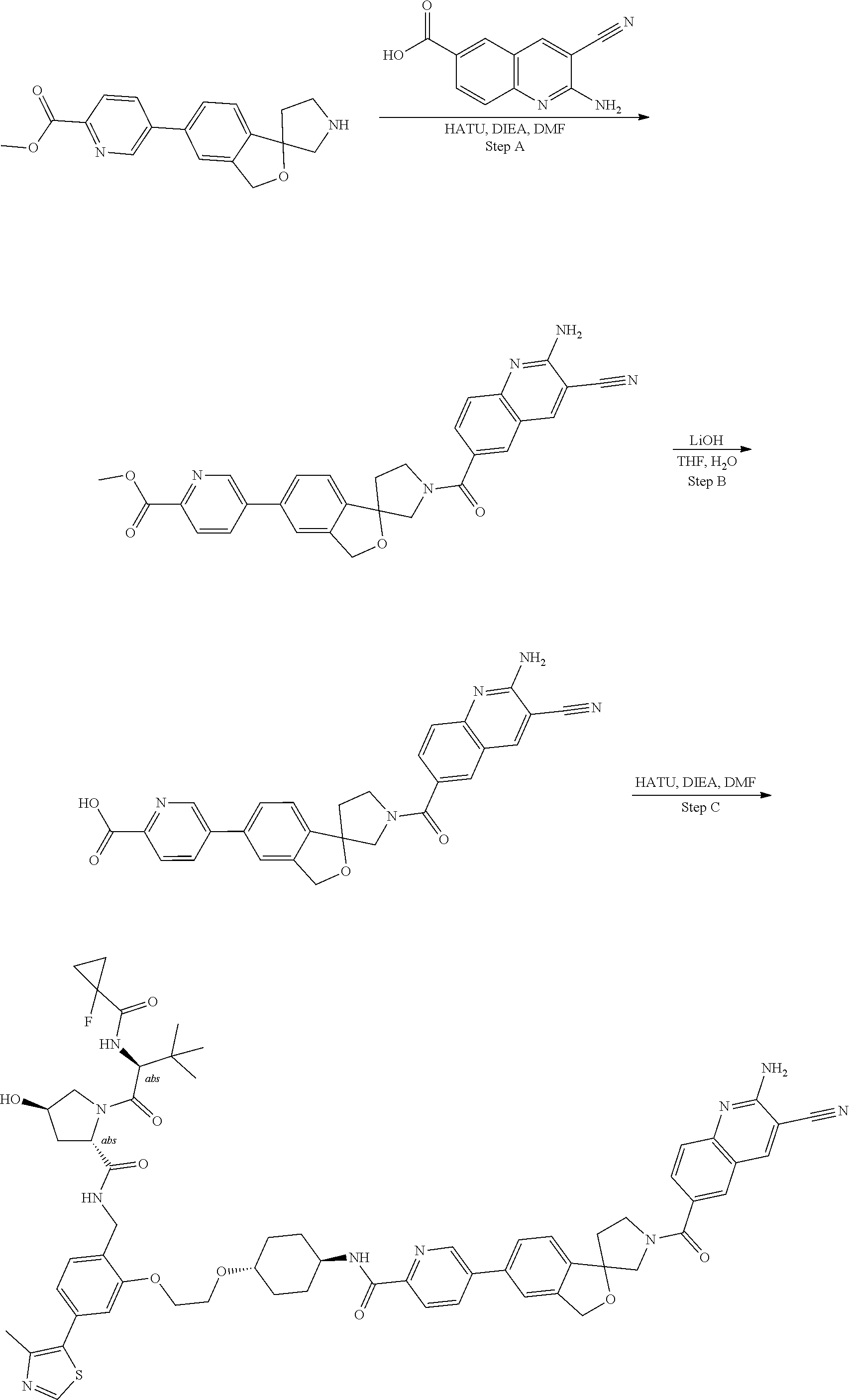 [SMILES: COC(=O)c1ccc(-c2ccc3c(c2)COC32CCNC2)cn1 ; N#Cc1cc2cc(C(=O)O)ccc2nc1N ; COC(=O)c1ccc(-c2ccc3c(c2)COC32CCN(C(=O)c3ccc4nc(N)c(C#N)cc4c3)C2)cn1 ; "LiOH" ; N#Cc1cc2cc(C(=O)N3CCC4(C3)OCc3cc(-c5ccc(C(=O)O)nc5)ccc34)ccc2nc1N ; Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)COC54CCN(C(=O)c5ccc6nc(N)c(C#N)cc6c5)C4)cn3)CC2)c1]

Example B44 was synthesized following the same procedures as described in Example B4 (1.0 mg, 4% yield over 3 steps) as a white solid. LC-MS (ESI): mass calcd, for C₆₂H₆₇FN₁₀O₉S, 1146.48/z found, 1147.6 [M+H]⁺. ¹H NMR (400 MHz, DMSO-d₆) δ 8.99 (s, 1H), 8.95-8.77 (m, 2H), 8.49 (s, 2H), 8.32-8.19 (m, 1H), 8.11-8.01 (m, 2H), 7.88 (s, 1H), 7.79-7.68 (m, 2H), 7.66-7.52 (m, 3H), 7.50-7.43 (m, 1H), 7.41 (d, J=7.5 Hz, 1H), 7.29 (d, J=8.0 Hz, 1H), 7.05 (s, 1H), 6.97 (d, J=7.6 Hz, 1H), 5.23-4.95 (m, 2H), 4.64-4.48 (m, 2H), 4.40-4.10 (m, 6H), 4.07-3.78 (m, 9H), 2.46 (s, 3H), 2.15-1.98 (m, 3H), 1.97-1.79 (m, 3H), 1.61-1.45 (m, 2H), 1.40-1.16 (m, 8H), 0.95 (s, 9H).

Example B45. 5-((R)-1-(2-amino-3-methoxyquinoline-6-carbonyl)-6′H-spiro[pyrrolidine-3,4′-thieno[2,3-c]furan]-2′-yl)-N-((1S,4R)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)picolinamide

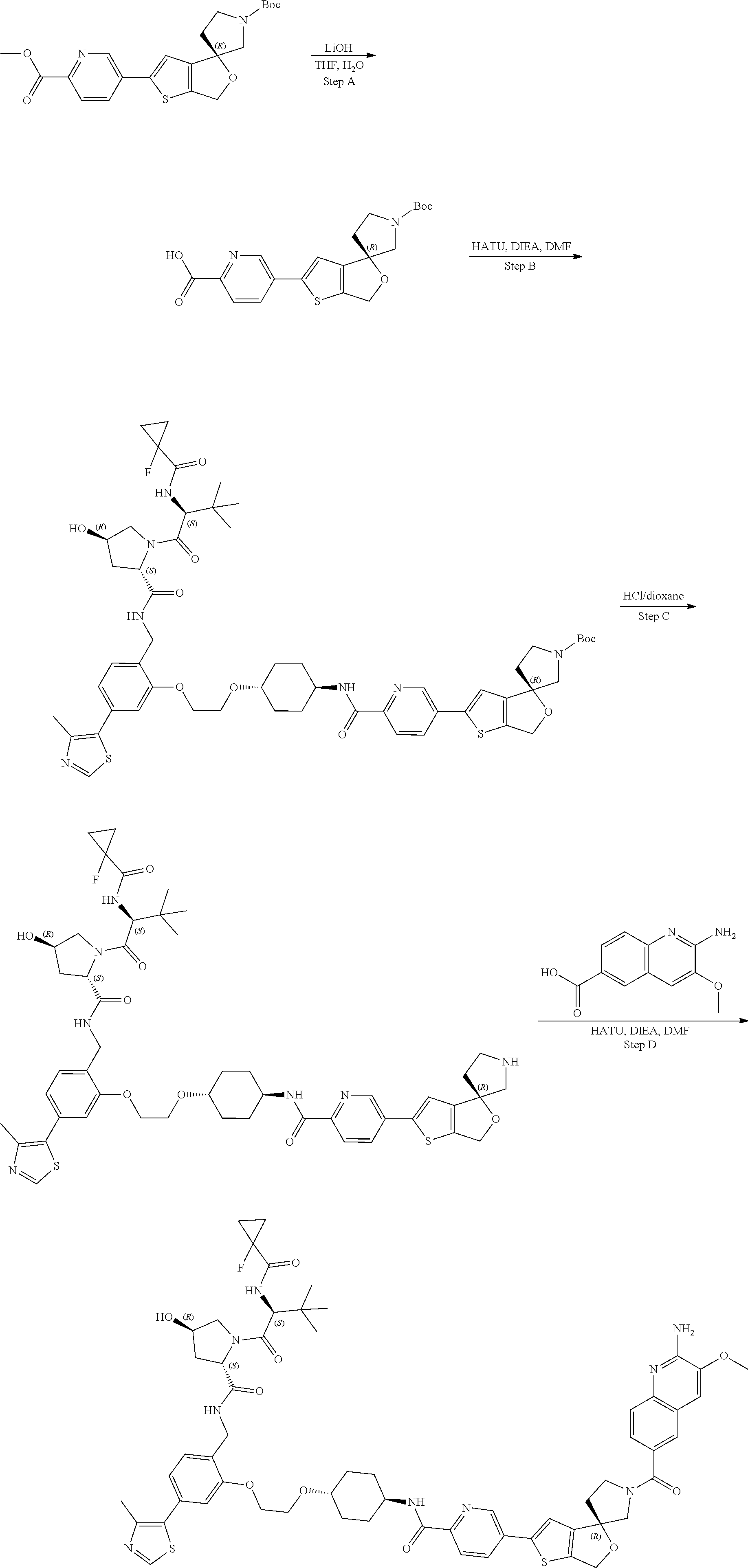

Example B45 was synthesized following the same procedures as described in Example B11 (13.0 mg, 5% yield over 4 steps) as a yellow solid. LC-MS (ESI): mass calcd. for C₆₀H₆₈FN₉O₁₀S₂, 1157.45; m/z found, 1159.0 [M+H]⁺. ¹HNMR (400 MHz, DMSO-d₆) δ 9.05-8.89 (m, 3H), 8.86 (d, J=13.9 Hz, 1H), 8.50 (t, J=5.9 Hz, 1H), 8.46-8.40 (m, 1H), 8.16-8.04 (m, 3H), 7.94-7.86 (m, 1H), 7.79 (t, J=9.5 Hz, 1H), 7.72 (d, J=7.5 Hz, 1H), 7.69-7.65 (m, 1H), 7.41 (d, J=7.8 Hz, 1H), 7.29 (dd, J=9.2, 2.6 Hz, 1H), 7.05 (s, 1H), 6.97 (d, J=7.7 Hz, 1H), 5.15-4.97 (m, 2H), 4.59 (d, J=9.4 Hz, 1H), 4.52 (t, J=8.1 Hz, 1H), 4.39-4.14 (m, 6H), 4.05-4.01 (m, 3H), 3.97 (d, J=11.0 Hz, 1H), 3.90-3.70 (m, 8H), 2.47 (s, 3H), 2.44-2.35 (m, 1H), 2.25-2.16 (m, 1H), 2.13-2.09 (m, 1H), 2.07-2.02 (m, 2H), 1.96-1.90 (m, 1H), 1.88-1.82 (m, 2H), 1.57-1.44 (m, 2H), 1.39-1.28 (m, 4H), 1.23-1.19 (m, 2H), 0.95 (s, 9H).

Example B46. 5-((R)-1-(2-amino-3-cyanoquinoline-6-carbonyl)-6′H-spiro[pyrrolidine-3,4′-thieno[2,3-c]furan]-2′-yl)-N-((1S,4R)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)picolinamide

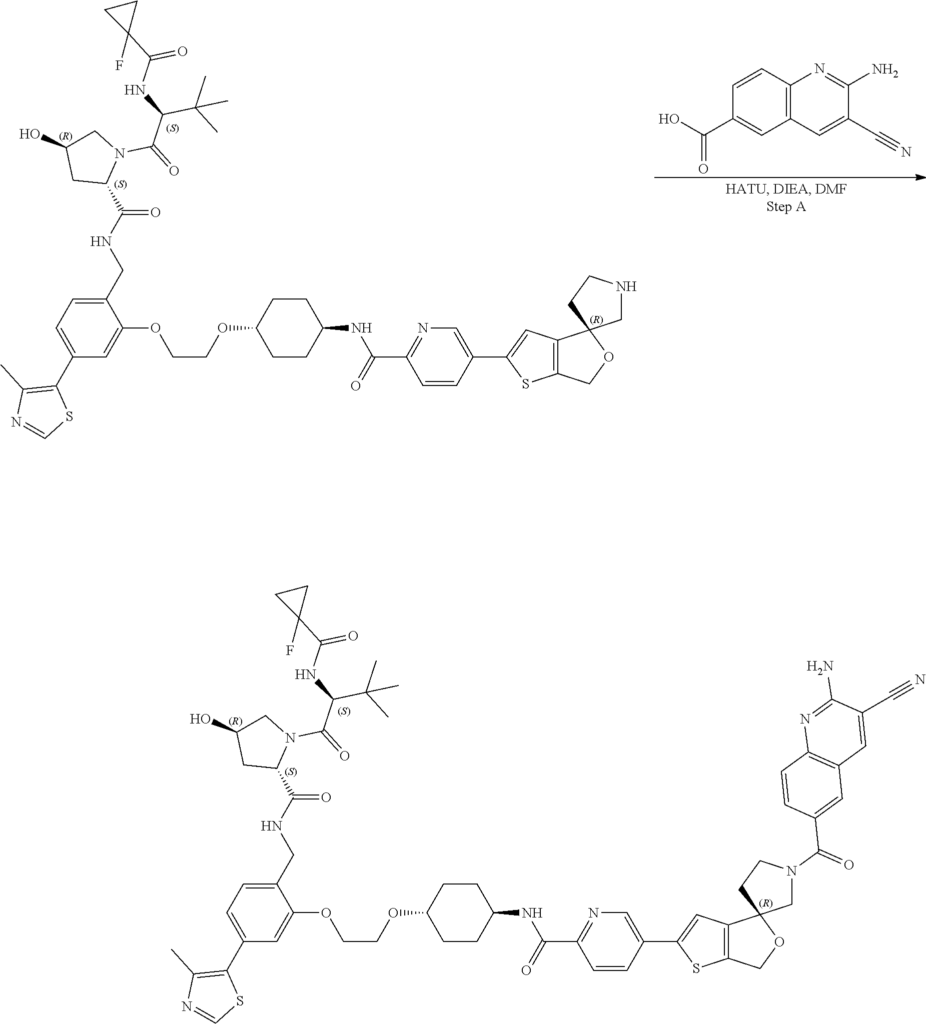

Example B46 was synthesized following the same procedures as described in Example B45 (8.0 mg, 15% yield) as a yellow solid. LC-MS (ESI): mass calcd, for C₆₀H₆₅FN₁₀O₉S₂, 1152.44; m/z found, 1153.1 [M+H]⁺. ¹HNMR (400 MHz, DMSO-d₆) δ 8.99 (s, 1H), 8.90-8.80 (m, 2H), 8.49 (t, J=5.9 Hz, 1H), 8.47-8.40 (m, 1H), 8.18-8.03 (m, 3H), 7.88 (t, J=9.8 Hz, 1H), 7.73-7.65 (m, 1H), 7.61-7.52 (m, 2H), 7.41 (d, J=7.8 Hz, 1H), 7.29 (dd, J=9.2, 2.3 Hz, 1H), 7.05 (s, 1H), 6.97 (d, J=8.0 Hz, 1H), 5.19-4.96 (m, 2H), 4.59 (d, J=9.3 Hz, 1H), 4.52 (t, J=8.2 Hz, 1H), 4.40-4.11 (m, 6H), 3.99 (d, J=10.9 Hz, 1H), 3.89-3.72 (m, 6H), 3.41-3.28 (m, 2H), 2.47 (s, 3H), 2.44-2.35 (m, 1H), 2.23-2.14 (m, 1H), 2.14-2.08 (m, 1H), 2.07-2.02 (m, 2H), 1.97-1.90 (m, 1H), 1.85 (d, J=10.2 Hz, 2H), 1.56-1.44 (m, 2H), 1.39-1.28 (m, 4H), 1.23-1.20 (m, 2H), 0.95 (s, 9H).

### II. Biological Assessment

The Binding Potency of Representative Compounds to PRMT5-MTA and SAM were Determined Using HTRF Assay Technology

PRMT5 binding potencies in the absence and presence of S-adenosyl-1-methionine (SAM) or methylthioadenosine (MTA) were measured in a displacement assay using a cyanine5 (CY5) labeled probe. Compound IC₅₀s were determined for Apo PRMT5, PRMT5/MTA and PRMT5/SAM. 7.5 nL of DMSO stock of compounds were dispensed into a 384-well microplate (Perkin Elmer 6008289) with 11-point 3-fold serial dilution starting at 2 μM. 10 nL CY5 labeled probe at final concentrations of 10 nM were dispensed by ECHO to measure IC₅₀s for Apo, PRMT5/MTA and PRMT5/SAM, respectively. To a solution of 0.125 nM of anti-His Tb cryptate and 0.25 nM PRMT5/MEP50, MTA or SAM were added to a final concentration of 50 AM and the mixtures were incubated at room temperature for 30 minutes. 2 μL of the resultant solutions were added to the compound plates sealed and centrifuged at 1500 G for 30 seconds. Final DMSO was 1%. The plates were incubated for 120 minutes at room temperature. The assay was run in 30 mM Bicine pH 8.0, 0.003% Tween-20, 1.5 mM DTT, 150 mM NaCl. The signal from the assay was examined on Envision (Perkin Elmer). The TR-FRET signal was calculated by determining the ratio of Emission 2 (665 nm) to Emission 1 (620 nm) following excitation at 337 nm for each data point. IC₅₀ values were then calculated using a non-linear fit variable slope model in Signals software (PerkinElmer) and Kis were calculated by using the equation IC₅₀=Kᵢ*{1+(probe/K_{d}, probe)} where K_{d} for the probe for apo, PRMT5/MTA and PRMT5/SAM were 1.3, 0.75, and 10 nM.

TABLE 2

| | MTA-PRMT5 | SAM-PRMT5 |
|---|---|---|
| Example No. | IC₅₀ (nM) | IC₅₀ (nM) |
| B1 | C | D |
| B2 | A | C |
| B3 | D | D |
| B4 | B | D |
| B5 | C | D |
| B6 | A | B |
| B7 | B | C |
| B8 | B | C |
| B9 | B | C |
| B10 | B | C |
| B11 | C | D |
| B12 | D | D |
| B13 | C | D |
| B14 | B | D |
| B15 | B | C |
| B16 | B | D |
| B17 | D | D |
| B18 | A | B |
| B19 | B | D |
| B20 | C | D |
| B21 | B | D |
| B22 | C | D |
| B23 | D | D |
| B24 | C | D |
| B25 | D | D |
| B26 | A | C |
| B27 | B | C |
| B28 | D | D |
| B30 | C | D |
| B31 | C | D |
| B33 | C | D |
| B34 | D | D |
| B35 | C | D |
| B36 | C | D |
| B37 | D | D |
| B38 | D | E |
| B39 | A | B |
| B40 | B | C |
| B41 | C | D |
| B42 | C | D |
| B44 | D | E |

Definitions:
IC₅₀: “A”: <1 nM;
“B”: 1-9.9 nM;
“C”: 10-99 nM;
“D”: 100-999 nM;
“E”: >1000 nM

PRMT5 Degradation Potency and Dₘₐₓ of Representative Compounds were Determined Using in HiBit Technology in HiBit MTAP KO and MTAP WT Cells.

HCT116 cells+/−MTAP (Creative Biogene (CSC-RT2701) were genetically modified via CRISPR/Cas9 to fuse HiBiT to the carboxy terminus of PRMT5. Cells were cultured in McCoy's 5A containing 10% FBS and 1% (Vol:Vol) penicillin-streptomycin. At the time of experiment, cells were seeded at a density of 1250/25 L/well in a 384-well plate (Corning; cat #3571) 24 hrs prior to compound treatment. Investigational compounds were prepared at 3 mM top concentration in 100% DMSO and an 11-point 3-fold serial dilution done in DMSO. 25 nL of serial diluted compounds were transferred to wells containing cell in 25 μL of media for a final top concentration of 3 μM. An equal volume of DMSO was added to control wells. Each treatment was performed in duplicate. Cells were incubated at 37° C.±5% CO₂ in a cell culture incubator for 18 hours, as indicated in Table 2. At the end of experiment, levels of PRMT5 were evaluated with Nano-Glo® HiBiT Lytic Detection Assay (Promega N3050). Assay reagent was prepared according to the manual provided with the Promega kit. 25 μL of this assay solution was added to each well. Luminescence from each well was measured with Envision plate reader (Perkin Elmer). Dose response curves were fit using Perkin Elmer Signals Lead Discovery software.

TABLE 3

| | MTAP KO | MTAP | MTAP WT | MTAP |
|---|---|---|---|---|
| | HiBit | KO HiBit | HiBit | WT HiBit |
| Example No. | DC₅₀ (nM) | Dₘₐₓ (%) | DC₅₀ (nM) | Dₘₐₓ (%) |
| B1 | D | D | | |
| B2 | D | D | | |
| B3 | C | B | C | B |
| B4 | C | C | | |
| B5 | D | C | D | B |
| B6 | C | B | C | B |
| B7 | C | B | C | C |
| B8 | C | B | C | C |
| B9 | C | C | D | D |
| B10 | B | B | C | C |
| B11 | B | B | C | D |
| B12 | D | D | | |
| B13 | B | B | C | D |
| B14 | B | A | C | B |
| B15 | B | A | B | B |
| B16 | B | B | C | D |
| B17 | D | D | | |
| B18 | C | C | D | D |
| B19 | B | A | B | A |
| B20 | B | A | C | B |
| B21 | B | A | B | A |
| B22 | C | B | D | D |
| B23 | C | B | C | D |
| B24 | B | A | C | C |
| B25 | B | B | C | C |
| B26 | C | B | C | D |
| B27 | C | B | C | D |
| B28 | C | B | D | D |
| B29 | C | D | D | D |
| B30 | B | A | B | B |
| B31 | C | A | C | B |
| B32 | D | D | D | D |
| B33 | C | C | C | D |
| B34 | C | C | D | D |
| B35 | C | B | D | D |
| B36 | B | B | B | D |
| B37 | B | A | B | B |
| B38 | E | E | | |
| B39 | B | A | B | A |
| B40 | B | A | A | A |
| B41 | C | A | C | B |
| B42 | C | D | D | D |
| B43 | B | B | C | B |
| B44 | E | E | E | C |
| B45 | C | D | | |
| B46 | D | C | | |

Definitions:
DC₅₀: “A”: <1 nM; “B”: 1-9.9 nM; “C”: 10-99 nM; “D”: 100-999 nM; “E”: >1000 nM D_{Max}: “A”: >80%; “B”: 60-79%; “C”: 40-59%; “D”: 20-39%; “E”: <20%.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound of Formula I 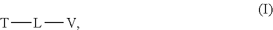
or a pharmaceutically acceptable salt, solvate, or stereoisomer thereof, wherein:
T is of Formula I-1 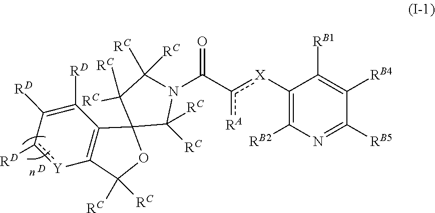
wherein:
each [symbol] is independently a single bond or a double bond, as valency permits;
X is N(R^{X1}), and R^{A} is oxo; or
X is C(R^{X2}), and R^{A} and R^{B2}, together with the intervening atoms to which they are attached, form C₆ aryl or 6-membered heteroaryl, wherein the C₆ aryl or 6-membered heteroaryl is optionally substituted with one or more halogen, —CN, —NO₂, —OH, or —NH₂;
R^{X1} is hydrogen or C₁₋₆ alkyl, wherein the C₁₋₆ alkyl is optionally substituted with one or more halogen, —CN, —NO₂, —OH, or —NH₂;
R^{X2} is hydrogen, halogen, —CN, —NO₂, —OH, —NH₂, or C₁₋₆ alkyl, wherein the C₁₋₆ alkyl is optionally substituted with one or more halogen, —CN, —NO₂, —OH, or —NH₂;
R^{B1}, R^{B2}, R^{B4}, and R^{B5} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, or —NH₂; or
R^{B1} and R^{B4}, together with the intervening atoms to which they are attached, form 3- to 8-membered heterocyclyl or 5- to 6-membered heteroaryl, wherein the 3- to 8-membered heterocyclyl or 5- to 6-membered heteroaryl is optionally substituted with one or more halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl;
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
n^{D} is 0 or 1;
Y is O, S, or C(R^{D}), as valency permits; and
each R^{D} is independently 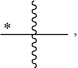
hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂, wherein * denotes attachment to L, and one R^{D} is 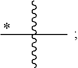
L is of Formula I-2 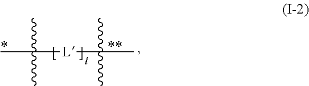
wherein:
* denotes attachment to T, and ** denotes attachment to V;
each L′ is independently —C(═O)—, —C(═O) N(R^{L′})—, —N(R^{L′})C(═O)—, —C(═O)O—, —OC(═O)—, —N(R^{L′})—, —O—, —O—(C₁₋₆ alkylene)-, —(C₁₋₆ alkylene)-O—, —S—, —S(═O)₂—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene, wherein the —O—(C₁₋₆ alkylene)-, —(C₁₋₆ alkylene)-O—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene is optionally substituted with one or more halogen, —CN, —NO₂, —OH, or —NH₂;
each occurrence of R^{L′} is independently hydrogen, C₁₋₆ alkyl, C₂₋₆ alkenyl, C₂₋₆ alkynyl, C₃₋₈ carbocyclyl, 3- to 8-membered heterocyclyl, —S(═O)₂R^{a}, —S(═O)₂OR^{a}, —S(═O)₂N(R^{a})₂, —C(═O)R^{a}, —C(═O)OR^{a}, or —C(═O)N(R^{a})₂, wherein the C₁₋₆ alkyl, C₂₋₆ alkenyl, C₂₋₆ alkynyl, C₃₋₈ carbocyclyl, or 3- to 8-membered heterocyclyl is optionally substituted with one or more halogen, —CN, —NO₂, —OH, or —NH₂; and
each R^{a} independently is hydrogen, C₁₋₆ alkyl, C₂₋₆ alkenyl, or C₂₋₆ alkynyl, wherein the C₁₋₆ alkyl, C₂₋₆ alkenyl, or C₂₋₆ alkynyl is optionally substituted with one or more halogen, —CN, —NO₂, —OH, or —NH₂;
l is an integer selected from 0 to 10;
V is of Formula I-3 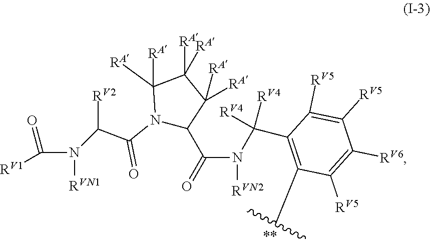
wherein:
** denotes attachment to L;
R^{V1} is hydrogen, C₁₋₆ alkyl, C₃₋₆ carbocyclyl, or 3- to 6-membered heterocyclyl, wherein the C₁₋₆ alkyl, C₃₋₆ carbocyclyl, or 3- to 6-membered heterocyclyl is optionally substituted with one or more halogen, —CN, —NO₂, —OH, or —NH₂;
R^{VN1} is hydrogen or C₁₋₆ alkyl, wherein the C₁₋₆ alkyl is optionally substituted with one or more halogen, —CN, —NO₂, —OH, or —NH₂;
R^{V2} is hydrogen or C₁₋₆ alkyl, wherein the C₁₋₆ alkyl is optionally substituted with one or more halogen, —CN, —NO₂, —OH, or —NH₂;
each R^{A′} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
R^{VN2} is hydrogen or C₁₋₆ alkyl, wherein the C₁₋₆ alkyl is optionally substituted with one or more halogen, —CN, —NO₂, —OH, or —NH₂;
each R^{V4} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
each R^{V5} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂; and
R^{V6} is C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, —NH₂, C₁₋₆ alkyl, C₂₋₆ alkenyl, or C₂₋₆ alkynyl.

2. The compound of claim 1, wherein X is N(R^{X1}), and R^{A} is oxo.

3. The compound of claim 1, wherein X is C(R^{X2}), and R^{A} and R^{B2}, together with the intervening atoms to which they are attached, form C₆ aryl or 6-membered heteroaryl, wherein the C₆ aryl or 6-membered heteroaryl is optionally substituted with one or more halogen, —CN, —NO₂, —OH, or —NH₂.

4. The compound of claim 3, wherein R^{A} and R^{B2}, together with the intervening atoms to which they are attached, form C₆ aryl or pyridinyl.

5. The compound of any one of claims 1-4, wherein R^{B1} and R^{B4}, together with the intervening atoms to which they are attached, form 3- to 8-membered heterocyclyl or 5- to 6-membered heteroaryl, wherein the 3- to 8-membered heterocyclyl or 5- to 6-membered heteroaryl is optionally substituted with one or more halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl.

6. The compound of claim 5, wherein R^{B1} and R^{B4}, together with the intervening atoms to which they are attached, form dihydrofuran or pyrazole optionally substituted with one or more C₁₋₆ alkyl.

7. The compound of any one of claims 1-4, wherein R^{B1} is hydrogen, and R^{B4} is C₁₋₆ alkyl or C₃₋₁₂ carbocyclyl.

8. The compound of any one of claims 1-7, wherein R^{B5} is —NH₂.

9. The compound of any one of claims 1-8, wherein each R^{C} is independently hydrogen.

10. The compound of any one of claims 1-9, wherein n^{D} is 0, and Y is S.

11. The compound of any one of claims 1-9, wherein n^{D} is 1, and Y is C(R^{D}).

12. The compound of any one of claims 1-11, wherein one R^{D} is 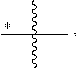
and each of the remaining R^{D} is independently hydrogen.

13. The compound of any one of claims 1-12, wherein T is of Formula I-1-a, I-1-b, I-1-c, or I-1-d 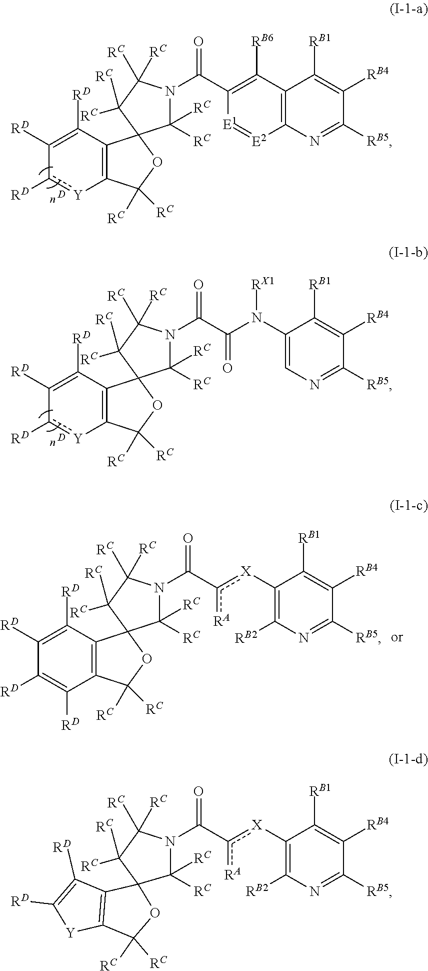
wherein:
E¹ is N or CR^{B7},
E² is N or CR^{B8};
R^{B6}, R^{B7}, and R^{B8} are independently hydrogen, halogen, —CN, —NO₂, —OH, or —NH₂; and
when T is of Formula I-1-d, Y is O or S.

14. The compound of any one of claims 1-13, wherein each L′ is independently selected from —C(═O)—, —C(═O)N(R^{L′})—, —N(R^{L′})C(═O)—, —N(R^{L′})—, —O—, —O—(C₁₋₆ alkylene)-, —(C₁₋₆ alkylene)-O—, C₁₋₆ alkylene, C₃₋₁₂ carbocyclylene, and 5- to 10-membered heteroarylene.

15. The compound of any one of claims 1-14, wherein L is **—[O—(C₁₋₆ alkylene)]₁₋₆—N(R^{L′})C(═O)—*, **—O—[(C₁₋₆ alkylene)]₁₋₃—N(R^{L′})C(═O)—*, *-(5- to 6-membered heteroarylene)-C(═O)NH—[(C₁₋₆ alkylene)-O]₁₋₆—**, *-(5- to 6-membered heteroarylene)-C(═O)N(R^{L′})—(C₃₋₁₂ carbocyclylene)-O—[(C₁₋₆ alkylene)-O]₁₋₃—**, or *-(5- to 6-membered heteroarylene)-C(═O)N(R^{L′})—[(C₁₋₆ alkylene)-O]₁₋₃—**.

16. The compound of claim 15, wherein L is **—[O—(C₁₋₆ alkylene)]₁₋₆-N(H)C(═O)—*, **—O—[(C₁₋₆ alkylene)]₁₋₃-N(H)C(═O)—*, *-(pyridinylene)-C(═O)NH—[(C₁₋₆ alkylene)-O]₁₋₆—**, *-(pyridinylene)-C(═O)NH—(C₃₋₁₂ carbocyclylene)-O—[(C₁₋₆ alkylene)-O]₁₋₃—**, or *-(pyridinylene)-C(═O)NH—[(C₁₋₆ alkylene)-O]₁₋₃—**.

17. The compound of any one of claims 1-16, wherein R^{V1} is C₃₋₆ carbocyclyl optionally substituted with one or more halogen.

18. The compound of any one of claims 1-17, wherein R^{VN1} and R^{VN2} are each independently hydrogen.

19. The compound of any one of claims 1-18, wherein R^{V2} is C₁₋₆ alkyl.

20. The compound of any one of claims 1-19, wherein one R^{A′} is —OH, and each of the remaining R^{A′} is independently hydrogen.

21. The compound of any one of claims 1-20, wherein each R^{V4} is independently hydrogen.

22. The compound of any one of claims 1-21, wherein each R^{V5} is independently hydrogen.

23. The compound of any one of claims 1-22, wherein R^{V6} is C₂₋₆ alkynyl or 5- to 10-membered heteroaryl optionally substituted with one or more C₁₋₆ alkyl.

24. The compound of any one of claims 1-23, wherein V is of Formula I-3-a

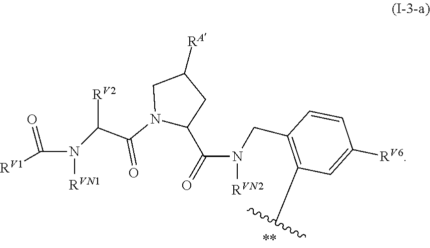

25. The compound of claim 1, wherein the compound is of Formula (I-a) 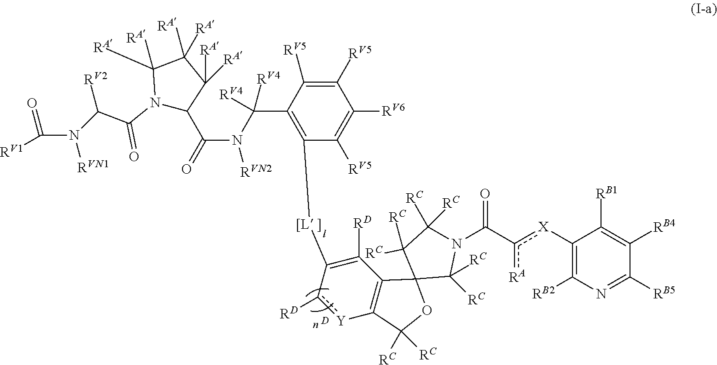
or a pharmaceutically acceptable salt, solvate, or stereoisomer thereof.

26. The compound of claim 1, wherein the compound is of Formula (I-b) 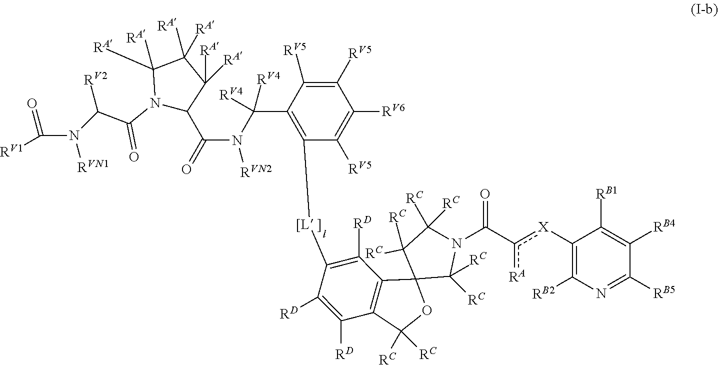
or a pharmaceutically acceptable salt, solvate, or stereoisomer thereof.

27. The compound of claim 1, wherein the compound is of Formula (I-b-i), (I-b-ii), or (I-b-iii) 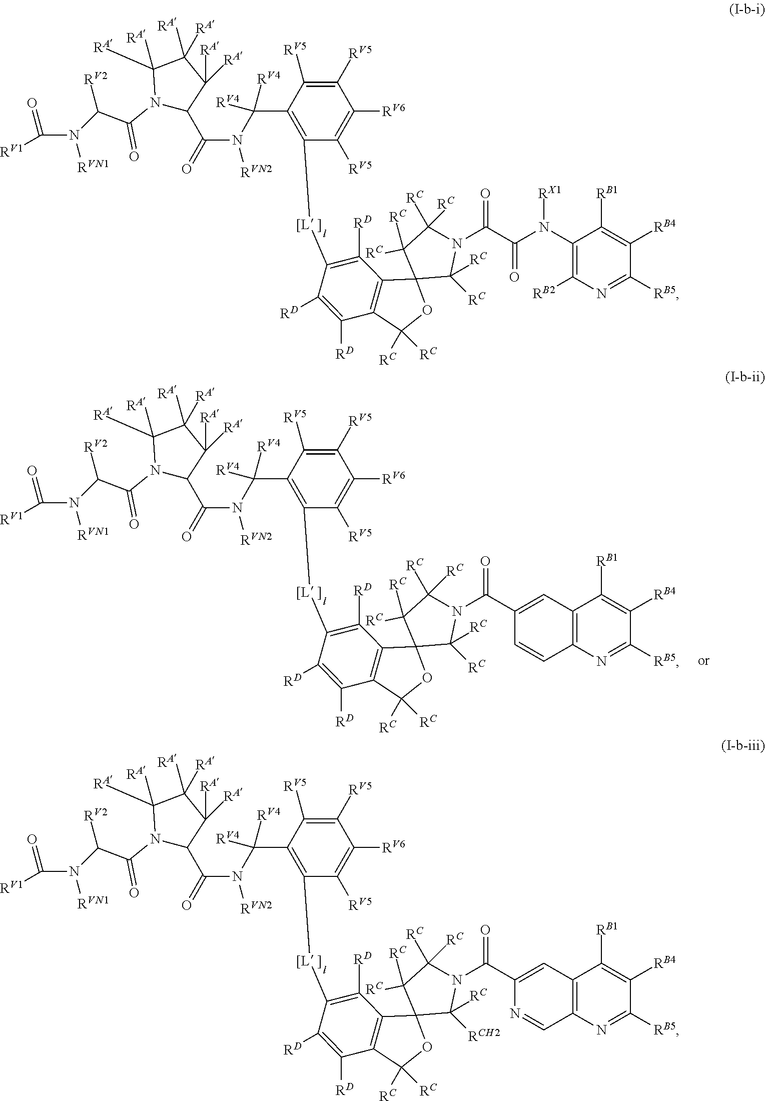
or a pharmaceutically acceptable salt, solvate, or stereoisomer thereof.

28. The compound of claim 1, wherein the compound is of Formula (I-c) 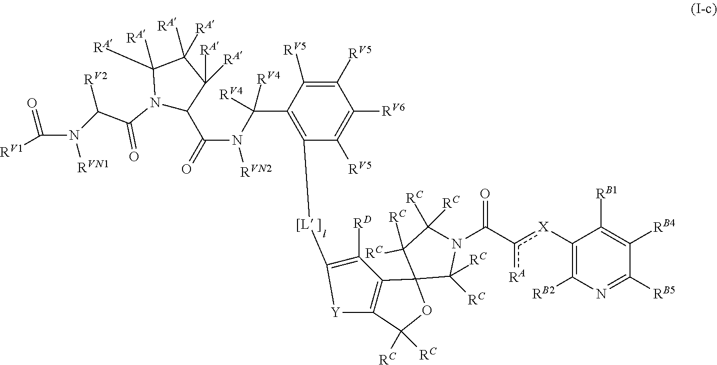
or a pharmaceutically acceptable salt, solvate, or stereoisomer thereof.

29. The compound of claim 1, wherein the compound is of Formula (I-c-i), (I-c-ii), or (I-c-iii) 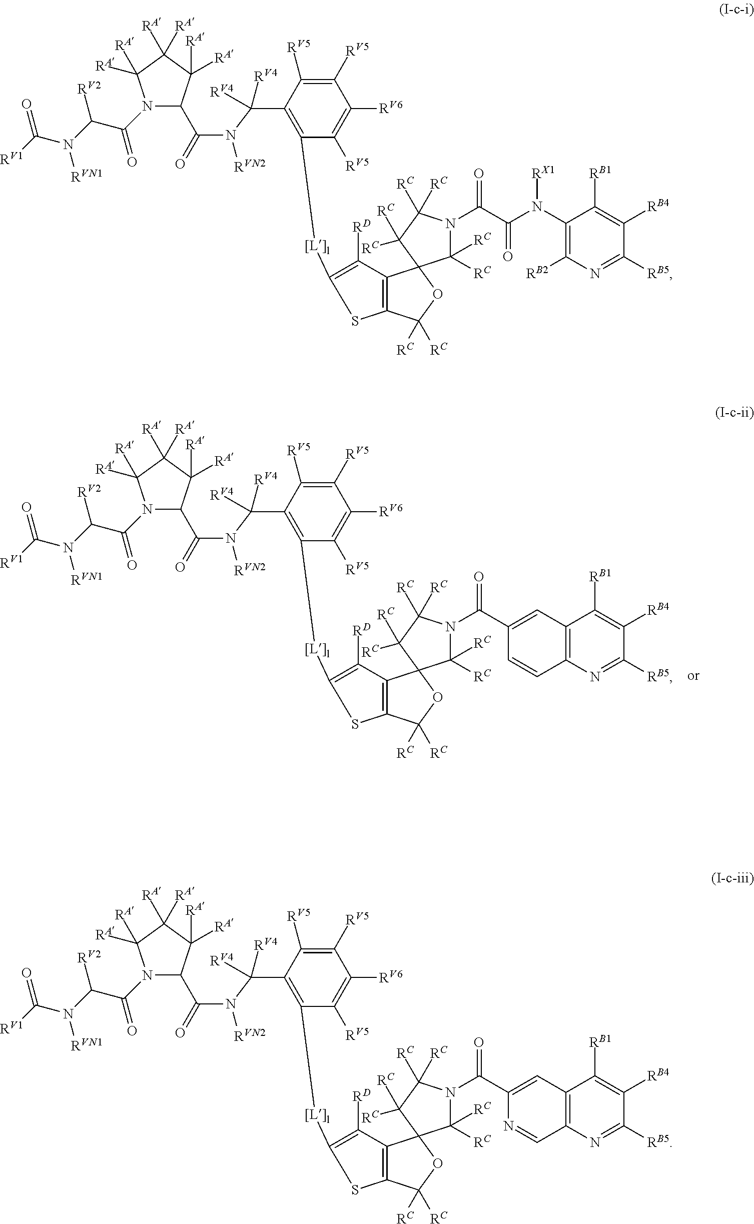
or a pharmaceutically acceptable salt, solvate, or stereoisomer thereof.

30. A compound selected from the compounds in Table 1, or a pharmaceutically acceptable salt thereof.

31. A pharmaceutical composition comprising the compound of any one of claims 1-30, and one or more pharmaceutically acceptable excipient.

32. A method of treating a disease or disorder comprising administering to a patient in need thereof a compound of any one of claims 1-30.

33. Use of a compound of any one of claims 1-30 in the manufacture of a medicament for treating a disease or disorder.

34. A compound of any one of claims 1-30 for use in treating a disease or disorder.

35. The method, use, or compound for use of any one of claims 32-34, wherein the disease or disorder is a PRMT5 protein-mediated disease or disorder.

36. The method, use, or compound for use of any one of claims 32-35, wherein the disease or disorder is cancer.

37. The compound of any one of claims 1-30, wherein the compound is conjugated to a conjugate partner.

38. A conjugate comprising the compound of any one of claims 1-30 and a conjugate partner.

39. The compound of claim 37 or the conjugate of claim 38, wherein the conjugate partner is an antibody.

40. The compound of claim 37 or the conjugate of claim 38, wherein the conjugate partner is a half-life extending moiety.

41. A method of preparing a compound according to any one of claims 1-30, or any intermediate in the Examples, according to a scheme of the present disclosure or a synthetic description in the Examples.

42. An intermediate described in one or more Examples.
